(19) United States
(12) Patent Application Publication
(10) Pub. No.: US 2025/0034528 A1
(43) Pub. Date: Jan. 30, 2025

(54) SYNCHRONIZED MESENCHYMAL STROMAL CELLS

(71) Applicant: Ossium Health, Inc., San Francisco, CA (US)

(72) Inventors: Erik J. Woods, Carmel, IN (US); Brian H. Johnstone, Fishers, IN (US); Dongsheng Gu, Indianapolis, IN (US); Jianguang Du, Carmel, IN (US)

(21) Appl. No.: 18/913,087

(22) Filed: Oct. 11, 2024

Related U.S. Application Data
(63) Continuation of application No. PCT/US2023/065824, filed on Apr. 14, 2023.
(60) Provisional application No. 63/331,632, filed on Apr. 15, 2022.

(51) Int. Cl. C12N 5/077

(52) U.S. Cl. CPC C12N 5/0669

(57) ABSTRACT

The present disclosure provides composition and methods preparing a population of isolated MSCs for pharmaceutical application, the method comprising: providing a population of isolated MSCs; and synchronizing the population of isolated MSCs, wherein no more than 25% of the population of isolated MSCs are in the S phase of the cell cycle.

### CROSS-REFERENCE TO RELATED APPLICATIONS

This application is a continuation application of PCT Application No. PCT/US2023/065824, filed Apr. 14, 2023, which claims priority to U.S. Provisional Patent Application Ser. No. 63/331,632, filed Apr. 15, 2022. The entire contents of each of the aforementioned patent applications is incorporated herein by reference.

### STATEMENT AS TO FEDERALLY SPONSORED RESEARCH

This invention was made with government support under AI 138334 awarded by the National Institutes of Health. The government has certain rights in the invention.

### BACKGROUND

Mesenchymal stromal cells (MSCs) are widely utilized in clinical trials based on proven in vitro and animal studies of their immunomodulatory and regenerative properties. However, while clinical trials have demonstrated clear safety of utilizing MSCs, the overwhelmingly positive results obtained through in vitro and preclinical studies have not been realized in clinical efficacy at scale. Clinical use of MSCs typically relies on effective storage (e.g., cryopreservation) of the intermediary and final products, which greatly facilitates distribution. However, cryopreservation has been attributed to impaired function of cryopreserved MSCs.

### SUMMARY

Clinical development of cellular therapies, including mesenchymal stem/stromal cell (MSC) treatments, has been hindered by ineffective cryopreservation methods that result in substantial loss of post-thaw cell viability and function. Proposed solutions to generate high potency MSC for clinical testing include priming cells with potent cytokines such as interferon gamma (IFNγ) prior to cryopreservation, which has been shown to enhance post-thaw function, or briefly culturing to allow recovery from cryopreservation injury prior to administering to patients. However, both solutions have disadvantages: cryorecovery increases the complexity of manufacturing and distribution logistics, while the pleiotropic effects of IFNγ may have uncharacterized and unintended consequences on MSC function. The present disclosure discloses specific cellular functions that impacted by cryoinjury, and specifically, in the cell cycle. Experimental evidence disclosed herein teaches that S phase MSC are exquisitely sensitive to cryoinjury, demonstrating heightened levels of delayed apoptosis post-thaw and reduced immunomodulatory function. Blocking cell cycle progression at G0/G1 by growth factor deprivation (commonly known as serum starvation) greatly reduces post-thaw dysfunction of MSC by preventing apoptosis induced by double-stranded breaks in labile replicating DNA that form during the cryopreservation and thawing processes. Viability, clonal growth and T cell suppression function are preserved at pre-cryopreservation levels and are no different than cells prior to freezing or frozen after priming with IFNγ. Thus, the present disclosure provides a robust and effective strategy to enhance post-thaw recovery of therapeutic MSC has benefits for other cellular therapies. Moreover, this process makes MSC therapies safer by preventing genetic instability in post-thaw cells; thereby, reducing the potential for administering cells harboring mutations.

An aspect of the present disclosure is a method for preparing a population of isolated MSCs for pharmaceutical application, the method comprising: providing a population of isolated MSCs; and synchronizing the population of isolated MSCs, wherein no more than 25% of the population of isolated MSCs are in the S phase of the cell cycle. Another aspect of the present disclosure is a method for storing a population of isolated MSCs, the method comprising: providing a population of isolated MSCs; and synchronizing the population of isolated MSCs, wherein no more than 25% of the population of isolated MSCs are in the S phase of the cell cycle. In some embodiments, no more than 5% of the population of isolated MSCs are in the S phase of the cell cycle. In some embodiments, no more than 2% of the population of isolated MSCs are in the S phase of the cell cycle. In some embodiments, at least 80% of the population of isolated MSCs express CD73, CD90, CD105, or any combination thereof. The method of any one of the embodiments disclosed herein, wherein at least 90% of the population of isolated MSCs express CD73, CD90, CD105, or any combination thereof. In some embodiments, at least 80% of the population of isolated MSCs maintain responsiveness to interferon gamma (IFNγ) stimulation. In some embodiments, at least 90% of the population of isolated MSCs maintain responsiveness to interferon gamma (IFNγ) stimulation. In some embodiments, at least 80% of the population of isolated MSCs express PD-L1, HLA-DR, or both, when stimulated with IFNγ. In some embodiments, at least 90% of the population of isolated MSCs express PD-L1, HLA-DR, or both, when stimulated with IFNγ. In some embodiments, at least 80% of the population of isolated MSCs overexpress PD-L1, HLA-DR, or both, when stimulated with IFNγ, relative to an unstimulated MSC. In some embodiments, at least 90% of the population of isolated MSCs overexpress PD-L1, HLA-DR, or both, when stimulated with IFNγ, relative to an unstimulated MSC. In some embodiments, at least 80% of the population of isolated MSCs overexpress Indoleamine 2,3-dioxygenase (IDO), when stimulated with IFNγ, relative to an unstimulated MSC. In some embodiments, at least 80% of the population of isolated MSCs overexpress Indoleamine 2,3-dioxygenase (IDO), when stimulated with IFNγ, relative to an unstimulated MSC. In some embodiments, synchronizing comprises serum starving the population of isolated MSCs for a period of time prior to exposing the population of isolated MSCs to a freezing temperature. In some embodiments, serum starving comprises culturing the population of stromal cells in media comprising less than 1% serum. In some embodiments, serum starving comprises culturing the population of stromal cells in media comprising less than 0.5% serum. In some embodiments, the serum is human platelet lysate (hPL). In some embodiments, the serum is human serum albumin (HSA). In some embodiments, serum starving comprises culturing the population of stromal cells in serum free media or in a medium having reduced amounts of serum. In some embodiments, the period of time prior to exposing the population of isolated MSCs to a freezing temperature is less than 72 hours. In some embodiments, the period of time prior to exposing the population of isolated MSCs to a freezing temperature is less than 48 hours. In some embodiments, the period of time prior to exposing the population of isolated MSCs to a freezing temperature is 24 hours. In some embodiments, the period of time prior to exposing the population of isolated MSCs to a freezing temperature is less than 24 hours. In various embodiments, the period of time is sufficient to synchronized at least 75% of the MSCs in the population into S phase of the cell cycle. In some embodiments, no more than 6% of the population of isolated MSCs undergo early apoptosis during the serum starvation. In some embodiments, no more than 2% of the population of isolated MSCs undergo early apoptosis during the serum starvation. In some embodiments, the method further comprising stimulating the population of isolated MSCs with IFNγ. In some embodiments, the method further comprising stimulating the population of isolated MSCs with IFNγ contemporaneously with the serum starving. In some embodiments, the population of isolated MSCs are vertebral bone adherent MSCs (vBA-MSCs). In some embodiments, the population of isolated MSCs are vertebral bone marrow derived MSCs (vBM-MSCs). In some embodiments, the population of isolated MSCs are cadaveric MSCs.

Another aspect of the present disclosure is a composition comprising a population of isolated mesenchymal stromal cells (MSCs), wherein no more than 25% of the population of isolated MSCs are in the S phase of the cell cycle. In some embodiments, no more than 5% of the population of isolated MSCs are in the S phase of the cell cycle. In some embodiments, no more than 2% of the population of isolated MSCs are in the S phase of the cell cycle. In some embodiments, at least 80% of the population of isolated MSCs express CD73, CD90, CD105, or any combination thereof. The composition of any one of the embodiments disclosed herein, wherein at least 90% of the population of isolated MSCs express CD73, CD90, CD105, or any combination thereof. In some embodiments, at least 80% of the population of isolated MSCs maintain responsiveness to interferon gamma (IFNγ) stimulation. In some embodiments, at least 90% of the population of isolated MSCs maintain responsiveness to interferon gamma (IFNγ) stimulation. In some embodiments, at least 80% of the population of isolated MSCs express PD-L1, HLA-DR, or both, when stimulated with IFNγ. In some embodiments, at least 90% of the population of isolated MSCs express PD-L1, HLA-DR, or both, when stimulated with IFNγ. In some embodiments, at least 80% of the population of isolated MSCs overexpress PD-L1, HLA-DR, or both, when stimulated with IFNγ, relative to an unstimulated MSC. In some embodiments, at least 90% of the population of isolated MSCs overexpress PD-L1, HLA-DR, or both, when stimulated with IFNγ, relative to an unstimulated MSC. In some embodiments, at least 80% of the population of isolated MSCs overexpress Indoleamine 2,3-dioxygenase (IDO), when stimulated with IFNγ, relative to an unstimulated MSC. In some embodiments, at least 80% of the population of isolated MSCs overexpress Indoleamine 2,3-dioxygenase (IDO), when stimulated with IFNγ, relative to an unstimulated MSC. In some embodiments, the population of isolated MSCs are vertebral bone adherent MSCs (vBA-MSCs). In some embodiments, the population of isolated MSCs are vertebral bone marrow derived MSCs (vBM-MSCs). In some embodiments, the population of isolated MSCs are cadaveric MSCs. A pharmaceutical composition comprising: the population of isolated MSCs from any one of the embodiments disclosed herein; and a pharmaceutically acceptable excipient.

In various embodiments, the MSCs are obtained from one or more vertebral bodies and the MSCs comprise vertebral bone marrow MSCs (vBM-MSCs), vertebral bone-adherent MSCs (vBA-MSCs), or both. In embodiments, the MSCs comprise less than about 5% CD45+ cells (e.g., less than about 5% CD45+ cells, less than about 4% CD45+ cells, less than about 3% CD45+ cells, less than about 2% CD45+ cells, less than about 1% CD45+ cells, or about 0% CD45+ cells), comprises at least about 90% CD105+ cells (e.g., at least about 90% CD105+ cells, at least about 91% CD105+ cells, at least about 92% CD105+ cells, at least about 93% CD105+ cells, at least about 94% CD105+ cells, at least about 95% CD105+ cells, at least about 96% CD105+ cells, at least about 97% CD105+ cells, at least about 98% CD105+ cells, or at least about 99% CD105+ cells), and/or comprises at least about 90% CD166+ cells. In some embodiments, the γMSCs were cultured under a serum starved condition and/or were exposed to a temperature shock during culturing. In various embodiments, the MSCs comprise less than about 10% CD45+ cells, at least about 90% CD105+ cells, and/or at least about 90% CD166+ cells.

In embodiments, the MSCs are obtained from one deceased human. In some embodiments, the MSCs are obtained from one or more vertebral bodies. In various embodiments, the MSCs comprise vertebral bone marrow MSCs (vBM-MSCs), vertebral bone-adherent MSCs (vBA-MSCs), or both.

In embodiments, the MSCs were expanded in culture prior to being interferon γ-primed. In some embodiments, the MSCs have undergone a primary expansion followed by cryopreservation. In various embodiments, the cryopreserved MSCs were thawed and then underwent a second expansion, e.g., the second expansion occurred for about seven days. In embodiments, the MSCs were primed with interferon gamma (IFNγ) during the second expansion, e.g., during the final day, the final two days, or the final three days of the second expansion, IFNγ was added to the second expansion media. In some embodiments, the IFNγ is present in the second expansion media at a concentration of from about 100 U/ml to about 1000 U/ml and/or from about 1 ng/mL to about 30 ng/ml.

In any of the herein disclosed aspects or embodiments, rather than cryopreserving MSCs or γMSCs and thawing the MSCs (which are subsequently primed) or thawing the γMSCs prior to use (either immediately or after one or more culturing steps), fresh MSCs or fresh γMSCs may be used.

In various embodiments, the composition comprises less than about 5% CD45+ cells, less than about 4% CD45+ cells, less than about 3% CD45+ cells, less than about 2% CD45+ cells, less than about 1% CD45+ cells, or about 0% CD45+ cells. In embodiments, the composition comprises at least about 90% CD105+ cells, at least about 91% CD105+ cells, at least about 92% CD105+ cells, at least about 93% CD105+ cells, at least about 94% CD105+ cells, at least about 95% CD105+ cells, at least about 96% CD105+ cells, at least about 97% CD105+ cells, at least about 98% CD105+ cells, or at least about 99% CD105+ cells. In some embodiments, the composition comprises at least about 90% CD166+ cells.

In various embodiments, the γMSCs are obtained from a matched unrelated donor to a subject for transplant. In embodiments, the γMSCs are obtained from an unmatched donor to the subject for transplant.

In various embodiments, the MSCs are expanded in culture prior to being interferon γ-primed. In embodiments, the MSCs undergo a primary expansion followed by cryopreservation. In some embodiments, the cryopreserved MSCs are thawed and then undergo a second expansion, e.g., the second expansion occurs for about seven days. In various embodiments, the MSCs are primed with interferon gamma (IFNγ) during the second expansion, e.g., during the final day, the final two days, or the final three days of the second expansion, IFNγ is added to the second expansion media. In embodiments, the IFNγ is present in the second expansion media at a concentration of from about 100 U/ml to about 1000 U/ml or from about 1 ng/mL to about 30 ng/ml. In some embodiments, the IFNγ is present in the second expansion media at a concentration of about 500 U/ml.

In various embodiments, the MSCs are obtained from one deceased human. In embodiments, the MSCs are obtained from one or more vertebral bodies and the MSCs comprise vertebral bone marrow MSCs (vBM-MSCs), vertebral bone-adherent MSCs (vBA-MSCs), or both. In some embodiments, when a hypoxic condition is due to the presence of decreased levels of O₂, increased levels of CO₂, and/or a hypoxia mimetic. In various embodiments, the γMSCs were cultured under a serum starved condition and/or were exposed to a temperature shock during culturing.

In any of the herein disclosed aspects or embodiments, rather than cryopreserving MSCs or γMSCs and thawing the MSCs (which are subsequently primed) or thawing the γMSCs prior to use (either immediately or after one or more culturing steps), fresh MSCs or fresh γMSCs may be used.

Another aspect of the present disclosure is a method for treating a disease or disorder in a subject in need thereof. The method comprising administering to the subject a therapeutically-effective amount of any herein-disclosed pharmaceutical composition. The pharmaceutical composition comprises any herein disclosed population of isolated MSCs and a pharmaceutically acceptable excipient.

Any herein disclosed aspect or embodiment can be combined with any other aspect or embodiment.

### BRIEF DESCRIPTION OF THE DRAWINGS

FIG. 1A to FIG. 1E show that cryopreservation of MSCs induces cell death during post-thaw recovery in culture. FIG. 1A includes illustrative flow cytometry plots showing early apoptosis (lower right quadrant and death (upper quadrants) of post-thaw MSCs compared to cells prior to cryopreservation (“cultured”). Quantitation of post-thaw (FIG. 1B) apoptotic, (FIG. 1C) dead and (FIG. 1D) live cells at the times indicated after thawing and culturing compared to freshly cultured cells. (FIG. 1E) Colony forming unit-fibroblast (CFU-F) counts after 14 days of culturing MSC prior to (fresh) and after (post-thaw) cryopreservation. Data are displayed as mean±SD from 3 independent experiments. Asterisks indicate statistical significance of the indicated comparisons: *, P<0.05; **, p<0.01; *** P<0.001, ****, P<0.0001. Non-significance (ns) is only indicated for comparison of fresh versus immediately post-thaw cells but is implied if comparisons did not achieve significance (p<0.05). Fresh and 0 hours post-thaw data as well as CFU data compared by unpaired t test. Post-thaw recovery timepoints (FIG. 1B to FIG. 1D) compared by one-way ANOVA with Sidak's multiple comparisons test.

FIG. 2A and FIG. 2B includes illustrative flow cytometry plots showing that cryo-injury is dependent on freezing and is independent of cryoprotective agents tested.

FIG. 3A to FIG. 3C are graphs showing that replicating S phase MSCs demonstrated reduced DNA synthesis and higher levels of apoptosis after cryopreservation and subsequent thawing. FIG. 3A includes illustrative flow plots showing 5-Ethynyl-2′-deoxyuridine (EdU) incorporation efficiency in S-phase MSCs during post-thaw recovery. FIG. 3B includes illustrative gating strategy for enumerating S phase cells in Annexin V−(AV−) and Annexin V+ (AV⁺) populations at 4 hr post-thaw. Gates and percentages shown on contour plots represent cells in S phase. FIG. 3C shows quantitation of the percentage of S phase cells in the total AV− and AV⁺ populations obtained from four independent experiments. All data are presented as means±SD. **, P<0.01; paired, two-tailed t test.

FIG. 4A to FIG. 4D shows that freezing and thawing induces DNA double-stranded breaks in replicating MSCs. FIG. 4A includes flow cytometry scatter plots of gH2AX positive MSCs at baseline (control) and following etoposide (100 mM) treatment. FIG. 4B includes immunofluorescent images of fresh and post-thaw MSC stained with EdU (green), gH2AX (red) and Hoechst 33342 nuclear stain (blue), shown individually and merged as indicated. The scale bar is 20 mm. FIG. 4C includes illustrative scatter plots of gH2AX+ cells at the indicated timepoints after thawing cryopreserved MSCs. FIG. 4D includes a quantitation of gH2AX+ cells in thawed MSCs isolated from 3 independent deceased organ donors (mean±SD).

FIG. 5A to FIG. 5F show hPL experiments which characterize the growth factor deprivation effects on MSC. MSC growing in rich medium were washed and cultured in minimal medium (a MEM) containing either 0.1% or no hPL for 24 or 48 hr. FIG. 5A include illustrative photomicrographs (20× magnification) of cells prior to (non-starved) or following starvation (starved). FIG. 5B is a graph showing average cell volumes of MSCs prior to starvation or after starvation in 0% of 0.1% hPL for 24 or 48 hr. FIG. 5C includes illustrative flow cytometry scatter plots showing a determination of apoptosis by Annexin V staining in combination with DAPI. FIG. 5D is a graph showing quantitation (mean±SD) of early apoptotic cells (Annexin v⁺/DAPI⁻) cultured under the indicated conditions. FIG. 5E is a graph showing cell numbers recovered from each well of a 6 well culture plate. FIG. 5F includes illustrative flow cytometry scatter plots showing DNA synthesis activity (EdU⁺) and DNA content (DAPI) of cells cultured under the various conditions. The boxed region represents S phase cells.

FIG. 6A to FIG. 6D show that classical MSC cell surface markers and IDO1 expression in normally cultured and starved cells. FIG. 6A are plots showing MSC which were grown in complete medium and starved for 24 and 48 hr were labeled with fluorescently conjugated antibodies to CD73, CD90 or CD105 surface epitopes and analyzed by flow cytometry. Non-stained (NS) controls were used to set the detection gates. The histograms for cells grown in complete medium are NS controls (top line) and antibody stained. The order (top to bottom) for both 24 and 48 hr starved MSC is NS controls, 0.1% hPL and 0% hPL). FIG. 6B are plots showing MSC which were cultured in complete medium with 10% hPL, 0.1% hPL and 0% hPL in the absence (plots 1, 3 and 5) or 25 ng/ml IFNγ (plots 2, 4 and 6) and stained for PD-L1 and HLA-DR surface expression. FIG. 6C are graphs showing RT-PCR analysis of MSC which were cultured for 24 hr under the indicated conditions. FIG. 6D are flow plots from three representative cultures of MSC grown in complete medium (green) and starvation medium (blue) and intracellularly stained with antibodies to IDO protein. Mean fluorescent intensities IDO+ populations in the independent cultures are shown in FIG. 6E. * P<0.05; ***, P<0.001; One-way ANOVA.

FIG. 7A to FIG. 7F show that growth factor withdrawal induces G0/G1 arrest prior to cryopreservation prevents post-thaw delayed apoptosis and death due to DNA double stranded breaks (DSB). FIG. 7A shows cell cycle arrest prior to S phase was confirmed staining for EdU incorporation, which demonstrated that DNA synthesis was absent. FIG. 7B includes illustrative flow cytometry scatter plots comparing delayed apoptosis and death in thawed MSCs that were either cultured in complete medium (non-starved) or minimal medium without hPL (starved) prior to cryopreservation. FIG. 7C is a graph quantitating apoptosis at the indicated timepoints post-thaw (mean±sd). FIG. 7D is a graph quantitating cell death at the indicated timepoints post-thaw (mean±sd). FIG. 7E shows quantitation of DSB by yH2AX staining of starved MSCs following cryopreservation (compare to FIG. 4C). FIG. 7F is a graph showing CFU-F analysis of starved and non-starved MSC prior to or following cryopreservation. *, p<0.05; **, p<0.01, ***, p<0.001; ****, p<0.0001. Two-way ANOVA with Sidak's multiple comparisons test (FIG. 7C and FIG. 7D) and one-way ANOVA with Tukey's multiple comparisons test.

FIG. 8 includes plots showing reduced post-thaw apoptosis is also observed in MSC from sources other than deceased organ donor vertebral body bone marrow. MSC from single adipose tissues and cord blood donors and three living donor aspirated bone marrows (all at passage 4) were cultured in complete medium or 0% hPL for 24 hr, cryopreserved and evaluated for apoptosis by flow cytometry at 4 hr after thawing.

FIG. 9A to FIG. 9D show the T cell suppression function of MSCs is greatly diminished by cryopreservation but can be rescued by growth factor starving the cells prior to freezing. After thawing, 2×10⁴ cells were added to wells of 96 well plates and incubated for 24 hr. The medium was removed and 4×10⁵ CSFE-labeled PBMCs were added to each well. PBMCs were stimulated by adding CD3e antibody and the plate was incubated for 4 days before analyzing T cell proliferation by flow cytometry. Proliferation of CD4⁺ (FIG. 9A) and CD8⁺ T cells (FIG. 9B) with different ratios of MSC to T cells which comprised 50% of the PBMCs. The ratio of MSCs to T cells was as follows: 0.5×10⁴, 1:80; 1×10⁴, 1:40; 2×10⁴, 1:20; 4×10⁴, 1:10. Representative flow cytometry plots are shown below the bar graphs and in FIG. 9C; in FIG. 9C, shown is the gating strategy for defining lymphocytes based on FSC and SSC (first column), viability by FVD780 dye (second column), and CD4⁺ T cells (third column). The last column represents generations of T cell clones identified by CFSE intensity, with the parental (unstimulated) population shaded blue, first generation yellow, third generation green, etc. FIG. 9D shows the mean values for each condition tested were normalized to the maximum proliferation value obtained from the stimulated PBMC control. IC₅₀ is the number of MSC that suppress 50% of activated T cell proliferation. *, p<0.05; **, p<0.01; ***, p<0.001; ****, p<0.0001. Two-way ANOVA with Sidak's multiple comparisons test.

FIG. 10 shows priming with IFNγ prior to cryopreservation further enhances post-thaw in vitro T cell suppression activity of growth factor starved MSC. Both starved and non-starved MSC were cryopreserved with and without additionally treating with IFNγ (25 ng/ml) for 24 hr. Cocultures of MSC (2×I 04/well) and PBMC (4×I 05/well) were as described in the Materials & methods and as described in FIG. 9A to FIG. 9D. *, p<0.05; **, p<0.01; ***, p<0.001; ****, p<0001. One-way ANOVA with Tukey's multiple comparisons test

FIG. 11A shows a schematic for the experimental groups used in subsequent studies to optimize conditions for starvation and priming utilized in the Examples described herein. FIG. 11B to FIG. 11E are schematics showing illustrative manufacturing processes for interferon γ-primed mesenchymal stromal cells (γMSCs).

FIG. 12 is a flow diagram of product process and controls.

FIG. 13 is a graph demonstrating differential gene expression of MSCs (open shapes) and γ-MSC (filled shapes) from each donor (same color and at similar verticality).

FIG. 14 is a graph showing Euclidean distance calculated to assess the overall similarity between samples.

FIG. 15A and FIG. 15B show representative karyotypes of MSCs (FIG. 5A) and γ-IFN-primed MSCs (FIG. 5B) from cells that have undergone primary expansion, cryopreservation, and secondary expansion, and are appropriate to infuse into a subject.

FIG. 16A to FIG. 16E show phenotypic and functional characterization of vBA-MSC. FIG. 16A shows expression of MHC and other surface markers with or without IFNγ for 2 days. FIG. 16B shows a chemotaxis assay showing increased number of MSCs in lower chamber in response to increasing amounts of CCLI 9, one of the CCR7 ligands. FIG. 16C are histograms showing flow cytometric analyses of interferon γ primed BM-MSCs for the indicated antigens (blue solid line) or isotype control (red dashed line). FIG. 16D are scatter plots showing three γ primed BM-MSCs expansions showing percentages of CD45 and CD105 cells. FIG. 16E are flow cytometric histograms showing the dilution of cell trace far red membrane dye (to the left) with expansion compared with a uniform bright signal (to the right) of nondividing cells.

FIG. 17 is a flowchart illustrating a study schema of the adult cohort used for determining an effective dosage of γMSCs for human administration.

FIG. 18 is a flowchart illustrating the overview of study design of the adolescent cohort if dose-limiting toxicity is observed in the adult cohort.

FIG. 19A is a graph showing live, apoptotic, and dead cell populations (%) averaged across the five (5) post-thaw time points (0, 2, 4, 6, and 24 hours) for the P4 MSC samples of Example 10: Intermediate Control (Freeze Medium), Intermediate (MSC Medium), and Final Product (Intermediate refrozen in Freeze Medium). Error bars are ±one (1) standard deviation. FIG. 19B and FIG. 19C are graphs showing total cells, live cells, and percent cell viability of the pre-freeze and (post-thaw) post-wash P4 vBM-MSC samples: Intermediate (MSC Medium) and Final Product (Intermediate refrozen in Freeze Medium). FIG. 19D are illustrative flow plots showing Apoptosis among cell groups characterized in Example 10.

### DETAILED DESCRIPTION

### Introduction

Mesenchymal stromal cells (MSCs) are cells of non-hematopoietic stromal origin that reside in bone marrow as well as in a variety of tissues including adipose and placenta. MSCs have potent immunosuppressive activity mediated via a variety of cell-to-cell contacts and soluble factors such as Indoleamine 2,3-dioxygenase (IDO), PGE2, TSG-6, CCL-2 and PD-L1, and TGF. These mediators inhibit T and B lymphocyte, NK cell and dendritic cell activation and function. MSCs also facilitate endogenous tissue repair and regeneration through secretion of cytokines such as VEGF, IL-6, IL-11, GM-CSF and SCF. As MSCs seem to be hypoimmunogenic and do not express co-stimulatory molecules, they can be infused into major histocompatibility complex disparate recipients.

MSCs have been widely exploited for their potent immunosuppressive activity in a variety of therapeutic indications, including inflammatory conditions, including immune-modulating conditions like Graft-versus-Host-Disease (GvHD). Particularly GvHD following an organ and/or cellular transplant.

Effective cryopreservation would enable more robust cellular therapies utilizing MSCs. Previous reports of attenuated fitness of MSCs post cryopreservation and thaw have speculated that intrinsic properties of MSCs were likely modified by the cryoprotectant system. Historically dimethyl sulfoxide (DMSO) has been the most widely used cryoprotectant for MSCs, and several reports have focused on potential direct effects of the DMSO on post thaw outcomes. To some degree, these reports have pointed to improved intrinsic functions of MSCs and viability post-thaw, however interactions with immune responders and functional fitness has not been demonstrated, and delayed onset cell death due to apoptosis, is still observed.

Recent work evaluating proliferating cell lines has pointed to DNA defects and alterations of higher-order chromatin structure of frozen and thawed cells with and without cryoprotectant treatment. These studies pointed out that that in replicating (S phase) cells, DNA was preferentially damaged by replication fork collapse, potentially leading to DNA double strand breaks (DSBs), which represent an important source of both genome instability and defects in epigenome maintenance and can lead to apoptosis. Thus, it is hypothesized that such DNA damage could be the primary driver of the delayed onset death which was observed in post thaw culture.

Thus, modifying the cell cycle process immediately prior to cryopreservation to prevent cells from entering the S phase would mitigate cryoinjury. Scrum starvation is a mechanism for blocking the cell cycle at the G1 to S phase transition for studying cell cycle. The process involves transferring cells grown in complex medium containing blood components to provide growth factors to a medium that substantially lacks these factors. Here, culture medium containing fetal bovine serum (FBS) or equivalent is removed and replaced with a medium containing no or very low levels (e.g., 1% or less) of FBS. The cells are not able to progress past G1 to S phase and are thus effectively blocked at this transition point. For this reason, cells in the culture are synchronized at the same phase.

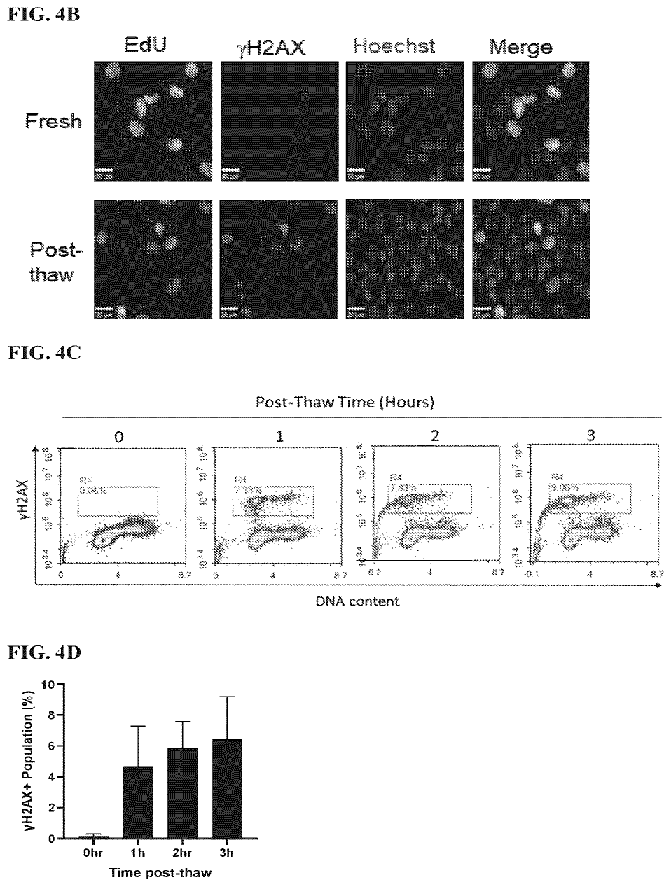

The compositions and methods provided herein dramatically reduced the percentage of MSCs in S phase from 25% to <2% (FIG. 4). These techniques can greatly improve current cryopreservation methods for storage MSCs for pharmaceutical use.

Further, the immunoregulatory properties of MSCs are also impacted by inflammatory processes. For example, immunosuppression by MSCs is enhanced after exposure to cytokines such as IFNγ and TNF-α. For this reason, a number of priming approaches have been employed to improve the efficacy of MSC therapies for clinical application. For example, multi-cytokine priming of human MSCs by TNF-α, IL-1β and IFNγ enhances NF-KB, STATS AND P38-MAPK signaling and augments recruitment of polymorphonuclear granulocytes. Others have similarly shown that priming of MSCs with cytokines enhances the downregulation of proinflammatory cytokines, induces regulatory T cells, inhibits T-cell proliferation, and polarizes M2-type macrophages.

MSCs have potent immune modulatory activity, including prophylactic activity of asthma, which is markedly enhanced by exposure to IFNγ. In vivo, MSCs are stimulated in response to abnormal conditions, e.g., caused by infection, cancer, and injury. The presence of circulating IFNγ can be a signal to MSCs which identifies an abnormal condition. In response to the circulating IFNγ, the MSCs are primed and secrete, at least, factors and proteins that help remedy the abnormal condition. In various aspects of the present disclosure, MSCs are primed in vitro or ex vivo by contact with IFNγ to transform the cells into γMSCs. As used herein, IFNγ-primed MSCs are referred to as “γMSCs”.

Although several MSC priming approaches have been used, priming with IFNγ has been widely studied and enhances the immunosuppressive properties of the MSCs. γMSCs upregulate expression of immunosuppressive factors such as IDO and secrete PGE2, HGF, TGF and CCL2. γMSCs also inhibit T cell effector functions and suppress NK activation more efficiently than non-primed MSCs. In an experimental animal model of colitis, γMSCs also had a higher rate of migration to sites of inflammation and showed greater efficacy in the reduction of mucosal damage and inflammatory responses compared to non-primed bone marrow MSCs.

The present disclosure also provides for compositions and methods that effectively cryopreserve MSCs and/or γMSCs while maintaining the MSCs and/or γMSCs immunosuppressive properties.

### Overview of Manufacturing Processes of Interferon γ-Primed Mesenchymal Stromal Cells

FIG. 11A to FIG. 11E are schematics showing illustrative manufacturing processes for interferon γ-primed mesenchymal stromal cells (γMSCs). The schematics are to be understood according to the teachings presented in the present disclosure. As example, prior sections of this disclosure describe various processes involved in the “bone marrow collection” step. Any processes disclosed herein that relate to obtaining MSCs from bone marrow are include in the “bone marrow collection”. Similarly, as examples, P0, P1, P2, P3, and P4 cell culturing steps are disclosed below as well as method for priming MSCs with y-INF. Accordingly, FIG. 11A to FIG. 11E provide general methodologies that include the specific methods, steps, reagents, and the like that are disclosed in detail herein.

Methods for obtaining bone marrow cells from, comprising one or more of HSCs and MSCs (including vBM-MSC and vBA-MSCs) are described elsewhere in this disclosure. Additionally, teachings relevant to these methods are disclosed in WO2022226356A1, WO2022221672A1, WO2022159824A1, WO2022140296A1, WO2022140613A1, WO2022133282A1, WO2022081909A1, and WO2022081896A1, the contents of which are herein incorporated by reference to the same extent as if each individual patent application was specifically and individually indicated to be incorporated by reference.

### Culturing of MSCs

The MSCs may be expanded in culture prior to being IFNγ primed. Culture-expanded MSCs have potent immunosuppressive activity mediated via a variety of cell-to-cell contact and soluble factors such as IDO, PGE2, TSG-6, CCL-2 and PD-L1, and TGFβ. These mediators inhibit T and B lymphocyte, NK cell and dendritic cell activation and function. MSCs also facilitate endogenous tissue repair and regeneration through secretion of cytokines such as VEGF, IL-6, IL-11, GM-CSF and SCF. As MSCs seem to be hypoimmunogenic and do not express co-stimulatory molecules, they can be infused into major histocompatibility complex (MHC) disparate recipients. Without wishing to be bound by theory, these beneficial properties suggest that MSCs may be useful for prevention and treatment of inflammatory lung diseases such as inflammatory conditions, including auto-immune conditions.

In one aspect of the present disclosure, extracted MSCs may be (e.g., vBA-MSCs and/or vBM-MSCs) cultured and passaged to realize clinical scale MSC preparation having a desired number of MSCs with the antigen profiles taught herein. In some embodiments, a clinical scale preparation may be obtained by serial passage expansion where each passage includes a step of splitting the previous culture into a plurality of cultures at a given ratio. Each passaging step increases the number of concurrent cultures in the preparation. In some embodiments, clinical scale preparations having the instant preparation profiles, e.g., antigen profile, TNFRI profile, cryopreservation profile, differentiation profile, and/or sterility (with respect to pathogens) are successfully produced.

In some embodiments, extracted MSCs are cultured in a medium wherein the medium is configured to generate MSCs having the instant preparation profiles, e.g., antigen profile, TNFRI profile, cryopreservation profile, differentiation profile, and/or sterility (with respect to pathogens). In some embodiments, the medium comprises minimal essential medium (MEM). In some embodiments, the medium comprises alpha MEM. In some embodiments, the medium comprises human platelet lysate (hPL), e.g., Stemulate™. In some embodiments, the medium comprises fibroblast growth factor (FGF, e.g., carrier free FGF and/or FGF-2). In some embodiments, the medium comprises epidermal growth factor (EGF; e.g., carrier free EGF). In some embodiments, the medium comprises alpha MEM, hPL, FGF, EGF, or any combination thereof. In some embodiments, the medium comprises alpha MEM, hPL, FGF, and EGF. In some embodiments, the medium does not further require heparin.

In some embodiments, hPL is present in the medium at about 1% to about 21%. In some embodiments, hPL is present in the medium at about 1% to about 3%, about 1¾ to about s %, about 1% to about 7%, about 1% to about 9%, about 1% to about 10%, about 1% to about 11%, about 1% to about 13%, about 1% to about 15%, about 1% to about 17%, about 1% to about 19%, about 1% to about 21%, about 3% to about 5%, about 3% to about 7%, about 3% to about 9%, about 3% to about 10%, about 3% to about 11%, about 3% to about 13%, about 3% to about 15%, about 3% to about 17%, about 3% to about 19%, about 3% to about 21%, about 5% to about 7%, about 5% to about 9%, about 5% to about 10%, about 5% to about 11%, about 5% to about 13%, about 5% to about 15%, about 5% to about 17%, about 5% to about 19%, about 5% to about 21%, about 7% to about 9%, about 7% to about 10%, about 7% to about 11%, about 7% to about 13%, about 7% to about 15%, about 7% to about 17%, about 7% to about 19%, about 7% to about 21%, about 9% to about 10%, about 9% to about 11%, about 9% to about 13%, about 9% to about 15%, about 9% to about 17%, about 9% to about 19%, about 9% to about 21%, about 10% to about 11%, about 10% to about 13%, about 10% to about 15%, about 10% to about 17%, about 10% to about 19%, about 10% to about 21%, about 11% to about 13%, about 11% to about 15%, about 11% to about 17%, about 11% to about 19%, about 11% to about 21%, about 13% to about 15%, about 13% to about 17%, about 13% to about 19%, about 13% to about 21%, about 15% to about 17%, about 15% to about 19%, about 15% to about 21%, about 17% to about 19%, about 17% to about 21%, or about 19% to about 21%. In some embodiments, hPL is present in the medium at about 1%, about 3%, about 5%, about 7%, about 9%, about 10%, about 11%, about 13%, about 15%, about 17%, about 19%, or about 21%. In some embodiments, hPL is present in the medium at least about 1%, about 3%, about 5%, about 7%, about 9%, about 10%, about 11%, about 13%, about 15%, about 17%, or about 19%. In some embodiments, hPL is present in the medium at most about 3%, about 5%, about 7%, about 9%, about 10%, about 11%, about 13%, about 15%, about 17%, about 19%, or about 21%. In some embodiments, FGF is present in the medium at about 0.5 ng/ml to about 5 ng/ml. In some embodiments, FGF is present in the medium at about 0.5 ng/ml to about 1 ng/ml, about 0.5 ng/ml to about 1.5 ng/ml, about 0.5 ng/ml to about 2 ng/ml, about 0.5 ng/ml to about 2.5 ng/ml, about 0.5 ng/ml to about 3 ng/ml, about 0.5 ng/ml to about 3.5 ng/ml, about 0.5 ng/ml to about 4 ng/ml, about 0.5 ng/ml to about 4.5 ng/ml, about 0.5 ng/ml to about 5 ng/ml, about 1 ng/ml to about 1.5 ng/ml, about 1 ng/ml to about 2 ng/ml, about 1 ng/ml to about 2.5 ng/ml, about 1 ng/ml to about 3 ng/ml, about 1 ng/ml to about 3.5 ng/ml, about 1 ng/ml to about 4 ng/ml, about 1 ng/ml to about 4.5 ng/ml, about 1 ng/ml to about 5 ng/ml, about 1.5 ng/ml to about 2 ng/ml, about 1.5 ng/ml to about 2.5 ng/ml, about 1.5 ng/ml to about 3 ng/ml, about 1.5 ng/ml to about 3.5 ng/ml, about 1.5 ng/ml to about 4 ng/ml, about 1.5 ng/ml to about 4.5 ng/ml, about 1.5 ng/ml to about 5 ng/ml, about 2 ng/ml to about 2.5 ng/ml, about 2 ng/ml to about 3 ng/ml, about 2 ng/ml to about 3.5 ng/ml, about 2 ng/ml to about 4 ng/ml, about 2 ng/ml to about 4.5 ng/ml, about 2 ng/ml to about 5 ng/ml, about 2.5 ng/ml to about 3 ng/ml, about 2.5 ng/ml to about 3.5 ng/ml, about 2.5 ng/ml to about 4 ng/ml, about 2.5 ng/ml to about 4.5 ng/ml, about 2.5 ng/ml to about 5 ng/ml, about 3 ng/ml to about 3.5 ng/ml, about 3 ng/ml to about 4 ng/ml, about 3 ng/ml to about 4.5 ng/ml, about 3 ng/ml to about 5 ng/ml, about 3.5 ng/ml to about 4 ng/ml, about 3.5 ng/ml to about 4.5 ng/ml, about 3.5 ng/ml to about 5 ng/ml, about 4 ng/ml to about 4.5 ng/ml, about 4 ng/ml to about 5 ng/ml, or about 4.5 ng/ml to about 5 ng/ml. In some embodiments, FGF is present in the medium at about 0.5 ng/ml, about 1 ng/ml, about 1.5 ng/ml, about 2 ng/ml, about 2.5 ng/ml, about 3 ng/ml, about 3.5 ng/ml, about 4 ng/ml, about 4.5 ng/ml, or about 5 ng/ml. In some embodiments, FGF is present in the medium at least about 0.5 ng/ml, about 1 ng/ml, about 1.5 ng/ml, about 2 ng/ml, about 2.5 ng/ml, about 3 ng/ml, about 3.5 ng/ml, about 4 ng/ml, or about 4.5 ng/ml. In some embodiments, FGF is present in the medium at most about 1 ng/ml, about 1.5 ng/ml, about 2 ng/ml, about 2.5 ng/ml, about 3 ng/ml, about 3.5 ng/ml, about 4 ng/ml, about 4.5 ng/ml, or about 5 ng/ml.

In some embodiments, EGF is present in the medium at about 0.5 ng/ml to about 5 ng/ml. In some embodiments, EGF is present in the medium at about 0.5 ng/ml to about 1 ng/ml, about 0.5 ng/ml to about 1.5 ng/ml, about 0.5 ng/ml to about 2 ng/ml, about 0.5 ng/ml to about 2.5 ng/ml, about 0.5 ng/ml to about 3 ng/ml, about 0.5 ng/ml to about 3.5 ng/ml, about 0.5 ng/ml to about 4 ng/ml, about 0.5 ng/ml to about 4.5 ng/ml, about 0.5 ng/ml to about 5 ng/ml, about 1 ng/ml to about 1.5 ng/ml, about 1 ng/ml to about 2 ng/ml, about 1 ng/ml to about 2.5 ng/ml, about 1 ng/ml to about 3 ng/ml, about 1 ng/ml to about 3.5 ng/ml, about 1 ng/ml to about 4 ng/ml, about 1 ng/ml to about 4.5 ng/ml, about 1 ng/ml to about 5 ng/ml, about 1.5 ng/ml to about 2 ng/ml, about 1.5 ng/ml to about 2.5 ng/ml, about 1.5 ng/ml to about 3 ng/ml, about 1.5 ng/ml to about 3.5 ng/ml, about 1.5 ng/ml to about 4 ng/ml, about 1.5 ng/ml to about 4.5 ng/ml, about 1.5 ng/ml to about 5 ng/ml, about 2 ng/ml to about 2.5 ng/ml, about 2 ng/ml to about 3 ng/ml, about 2 ng/ml to about 3.5 ng/ml, about 2 ng/ml to about 4 ng/ml, about 2 ng/ml to about 4.5 ng/ml, about 2 ng/ml to about 5 ng/ml, about 2.5 ng/ml to about 3 ng/ml, about 2.5 ng/ml to about 3.5 ng/ml, about 2.5 ng/ml to about 4 ng/ml, about 2.5 ng/ml to about 4.5 ng/ml, about 2.5 ng/ml to about 5 ng/ml, about 3 ng/ml to about 3.5 ng/ml, about 3 ng/ml to about 4 ng/ml, about 3 ng/ml to about 4.5 ng/ml, about 3 ng/ml to about 5 ng/ml, about 3.5 ng/ml to about 4 ng/ml, about 3.5 ng/ml to about 4.5 ng/ml, about 3.5 ng/ml to about 5 ng/ml, about 4 ng/ml to about 4.5 ng/ml, about 4 ng/ml to about 5 ng/ml, or about 4.5 ng/ml to about 5 ng/ml. In some embodiments, EGF is present in the medium at about 0.5 ng/ml, about 1 ng/ml, about 1.5 ng/ml, about 2 ng/ml, about 2.5 ng/ml, about 3 ng/ml, about 3.5 ng/ml, about 4 ng/ml, about 4.5 ng/ml, or about 5 ng/ml. In some embodiments, EGF is present in the medium at least about 0.5 ng/ml, about 1 ng/ml, about 1.5 ng/ml, about 2 ng/ml, about 2.5 ng/ml, about 3 ng/ml, about 3.5 ng/ml, about 4 ng/ml, or about 4.5 ng/ml. In some embodiments, EGF is present in the medium at most about 1 ng/ml, about 1.5 ng/ml, about 2 ng/ml, about 2.5 ng/ml, about 3 ng/ml, about 3.5 ng/ml, about 4 ng/ml, about 4.5 ng/ml, or about 5 ng/ml.

In some embodiments, the medium comprises a modified alpha MEM. In some embodiments, the modified alpha MEM comprises one or more inorganic salts, one or more amino acids, one or more vitamins, glucose, lipoic acid, sodium bicarbonate, sodium pyruvate, or any combination thereof.

In some embodiments, the one or more inorganic salts comprise calcium chloride (dihydrate), magnesium sulfate (heptahydrate), potassium chloride, sodium chloride, sodium phosphate monobasic (dehydrate), or any combination thereof. In some embodiments, each inorganic salt present in the medium is present at about 100 mg/Liter to about 800 mg/Liter. In some embodiments, each inorganic salt present in the medium is present at about 100 mg/Liter to about 200 mg/Liter, about 100 mg/Liter to about 300 mg/Liter, about 100 mg/Liter to about 400 mg/Liter, about 100 mg/Liter to about 500 mg/Liter, about 100 mg/Liter to about 600 mg/Liter, about 100 mg/Liter to about 700 mg/Liter, about 100 mg/Liter to about 800 mg/Liter, about 200 mg/Liter to about 300 mg/Liter, about 200 mg/Liter to about 400 mg/Liter, about 200 mg/Liter to about 500 mg/Liter, about 200 mg/Liter to about 600 mg/Liter, about 200 mg/Liter to about 700 mg/Liter, about 200 mg/Liter to about 800 mg/Liter, about 300 mg/Liter to about 400 mg/Liter, about 300 mg/Liter to about 500 mg/Liter, about 300 mg/Liter to about 600 mg/Liter, about 300 mg/Liter to about 700 mg/Liter, about 300 mg/Liter to about 800 mg/Liter, about 400 mg/Liter to about 500 mg/Liter, about 400 mg/Liter to about 600 mg/Liter, about 400 mg/Liter to about 700 mg/Liter, about 400 mg/Liter to about 800 mg/Liter, about 500 mg/Liter to about 600 mg/Liter, about 500 mg/Liter to about 700 mg/Liter, about 500 mg/Liter to about 800 mg/Liter, about 600 mg/Liter to about 700 mg/Liter, about 600 mg/Liter to about 800 mg/Liter, or about 700 mg/Liter to about 800 mg/Liter. In some embodiments, each inorganic salt present in the medium is present at about 100 mg/Liter, about 200 mg/Liter, about 300 mg/Liter, about 400 mg/Liter, about 500 mg/Liter, about 600 mg/Liter, about 700 mg/Liter, or about 800 mg/Liter. In some embodiments, each inorganic salt present in the medium is present at least about 100 mg/Liter, about 200 mg/Liter, about 300 mg/Liter, about 400 mg/Liter, about 500 mg/Liter, about 600 mg/Liter, or about 700 mg/Liter. In some embodiments, each inorganic salt present in the medium is present at most about 200 mg/Liter, about 300 mg/Liter, about 400 mg/Liter, about 500 mg/Liter, about 600 mg/Liter, about 700 mg/Liter, or about 800 mg/Liter.

In some embodiments, the one or more amino acids comprise glycine, alanine, alanyl-glutamine, arginine (HCl), asparaginc (monohydrate), aspartic acid, cysteine (HCl) (monohydrate), cystine, glutamic acid, histidine, isoleucine, leucine, lysine, methionine, phenylalanine, praline, serine, threonine, tryptophan, tyrosine, valine, or any combination thereof. In some embodiments, the one or more amino acids are present in the L isoform. In some embodiments, the one or more amino acids are present in the D isoform. In some embodiments, the one or more amino acids are present in both isoforms. In some embodiments, each amino acid present in the medium is present at about 10 mg/Liter to about 100 mg/Liter. In some embodiments, each amino acid present in the medium is present at about 10 mg/Liter to about 20 mg/Liter, about 10 mg/Liter to about 30 mg/Liter, about 10 mg/Liter to about 40 mg/Liter, about 10 mg/Liter to about 50 mg/Liter, about 10 mg/Liter to about 60 mg/Liter, about 10 mg/Liter to about 70 mg/Liter, about 10 mg/Liter to about 80 mg/Liter, about 10 mg/Liter to about 90 mg/Liter, about 10 mg/Liter to about 100 mg/Liter, about 20 mg/Liter to about 30 mg/Liter, about 20 mg/Liter to about 40 mg/Liter, about 20 mg/Liter to about 50 mg/Liter, about 20 mg/Liter to about 60 mg/Liter, about 20 mg/Liter to about 70 mg/Liter, about 20 mg/Liter to about 80 mg/Liter, about 20 mg/Liter to about 90 mg/Liter, about 20 mg/Liter to about 100 mg/Liter, about 30 mg/Liter to about 40 mg/Liter, about 30 mg/Liter to about 50 mg/Liter, about 30 mg/Liter to about 60 mg/Liter, about 30 mg/Liter to about 70 mg/Liter, about 30 mg/Liter to about 80 mg/Liter, about 30 mg/Liter to about 90 mg/Liter, about 30 mg/Liter to ab out 100 mg/Liter, about 40 mg/Liter to about 50 mg/Liter, about 40 mg/Liter to about 60 mg/Liter, about 40 mg/Liter to about 70 mg/Liter, about 40 mg/Liter to about 80 mg/Liter, about 40 mg/Liter to about 90 mg/Liter, about 40 mg/Liter to about 100 mg/Liter, about 50 mg/Liter to about 60 mg/Liter, about 50 mg/Liter to about 70 mg/Liter, about 50 mg/Liter to about 80 mg/Liter, about 50 mg/Liter to about 90 mg/Liter, about 50 mg/Liter to about 100 mg/Liter, about 60 mg/Liter to about 70 mg/Liter, about 60 mg/Liter to about 80 mg/Liter, about 60 mg/Liter to about 90 mg/Liter, about 60 mg/Liter to about 100 mg/Liter, about 70 mg/Liter to about 80 mg/Liter, about 70 mg/Liter to about 90 mg/Liter, about 70 mg/Liter to about 100 mg/Liter, about 80 mg/Liter to about 90 mg/Liter, about 80 mg/Liter to about 100 mg/Liter, or about 90 mg/Liter to about 100 mg/Liter. In some embodiments, each amino acid present in the medium is present at about 10 mg/Liter, about 20 mg/Liter, about 30 mg/Liter, about 40 mg/Liter, about 50 mg/Liter, about 60 mg/Liter, about 70 mg/Liter, about 80 mg/Liter, about 90 mg/Liter, or about 100 mg/Liter. In some embodiments, each amino acid present in the medium is present at least about 10 mg/Liter, about 20 mg/Liter, about 30 mg/Liter, about 40 mg/Liter, about 50 mg/Liter, about 60 mg/Liter, about 70 mg/Liter, about 80 mg/Liter, or about 90 mg/Liter. In some embodiments, each amino acid present in the medium is present at most about 20 mg/Liter, about 30 mg/Liter, about 40 mg/Liter, about 50 mg/Liter, about 60 mg/Liter, about 70 mg/Liter, about 80 mg/Liter, about 90 mg/Liter, or about 100 mg/Liter. In some embodiments, each amino acid present in the medium is present at about 100 mg/Liter to about 500 mg/Liter. In some embodiments, each amino acid present in the medium is present at about 100 mg/Liter to about 200 mg/Liter, about 100 mg/Liter to about 300 mg/Liter, about 100 mg/Liter to about 400 mg/Liter, about 100 mg/Liter to about 500 mg/Liter, about 200 mg/Liter to about 300 mg/Liter, about 200 mg/Liter to about 400 mg/Liter, about 200 mg/Liter to about 500 mg/Liter, about 300 mg/Liter to about 400 mg/Liter, about 300 mg/Liter to about 500 mg/Liter, or about 400 mg/Liter to about 500 mg/Liter. In some embodiments, each amino acid present in the medium is present at about 100 mg/Liter, about 200 mg/Liter, about 300 mg/Liter, about 400 mg/Liter, or about 500 mg/Liter. In some embodiments, each amino acid present in the medium is present at least about 100 mg/Liter, about 200 mg/Liter, about 300 mg/Liter, or about 400 mg/Liter. In some embodiments, each amino acid present in the medium is present at most about 200 mg/Liter, about 300 mg/Liter, about 400 mg/Liter, or about 500 mg/Liter.

In some embodiments, the one or more vitamins comprise ascorbic acid, biotin, choline chloride, calcium pantothenate, folic acid, myo-inositol, niacinamide, pyridoxal (HCl), pyruvic acid (sodium salt), riboflavin, thiamine (HCl), vitamin B12, or any combination thereof. In some embodiments, the one or more vitamins are present in the L isoform. In some embodiments, the one or more vitamins are present in the D isoform. In some embodiments, the one or more vitamins are present in both isoforms. In some embodiments, each vitamin present in the medium is present at about 0.1 mg/Liter to about 2 mg/Liter. In some embodiments, each vitamin present in the medium is present at about 0.1 mg/Liter to about 0.3 mg/Liter, about 0.1 mg/Liter to about 0.5 mg/Liter, about 0.1 mg/Liter to about 0.7 mg/Liter, about 0.1 mg/Liter to about 0.9 mg/Liter, about 0.1 mg/Liter to about 1.1 mg/Liter, about 0.1 mg/Liter to about 1.3 mg/Liter, about 0.1 mg/Liter to about 1.5 mg/Liter, about 0.1 mg/Liter to about 1.7 mg/Liter, about 0.1 mg/Liter to about 1.9 mg/Liter, about 0.1 mg/Liter to about 2 mg/Liter, about 0.3 mg/Liter to about 0.5 mg/Liter, about 0.3 mg/Liter to about 0.7 mg/Liter, about 0.3 mg/Liter to about 0.9 mg/Liter, about 0.3 mg/Liter to about 1.1 mg/Liter, about 0.3 mg/Liter to about 1.3 mg/Liter, about 0.3 mg/Liter to about 1.5 mg/Liter, about 0.3 mg/Liter to about 1.7 mg/Liter, about 0.3 mg/Liter to about 1.9 mg/Liter, about 0.3 mg/Liter to about 2 mg/Liter, about 0.5 mg/Liter to about 0.7 mg/Liter, about 0.5 mg/Liter to about 0.9 mg/Liter, about 0.5 mg/Liter to about 1.1 mg/Liter, about 0.5 mg/Liter to about 1.3 mg/Liter, about 0.5 mg/Liter to about 1.5 mg/Liter, about 0.5 mg/Liter to about 1.7 mg/Liter, about 0.5 mg/Liter to about 1.9 mg/Liter, about 0.5 mg/Liter to about 2 mg/Liter, about 0.7 mg/Liter to about 0.9 mg/Liter, about 0.7 mg/Liter to about 1.1 mg/Liter, about 0.7 mg/Liter to about 1.3 mg/Liter, about 0.7 mg/Liter to about 1.5 mg/Liter, about 0.7 mg/Liter to about 1.7 mg/Liter, about 0.7 mg/Liter to about 1.9 mg/Liter, about 0.7 mg/Liter to about 2 mg/Liter, about 0.9 mg/Liter to about 1.1 mg/Liter, about 0.9 mg/Liter to about 1.3 mg/Liter, about 0.9 mg/Liter to about 1.5 mg/Liter, about 0.9 mg/Liter to about 1.7 mg/Liter, about 0.9 mg/Liter to about 1.9 mg/Liter, about 0.9 mg/Liter to about 2 mg/Liter, about 1.1 mg/Liter to about 1.3 mg/Liter, about 1.1 mg/Liter to about 1.5 mg/Liter, about 1.1 mg/Liter to about 1.7 mg/Liter, about 1.1 mg/Liter to about 1.9 mg/Liter, about 1.1 mg/Liter to about 2 mg/Liter, about 1.3 mg/Liter to about 1.5 mg/Liter, about 1.3 mg/Liter to about 1.7 mg/Liter, about 1.3 mg/Liter to about 1.9 mg/Liter, about 1.3 mg/Liter to about 2 mg/Liter, about 1.5 mg/Liter to about 1.7 mg/Liter, about 1.5 mg/Liter to about 1.9 mg/Liter, about 1.5 mg/Liter to about 2 mg/Liter, about 1.7 mg/Liter to about 1.9 mg/Liter, about 1.7 mg/Liter to about 2 mg/Liter, or about 1.9 mg/Liter to about 2 mg/Liter. In some embodiments, each vitamin present in the medium is present at about 0.1 mg/Liter, about 0.3 mg/Liter, about 0.5 mg/Liter, about 0.7 mg/Liter, about 0.9 mg/Liter, about 1.1 mg/Liter, about 1.3 mg/Liter, about 1.5 mg/Liter, about 1.7 mg/Liter, about 1.9 mg/Liter, or about 2 mg/Liter. In some embodiments, each vitamin present in the medium is present at least about 0.1 mg/Liter, about 0.3 mg/Liter, about 0.5 mg/Liter, about 0.7 mg/Liter, about 0.9 mg/Liter, about 1.1 mg/Liter, about 1.3 mg/Liter, about 1.5 mg/Liter, about 1.7 mg/Liter, or about 1.9 mg/Liter. In some embodiments, each vitamin present in the medium is present at most about 0.3 mg/Liter, about 0.5 mg/Liter, about 0.7 mg/Liter, about 0.9 mg/Liter, about 1.1 mg/Liter, about 1.3 mg/Liter, about 1.5 mg/Liter, about 1.7 mg/Liter, about 1.9 mg/Liter, or about 2 mg/Liter. In some embodiments, each vitamin present in the medium is present at about 10 mg/Liter to about 120 mg/Liter. In some embodiments, each vitamin present in the medium is present at about 10 mg/Liter to about 20 mg/Liter, about 10 mg/Liter to about 30 mg/Liter, about 10 mg/Liter to about 40 mg/Liter, about 10 mg/Liter to about 50 mg/Liter, about 10 mg/Liter to about 60 mg/Liter, about 10 mg/Liter to about 70 mg/Liter, about 10 mg/Liter to about 80 mg/Liter, about 10 mg/Liter to about 90 mg/Liter, about 10 mg/Liter to about 100 mg/Liter, about 10 mg/Liter to about 110 mg/Liter, about 10 mg/Liter to about 120 mg/Liter, about 20 mg/Liter to about 30 mg/Liter, about 20 mg/Liter to about 40 mg/Liter, about 20 mg/Liter to about 50 mg/Liter, about 20 mg/Liter to about 60 mg/Liter, about 20 mg/Liter to about 70 mg/Liter, about 20 mg/Liter to about 80 mg/Liter, about 20 mg/Liter to about 90 mg/Liter, about 20 mg/Liter to about 100 mg/Liter, about 20 mg/Liter to about 110 mg/Liter, about 20 mg/Liter to about 120 mg/Liter, about 30 mg/Liter to about 40 mg/Liter, about 30 mg/Liter to about 50 mg/Liter, about 30 mg/Liter to about 60 mg/Liter, about 30 mg/Liter to about 70 mg/Liter, about 30 mg/Liter to about 80 mg/Liter, about 30 mg/Liter to about 90 mg/Liter, about 30 mg/Liter to about 100 mg/Liter, about 30 mg/Liter to about 110 mg/Liter, about 30 mg/Liter to about 120 mg/Liter, about 40 mg/Liter to about 50 mg/Liter, about 40 mg/Liter to about 60 mg/Liter, about 40 mg/Liter to about 70 mg/Liter, about 40 mg/Liter to about 80 mg/Liter, about 40 mg/Liter to about 90 mg/Liter, about 40 mg/Liter to about 100 mg/Liter, about 40 mg/Liter to about 110 mg/Liter, about 40 mg/Liter to about 120 mg/Liter, about 50 mg/Liter to about 60 mg/Liter, about 50 mg/Liter to about 70 mg/Liter, about 50 mg/Liter to about 80 mg/Liter, about 50 mg/Liter to about 90 mg/Liter, about 50 mg/Liter to about 100 mg/Liter, about 50 mg/Liter to about 110 mg/Liter, about 50 mg/Liter to about 120 mg/Liter, about 60 mg/Liter to about 70 mg/Liter, about 60 mg/Liter to about 80 mg/Liter, about 60 mg/Liter to about 90 mg/Liter, about 60 mg/Liter to about 100 mg/Liter, about 60 mg/Liter to about 110 mg/Liter, about 60 mg/Liter to about 120 mg/Liter, about 70 mg/Liter to about 80 mg/Liter, about 70 mg/Liter to about 90 mg/Liter, about 70 mg/Liter to about 100 mg/Liter, about 70 mg/Liter to about 110 mg/Liter, about 70 mg/Liter to about 120 mg/Liter, about 80 mg/Liter to about 90 mg/Liter, about 80 mg/Liter to about 100 mg/Liter, about 80 mg/Liter to about 110 mg/Liter, about 80 mg/Liter to about 120 mg/Liter, about 90 mg/Liter to about 100 mg/Liter, ab out 90 mg/Liter to about 110 mg/Liter, about 90 mg/Liter to about 120 mg/Liter, about 100 mg/Liter to about 110 mg/Liter, about 100 mg/Liter to about 120 mg/Liter, or about 110 mg/Liter to about 120 mg/Liter. In some embodiments, each vitamin present in the medium is present at about 10 mg/Liter, about 20 mg/Liter, about 30 mg/Liter, about 40 mg/Liter, about 50 mg/Liter, about 60 mg/Liter, about 70 mg/Liter, about 80 mg/Liter, about 90 mg/Liter, about 100 mg/Liter, about 110 mg/Liter, or about 120 mg/Liter. In some embodiments, each vitamin present in the medium is present at least about 10 mg/Liter, about 20 mg/Liter, about 30 mg/Liter, about 40 mg/Liter, about 50 mg/Liter, about 60 mg/Liter, about 70 mg/Liter, about 80 mg/Liter, about 90 mg/Liter, about 100 mg/Liter, or about 110 mg/Liter. In some embodiments, each vitamin present in the medium is present at most about 20 mg/Liter, about 30 mg/Liter, about 40 mg/Liter, about 50 mg/Liter, about 60 mg/Liter, about 70 mg/Liter, about 80 mg/Liter, about 90 mg/Liter, about 100 mg/Liter, about 110 mg/Liter, or about 120 mg/Liter.

In some embodiments, the glucose comprised in the medium is anhydrous. In some embodiments, the glucose is present in the L isoform. In some embodiments, the glucose is present in the D isoform. In some embodiments, the glucose is present in both isoforms. In some embodiments, glucose present in the medium is present at about 500 mg/Liter to about 1,600 mg/Liter. In some embodiments, glucose present in the medium is present at about 500 mg/Liter to about 600 mg/Liter, about 500 mg/Liter to about 700 mg/Liter, about 500 mg/Liter to about 800 mg/Liter, about 500 mg/Liter to about 900 mg/Liter, about 500 mg/Liter to about 1,000 mg/Liter, about 500 mg/Liter to about 1,100 mg/Liter, about 500 mg/Liter to about 1,200 mg/Liter, about 500 mg/Liter to about 1,300 mg/Liter, about 500 mg/Liter to about 1,400 mg/Liter, about 500 mg/Liter to about 1,500 mg/Liter, about 500 mg/Liter to about 1,600 mg/Liter, about 600 mg/Liter to about 700 mg/Liter, about 600 mg/Liter to about 800 mg/Liter, about 600 mg/Liter to about 900 mg/Liter, about 600 mg/Liter to about 1,000 mg/Liter, about 600 mg/Liter to about 1,100 mg/Liter, about 600 mg/Liter to about 1,200 mg/Liter, about 600 mg/Liter to about 1,300 mg/Liter, ab out 600 mg/Liter to about 1,400 mg/Liter, about 600 mg/Liter to about 1,500 mg/Liter, about 600 mg/Liter to about 1,600 mg/Liter, about 700 mg/Liter to about 800 mg/Liter, about 700 mg/Liter to about 900 mg/Liter, about 700 mg/Liter to about 1,000 mg/Liter, about 700 mg/Liter to about 1,100 mg/Liter, about 700 mg/Liter to about 1,200 mg/Liter, about 700 mg/Liter to about 1,300 mg/Liter, about 700 mg/Liter to about 1,400 mg/Liter, about 700 mg/Liter to about 1,500 mg/Liter, about 700 mg/Liter to about 1,600 mg/Liter, about 800 mg/Liter to about 900 mg/Liter, about 800 mg/Liter to about 1,000 mg/Liter, about 800 mg/Liter to about 1,100 mg/Liter, about 800 mg/Liter to about 1,200 mg/Liter, about 800 mg/Liter to about 1,300 mg/Liter, about 800 mg/Liter to about 1,400 mg/Liter, about 800 mg/Liter to about 1,500 mg/Liter, about 800 mg/Liter to about 1,600 mg/Liter, about 900 mg/Liter to about 1,000 mg/Liter, about 900 mg/Liter to about 1,100 mg/Liter, about 900 mg/Liter to about 1,200 mg/Liter, about 900 mg/Liter to about 1,300 mg/Liter, about 900 mg/Liter to about 1,400 mg/Liter, about 900 mg/Liter to about 1,500 mg/Liter, about 900 mg/Liter to about 1,600 mg/Liter, about 1,000 mg/Liter to about 1,100 mg/Liter, about 1,000 mg/Liter to about 1,200 mg/Liter, about 1,000 mg/Liter to about 1,300 mg/Liter, about 1,000 mg/Liter to about 1,400 mg/Liter, about 1,000 mg/Liter to about 1,500 mg/Liter, about 1,000 mg/Liter to about 1,600 mg/Liter, about 1,100 mg/Liter to about 1,200 mg/Liter, about 1,100 mg/Liter to about 1,300 mg/Liter, about 1,100 mg/Liter to about 1,400 mg/Liter, about 1,100 mg/Liter to about 1,500 mg/Liter, about 1,100 mg/Liter to about 1,600 mg/Liter, about 1,200 mg/Liter to about 1,300 mg/Liter, about 1,200 mg/Liter to about 1,400 mg/Liter, about 1,200 mg/Liter to about 1,500 mg/Liter, about 1,200 mg/Liter to about 1,600 mg/Liter, about 1,300 mg/Liter to about 1,400 mg/Liter, about 1,300 mg/Liter to about 1,500 mg/Liter, about 1,300 mg/Liter to about 1,600 mg/Liter, about 1,400 mg/Liter to about 1,500 mg/Liter, about 1,400 mg/Liter to about 1,600 mg/Liter, or about 1,500 mg/Liter to about 1,600 mg/Liter. In some embodiments, glucose present in the medium is present at about 500 mg/Liter, about 600 mg/Liter, about 700 mg/Liter, about 800 mg/Liter, about 900 mg/Liter, about 1,000 mg/Liter, about 1,100 mg/Liter, about 1,200 mg/Liter, about 1,300 mg/Liter, about 1,400 mg/Liter, about 1,500 mg/Liter, or about 1,600 mg/Liter. In some embodiments, glucose present in the medium is present at least about 500 mg/Liter, about 600 mg/Liter, about 700 mg/Liter, about 800 mg/Liter, about 900 mg/Liter, about 1,000 mg/Liter, about 1,100 mg/Liter, about 1,200 mg/Liter, about 1,300 mg/Liter, about 1,400 mg/Liter, or about 1,500 mg/Liter. In some embodiments, glucose present in the medium is present at most about 600 mg/Liter, about 700 mg/Liter, about 800 mg/Liter, about 900 mg/Liter, about 1,000 mg/Liter, about 1,100 mg/Liter, about 1,200 mg/Liter, about 1,300 mg/Liter, about 1,400 mg/Liter, about 1,500 mg/Liter, or about 1,600 mg/Liter.

In some embodiments, lipoic acid present in the medium is present at about 0.05 mg/Liter to about 0.5 mg/Liter. In some embodiments, the lipoic acid is present in the medium in the form of DL-thiotic acid. In some embodiments, lipoic acid present in the medium is present at about 0.05 mg/Liter to about 0.1 mg/Liter, about 0.05 mg/Liter to about 0.15 mg/Liter, about 0.05 mg/Liter to about 0.2 mg/Liter, about 0.05 mg/Liter to about 0.25 mg/Liter, about 0.05 mg/Liter to about 0.3 mg/Liter, about 0.05 mg/Liter to about 0.35 mg/Liter, about 0.05 mg/Liter to about 0.4 mg/Liter, about 0.05 mg/Liter to about 0.45 mg/Liter, about 0.05 mg/Liter to about 0.5 mg/Liter, about 0.1 mg/Liter to about 0.15 mg/Liter, about 0.1 mg/Liter to about 0.2 mg/Liter, about 0.1 mg/Liter to about 0.25 mg/Liter, about 0.1 mg/Liter to about 0.3 mg/Liter, about 0.1 mg/Liter to about 0.35 mg/Liter, about 0.1 mg/Liter to about 0.4 mg/Liter, about 0.1 mg/Liter to about 0.45 mg/Liter, about 0.1 mg/Liter to about 0.5 mg/Liter, about 0.15 mg/Liter to about 0.2 mg/Liter, about 0.15 mg/Liter to about 0.25 mg/Liter, about 0.15 mg/Liter to about 0.3 mg/Liter, about 0.15 mg/Liter to about 0.35 mg/Liter, about 0.15 mg/Liter to about 0.4 mg/Liter, about 0.15 mg/Liter to about 0.45 mg/Liter, about 0.15 mg/Liter to about 0.5 mg/Liter, about 0.2 mg/Liter to about 0.25 mg/Liter, about 0.2 mg/Liter to about 0.3 mg/Liter, about 0.2 mg/Liter to about 0.35 mg/Liter, about 0.2 mg/Liter to about 0.4 mg/Liter, about 0.2 mg/Liter to about 0.45 mg/Liter, about 0.2 mg/Liter to about 0.5 mg/Liter, about 0.25 mg/Liter to about 0.3 mg/Liter, about 0.25 mg/Liter to about 0.35 mg/Liter, about 0.25 mg/Liter to about 0.4 mg/Liter, about 0.25 mg/Liter to about 0.45 mg/Liter, about 0.25 mg/Liter to about 0.5 mg/Liter, about 0.3 mg/Liter to about 0.35 mg/Liter, about 0.3 mg/Liter to about 0.4 mg/Liter, about 0.3 mg/Liter to about 0.45 mg/Liter, about 0.3 mg/Liter to about 0.5 mg/Liter, about 0.35 mg/Liter to about 0.4 mg/Liter, about 0.35 mg/Liter to about 0.45 mg/Liter, about 0.35 mg/Liter to about 0.5 mg/Liter, about 0.4 mg/Liter to about 0.45 mg/Liter, about 0.4 mg/Liter to about 0.5 mg/Liter, or about 0.45 mg/Liter to about 0.5 mg/Liter. In some embodiments, lipoic acid present in the medium is present at about 0.05 mg/Liter, about 0.1 mg/Liter, about 0.15 mg/Liter, about 0.2 mg/Liter, about 0.25 mg/Liter, about 0.3 mg/Liter, about 0.35 mg/Liter, about 0.4 mg/Liter, about 0.45 mg/Liter, or about 0.5 mg/Liter. In some embodiments, lipoic acid present in the medium is present at least about 0.05 mg/Liter, about 0.1 mg/Liter, about 0.15 mg/Liter, about 0.2 mg/Liter, about 0.25 mg/Liter, about 0.3 mg/Liter, about 0.35 mg/Liter, about 0.4 mg/Liter, or about 0.45 mg/Liter. In some embodiments, lipoic acid present in the medium is present at most about 0.1 mg/Liter, about 0.15 mg/Liter, about 0.2 mg/Liter, about 0.25 mg/Liter, about 0.3 mg/Liter, about 0.35 mg/Liter, about 0.4 mg/Liter, about 0.45 mg/Liter, or about 0.5 mg/Liter.

In some embodiments, sodium bicarbonate present in the medium is present at about 250 mg/Liter to about 2,000 mg/Liter. In some embodiments, sodium bicarbonate present in the medium is present at about 250 mg/Liter to about 500 mg/Liter, about 250 mg/Liter to about 750 mg/Liter, about 250 mg/Liter to about 1,000 mg/Liter, about 250 mg/Liter to about 1,250 mg/Liter, about 250 mg/Liter to about 1,500 mg/Liter, about 250 mg/Liter to about 1,750 mg/Liter, about 250 mg/Liter to about 2,000 mg/Liter, about 500 mg/Liter to about 750 mg/Liter, about 500 mg/Liter to about 1,000 mg/Liter, about 500 mg/Liter to about 1,250 mg/Liter, about 500 mg/Liter to about 1,500 mg/Liter, about 500 mg/Liter to about 1,750 mg/Liter, about 500 mg/Liter to about 2,000 mg/Liter, about 750 mg/Liter to about 1,000 mg/Liter, about 750 mg/Liter to about 1,250 mg/Liter, about 750 mg/Liter to about 1,500 mg/Liter, about 750 mg/Liter to about 1,750 mg/Liter, about 750 mg/Liter to about 2,000 mg/Liter, about 1,000 mg/Liter to about 1,250 mg/Liter, about 1,000 mg/Liter to about 1,500 mg/Liter, about 1,000 mg/Liter to about 1,750 mg/Liter, about 1,000 mg/Liter to about 2,000 mg/Liter, about 1,250 mg/Liter to about 1,500 mg/Liter, about 1,250 mg/Liter to about 1,750 mg/Liter, about 1,250 mg/Liter to about 2,000 mg/Liter, about 1,500 mg/Liter to about 1,750 mg/Liter, about 1,500 mg/Liter to about 2,000 mg/Liter, or about 1,750 mg/Liter to about 2,000 mg/Liter. In some embodiments, sodium bicarbonate present in the medium is present at about 250 mg/Liter, about 500 mg/Liter, about 750 mg/Liter, about 1,000 mg/Liter, about 1,250 mg/Liter, about 1,500 mg/Liter, about 1,750 mg/Liter, or about 2,000 mg/Liter. In some embodiments, sodium bicarbonate present in the medium is present at least about 250 mg/Liter, about 500 mg/Liter, about 750 mg/Liter, about 1,000 mg/Liter, about 1,250 mg/Liter, about 1,500 mg/Liter, or about 1,750 mg/Liter. In some embodiments, sodium bicarbonate present in the medium is present at most about 500 mg/Liter, about 750 mg/Liter, about 1,000 mg/Liter, about 1,250 mg/Liter, ab out 1,500 mg/Liter, about 1,750 mg/Liter, or about 2,000 mg/Liter.

In some embodiments, sodium pyruvate present in the medium is present at about 50 mg/Liter to about 160 mg/Liter. In some embodiments, sodium pyruvate present in the medium is present at about 50 mg/Liter to about 60 mg/Liter, about 50 mg/Liter to about 70 mg/Liter, about 50 mg/Liter to about 80 mg/Liter, about 50 mg/Liter to about 90 mg/Liter, about 50 mg/Liter to about 100 mg/Liter, about 50 mg/Liter to about 110 mg/Liter, about 50 mg/Liter to about 120 mg/Liter, about 50 mg/Liter to about 130 mg/Liter, about 50 mg/Liter to about 140 mg/Liter, about 50 mg/Liter to about 150 mg/Liter, about 50 mg/Liter to about 160 mg/Liter, about 60 mg/Liter to about 70 mg/Liter, about 60 mg/Liter to about 80 mg/Liter, about 60 mg/Liter to about 90 mg/Liter, about 60 mg/Liter to about 100 mg/Liter, about 60 mg/Liter to about 110 mg/Liter, about 60 mg/Liter to about 120 mg/Liter, about 60 mg/Liter to about 130 mg/Liter, about 60 mg/Liter to about 140 mg/Liter, about 60 mg/Liter to about 150 mg/Liter, about 60 mg/Liter to about 160 mg/Liter, about 70 mg/Liter to about 80 mg/Liter, about 70 mg/Liter to about 90 mg/Liter, about 70 mg/Liter to about 100 mg/Liter, about 70 mg/Liter to about 110 mg/Liter, about 70 mg/Liter to about 120 mg/Liter, about 70 mg/Liter to about 130 mg/Liter, about 70 mg/Liter to about 140 mg/Liter, about 70 mg/Liter to about 150 mg/Liter, about 70 mg/Liter to about 160 mg/Liter, about 80 mg/Liter to about 90 mg/Liter, about 80 mg/Liter to about 100 mg/Liter, about 80 mg/Liter to about 110 mg/Liter, about 80 mg/Liter to about 120 mg/Liter, about 80 mg/Liter to about 130 mg/Liter, about 80 mg/Liter to about 140 mg/Liter, about 80 mg/Liter to about 150 mg/Liter, about 80 mg/Liter to about 160 mg/Liter, about 90 mg/Liter to about 100 mg/Liter, about 90 mg/Liter to about 110 mg/Liter, about 90 mg/Liter to about 120 mg/Liter, about 90 mg/Liter to about 130 mg/Liter, about 90 mg/Liter to about 140 mg/Liter, about 90 mg/Liter to about 150 mg/Liter, about 90 mg/Liter to about 160 mg/Liter, about 100 mg/Liter to about 110 mg/Liter, about 100 mg/Liter to about 120 mg/Liter, about 100 mg/Liter to about 130 mg/Liter, about 100 mg/Liter to about 140 mg/Liter, about 100 mg/Liter to about 150 mg/Liter, about 100 mg/Liter to about 160 mg/Liter, about 110 mg/Liter to about 120 mg/Liter, about 110 mg/Liter to about 130 mg/Liter, about 110 mg/Liter to about 140 mg/Liter, about 110 mg/Liter to about 150 mg/Liter, about 110 mg/Liter to about 160 mg/Liter, about 120 mg/Liter to about 130 mg/Liter, about 120 mg/Liter to about 140 mg/Liter, about 120 mg/Liter to about 150 mg/Liter, about 120 mg/Liter to about 160 mg/Liter, about 130 mg/Liter to about 140 mg/Liter, about 130 mg/Liter to about 150 mg/Liter, about 130 mg/Liter to about 160 mg/Liter, about 140 mg/Liter to about 150 mg/Liter, about 140 mg/Liter to about 160 mg/Liter, or about 150 mg/Liter to about 160 mg/Liter. In some embodiments, sodium pyruvate present in the medium is present at about 50 mg/Liter, about 60 mg/Liter, about 70 mg/Liter, about 80 mg/Liter, about 90 mg/Liter, about 100 mg/Liter, about 110 mg/Liter, about 120 mg/Liter, about 130 mg/Liter, about 140 mg/Liter, about 150 mg/Liter, or about 160 mg/Liter. In some embodiments, sodium pyruvate present in the medium is present at least about 50 mg/Liter, about 60 mg/Liter, about 70 mg/Liter, about 80 mg/Liter, about 90 mg/Liter, about 100 mg/Liter, about 110 mg/Liter, about 120 mg/Liter, about 130 mg/Liter, about 140 mg/Liter, or about 150 mg/Liter. In some embodiments, sodium pyruvate present in the medium is present at most about 60 mg/Liter, about 70 mg/Liter, about 80 mg/Liter, about 90 mg/Liter, about 100 mg/Liter, about 110 mg/Liter, about 120 mg/Liter, about 130 mg/Liter, about 140 mg/Liter, about 150 mg/Liter, or about 160 mg/Liter.

In some embodiments, the pH of the alpha MEM is between 7.0 and 7.4.

In some embodiments, the alpha MEM comprises the ingredients are presented in Table 1.

TABLE 1

| Components | mg/Liter | Components | mg/Liter |
|---|---|---|---|
| INORGANIC SALTS | | AMINO ACIDS | |
| Calcium Chloride dihydrate | 264 | L-Threonine (C₄H₉NO₃) | 480 |
| Magnesium Sulfate heptahydrate | 200 | -Tryptophan | 100 |
| Potassium Chloride (KCl) | 400 | -Tyrosine (C₉H₉NO₃) | 360 |
| Sodium Chloride (NaCl) | 6800 | L-Valine (C₅H₁₁NO₇) | 460 |
| Sodium Phosphate Monobasic, | 158 | | |
| dihydrate (NaH₂P₂O₄, 2H₂O) | | | |
| OTHER COMPONENTS | | VITAMINS | |
| D-Glucose Anhydrous (C₆11₁₂O₆) | 1000 | L-Ascorbic Acid (C₆H₈O₆) | 500 |
| Lipoic Acid (DL-Thiotic Acid) | 0.2 | D-Biotin (C₁₀H₁₆N₂O₃S) | 0.1 |
| C₈H₁₄O₂S₂) | | | |
| Sodium Bicarbonate (NaHCO₃) | 2200 | Choline Chloride | 10 |
| Sodium Pyruvate (C₃H₃NaO₃) | 110 | D-Calcium Pantothenate | 10 |
| | | Folic Acid (C₁₉H₁₉N₇O₆) | 10 |
| AMINO ACIDS | | Myo-Inositol (C₆H₁₂O₆) | 20 |
| Glycine (C₂H₅NO₂) | 500 | Niacinamide (C₆H₆N₂O) | 10 |
| L-Alanine (C₃H₇NO₂) | 250 | Pyridoxal HCI | 10 |
| L-Alanyl-Glutamine (C₉H₁₅N₃O₄) | 406 | Pyruvic Acid Sodium Salt | 110 |
| L-Arginine HCl (C₆H₁₅ClN₄O₂) | 105 | Riboflavin (C₁₇H₂₀N₄O₆) | 0.1 |
| L-Asparagine monohydrate (C₄H₈N₂O₃, | 500 | Thiamine HCI | 10 |
| H₂O) | | | |
| L-Aspartic Acid (C₄H₇NO₄) | 300 | Vitamin B12 | 1.3 |
| L-Cysteine HCl monohydrate | 100 | | |
| L-Cystine (C6H12N2O4S2) | 240 | | |
| L-Glutamic Acid (C₅H₉NO₄) | 750 | | |
| L-Histidine (C₆H₉N₃O₂) | 310 | | |
| L-Isoleucine (C₆H₁₃NO₂) | 52.4 | | |
| L-Leucine (C₆H₁₃NO₂) | 52.4 | | |
| L-Lysine (C₆H₁₄N₂O₂) | 580 | | |
| L-Methionine (C₅H₁₁NO₂S) | 150 | | |
| L-Phenylalanine (C₉H₁₁NO₂) | 320 | | |
| L-Proline (C₅H₉NO₂) | 400 | | |
| L-Serine (C₃H₇NO₃) | 250 | | |

In some embodiments, the vBM-MSCs and/or vBA-MSCs (collectively MSCs) are cultured in an MSC Culture medium comprising alpha MEM as described in Table 1, 10% hPL (e.g., Stemulate™, 2 ng/ml recombinant, FGF (e.g., basic fibroblast growth factor (FGF-2) and/or carrier free FGF), and 2 ng/ml recombinant, epidermal growth factor (EGF, e.g., carrier free EGF). In some embodiments, no heparin is included in the culturing medium. In various embodiments, the MSCs are cultured for about 14 days with fresh media changes occurring every 3 to 4 days. Approximately 3.9 billion mononuclear cells (e.g., 2 to 5 billion, 3 to 4 billion, and any number of cells therebetween) are plated in culture dishes, e.g., six CellBIND® 10-chamber CellSTACKS®. In some embodiments, no bovine or porcine components and no antibiotics/mycotics are used in culturing process. The MSCs at this stage are termed Passage 0 (P0) cells.

When the PO MSCs have achieved about 75% confluence or greater, the cells are detached and re-plated in MSC Culture Media. The MSCs at this stage are termed Passage 1 (P1) cells. Detachment, at this passage and/or any future passage, may comprise either or both of mechanical and enzymatic methods. The enzymatic method may comprise use of a trypsin or a commercially-available trypsin replacement, e.g., TrypLE™. In some embodiments, the P1 cells (about 410 million cells, e.g., 250 million to 500 million, 300 million to 450 million, or any number of cells therebetween) are cultured for 4 to 5 days and when the culture has reached about 75% confluence or greater, the cells are detached and resuspended in PLASMA LYTE A+2.5% human serum albumin (HSA)+5% dimethyl sulfoxide (DMSO) at about 13 million cells/ml and packaged in about 220 2-mL CellSeal® closed-system cryovials at 2 mL per cryovial using an automatic filler. The cryovials are then cryopreserved and placed into the vapor phase above liquid nitrogen in cryogenic tanks for storage at less than or equal to −140° C.

In alternate embodiments, the P1 cells are not cryopreserved; instead, are detached and replated in MSC Culture media to become the Passage 2 (P2) cells.

Table 2 lists testing optional performed on the P1 cells and preferable features that cultured MSCs may possess. In some cases, only subset of the tests listed in Table 2 are performed. As an example, Quantitative Transmission Electron Microscopy may be omitted.

TABLE 2
P1 MSC Characterization

| Attributes | Test | Preferable Features |
|---|---|---|
| Viability | Trypan Blue or Acridine | ≥70% |
| | Orange/Propidium Iodide | |
| Cell Product | Direct Inoculation Method | No Growth |
| Sterility | (determined suitable through | |
| | Bacteriostasis and Fungistasis testing | |
| | on test article) | |
| Attributes | Test | Preferable Features |
| Mycoplasma | Rapid Detection of Mycoplasma by | Negative for Mycoplasma |
| | Real-Time PCR | |
| Adventitious | 28-Day In vitro Assay (utilizing 3 cell | Negative for the Presence of |
| Agents | lines) | Viral Contaminants |
| | CoA Detection of 14 viruses by RT- | Negative |
| | PCR (Human Panel 1) | |
| | Quantitative Transmission Electron | No extraneous agents observed |
| | Microscopy of Sections for the | in the 200 cell profiles examined |
| | Detection of Viruses, Fungi, Yeasts, | |
| | Bacteria, and Mycoplasmas (200 cell | |
| | profiles) | |
| Cell Product | Flow Cytometry | CD90, CD105 positive (≥90%), |
| Identity and | | CD45, CD14 negative (≤5%) |
| Cellular Purity | | |
| Cell Product | Colony forming unit-fibroblast (CFU- | Greater than 20,000 colonies per |
| Activity | F) analysis | million cells plated in preferred. |

Potency of P1 cells can be evaluated through colony forming unit-fibroblast (CFU-F) analysis. For this assay, cells are resuspended in medium and counted using a Cellometer or via Trypan Blue Manual Counting. Next, 50 cells each are pipetted into 2 wells of a 6 well tissue culture treated plate. The plate is incubated for 10-14 days or until colonies are above 75% confluence (e.g., 80% confluence), refeeding with prewarmed medium every 3-4 days. Once the desired confluence is met, medium is aspirated, the plate is washed twice with phosphate buffered saline (PBS), and 5 mL of Methanol is added to each well. This is allowed to sit for 5 minutes to fix the cells, then it is removed, and the wells are stained with Crystal Violet for 20 minutes. Finally, the Crystal Violet is removed, and the plates are washed 3 times with DI water, and colonies are counted (a colony must have 50 or more cells). An average of the two wells is taken and the average is used to calculate CFU-F colonies per 1M cells plated.

For P1 cells (or later stages), various methodologies can be used to detect mycoplasma including PCR, ELISA and biochemical reaction. All methods have proven reliable; however, PCR and ELISA methods are more labor intensive than biochemical methods. In some embodiments, the MycoAlert® Assay system (Lonza) is used to detect mycoplasma. The MycoAlert® Assay is a selective biochemical test that exploits the activity of certain mycoplasmal enzymes. The presence of these enzymes provides a rapid screening procedure, allowing sensitive detection of contaminating mycoplasma in a test sample. The viable mycoplasma is lysed, and the enzymes react with the MycoAlert® Substrate catalyzing the conversion of ADP to ATP. By measuring the level of ATP in a sample both before and after the addition of the MycoAlert® Substrate, a ratio can be obtained which is indicative of the presence or absence of mycoplasma. If these enzymes are not present, the second reading shows no increase over the first, while reaction of mycoplasmal enzymes with their specific substrates in the MycoAlert® substrate leads to elevated ATP levels. Changes in ATP are detected using a bioluminescent reaction.

P1 cells, either thawed from frozen cryovials or freshly detached from the P1 culture vessel, are re-plated in MSC Culture media to become the Passage 2 (P2) cells. When the P2 cells have achieved about 75% confluence or greater, e.g., around four to five days, the cells are detached and re-plated in MSC Culture Media. In some embodiments, cells (about 410 million cells, e.g., 250 million to 500 million, 300 million to 450 million, or any number of cells therebetween) are replated in about 16 CellBind® 10-chamber CellStacks®). These cells are now termed Passage 3 or P3 cells.

In some embodiments, the P3 cells are cultured for 4-5 days and once the culture vessels have achieved about 75% confluence or greater, the cells are detached, resuspended in PLASMA-LYTE A+2.5% HSA+5% DMSO at about 20 million cells/ml and packaged in 5-mL CellSeal® closed-system cryovials at 5 mL per cryovial using an automatic filler. The cryovials are then cryopreserved, which may comprise a precooling step or equilibration at 4° C. followed by passive cryopreservation at a rate of −1° C. per minute to about −80° C. or less; then the cryovials are placed into the vapor phase above liquid nitrogen in cryogenic tanks for storage at less than or equal to −140° C. Cryopreserved P3 cells may be shipped to a treatment site for further culturing and final product formulation to γ-MSC. See, e.g., FIG. 11B. In some cases, the MSCs are collected from donor bone and initially cultured (PO through P3) at the same location as the treatment site; in these cases, cryopreserved P3 cells do not need to be shipped to another site. These embodiments are variants of FIG. 11C and FIG. 11E in which the “Shipping” may be within a facility, campus, or healthcare complex.

In alternate embodiments, the P3 cells are not cryopreserved; instead, are detached and replated in MSC Culture media to become the Passage 4 (P4) cells. Sec, e.g., FIG. 11C and FIG. 11E.

Table 3 lists optional tests performed on the P3 cells and preferable features that cultured MSCs may possess. In some cases, only subset of the tests listed in Table 3 are performed.

TABLE 3
P3 MSC Characterization

| Attributes | Test | Preferable Features |
|---|---|---|
| Viability | Trypan Blue or Acridine | ≥70% |
| | Orange/Propidium Iodide | |
| Cell Product Sterility | Direct Inoculation Method (determined | No Growth |
| | suitable through Bacteriostasis/ | |
| | Fungistasis testing on test article), e.g., | |
| | USP/CFR 610.12 GMP | |
| Expended Media | Filtration method (determined suitable | No Growth |
| Sterility | through Bacteriostasis/Fungistasis | |
| | testing on test article) | |
| Mycoplasma | Rapid Detection of Mycoplasma by | Negative for Mycoplasma |
| | Real-Time PCR | |
| Cell Product Identity | Flow Cytometry | CD90, 105 positive (≥90%), |
| and Purity | | CD45, CD14 negative (≤5%) |
| Cell Product Activity | Colony forming unit-fibroblast (CFU-F) | >20k colonies per 1M cells plated |

Potency of P3 cells can be evaluated through colony forming unit-fibroblast (CFU-F) analysis. For this assay, cells are resuspended in medium and counted using a Cellometer or via Trypan Blue Manual Counting. Next, 50 cells each are pipetted into 2 wells of a 6 well tissue culture treated plate. The plate is incubated for 10-14 days or until colonies are at 80% confluence, refeeding with prewarmed medium every 3-4 days. Once confluent, medium is aspirated, the plate is washed twice with phosphate buffered saline (PBS), and 5 mL of Methanol is added to each well. This is allowed to sit for 5 minutes to fix the cells, then it is removed, and the wells are stained with Crystal Violet for 20 minutes. Finally, the Crystal Violet is removed, and the plates are washed 3 times with DI water, and colonies are counted (a colony must have 50 or more cells). An average of the two wells is taken and the average is used to calculate CFU-F colonies per 1M cells plated.

The time elapsed through each culture step is variable, with >75% confluence generally being achieved in about four to about five days. Time elapsed from a culture harvest to the start of the cryopreservation may be less than about six hours. Upon cryopreservation, cells are expected to be indefinitely stable.

In some embodiments, the primary MSCs may be further passaged to non-primary cells (e.g., removed from the culture surface and expanded into additional area) by seeding at a density of about 1,000 to about one million nucleated cells/cm² of culture dish (e.g., about 5,900 cells/cm² plus and minus about 1,200), and then culturing for additional days, e.g., about 14±about 2 days. In suitable embodiments, the primary cells may be grown to confluence, and in some instances may be passaged to a second culture of non-primary cells by seeding the primary cells from a confluent primary cell culture in the second culture surface in an amount below confluence and growing the non-primary culture to confluence. This method can be repeated for additional passages.

In some embodiments, the MSCs in the treatment composition may originate from sequential generation number (i.e., they are within about 1 or about 2 or about 3 or about 4 cell doublings of each other). Optionally, the average number of cell doublings in the present composition treatment composition may be about 20 to about 25 doublings. Optionally, the average number of cell doublings in the present treatment composition may be about 9 to about 13 (e.g., about 11 or about 11.2) doublings arising from the primary culture, plus about 1, about 2, about 3, or about 4 doublings per passage (for example, about 2.5 doublings per passage). Average cell doublings in present preparations may be of about 13.5, about 16, about 18.5, about 21, about 23.5, about 26, about 28.5, about 31, about 33.5, or about 36 when produced by about 1, about 2, about 3, about 4, about 5, about 6, about 7, about 8, about 9, or about 10 passages, respectively.

In some embodiments, notwithstanding one or more population doublings, the MSCs in the treatment composition (e.g., vBM-MSCs and/or vBA-MSCs; collectively MSCs) may originate from MSCs that were cultured through about 1, about 2, about 3, about 4, about 5, about 6, about 7, about 8, about 9, or about 10 passages.

Confluence refers to the percentage of the surface of a culture dish that is covered by adherent cells. For example, 50 percent confluence means roughly half of the surface is covered, while 100 percent confluence means the surface is completely covered by the cells, and no more room is left for the cells to grow as a mono layer. By “75% confluence or greater” is meant about 75% confluence, about 76% confluence, about 77% confluence, about 78% confluence, about 79% confluence, about 80% confluence, about 81% confluence, about 82% confluence, about 83% confluence, about 84% confluence, about 85% confluence, about 86% confluence, about 87% confluence, about 88% confluence, about 89% confluence, about 90% confluence, about 91% confluence, about 92% confluence, about 93% confluence, about 94% confluence, about 95% confluence, about 96% confluence, about 97% confluence, about 98% confluence, about 99% confluence, or about 100% confluence. Confluence can be determined by any standard method used in the field. See, e.g., Haenel, Frauke, and Norbert Garbow. “Cell counting and confluency analysis as quality controls in cell-based assays.” Multimode Detection (2014): 1-5.

### Cryopreservation and Rinse Media

The MSCs may undergo cryopreservation after completing a primary expansion. In some embodiments, for a method provided herein, said cell culture was cryopreserved in a cryopreservation media, wherein said cryopreservation media comprises an electrolyte formulation, human serum albumin (HSA), dimethyl sulfoxide (DMSO), or any combination thereof.

In some embodiments, cryovials are cryopreserved in a method comprising a precooling step or equilibration at 4° C. followed by passive cryopreservation at a rate of −1° C. per minute to about −80° corless; then the cryovials are placed into the vapor phase above liquid nitrogen in cryogenic tanks for storage at less than or equal to −140° C.

In some embodiments, said cryopreservation media comprises about 1% to about 10%, about 1% to about 9%, about 1% to about 8%, about 1% to about 7%, about 1% to about 6%, about 1% to about 5%, about 1% to about 4%, about 1% to about 3%, about 1% to about 2%, about 2% to about 10%, about 2% to about 9%, about 2% to about 8%, about 2% to about 7%, about 2% to about 6%, about 2% to about 5%, about 2% to about 4%, about 2% to about 3%, about 3% to about 10%, about 3% to about 9%, about 3% to about 8%, about 3% to about 7%, about 3% to about 6%, about 3% to about 5%, about 3% to about 4%, about 4% to about 10%, about 4% to about 9%, about 4% to about 8%, about 4% to about 7%, about 4% to about 6%, about 4% to about 5%, about 5% to about 10%, about 5% to about 9%, about 5% to about 8%, about 5% to about 7%, about 5% to about 6%, about 6% to about 10%, about 6% to about 9%, about 6% to about 8%, about 6% to about 7%, about 7% to about 10%, about 7% to about 9%, about 7% to about 8%, about 8% to about 10%, about 8% to about 9%, or about 9% to about 10% HSA. In some embodiments, said cryopreservation media comprises about 1% to about 5% HSA. In some embodiments, said cryopreservation media comprises about 2.5% HSA.

In some embodiments, said cryopreservation media comprises about 1% to about 10%, about 1% to about 9%, about 1% to about 8%, about 1% to about 7%, about 1% to about 6%, about 1% to about 5%, about 1% to about 4%, about 1% to about 3%, about 1% to about 2%, about 2% to about 10%, about 2% to about 9%, about 2% to about 8%, about 2% to about 7%, about 2% to about 6%, about 2% to about 5%, about 2% to about 4%, about 2% to about 3%, about 3% to about 10%, about 3% to about 9%, about 3% to about 8%, about 3% to about 7%, about 3% to about 6%, about 3% to about 5%, about 3% to about 4%, about 4% to about 10%, about 4% to about 9%, about 4% to about 8%, about 4% to about 7%, about 4% to about 6%, about 4% to about 5%, about 5% to about 10%, about 5% to about 9%, about 5% to about 8%, about 5% to about 7%, about 5% to about 6%, about 6% to about 10%, about 6% to about 9%, about 6% to about 8%, about 6% to about 7%, about 7% to about 10%, about 7% to about 9%, about 7% to about 8%, about 8% to about 10%, about 8% to about 9%, or about 9% to about 10% DMSO. In some embodiments, said cryopreservation media comprises about 1% to about 10% DMSO. In some embodiments, said cryopreservation media comprises about 5% DMSO.

In some embodiments, said electrolyte formulation is PLASMA-LYTEA.

In some embodiments, for a method provided herein, the method further comprises, resuspending said cell culture in a rinse media, wherein said rinse media comprises an electrolyte formulation, human serum albumin (HSA), or both. In some embodiments, for a method provided herein, the method further comprises resuspending said cell culture in a rinse media, wherein said rinse media comprises an electrolyte formulation, human serum albumin (HSA), or both.

In some embodiments, the rinse media is fresh.

In some embodiments, said rinse media comprises about 1% to about 10%, about 1% to about 9%, about 1% to about 8%, about 1% to about 7%, about 1% to about 6%, about 1% to about 5%, about 1% to about 4%, about 1% to about 3%, about 1% to about 2%, about 2% to about 10%, about 2% to about 9%, about 2% to about 8%, about 2% to about 7%, about 2% to about 6%, about 2% to about 5%, about 2% to about 4%, about 2% to about 3%, about 3% to about 10%, about 3% to about 9%, about 3% to about 8%, about 3% to about 7%, about 3% to about 6%, about 3% to about 5%, about 3% to about 4%, about 4% to about 10%, about 4% to about 9%, about 4% to about 8%, about 4% to about 7%, about 4% to about 6%, about 4% to about 5%, about 5% to about 10%, about 5% to about 9%, about 5% to about 8%, about 5% to about 7%, about 5% to about 6%, about 6% to about 10%, about 6% to about 9%, about 6% to about 8%, about 6% to about 7%, about 7% to about 10%, about 7% to about 9%, about 7% to about 8%, about 8% to about 10%, about 8% to about 9%, or about 9% to about 10% HSA. In some embodiments, said rinse media comprises about 1% to about 5% HSA. In some embodiments, said rinse media comprises about 2.5% HSA.

In some embodiments, the systems and methods described herein have the ability to generate about 220 cryovials (26 million cells/vial) of passage I (PI) master cell bank (MCB), which can generate approximately 193,600 vials (100 million cells/vial) of passage 4 (P4) marrow-derived mesenchymal stem cell final product with the potential to treat about 193,600 subjects at a dose of 100 million cells/dose. In some embodiments, the systems and methods described herein generate between about 100, 120, 140, 160, 180, 200, 220, 240, 260, 280, 300, or more than 300 cryovials of passage I master cell bank. In some embodiments, each cryovial comprises about 10 million, about 11 million, about 12 million, about 13 million, about 14 million, about 15 million, ab out 16 million, about 17 million, about 18 million, about 19 million, about 20 million, about 21 million, about 22 million, about 23 million, about 24 million, about 25 million, about 26 million, about 27 million, about 28 million, about 29 million, about 30 million, about 32 million, about 32 million, about 33 million, about 34 million, about 35 million, about 36 million, about 37 million, about 38 million, about 48 million, or 40 million cells per vial of passage 1 master bank cells. In some embodiments, this allows the generation of about 100,000, about 110,000, about 120,000, about 130,000, about 140,000, about 150,000, about 160,000, about 170,000, about 180,000, about 190,000, about 200,000, about 210,000, about 220,000, about 230,000, about 240,000, ab out 250,000, about 260,000, about 270,000, about 280,000, about 290,000, about or 300,000 vials of passage 4 (P4) cells. In some embodiments, this allows the generation of at least about 100,000, about 110,000, about 120,000, about 130,000, about 140,000, about 150,000, about 160,000, about 170,000, about 180,000, about 190,000, about 200,000, about 210,000, about 220,000, about 230,000, about 240,000, about 250,000, about 260,000, about 270,000, about 280,000, about 290,000, about or 300,000 vials of passage 4 (P4) cells. In some embodiments, each vial of P4 cells comprises about 10 million, about 20 million, about 30 million, about 40 million, about 50 million, about 60 million, about 70 million, about 80 million, about 90 million, about 100 million, about 110 million, about 120 million, about 130 million, about 140, 150 million, about 160 million, about 170 million, about 180 million, about 290 million, about 200 million or more than 200 million cells. In some embodiments, each vial of P4 cells comprises at least about 10 million, about 20 million, about 30 million, about 40 million, about 50 million, about 60 million, about 70 million, about 80 million, about 90 million, about 100 million, about 110 million, about 120 million, about 130 million, about 140, 150 million, about 160 million, about 170 million, about 180 million, about 290 million, about 200 million or more than 200 million cells. In some embodiments, this allows the treatment of about 100,000, about 110,000, about 120,000, about 130,000, about 140,000, about 150,000, about 160,000, about 170,000, about 180,000, about 190,000, about 200,000, about 210,000, about 220,000, about 230,000, about 240,000, about 250,000, about 260,000, about 270,000, about 280,000, about 290,000, about or 300,000 patients. In some embodiments, this allows the treatment of at least about 100,000, about 110,000, about 120,000, about 130,000, about 140,000, about 150,000, about 160,000, about 170,000, about 180,000, about 190,000, about 200,000, about 210,000, about 220,000, about 230,000, about 240,000, about 250,000, about 260,000, about 270,000, about 280,000, about 290,000, about or 300,000 patients.

### Pre-Cryopreservation Processing

In some embodiments, the MSCs are processed prior cryopreservation. Recent work evaluating proliferating cell lines has pointed to DNA defects and alterations of higher-order chromatin structure of frozen and thawed cells with and without cryoprotectant treatment. These studies pointed out that that in replicating (S phase) cells, DNA was preferentially damaged by replication fork collapse, potentially leading to DNA double strand breaks (DSBs), which represent an important source of both genome instability and defects in epigenome maintenance and can lead to apoptosis. Such DNA damage could be the primary driver of the delayed onset death which was observed in post thaw culture. Unmanipulated, non-synchronized, exponentially growing MSC and demonstrated that 20-30% of the cells were in S phase.

An aspect of the present disclosure is a method for preparing a population of isolated MSCs for pharmaceutical application, the method comprising: providing a population of isolated MSCs; and synchronizing the population of isolated MSCs, wherein no more than 25% of the population of isolated MSCs are in the S phase of the cell cycle. Another aspect of the present disclosure is a method for storing a population of isolated MSCs, the method comprising: providing a population of isolated MSCs; and synchronizing the population of isolated MSCs, wherein no more than 25% of the population of isolated MSCs are in the S phase of the cell cycle. In some embodiments, the synchronizing comprises the serum starvation methods described herein. In some embodiments, the synchronizing comprises the methods of γMSCs described herein.

In some embodiments, no more than 5% of the population of isolated MSCs are in the S phase of the cell cycle. In some embodiments, no more than 2% of the population of isolated MSCs are in the S phase of the cell cycle. In some embodiments, no more than about 0.1% of the population of isolated MSCs are in the S phase of the cell cycle to about 5% of the population of isolated MSCs are in the S phase of the cell cycle. In some embodiments, no more than about 5% of the population of isolated MSCs are in the S phase of the cell cycle to about 4% of the population of isolated MSCs are in the S phase of the cell cycle, about 5% of the population of isolated MSCs are in the S phase of the cell cycle to about 3% of the population of isolated MSCs are in the S phase of the cell cycle, about 5% of the population of isolated MSCs are in the S phase of the cell cycle to about 2% of the population of isolated MSCs are in the S phase of the cell cycle, about 5% of the population of isolated MSCs are in the S phase of the cell cycle to about 1% of the population of isolated MSCs are in the S phase of the cell cycle, about 5% of the population of isolated MSCs are in the S phase of the cell cycle to about 0.8% of the population of isolated MSCs are in the S phase of the cell cycle, about 5% of the population of isolated MSCs are in the S phase of the cell cycle to about 0.5% of the population of isolated MSCs are in the S phase of the cell cycle, about 5% of the population of isolated MSCs are in the S phase of the cell cycle to about 0.3% of the population of isolated MSCs are in the S phase of the cell cycle, about 5% of the population of isolated MSCs are in the S phase of the cell cycle to about 0.2% of the population of isolated MSCs are in the S phase of the cell cycle, about 5% of the population of isolated MSCs are in the S phase of the cell cycle to about 0.1% of the population of isolated MSCs are in the S phase of the cell cycle, about 4% of the population of isolated MSCs are in the S phase of the cell cycle to about 3% of the population of isolated MSCs are in the S phase of the cell cycle, about 4% of the population of isolated MSCs are in the S phase of the cell cycle to about 2% of the population of isolated MSCs are in the S phase of the cell cycle, about 4% of the population of isolated MSCs are in the S phase of the cell cycle to about 1% of the population of isolated MSCs are in the S phase of the cell cycle, about 4% of the population of isolated MSCs are in the S phase of the cell cycle to about 0.8% of the population of isolated MSCs are in the S phase of the cell cycle, about 4% of the population of isolated MSCs are in the S phase of the cell cycle to about 0.5% of the population of isolated MSCs are in the S phase of the cell cycle, about 4% of the population of isolated MSCs are in the S phase of the cell cycle to about 0.3% of the population of isolated MSCs are in the S phase of the cell cycle, about 4% of the population of isolated MSCs are in the S phase of the cell cycle to about 0.2% of the population of isolated MSCs are in the S phase of the cell cycle, about 4% of the population of isolated MSCs are in the S phase of the cell cycle to about 0.1% of the population of isolated MSCs are in the S phase of the cell cycle, about 3% of the population of isolated MSCs are in the S phase of the cell cycle to about 2% of the population of isolated MSCs are in the S phase of the cell cycle, about 3% of the population of isolated MSCs are in the S phase of the cell cycle to about 1% of the population of isolated MSCs are in the S phase of the cell cycle, about 3% of the population of isolated MSCs are in the S phase of the cell cycle to about 0.8% of the population of isolated MSCs are in the S phase of the cell cycle, about 3% of the population of isolated MSCs are in the S phase of the cell cycle to about 0.5% of the population of isolated MSCs are in the S phase of the cell cycle, about 3% of the population of isolated MSCs are in the S phase of the cell cycle to about 0.3% of the population of isolated MSCs are in the S phase of the cell cycle, about 3% of the population of isolated MSCs are in the S phase of the cell cycle to about 0.2% of the population of isolated MSCs are in the S phase of the cell cycle, about 3% of the population of isolated MSCs are in the S phase of the cell cycle to about 0.1% of the population of isolated MSCs are in the S phase of the cell cycle, about 2% of the population of isolated MSCs are in the S phase of the cell cycle to about 1% of the population of isolated MSCs are in the S phase of the cell cycle, about 2% of the population of isolated MSCs are in the S phase of the cell cycle to about 0.8% of the population of isolated MSCs are in the S phase of the cell cycle, about 2% of the population of isolated MSCs are in the S phase of the cell cycle to about 0.5% of the population of isolated MSCs are in the S phase of the cell cycle, about 2% of the population of isolated MSCs are in the S phase of the cell cycle to about 0.3% of the population of isolated MSCs are in the S phase of the cell cycle, about 2% of the population of isolated MSCs are in the S phase of the cell cycle to about 0.2% of the population of isolated MSCs are in the S phase of the cell cycle, about 2% of the population of isolated MSCs are in the S phase of the cell cycle to about 0.1% of the population of isolated MSCs are in the S phase of the cell cycle, about 1% of the population of isolated MSCs are in the S phase of the cell cycle to about 0.8% of the population of isolated MSCs are in the S phase of the cell cycle, about 1% of the population of isolated MSCs are in the S phase of the cell cycle to about 0.5% of the population of isolated MSCs are in the S phase of the cell cycle, about 1% of the population of isolated MSCs are in the S phase of the cell cycle to about 0.3% of the population of isolated MSCs are in the S phase of the cell cycle, about 1% of the population of isolated MSCs are in the S phase of the cell cycle to about 0.2% of the population of isolated MSCs are in the S phase of the cell cycle, about 1% of the population of isolated MSCs are in the S phase of the cell cycle to about 0.1% of the population of isolated MSCs are in the S phase of the cell cycle, about 0.8% of the population of isolated MSCs are in the S phase of the cell cycle to about 0.5% of the population of isolated MSCs are in the S phase of the cell cycle, about 0.8% of the population of isolated MSCs are in the S phase of the cell cycle to about 0.3% of the population of isolated MSCs are in the S phase of the cell cycle, about 0.8% of the population of isolated MSCs are in the S phase of the cell cycle to about 0.2% of the population of isolated MSCs are in the S phase of the cell cycle, about 0.8% of the population of isolated MSCs are in the S phase of the cell cycle to about 0.1% of the population of isolated MSCs are in the S phase of the cell cycle, about 0.5% of the population of isolated MSCs are in the S phase of the cell cycle to about 0.3% of the population of isolated MSCs are in the S phase of the cell cycle, about 0.5% of the population of isolated MSCs are in the S phase of the cell cycle to about 0.2% of the population of isolated MSCs are in the S phase of the cell cycle, about 0.5% of the population of isolated MSCs are in the S phase of the cell cycle to about 0.1% of the population of isolated MSCs are in the S phase of the cell cycle, about 0.3% of the population of isolated MSCs are in the S phase of the cell cycle to about 0.2% of the population of isolated MSCs are in the S phase of the cell cycle, about 0.3% of the population of isolated MSCs are in the S phase of the cell cycle to about 0.1% of the population of isolated MSCs are in the S phase of the cell cycle, or about 0.2% of the population of isolated MSCs are in the S phase of the cell cycle to about 0.1% of the population of isolated MSCs are in the S phase of the cell cycle. In some embodiments, no more than about 5% of the population of isolated MSCs are in the S phase of the cell cycle, about 4% of the population of isolated MSCs are in the S phase of the cell cycle, about 3% of the population of isolated MSCs are in the S phase of the cell cycle, about 2% of the population of isolated MSCs are in the S phase of the cell cycle, about 1% of the population of isolated MSCs are in the S phase of the cell cycle, about 0.8% of the population of isolated MSCs are in the S phase of the cell cycle, about 0.5% of the population of isolated MSCs are in the S phase of the cell cycle, about 0.3% of the population of isolated MSCs are in the S phase of the cell cycle, about 0.2% of the population of isolated MSCs are in the S phase of the cell cycle, or about 0.1% of the population of isolated MSCs are in the S phase of the cell cycle. In some embodiments, no more than at least about 5% of the population of isolated MSCs are in the S phase of the cell cycle, about 4% of the population of isolated MSCs are in the S phase of the cell cycle, about 3% of the population of isolated MSCs are in the S phase of the cell cycle, about 2% of the population of isolated MSCs are in the S phase of the cell cycle, about 1% of the population of isolated MSCs are in the S phase of the cell cycle, about 0.8% of the population of isolated MSCs are in the S phase of the cell cycle, about 0.5% of the population of isolated MSCs are in the S phase of the cell cycle, about 0.3% of the population of isolated MSCs are in the S phase of the cell cycle, or about 0.2% of the population of isolated MSCs are in the S phase of the cell cycle. In some embodiments, no more than at most about 4% of the population of isolated MSCs are in the S phase of the cell cycle, about 3% of the population of isolated MSCs are in the S phase of the cell cycle, about 2% of the population of isolated MSCs are in the S phase of the cell cycle, about 1% of the population of isolated MSCs are in the S phase of the cell cycle, about 0.8% of the population of isolated MSCs are in the S phase of the cell cycle, about 0.5% of the population of isolated MSCs are in the S phase of the cell cycle, about 0.3% of the population of isolated MSCs are in the S phase of the cell cycle, about 0.2% of the population of isolated MSCs are in the S phase of the cell cycle, or about 0.1% of the population of isolated MSCs are in the S phase of the cell cycle.

In some embodiments, at least 80% of the population of isolated MSCs express CD73, CD90, CD105, or any combination thereof. The method of any one of the embodiments disclosed herein, wherein at least 90% of the population of isolated MSCs express CD73, CD90, CD105, or any combination thereof. In some embodiments, at least 80% of the population of isolated MSCs maintain responsiveness to interferon gamma (IFNγ) stimulation. In some embodiments, at least 90% of the population of isolated MSCs maintain responsiveness to interferon gamma (IFNγ) stimulation. In some embodiments, at least 80% of the population of isolated MSCs express PD-L1, HLA-DR, or both, when stimulated with IFNγ. In some embodiments, at least 90% of the population of isolated MSCs express PD-L1, HLA-DR, or both, when stimulated with IFNγ. In some embodiments, at least 80% of the population of isolated MSCs overexpress PD-L1, HLA-DR, or both, when stimulated with IFNγ, relative to an unstimulated MSC. In some embodiments, at least 90% of the population of isolated MSCs overexpress PD-L1, HLA-DR, or both, when stimulated with IFNγ, relative to an unstimulated MSC. In some embodiments, at least 80% of the population of isolated MSCs overexpress Indoleamine 2,3-dioxygenase (IDO), when stimulated with IFNγ, relative to an unstimulated MSC. In some embodiments, at least 80% of the population of isolated MSCs overexpress Indoleamine 2,3-dioxygenase (IDO), when stimulated with IFNγ, relative to an unstimulated MSC. In some embodiments, synchronizing comprises serum starving the population of isolated MSCs for a period of time prior to exposing the population of isolated MSCs to a freezing temperature. In some embodiments, serum starving comprises culturing the population of MSCs in media comprising less than 1% serum. In some embodiments, serum starving comprises culturing the population of MSCs in media comprising less than 0.5% serum. In some embodiments, the serum is human platelet lysate (hPL). In some embodiments, the serum is human serum albumin (HSA). In some embodiments, serum starving comprises culturing the population of MSCs in serum free media or in a medium having reduced amounts of serum. In some embodiments, the period of time prior to exposing the population of isolated MSCs to a freezing temperature is less than 72 hours. In some embodiments, the period of time prior to exposing the population of isolated MSCs to a freezing temperature is less than 48 hours. In some embodiments, the period of time prior to exposing the population of isolated MSCs to a freezing temperature is 24 hours. In some embodiments, the period of time prior to exposing the population of isolated MSCs to a freezing temperature is less than 24 hours. In various embodiments, the period of time is sufficient to synchronized at least 75% of the MSCs in the population into S phase of the cell cycle.

In some embodiments, no more than 6% of the population of isolated MSCs undergo early apoptosis during the serum starvation. In some embodiments, no more than 2% of the population of isolated MSCs undergo early apoptosis during the serum starvation. In some embodiments, no more than about 0.1% of the population of isolated MSCs undergo early apoptosis during the serum starvation to about 2% of the population of isolated MSCs undergo early apoptosis during the serum starvation. In some embodiments, no more than about 2% of the population of isolated MSCs undergo early apoptosis during the serum starvation to about 1.5% of the population of isolated MSCs undergo early apoptosis during the serum starvation, about 2% of the population of isolated MSCs undergo early apoptosis during the serum starvation to about 1% of the population of isolated MSCs undergo early apoptosis during the serum starvation, about 2% of the population of isolated MSCs undergo early apoptosis during the serum starvation to about 0.5% of the population of isolated MSCs undergo early apoptosis during the serum starvation, about 2% of the population of isolated MSCs undergo early apoptosis during the serum starvation to about 0.3% of the population of isolated MSCs undergo early apoptosis during the serum starvation, about 2% of the population of isolated MSCs undergo early apoptosis during the serum starvation to about 0.2% of the population of isolated MSCs undergo early apoptosis during the serum starvation, about 2% of the population of isolated MSCs undergo early apoptosis during the serum starvation to about 0.1% of the population of isolated MSCs undergo early apoptosis during the serum starvation, about 1.5% of the population of isolated MSCs undergo early apoptosis during the serum starvation to about 1% of the population of isolated MSCs undergo early apoptosis during the serum starvation, about 1.5% of the population of isolated MSCs undergo early apoptosis during the serum starvation to about 0.5% of the population of isolated MSCs undergo early apoptosis during the serum starvation, about 1.5% of the population of isolated MSCs undergo early apoptosis during the serum starvation to about 0.3% of the population of isolated MSCs undergo early apoptosis during the serum starvation, about 1.5% of the population of isolated MSCs undergo early apoptosis during the serum starvation to about 0.2% of the population of isolated MSCs undergo early apoptosis during the serum starvation, about 1.5% of the population of isolated MSCs undergo early apoptosis during the serum starvation to about 0.1% of the population of isolated MSCs undergo early apoptosis during the serum starvation, about 1% of the population of isolated MSCs undergo early apoptosis during the serum starvation to about 0.5% of the population of isolated MSCs undergo early apoptosis during the serum starvation, about 1% of the population of isolated MSCs undergo early apoptosis during the serum starvation to about 0.3% of the population of isolated MSCs undergo early apoptosis during the serum starvation, about 1% of the population of isolated MSCs undergo early apoptosis during the serum starvation to about 0.2% of the population of isolated MSCs undergo early apoptosis during the serum starvation, about 1% of the population of isolated MSCs undergo early apoptosis during the serum starvation to about 0.1% of the population of isolated MSCs undergo early apoptosis during the serum starvation, about 0.5% of the population of isolated MSCs undergo early apoptosis during the serum starvation to about 0.3% of the population of isolated MSCs undergo early apoptosis during the serum starvation, about 0.5% of the population of isolated MSCs undergo early apoptosis during the serum starvation to about 0.2% of the population of isolated MSCs undergo early apoptosis during the serum starvation, about 0.5% of the population of isolated MSCs undergo early apoptosis during the serum starvation to about 0.1% of the population of isolated MSCs undergo early apoptosis during the serum starvation, about 0.3% of the population of isolated MSCs undergo early apoptosis during the serum starvation to about 0.2% of the population of isolated MSCs undergo early apoptosis during the serum starvation, about 0.3% of the population of isolated MSCs undergo early apoptosis during the serum starvation to about 0.1% of the population of isolated MSCs undergo early apoptosis during the serum starvation, or about 0.2% of the population of isolated MSCs undergo early apoptosis during the serum starvation to about 0.1% of the population of isolated MSCs undergo early apoptosis during the serum starvation. In some embodiments, no more than about 2% of the population of isolated MSCs undergo early apoptosis during the serum starvation, about 1.5% of the population of isolated MSCs undergo early apoptosis during the serum starvation, about 1% of the population of isolated MSCs undergo early apoptosis during the serum starvation, about 0.5% of the population of isolated MSCs undergo early apoptosis during the serum starvation, about 0.3% of the population of isolated MSCs undergo early apoptosis during the serum starvation, about 0.2% of the population of isolated MSCs undergo early apoptosis during the serum starvation, or about 0.1% of the population of isolated MSCs undergo early apoptosis during the serum starvation. In some embodiments, no more than at least about 2% of the population of isolated MSCs undergo early apoptosis during the serum starvation, about 1.5% of the population of isolated MSCs undergo early apoptosis during the serum starvation, about 1% of the population of isolated MSCs undergo early apoptosis during the serum starvation, about 0.5% of the population of isolated MSCs undergo early apoptosis during the serum starvation, about 0.3% of the population of isolated MSCs undergo early apoptosis during the serum starvation, or about 0.2% of the population of isolated MSCs undergo early apoptosis during the serum starvation. In some embodiments, no more than at most about 1.5% of the population of isolated MSCs undergo early apoptosis during the serum starvation, about 1% of the population of isolated MSCs undergo early apoptosis during the serum starvation, about 0.5% of the population of isolated MSCs undergo early apoptosis during the serum starvation, about 0.3% of the population of isolated MSCs undergo early apoptosis during the serum starvation, about 0.2% of the population of isolated MSCs undergo early apoptosis during the serum starvation, or about 0.1% of the population of isolated MSCs undergo early apoptosis during the serum starvation.

In some embodiments, the method further comprising stimulating the population of isolated MSCs with IFNγ. In some embodiments, the method further comprising stimulating the population of isolated MSCs with IFNγ contemporaneously with the serum starving. In some embodiments, the population of isolated MSCs are vertebral bone adherent MSCs (vBA-MSCs). In some embodiments, the population of isolated MSCs are vertebral bone marrow derived MSCs (vBM-MSCs). In some embodiments, the population of isolated MSCs are cadaveric MSCs.

Another aspect of the present disclosure is a composition comprising a population of isolated mesenchymal stromal cells (MSCs), wherein no more than 25% of the population of isolated MSCs are in the S phase of the cell cycle. In some embodiments, no more than 5% of the population of isolated MSCs are in the S phase of the cell cycle. In some embodiments, no more than 2% of the population of isolated MSCs are in the S phase of the cell cycle. In some embodiments, at least 80% of the population of isolated MSCs express CD73, CD90, CD105, or any combination thereof. The composition of any one of the embodiments disclosed herein, wherein at least 90% of the population of isolated MSCs express CD73, CD90, CD105, or any combination thereof. In some embodiments, at least 80% of the population of isolated MSCs maintain responsiveness to interferon gamma (IFNγ) stimulation. In some embodiments, at least 90% of the population of isolated MSCs maintain responsiveness to interferon gamma (IFNγ) stimulation. In some embodiments, at least 80% of the population of isolated MSCs express PD-L1, HLA-DR, or both, when stimulated with IFNγ. In some embodiments, at least 90% of the population of isolated MSCs express PD-L1, HLA-DR, or both, when stimulated with IFNγ. In some embodiments, at least 80% of the population of isolated MSCs overexpress PD-L1, HLA-DR, or both, when stimulated with IFNγ, relative to an unstimulated MSC. In some embodiments, at least 90% of the population of isolated MSCs overexpress PD-L1, HLA-DR, or both, when stimulated with IFNγ, relative to an unstimulated MSC. In some embodiments, at least 80% of the population of isolated MSCs overexpress Indoleamine 2,3-dioxygenase (IDO), when stimulated with IFNγ, relative to an unstimulated MSC. In some embodiments, at least 80% of the population of isolated MSCs overexpress Indoleamine 2,3-dioxygenase (IDO), when stimulated with IFNγ, relative to an unstimulated MSC. In some embodiments, the population of isolated MSCs are vertebral bone adherent MSCs (vBA-MSCs). In some embodiments, the population of isolated MSCs are vertebral bone marrow derived MSCs (vBM-MSCs). In some embodiments, the population of isolated MSCs are cadaveric MSCs. A pharmaceutical composition comprising: the population of isolated MSCs from any one of the embodiments disclosed herein; and a pharmaceutically acceptable excipient.

### Cryorecovery

The cryopreserved MSCs may be thawed and undergo a second expansion. In some embodiments, MSCs and compositions comprising the same are provided to an end user (e.g., treatment facility) in a condition where they can immediately be used (i.e., injected into a subject) and with minimal processing. In practice, facilities where the use of MSCs would take place likely do not have personnel trained in MSC sample prep. Therefore, there is a need for methods and systems of MSC sample preparation for immediate injection by the end user and where the MSCs do not require further processing steps upon arrival to the treatment facility.

The methods and systems disclosed herein comprise sample preparation of MSCs to including thawing cryopreserved MSCs and maintaining the MSCs under specific conditions (e.g., specific temperature(s)) for a period of time. The methods and systems disclosed herein also comprise sample preparation of MSCs including thawing cryopreserved MSCs, maintaining the MSCs for a period of time under specific conditions (e.g., a specific/first temperature), and then maintaining the MSCs under different conditions (e.g., at a different temperature than the temperature under which the MSCs were maintained immediately post-thaw). In some embodiments, this change in temperature is a cooling. In some embodiments, the MSCs are maintained in hypothermic conditions post-thaw until direct infusion into a subject.

Provided herein, in some embodiments, is a method of warming a cryopreserved population of stem cells to a first temperature and storing said stem cells at a second temperature less than about 40° C. In some embodiments, the first temperature that the cryopreserved population of stem cells is warmed to is greater than about 0° C. In some embodiments, the first temperature is greater than about 20° C. In some embodiments, the second temperature that the stem cells are stored at is a hypothermic temperature.

In some embodiments, a frozen cryovial comprising cryopreserved cells (e.g., P3 cells) are placed into a 37° C. water bath. The vial will be kept in the water bath until approximately 80% of ice has melted (which takes about two to three minutes). A cryovial is then be sprayed with sterile 70% ethanol (EtOH), wiped with sterile wipes and transferred into a biosafety cabinet. Cells in the thawed vial can then transferred to a sterile conical tube, the cells diluted with PLASMA-LYTE A+0.5% HSA, centrifuged (e.g., at room temperature) to form a cell pellet, and the supernatant is removed, and the pellet is resuspended in a culturing medium, e.g., MSC Culture Media, for further plating and culturing, e.g., as Passage 4 (P4).

Provided herein, in one aspect, is a method for preparing stem cells for infusion, the method comprising: (a) providing a cryopreserved population of cells comprising said stem cells; (b) warming said stem cells to a first temperature and holding said stem cells at said first temperature for a first period of time; and (c) changing said first temperature to a second temperature and maintaining said stem cells at said second temperature for a second period of time.

In some embodiments, said first temperature is greater than about 0° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C. In some embodiments, said first temperature is about 0° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C. In some embodiments, said first temperature is greater than 0° C.

In some embodiments, said second temperature is less than about 0° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C. In some embodiments, said second temperature is about 0° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C. In some embodiments, said second temperature is hypothermic. In some embodiments, said second temperature is less than 40° C.

In some embodiments, said first time period is less than about one week. In some embodiments, said time period is less than about 7 days, about 6 days, about 5 days, about 4 days, about 3 days, about 2 days, or about 1 day. In some embodiments, said time period is less than about 5 days. In some embodiments, said time period is less than about 2 days. In some embodiments, said time period is less than about 1 day. In some embodiments, said time period is less than about 24 hours, about 23 hours, about 22 hours, about 21 hours, about 20 hours, about 19 hours, about 18 hours, about 17 hours, about 16 hours, about 15 hours, about 14 hours, about 13 hours, about 12 hours, about 11 hours, about 10 hours, about 9 hours, about 8 hours, about 7 hours, about 6 hours, about 5 hours, about 4 hours, about 3 hours, about 2 hours, or about 1 hour. In some embodiments, said time period is less than about 12 hours. In some embodiments, said time period is less than about 6 hours. In some embodiments, said time period is less than about 2 hours. In some embodiments, said time period is less than about 60 minutes, about 59 minutes, about 58 minutes, about 57 minutes, about 56 minutes, about 55 minutes, about 54 minutes, about 53 minutes, about 52 minutes, about 51 minutes, about 50 minutes, about 49 minutes, about 48 minutes, about 47 minutes, about 46 minutes, about 45 minutes, about 44 minutes, about 43 minutes, about 42 minutes, about 41 minutes, about 40 minutes, about 39 minutes, about 38 minutes, about 37 minutes, about 36 minutes, about 35 minutes, about 34 minutes, about 33 minutes, about 32 minutes, about 31 minutes, about 30 minutes, about 29 minutes, about 28 minutes, about 27 minutes, about 26 minutes, about 25 minutes, about 24 minutes, about 23 minutes, about 22 minutes, about 21 minutes, about 20 minutes, about 19 minutes, about 18 minutes, about 17 minutes, about 16 minutes, about 15 minutes, about 14 minutes, about 13 minutes, about 12 minutes, about 11 minutes, about 10 minutes, about 9 minutes, about 8 minutes, about 7 minutes, about 6 minutes, about 5 minutes, about 4 minutes, about 3 minutes, about 2 minutes, or about 1 minute.

In some embodiments, the MSCs are maintained after they are warmed/thawed. In some embodiments, this maintenance is not accounted for in the first period of time described herein. In some embodiments, this maintenance is accounted for in the first period of time. In some embodiments, this maintenance follows similar methods as the MSC culturing methods described herein. In some embodiments, the post-thaw maintenance methods comprise not allowing the MSCs to double in population. In some embodiments, the MSCs are recovered and/or packaged prior to doubling. In some embodiments, the post-thaw culturing maintenance methods comprise culturing plating said stem cells at about 500 cells/cm² to about 4,000 cells/cm². In some embodiments, the post-thaw culturing maintenance methods comprise culturing plating said stem cells at about 500 cells/cm² to about 1,000 cells/cm², about 500 cells/cm² to about 1,500 cells/cm², about 500 cells/cm² to about 2,000 cells/cm², about 500 cells/cm² to about 2,500 cells/cm², about 500 cells/cm² to about 3,000 cells/cm², about 500 cells/cm² to about 3,500 cells/cm², about 500 cells/cm² to about 4,000 cells/cm², about 1,000 cells/cm² to about 1,500 cells/cm², about 1,000 cells/cm² to about 2,000 cells/cm², about 1,000 cells/cm² to about 2,500 cells/cm², about 1,000 cells/cm² to about 3,000 cells/cm², about 1,000 cells/cm² to about 3,500 cells/cm², about 1,000 cells/cm² to about 4,000 cells/cm², about 1,500 cells/cm² to about 2,000 cells/cm², about 1,500 cells/cm² to about 2,500 cells/cm², about 1,500 cells/cm² to about 3,000 cells/cm², about 1,500 cells/cm² to about 3,500 cells/cm², about 1,500 cells/cm² to about 4,000 cells/cm², about 2,000 cells/cm² to about 2,500 cells/cm², about 2,000 cells/cm² to about 3,000 cells/cm², about 2,000 cells/cm² to about 3,500 cells/cm², about 2,000 cells/cm² to about 4,000 cells/cm², about 2,500 cells/cm² to about 3,000 cells/cm², about 2,500 cells/cm² to about 3,500 cells/cm², about 2,500 cells/cm² to about 4,000 cells/cm², about 3,000 cells/cm² to about 3,500 cells/cm², about 3,000 cells/cm² to about 4,000 cells/cm², or about 3,500 cells/cm² to about 4,000 cells/cm². In some embodiments, the post-thaw culturing maintenance methods comprise culturing plating said stem cells at about 500 cells/cm², about 1,000 cells/cm², about 1,500 cells/cm², about 2,000 cells/cm², about 2,500 cells/cm², about 3,000 cells/cm², about 3,500 cells/cm², or about 4,000 cells/cm². In some embodiments, the post-thaw culturing maintenance methods comprise culturing plating said stem cells at least about 500 cells/cm², about 1,000 cells/cm², about 1,500 cells/cm², about 2,000 cells/cm², about 2,500 cells/cm², about 3,000 cells/cm², or about 3,500 cells/cm². In some embodiments, the post-thaw culturing maintenance methods comprise culturing plating said stem cells at most about 1,000 cells/cm², about 1,500 cells/cm², about 2,000 cells/cm², about 2,500 cells/cm², about 3,000 cells/cm², about 3,500 cells/cm², or about 4,000 cells/cm². In some embodiments, the post-thaw maintenance methods comprise plating said stem cells at about 3,000 to about 10,000, about 3,000 to about 9,000, about 3,000 to about 8,000 about, about 3,000 to about 7,000, about 3,000 to about 6,000, about 3,000 to about 5,000, about 3,000 to about 4,000, about 10,000 to about 50,000, about 10,000 to about 40,000, about 10,000 to about 30,000, about 10,000 to about 20,000, about 20,000 to about 50,000, about 20,000 to about 40,000, about 20,000 to about 30,000, about 30,000 to about 50,000, about 30,000 to about 40,000, or about 40,000 to about 50,000 cells/cm² at said first temperature. In some embodiments, for a method provided herein, the method further comprises, prior to (c), maintaining said stem cells at about 3,000 cells/cm² to about 50,000 cells/cm² at said first temperature. In some embodiments, the post-thaw culturing maintenance methods comprise culturing plating said stem cells at about 10,000 cells/cm² to about 50,000 cells/cm². In some embodiments, the post-thaw culturing maintenance methods comprise culturing plating said stem cells at about 10,000 cells/cm² to about 15,000 cells/cm², about 10,000 cells/cm² to about 20,000 cells/cm², about 10,000 cells/cm² to about 25,000 cells/cm², about 10,000 cells/cm² to about 30,000 cells/cm², about 10,000 cells/cm² to about 35,000 cells/cm², about 10,000 cells/cm² to about 40,000 cells/cm², about 10,000 cells/cm² to about 45,000 cells/cm², about 10,000 cells/cm² to about 50,000 cells/cm², about 15,000 cells/cm² to about 20,000 cells/cm², about 15,000 cells/cm² to about 25,000 cells/cm², about 15,000 cells/cm² to about 30,000 cells/cm², about 15,000 cells/cm² to about 35,000 cells/cm², about 15,000 cells/cm² to about 40,000 cells/cm², about 15,000 cells/cm² to about 45,000 cells/cm², about 15,000 cells/cm² to about 50,000 cells/cm², about 20,000 cells/cm² to about 25,000 cells/cm², about 20,000 cells/cm² to about 30,000 cells/cm², about 20,000 cells/cm² to about 35,000 cells/cm², about 20,000 cells/cm² to about 40,000 cells/cm², about 20,000 cells/cm² to about 45,000 cells/cm², about 20,000 cells/cm² to about 50,000 cells/cm², about 25,000 cells/cm² to about 30,000 cells/cm², about 25,000 cells/cm² to about 35,000 cells/cm², about 25,000 cells/cm² to about 40,000 cells/cm², about 25,000 cells/cm² to about 45,000 cells/cm², about 25,000 cells/cm² to about 50,000 cells/cm², about 30,000 cells/cm² to about 35,000 cells/cm², about 30,000 cells/cm² to about 40,000 cells/cm², about 30,000 cells/cm² to about 45,000 cells/cm², about 30,000 cells/cm² to about 50,000 cells/cm², about 35,000 cells/cm² to about 40,000 cells/cm², about 35,000 cells/cm² to about 45,000 cells/cm², about 35,000 cells/cm² to about 50,000 cells/cm², about 40,000 cells/cm² to about 45,000 cells/cm², about 40,000 cells/cm² to about S0,000 cells/cm², or about 45,000 cells/cm² to about 50,000 cells/cm². In some embodiments, the post-thaw culturing maintenance methods comprise culturing plating said stem cells at about 10,000 cells/cm², about 15,000 cells/cm², about 20,000 cells/cm², about 25,000 cells/cm², about 30,000 cells/cm², about 35,000 cells/cm², about 40,000 cells/cm², about 45,000 cells/cm², or about 50,000 cells/cm². In some embodiments, the post-thaw culturing maintenance methods comprise culturing plating said stem cells at least about 10,000 cells/cm², about 15,000 cells/cm², about 20,000 cells/cm², about 25,000 cells/cm², about 30,000 cells/cm², about 35,000 cells/cm², about 40,000 cells/cm², or about 45,000 cells/cm². In some embodiments, the post-thaw culturing maintenance methods comprise culturing plating said stem cells at most about 15,000 cells/cm², about 20,000 cells/cm², about 25,000 cells/cm², about 30,000 cells/cm², about 35,000 cells/cm², about 40,000 cells/cm², about 45,000 cells/cm², or about 50,000 cells/cm². In some embodiments, for a method provided herein, the method further comprises, prior to (c), maintaining said stem cells at about 3,000, about 4,000, about 5,000, about 6,000, about 7,000, about 8,000, about 9,000, 10,000, about 20,000, about 30,000, about 40,000, or about 50,000 cells/cm² at said first temperature. In some embodiments, for a method provided herein, the method further comprises, prior to (c), maintaining said stem cells at about 32,000 cells/cm² at said first temperature. In some embodiments, the post-thaw maintaining takes place in a T25 flask. In some embodiments, the post-thaw maintaining takes place in a T75 flask. In some embodiments, the post-thaw maintaining takes place in another sized flasks.

In some embodiments, the post-thaw culturing maintenance methods increases the number of cells in a culture vessel from about 5% to about 500%. As examples, the post-thaw culturing maintenance methods may increase the number of cells in a culture vessel by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 120%, about 140%, about 160%, about 180%, about 200%, about 220%, about 240%, about 260%, about 280%, about 300%, about 320%, about 340%, about 360%, about 380%, about 400%, about 420%, about 440%, about 460%, about 480%, or about 500%. The post-thaw culturing maintenance methods may increase the number of cells in a culture vessel by about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, about 95% to about 100%, about 100% to about 200%, about 200% to about 300%, about 300% to about 400%, or about 400% to about 500%.

In some embodiments, the stem cells are packaged in 5 mL volumes of 10×10⁶ live cells/ml in PLASMA-LYTE A+2.5% HSA (Rinse Media). In some embodiments, the stem cells are packaged in volumes of about 1×10⁶ live cells/ml to about 12×10⁶ live cells/ml. In some embodiments, the stem cells are packaged in volumes of about 1×10⁶ live cells/ml to about 2×10⁶ live cells/ml, about 1×10⁶ live cells/ml to about 3×10⁶ live cells/ml, about 1×10⁶ live cells/ml to about 4×10⁶ live cells/ml, about 1×10⁶ live cells/ml to about 5×10⁶ live cells/ml, about 1×10⁶ live cells/ml to about 6×10⁶ live cells/ml, about 1×10⁶ live cells/ml to about 7×10⁶ live cells/ml, about 1×10⁶ live cells/ml to about 8×10⁶ live cells/ml, about 1×10⁶ live cells/ml to about 9×10⁶ live cells/ml, about 1×10⁶ live cells/ml to about 10×10⁶ live cells/ml, about 1×10⁶ live cells/ml to about 11×10⁶ live cells/ml, about 1×10⁶ live cells/ml to about 12×10⁶ live cells/ml, about 2×10⁶ live cells/ml to about 3×10⁶ live cells/ml, about 2×10⁶ live cells/ml to about 4×10⁶ live cells/ml, about 2×10⁶ live cells/ml to about 5×10⁶ live cells/ml, about 2×10⁶ live cells/ml to about 6×10⁶ live cells/ml, about 2×10⁶ live cells/ml to about 7×10⁶ live cells/ml, about 2×10⁶ live cells/ml to about 8×10⁶ live cells/ml, about 2×10⁶ live cells/ml to about 9×10⁶ live cells/ml, about 2×10⁶ live cells/ml to about 10×10⁶ live cells/ml, about 2×10⁶ live cells/ml to about 11×10⁶ live cells/ml, about 2×10⁶ live cells/ml to about 12×10⁶ live cells/ml, about 3×10⁶ live cells/ml to about 4×10⁶ live cells/ml, about 3×10⁶ live cells/ml to about 5×10⁶ live cells/ml, about 3×10⁶ live cells/ml to about 6×10⁶ live cells/ml, about 3×10⁶ live cells/ml to about 7×10⁶ live cells/ml, about 3×10⁶ live cells/ml to about 8×10⁶ live cells/ml, about 3×10⁶ live cells/ml to about 9×10⁶ live cells/ml, about 3×10⁶ live cells/ml to about 10×10⁶ live cells/ml, about 3×10⁶ live cells/ml to about 11×10⁶ live cells/ml, about 3×10⁶ live cells/ml to about 12×10⁶ live cells/ml, about 4×10⁶ live cells/ml to about 5×10⁶ live cells/ml, about 4×10⁶ live cells/ml to about 6×10⁶ live cells/ml, about 4×10⁶ live cells/ml to about 7×10⁶ live cells/ml, about 4×10⁶ live cells/ml to about 8×10⁶ live cells/ml, about 4×10⁶ live cells/ml to about 9×10⁶ live cells/ml, about 4×10⁶ live cells/ml to about 10×10⁶ live cells/ml, about 4×10⁶ live cells/ml to about 11×10⁶ live cells/ml, about 4×10⁶ live cells/ml to about 12×10⁶ live cells/ml, about 5×10⁶ live cells/ml to about 6×10⁶ live cells/ml, about 5×10⁶ live cells/ml to about 7×10⁶ live cells/ml, about 5×10⁶ live cells/ml to about 8×10⁶ live cells/ml, about 5×10⁶ live cells/ml to about 9×10⁶ live cells/ml, about 5×10⁶ live cells/ml to about 10×10⁶ live cells/ml, about 5×10⁶ live cells/ml to about 11×10⁶ live cells/ml, about 5×10⁶ live cells/ml to about 12×10⁶ live cells/ml, about 6×10⁶ live cells/ml to about 7×10⁶ live cells/ml, about 6×10⁶ live cells/ml to about 8×10⁶ live cells/ml, about 6×10⁶ live cells/ml to about 9×10⁶ live cells/ml, about 6×10⁶ live cells/ml to about 10×10⁶ live cells/ml, about 6×10⁶ live cells/ml to about 11×10⁶ live cells/ml, about 6×10⁶ live cells/ml to about 12×10⁶ live cells/ml, about 7×10⁶ live cells/ml to about 8×10⁶ live cells/ml, about 7×10⁶ live cells/ml to about 9×10⁶ live cells/ml, about 7×10⁶ live cells/ml to about 10×10⁶ live cells/ml, about 7×10⁶ live cells/ml to about 11×10⁶ live cells/ml, about 7×10⁶ live cells/ml to about 12×10⁶×10⁶ live cells/ml, about 8×10⁶ live cells/ml to about 9×10⁶ live cells/ml, about 8×10⁶ live cells/ml to about 10×10⁶ live cells/ml, about 8×10⁶ live cells/ml to about 11×10⁶ live cells/ml, about 8×10⁶ live cells/ml to about 12×10⁶ live cells/ml, about 9×10⁶ live cells/ml to about 10×10⁶ live cells/ml, about 9×10⁶ live cells/ml to about 11×10⁶ live cells/ml, about 9×10⁶ live cells/ml to about 12×10⁶ live cells/ml, about 10×10⁶ live cells/ml to about 11×10⁶ live cells/ml, about 10×10⁶ live cells/ml to about 12×10⁶ live cells/ml, or about 11×10⁶ live cells/ml to about 12×10⁶ live cells/ml. In some embodiments, the stem cells are packaged in volumes of about 1×10⁶ live cells/ml, about 2×10⁶ live cells/ml, about 3×10⁶ live cells/ml, about 4×10⁶ live cells/ml, about 5×10⁶ live cells/ml, about 6×10⁶ live cells/ml, about 7×10⁶ live cells/ml, about 8×10⁶ live cells/ml, about 9×10⁶ live cells/ml, about 10×10⁶ live cells/ml, about 11×10⁶ live cells/ml, or about 12×10⁶ live cells/ml. In some embodiments, the stem cells are packaged in volumes of at least about 1×10⁶ live cells/ml, about 2×10⁶ live cells/ml, about 3×10⁶ live cells/ml, about 4×10⁶ live cells/ml, about 5×10⁶ live cells/ml, about 6×10⁶ live cells/ml, about 7×10⁶ live cells/ml, about 8×10⁶ live cells/ml, about 9×10⁶ live cells/ml, about 10×10⁶ live cells/ml, or about 11×10⁶ live cells/ml. In some embodiments, the stem cells are packaged in volumes of at most about 2×10⁶ live cells/ml, about 3×10⁶ live cells/ml, about 4×10⁶ live cells/ml, about 5×10⁶ live cells/ml, about 6×10⁶ live cells/ml, about 7×10⁶ live cells/ml, about 8×10⁶ live cells/ml, about 9×10⁶ live cells/ml, about 10×10⁶ live cells/ml, about 11×10⁶ live cells/ml, or about 12×10⁶ live cells/ml.

Once the stem cells are packaged, the stem cells are put under the second temperature for the second time period. In some embodiments, the second temperature is less than about 40° C., about 39° C., about 38° C., about 37° C., about 36° C., about 35° C., about 34° C., about 33° C., about 32° C., about 31° C., about 30° C., about 29° C., about 28° C., about 27° C., about 26° C., about 25° C., about 24° C., about 23° C., about 22° C., about 21° C., about 20° C., about 19° C., about 18° C., about 17° C., about 16° C., about 15° C., about 14° C., about 13° C., about 12° C., about 11° C., about 10° C., about 9° C., about 8° C., about 7° C., about 6° C., about 5° C., about 4° C., about 3° C., about 2° C., or about 1° C. In some embodiments, said cell culture is maintained at about 40° C., about 39° C., about 38° C., about 37° C., about 36° C., about 35° C., about 34° C., about 33° C., about 32° C., about 31° C., about 30° C., about 29° C., about 28° C., about 27° C., about 26° C., about 25° C., about 24° C., about 23° C., about 22° C., about 21° C., about 20° C., about 19° C., about 18° C., about 17° C., about 16° C., about 15° C., about 14° C., about 13° C., about 12° C., about 11° C., about 10° C., about 9° C., about 8° C., about 7° C., about 6° C., about 5° C., about 4° C., about 3° C., about 2° C., or ab out 1° C. In some embodiments, the second temperature is less than 37° C. In some embodiments, the second temperature is less than 35° C. In some embodiments, the second temperature is less than 30° C. In some embodiments, the second temperature is less than 25° C. In some embodiments, said the second temperature is less than 20° C. In some embodiments, the second temperature is about 2° C. to about 8° C.

In some embodiments, said second time period is less than about one week. In some embodiments, said time period is less than about 7 days, about 6 days, about 5 days, about 4 days, about 3 days, about 2 days, or about 1 day. In some embodiments, said time period is less than about 5 days. In some embodiments, said time period is less than about 2 days. In some embodiments, said time period is less than about 1 day. In some embodiments, said time period is less than about 24 hours, about 23 hours, about 22 hours, about 21 hours, about 20 hours, about 19 hours, about 18 hours, about 17 hours, about 16 hours, about 15 hours, about 14 hours, about 13 hours, about 12 hours, about 11 hours, about 10 hours, about 9 hours, about 8 hours, about 7 hours, about 6 hours, about 5 hours, about 4 hours, about 3 hours, about 2 hours, or about 1 hour. In some embodiments, said time period is less than about 12 hours. In some embodiments, said time period is less than about 6 hours. In some embodiments, said time period is less than about 2 hours. In some embodiments, said time period is less than about 60 minutes, about 59 minutes, about 58 minutes, about 57 minutes, about 56 minutes, about 55 minutes, about 54 minutes, about 53 minutes, about 52 minutes, about 51 minutes, about 50 minutes, about 49 minutes, about 48 minutes, about 47 minutes, about 46 minutes, about 45 minutes, about 44 minutes, about 43 minutes, about 42 minutes, about 41 minutes, about 40 minutes, about 39 minutes, about 38 minutes, about 37 minutes, about 36 minutes, about 35 minutes, about 34 minutes, about 33 minutes, about 32 minutes, about 31 minutes, about 30 minutes, about 29 minutes, about 28 minutes, about 27 minutes, about 26 minutes, about 25 minutes, about 24 minutes, about 23 minutes, about 22 minutes, about 21 minutes, about 20 minutes, about 19 minutes, about 18 minutes, about 17 minutes, about 16 minutes, about 15 minutes, about 14 minutes, about 13 minutes, about 12 minutes, about 11 minutes, about 10 minutes, about 9 minutes, about 8 minutes, about 7 minutes, about 6 minutes, about 5 minutes, about 4 minutes, about 3 minutes, about 2 minutes, or about 1 minute.

### Methods of Manufacturing γMSCs

The present invention provides methods for manufacturing γMSCs, the method comprising obtaining a culture of MSCs and contacting the MSCs with IFNγ.

The MSCs may comprise vertebral bone marrow MSCs (vBM-MSCs), vertebral bone-adherent MSCs (vBA-MSCs), or both. The MSCs may be obtained from one or more vertebral bodies, and the vertebral bodies may be deceased humans.

MSCs may undergo a second expansion after cryopreservation. The second expansion may occur similarly to a first primary expansion, as described herein. The second expansion may also include additional components in the expansion medium. For example, the culture medium of the second expansion may comprise IFNγ. The IFNγ may be present in the expansion medium in order to prime the MSCs. Priming the MSCs with IFNγ creates γMSCs which may be used in human administration for the treatment of inflammatory conditions, including autoimmune conditions.

In some embodiments, P3 cells that have been cryfrozen are thawed or P3 that are detached from their culturing vessels are then further plated and cultured to form the Passage 4 (P4) cells. See, e.g., FIG. 11A to FIG. 11D.

P3 cells can be transferred to a new, sterile centrifuge tube and the cells may be diluted with about 2 volumes of PLASMA-LYTE A+0.5% HSA. The cells can be then centrifuged at about 500 RCF for 10 minutes at room temperature to form a cell pellet. The supernatant is removed, and the pellet is resuspended in a culturing medium, e.g., MSC Culture Media as described herein. Cell viability and count can be determined using Trypan Blue Exclusion via Manual Counting (or using AO/PI Method via automatic Cellometer). MSCs can be seeded at about 3,000-4,000 cells/cm² onto eight CellBIND® 5-chamber CellSTACKS® and placed into a 5% CO₂, 37° C., humidified tissue culture incubator. The plating step will be considered Day 0 and this cell culture step is considered Passage 4 (P4).

At Day 3, the MSC culture undergoes a media change to remove expended media and non-adherent cells. MSCs are then returned to the tissue culture incubator for two additional days. At Day 5, half of the media from a flask is removed and a sample for BACT/Alert (according to according to 21 CFR 610.12) is collected from this expended media from each flask. An aliquot (of about half of expended media that was removed) of culture media+50 ng/ml γ-IFN (R&D Systems, Minneapolis, USA) is made and added to each flask. The interferon gamma primed P4 cells are once again returned to the tissue culture incubator for two additional days. At Day 7, γ-MSCs cell density and morphology are inspected using a bright-field inverted microscope. γ-MSCs are harvested from culture via TrypLE™ Select, washed three times with phosphate buffered saline (PBS), and assessed for cell viability and count. Final product must meet release criteria as defined in Table 4.

Table 4 lists testing performed on the P4 cells and preferable features that γ-MSCs may possess.

TABLE 4
P4 γ-MSC Characterization

| Attribute | Test | Preferable Features |
|---|---|---|
| Expended Media | BacT/Alert according to 21 CFR | No Growth |
| Sterility from Day 5 | 610.12 | |
| Media Change | | |
| Viability and Cell Count | AO/PI via Cellometer or Trypan Blue | 85 ± 15%, and sufficient |
| | exclusion via Manual Count | number of cells for dose |
| | | (based on patient's ideal |
| | | body weight) |
| Rapid Final Cell Product | Gram Stain (Microscopy) | No organisms seen |
| Sterility | | |
| Mycoolasma | Lonza MycoAlert | Negative |
| Endotoxin | Charles River Endosafe ® PTS | <2.5 EU/ml |
| Final Cell Product | Flow Cytometry | CD105 positive (≥85%), |
| Identity | | CD45 negative (≤3%) |
| Final Cell Product | BacT/Alert according to 21 CFR | No Growth [will report |
| Sterility | 610.12 | after 14-days] |

The IFNγ may be present in the expansion medium at a concentration of about 100 U/ml to about 1000 U/ml. The IFNγ may be present in the expansion medium at a concentration of about 10 U/ml to about 500 U/ml, about 10 U/ml to about 1000 U/ml, about 10 U/ml to about 1500 U/ml, about 10 U/ml to about 2000 U/ml, about 50 U/ml to about 500 U/ml, about 50 U/ml to about 1000 U/ml, about 50 U/ml to about 1500 U/ml, about 50 U/ml to about 2000 U/ml, about 100 U/ml to about 500 U/ml, about 100 U/ml to about 1500 U/ml, and about 100 U/ml to about 2000 U/ml. The IFNγ may be present in the expansion medium at a concentration of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 1920, 1930, 1940, 1950, 1960, 1970, 1980, 1990, or 2000 U/ml.

The IFNγ may be present in the expansion medium at a concentration of about 1 to about 30 ng/ml. The IFNγ may be present in the expansion medium at a concentration of about 1 to about 15 ng/ml, 1 to about 45 ng/ml, or 1 to about 60 ng/ml. The IFNγ may be present in the expansion medium at a concentration of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 ng/ml.

The second expansion may occur for about 7 days. The second expansion may occur for about 1 to about 3 days, about 1 to about 5 days, about 1 to about 9 days, about 1 to about 11 days, about 1 to about 13 days, or about 1 to about 15 days. The second expansion may occur for about 1, 2, 3, 4, 5, 6, 7, 8, 9, 10, 11, 12, 13, 14, or 15 days.

The IFNγ may be added to the expansion media during the final day, the final 2 days, or the final 3 days of the second expansion. The IFNγ may also be added to the expansion media during the final 4 days, the final 5 days, the final 6 days, the final 7 days, the final 8 days, the final 9 days, the final 10 days, the final 11 days, the final 12 days, the final 13 days, or the final 14 days of the second expansion.

In some cases, when the MSCs were being interferon γ-primed, the culture was not in a hypoxic condition. As examples, the culture did not have decreased levels of 02, did not have increased levels of CO₂, and/or lacked the presence of a hypoxia mimetic. Decreased levels of O₂ may be from about 1% O₂ to about 5% O₂. Increased levels of CO₂ can be about 5% CO₂ or higher. Illustrative hypoxia mimetics include desferoxamine, cobalt chloride, hydralazine, nickel chloride, diazoxide, and dimethyloxalyglycine.

In embodiments, a clinical product comprising γ-IFN primed MSCs substantially lacks any residual γ-IFN. By substantially lacking residual γ-IFN can mean that an ELISA in unable to detect the cytokine in a wash media nor in a sample of the cell product itself. If any residual γ-IFN is detected in a clinical product the cells may be pelleted, washed and resuspended until γ-IFN becomes undetectable. By administering a clinical product that substantially lacks γ-IFN reduces the likelihood that a subject incurs a γ-IFN mediated toxicity (Table 4).

In some embodiments, the preparations and compositions of the present disclosure comprise interferon γ-primed human mesenchymal stromal cells (γMSCs) for preventing or reducing the likelihood of inflammatory conditions, including autoimmune conditions or a symptom thereof in a human subject. The MSCs may be obtained from one deceased human, as described herein. The MSCs may also be obtained from one or more vertebral bodies. The MSCs may comprise vertebral bone marrowMSCs (vBM-MSCs), vertebral bone-adherent MSCs (vBA-MSCs), or both. The vBM-MSCs and vBA-MSCs may be derived as is described herein.

In embodiments, Endotoxin testing is performed using the Endosafe®-PTS™ system according to SOP EPIC Limulus Amebocyte Lysate (LAL) Assay for Endotox in Detection Using the Endosafe®-PTS™. The Endosafe®-PTS™ is a rapid, point-of-use test system that provides quantitative LAL results within 15 minutes after specimen preparation. The PTS™ utilizes LAL reagents in an FDA-licensed disposable test cartridge with a handheld reader for a completely contained, real-time endotoxin testing system. The PTS™ can be used to get a quick read on raw materials and STAT samples that require immediate analysis. The flexibility of the PTS™ allows it to be used in conventional, quality control testing laboratories as well as at the point of sample collection. Preferably cells for clinical use have ≤2.5 E U/ml for release.

As shown in FIG. 11A, cultured P3 cells are shipped to a treatment site. The P3 cells may be cryopreserved and shipped accordingly. However, if the treatment site is local or within the same facility, campus, or healthcare complex, the P3 cells may not need to be cryopreserved and are instead shipped on ice, at room temperature, or at a culturing temperature (e.g., about 37° C.). The P3 cells are warmed (when cryopreserved or when shipped cold) or otherwise prepared for further culturing and are cultured to obtain P4 cells. The P4 cells are primed with IFNγ as disclosed herein. And, the final γ-MSCs are prepared (e.g., concentrated, diluted, and/or combined with pharmaceutically-acceptable solvents or carriers and the like) for transplant into a patient in need thereof.

As shown in FIG. 11B, cultured P4 cells are primed with IFNγ as disclosed herein. The γ-MSCs are shipped to a treatment site. The γ-MSCs may be cryopreserved and shipped accordingly. However, if the treatment site is local or within the same facility, campus, or healthcare complex, the γ-MSCs may not need to be cryopreserved and are instead shipped on ice, at room temperature, or at another temperature (e.g., about 37° C.). The γ-MSCs are warmed (when cryopreserved or when shipped cold) and/or otherwise prepared (e.g., concentrated, diluted, and/or combined with pharmaceutically-acceptable solvents or carriers and the like) for transplant to a patient in need thereof.

As shown in FIG. 11C, cultured P3 cells are primed with IFNγ as disclosed herein. The γ-MSCs are shipped to a treatment site. The γ-MSCs may be cryopreserved and shipped accordingly. However, if the treatment site is local or within the same facility, campus, or healthcare complex, the γ-MSCs may not need to be cryopreserved and are instead shipped on ice, at room temperature, or at a culturing temperature (e.g., about 37° C.). The γ-MSCs are warmed (when cryopreserved or when shipped cold) or otherwise prepared for further culturing and are cultured to obtain P4-stage γ-MSCs. The P4-stage γ-MSCs are prepared (e.g., concentrated, diluted, and/or combined with pharmaceutically-acceptable solvents or carriers and the like) for transplant to a patient in need thereof.

As shown in FIG. 11D, cultured P4 cells are shipped to a treatment site. The P4 cells may be cryopreserved and shipped accordingly. However, if the treatment site is local or within the same facility, campus, or healthcare complex, the P4 cells may not need to be cryopreserved and are instead shipped on ice, at room temperature, or at a culturing temperature (e.g., about 37° C.). The P4 cells are warmed (when cryopreserved or when shipped cold) or otherwise prepared for priming with IFNγ as disclosed herein. And, the final γ-MSCs are prepared (e.g., concentrated, diluted, and/or combined with pharmaceutically-acceptable solvents or carriers and the like) for transplant to a patient in need thereof.

An aspect of the present disclosure is a composition comprising interferon γ-primed human mesenchymal stromal cells (γMSCs) for preventing or reducing the likelihood of inflammatory conditions, including autoimmune conditions or a symptom thereof in a human subject. The composition comprising when the MSCs were being interferon γ-primed, the MSCs were not in a culture in a hypoxic condition.

In embodiments, the MSCs are obtained from one deceased human. In some embodiments, the MSCs are obtained from one or more vertebral bodies. In various embodiments, the MSCs comprise vertebral bone marrow MSCs (vBM-MSCs), vertebral bone-adherent MSCs (vBA-MSCs), or both.

In embodiments, the MSCs were expanded in culture prior to being interferon γ-primed. In some embodiments, the MSCs have undergone a primary expansion followed by cryopreservation. In various embodiments, the cryopreserved MSCs were thawed and then underwent a second expansion, e.g., the second expansion occurred for about seven days. In embodiments, the MSCs were primed with interferon gamma (IFNγ) during the second expansion, e.g., during the final day, the final two days, or the final three days of the second expansion, IFNγ was added to the second expansion media. In some embodiments, the IFNγ is present in the second expansion media at a concentration of from about 100 U/ml to about 1000 U/ml and/or from about 1 ng/mL to about 30 ng/ml.

In any of the herein disclosed aspects or embodiments, rather than cryopreserving MSCs or γMSCs and thawing the MSCs (which are subsequently primed) or thawing the γMSCs prior to use (either immediately or after one or more culturing steps), fresh MSCs or fresh γMSCs may be used.

In various embodiments, the composition comprises less than about 5% CD45+ cells, less than about 4% CD45+ cells, less than about 3% CD45+ cells, less than about 2% CD45+ cells, less than about 1% CD45+ cells, or about 0% CD45+ cells. In embodiments, the composition comprises at least about 90% CD105+ cells, at least about 91% CD105+ cells, at least about 92% CD105+ cells, at least about 93% CD105+ cells, at least about 94% CD105+ cells, at least about 95% CD105+ cells, at least about 96% CD105+ cells, at least about 97% CD105+ cells, at least about 98% CD105+ cells, or at least about 99% CD105+ cells. In some embodiments, the composition comprises at least about 90% CD166+ cells.

In various embodiments, the γMSCs are obtained from a matched unrelated donor to the subject. In embodiments, the γMSCs are obtained from an unmatched donor to the subject.

In some embodiments, the composition is formulated to comprise at least about 1×10⁶ γMSCs/kg of ideal body weight or actual bodyweight of the human subject. In various embodiments, the composition is formulated to comprise at least about 2×10⁶ γMSCs/kg of ideal body weight or actual body weight of the human subject. In embodiments, the composition is formulated to comprise at least about 5×10⁶ γMSCs/kg of ideal body weight or actual body weight of the human subject. In some embodiments, the composition is formulated to comprise at least about 10×10⁶ γMSCs/kg of ideal body weight or actual body weight of the human subject.

In another aspect, the present disclosure provides a method for manufacturing interferon γ-primed mesenchymal stromal cells (γMSCs) for use in treating inflammatory conditions, including autoimmune conditions or a symptom thereof. The method comprising obtaining a culture of MSCs and contacting the MSCs with interferon gamma (IFNγ), wherein when the MSCs were being interferon γ-primed, the MSCs were not in a culture in a hypoxic condition.

In various embodiments, the MSCs are expanded in culture prior to being interferon γ-primed. In embodiments, the MSCs undergo a primary expansion followed by cryopreservation. In some embodiments, the cryopreserved MSCs are thawed and then undergo a second expansion, e.g., the second expansion occurs for about seven days. In various embodiments, the MSCs are primed with interferon gamma (IFNγ) during the second expansion, e.g., during the final day, the final two days, or the final three days of the second expansion, IFNγ is added to the second expansion media. In embodiments, the IFNγ is present in the second expansion media at a concentration of from about 100 U/ml to about 1000 U/ml or from about 1 ng/mL to about 30 ng/ml. In some embodiments, the IFNγ is present in the second expansion media at a concentration of about 500 U/ml.

In any of the herein disclosed aspects or embodiments, rather than cryopreserving MSCs or γMSCs and thawing the MSCs (which are subsequently primed) or thawing the γMSCs prior to use (either immediately or after one or more culturing steps), fresh MSCs or fresh γMSCs may be used.

In various embodiments, the MSCs are obtained from one deceased human. In embodiments, the MSCs are obtained from one or more vertebral bodies and the MSCs comprise vertebral bone marrow MSCs (vBM-MSCs), vertebral bone-adherent MSCs (vBA-MSCs), or both. In some embodiments, when a hypoxic condition is due to the presence of decreased levels of O₂, increased levels of CO₂, and/or a hypoxia mimetic. In various embodiments, the γMSCs were cultured under a serum starved condition and/or were exposed to a temperature shock during culturing.

Alternates and/or Supplements to Priming MSCs with IFNγ

In vivo, MSCs are stimulated in response to abnormal conditions, e.g., caused by infection, cancer, and injury. The presence of circulating IFNγ can be a signal to MSCs which identifies an abnormal condition. In response to the circulating IFNγ, the MSCs are primed and secrete, at least, factors and proteins that help remedy the abnormal condition. In various aspects of the present disclosure, MSCs are primed in vitro or ex vivo by contact with IFNγ to transform the cells into γMSCs. In some cases, MSCs are only primed by contact with IFNγ. In other cases, MSCs are primed by contact with IFNγ and also stimulated with one or both of serum starvation and temperature shock. In yet other cases, MSCs are not primed by contact with IFNγ and are instead stimulated with one or both of serum starvation and temperature shock.

Serum starvation is the removal of serum (which is a source of proteins, including growth factors) from a culturing medium. Serum starvation, like the presence of IFNγ, informs the MSCs of an abnormal condition, which likewise will stimulate the MSCs. Serum starvation can occur contemporaneously with the IFNγ priming (as disclosed elsewhere herein). In certain methods, human platelet lysate (hPL) is omitted from the culturing medium to enact serum starvation. As examples, the serum starvation may occur during the final day or the final two days of culturing In some cases, MSCs are primed by contact with IFNγ and are stimulated by serum starvation.

In some embodiments, MSCs are not primed by contact with IFNγ and are instead stimulated by serum starvation.

Temperature shock is an increase in the temperature of a cell culture to a degree and duration that the cells are shocked, e.g., induce expression of heat shock proteins. Temperature shock is another indicator of an abnormal condition (e.g., suggesting an infection) which simulates MSCs.

In some embodiments, the temperature of the cell culture is raised to about 40° C. or higher. The duration may be for less than an hour, for about an hour, for about one or two hours, or for more than two hours. In various embodiments, the temperature shock occurs during the final day of culturing. In some embodiments, the temperature shock occurs in the final hour or final hours of culturing. In some cases, MSCs are primed by contact with IFNγ and are stimulated by temperature shock.

In some embodiments, MSCs are not primed by contact with IFNγ and are instead stimulated by temperature shock.

In various embodiments, MSCs are not primed by contact with IFNγ and are instead stimulated by serum starvation and by temperature shock.

In embodiments, MSCs are primed by contact with IFNγ and stimulated by serum starvation and by temperature shock.

In any herein disclosed method or composition, a γMSC may be an MSC that was only primed by contact with IFNγ. In any herein disclosed method or composition, a γMSC may be an MSC that was primed by contact with IFNγ and also stimulated with one or both of serum starvation and temperature shock. In any herein disclosed method or composition, a “γMSC” or “MSC” may be an MSC that was not primed by contact with IFNγ and was instead stimulated with one or both of serum starvation and temperature shock.

Compositions of γMSCs and/or MSCs

A clinical product may be tested analyzed to measure expression values of a subset of genes to determine if γ-IFN priming (as disclosed herein) introduced additional inter-donor variation other than what intrinsically exists in MSCs.

A clinical product may be assessed for the integrity of the chromosomes (karyotype). Twenty cells in metaphase from the standard MSC preparation and γ-IFN-primed MSCs (as disclosed herein) can be examined to detect the presence of 46 chromosomes including two sex chromosomes and any consistent structural or numerical abnormalities.

The surface phenotype of cells in the clinical product can be determined by flowcytometry. γ-IFN-primed MSCs should express HLA Class I and II molecules, as well as PD-L1 and PD-L2, but not CD80 or CD86.

MSCs and γ-MSCs (as disclosed herein) should differentiate in vitro into osteoblasts, adipocytes, and chondroblasts. Clinical products can be tested to determine if they can differentiate in vitro into, at least, these cell types. Differentiation of osteoblasts, adipocytes, and chondroblasts, respectively, can be detected morphologically using Alizarin Red S, Oil Red O, and Alcian Blue histochemical staining.

In some cases, a clinical product (or a previous-stage product) is tested for sterility by Gram stain and the final cellular material using the BacT/Alert system by inoculating aerobic and anaerobic test bottles and incubating for a 14-day culture period. The BacT/Alert uses a constant monitoring system that will alert presence of cellular growth at any point during the 14-day incubation.

The numbers of cells in clinical product and the number of viable cells can be assayed via the nucleic acid binding dyes acridine orange (AO) and propidium iodide (PI) using an automated cell counter (Cellometer), or via trypan blue exclusion method using manual count.

In various embodiment, a clinical product may comprise from about 1×10⁶ γMSCs/ml to about 1×10⁷ γMSCs/ml of infusion medium. As examples, the clinical product may comprise about 1×10⁶ γMSCs/ml, 2×10⁶ γMSCs/ml, 3×10⁶ γMSCs/ml, 4×10⁶ γMSCs/ml, 5×10⁶ γMSCs/ml, 6×10⁶ γMSCs/ml, 7×10⁶ γMSCs/ml, 8×10⁶ γMSCs/ml, 9×10⁶ γMSCs/ml, or 1×10⁷ γMSCs/ml, and any concentration of γMSCs therebetween. In one example, a clinical product comprises about 4×10⁶ γMSCs/ml of infusion media, e.g., about 3.5×10⁶ γMSCs/ml, 3.6×10⁶ γMSCs/ml, 3.7×10⁶ γMSCs/ml, 3.8×10⁶ γMSCs/ml, 3.9×10⁶ γMSCs/ml, 4×10⁶ γMSCs/ml, 4.1×10⁶ γMSCs/ml, 4.2×10⁶ γMSCs/ml, 4.3×10⁶ γMSCs/ml, 4.4×10⁶ γMSCs/ml, or 4.5×10⁶ γMSCs/ml, and any concentration of γMSCs therebetween.

An infusion medium may comprise about 0.5% human serum albumin (HSA).

In various clinical products, no antibiotics or anti-mycotics were used in the MSC manufacturing process, no bovine or porcine components were used in the MSC manufacturing process, and/or irradiation was not used during any step in the MSC manufacturing process.

In some embodiments, the preparations and compositions of the present disclosure may comprise at least 100 million γMSCs and/or MSCs having an antigen profile of more than about 1.75% CD45+ cells, at least about 95% CD105+ cells, and at least about 95% CD166+ cells and the cells may be expanded ex vivo from passage 2 until passage 4 while maintaining population uniformity based upon the antigen profile (i.e., more than about 1.75% CD45+ cells, at least about 95% CD105+ cells, and at least about 95% CD166+ cells).

In some embodiments, the preparations and compositions of the present disclosure may comprise at least 100 million γMSCs and/or MSCs having an antigen profile of less than about 5% CD45+ cells, at least about 95% CD105+ cells, and at least about 95% CD166+ cells and the cells may be expanded ex vivo from passage 2 until passage 4 while maintaining population uniformity based upon the antigen profile (i.e., less than about 1.75% CD45+ cells, at least about 95% CD105+ cells, and at least about 95% CD166+ cells). In embodiments, the γMSCs and/or MSCs have an antigen profile of less than about 4% CD45+ cells, less than about 3% CD45+ cells, less than about 2% CD45+ cells, less than about 1% CD45+ cells, or about 0% CD45+ cells.

In some embodiments, the preparations and compositions of the present disclosure may comprise γMSCs and/or MSCs having an antigen profile of reduced expression of one or more senescent cell markers, as compared to bone marrow-derived MSCs prepared according to known MSC culturing techniques. In some embodiments, the one or more senescent cell markers comprise MIC-A, MIC-B, ULBP2, or any combination thereof. NK cell-mediated immune responses are stimulated by MIC-A, MIC-B, and/or ULBP2.

In some embodiments, the γMSC and/or MSC preparations and compositions described herein comprise a number of cells that express one or more senescent cell markers of about 1% less than bone marrow-derived MSCs to about 100% less than bone marrow-derived MSCs. In some embodiments, the γMSC and/or MSC preparations and compositions described herein comprise an amount of cells that express one or more senescent cell markers of about 100% less than bone marrow-derived MSCs to about 90% less than bone marrow-derived MSCs, about 100% less than bone marrow-derived MSCs to about 80% less than bone marrow-derived MSCs, about 100% less than bone marrow-derived MSCs to about 70% less than bone marrow-derived MSCs, about 100% less than bone marrow-derived MSCs to about 60% less than bone marrow-derived MSCs, about 100% less than bone marrow-derived MSCs to about 50% less than bone marrow-derived MSCs, about 100% less than bone marrow-derived MSCs to about 40% less than bone marrow-derived MSCs, about 100% less than bone marrow-derived MSCs to about 30% less than bone marrow-derived MSCs, about 100% less than bone marrow-derived MSCs to about 20% less than bone marrow-derived MSCs, about 100% less than bone marrow-derived MSCs to about 10% less than bone marrow-derived MSCs, about 100% less than bone marrow-derived MSCs to about 5% less than bone marrow-derived MSCs, about 100% less than bone marrow-derived MSCs to about 1% less than bone marrow-derived MSCs, about 90% less than bone marrow-derived MSCs to about 80% less than bone marrow-derived MSCs, about 90% less than bone marrow-derived MSCs to about 70% less than bone marrow-derived MSCs, about 90% less than bone marrow-derived MSCs to about 60% less than bone marrow-derived MSCs, about 90% less than bone marrow-derived MSCs to about 50% less than bone marrow-derived MSCs, about 90% less than bone marrow-derived MSCs to about 40% less than bone marrow-derived MSCs, about 90% less than bone marrow-derived MSCs to about 30% less than bone marrow-derived MSCs, about 90% less than bone marrow-derived MSCs to about 20% less than bone marrow-derived MSCs, about 90% less than bone marrow-derived MSCs to about 10% less than bone marrow-derived MSCs, about 90% less than bone marrow-derived MSCs to about 5% less than bone marrow-derived MSCs, about 90% less than bone marrow-derived MSCs to about 1% less than bone marrow-derived MSCs, about 80% less than bone marrow-derived MSCs to about 70% less than bone marrow-derived MSCs, about 80% less than bone marrow-derived MSCs to about 60% less than bone marrow-derived MSCs, about 80% less than bone marrow-derived MSCs to about 50% less than bone marrow-derived MSCs, about 80% less than bone marrow-derived MSCs to about 40% less than bone marrow-derived MSCs, about 80% less than bone marrow-derived MSCs to about 30% less than bone marrow-derived MSCs, about 80% less than bone marrow-derived MSCs to about 20% less than bone marrow-derived MSCs, about 80% less than bone marrow-derived MSCs to about 10% less than bone marrow-derived MSCs, about 80% less than bone marrow-derived MSCs to about 5% less than bone marrow-derived MSCs, about 80% less than bone marrow-derived MSCs to about 1% less than bone marrow-derived MSCs, about 70% less than bone marrow-derived MSCs to about 60% less than bone marrow-derived MSCs, about 70% less than bone marrow-derived MSCs to about 50% less than bone marrow-derived MSCs, about 70% less than bone marrow-derived MSCs to about 40% less than bone marrow-derived MSCs, about 70% less than bone marrow-derived MSCs to about 30% less than bone marrow-derived MSCs, about 70% less than bone marrow-derived MSCs to about 20% less than bone marrow-derived MSCs, about 70% less than bone marrow-derived MSCs to about 10% less than bone marrow-derived MSCs, about 70% less than bone marrow-derived MSCs to about 5% less than bone marrow-derived MSCs, about 70% less than bone marrow-derived MSCs to about 1% less than bone marrow-derived MSCs, about 60% less than bone marrow-derived MSCs to about 50% less than bone marrow-derived MSCs, about 60% less than bone marrow-derived MSCs to about 40% less than bone marrow-derived MSCs, about 60% less than bone marrow-derived MSCs to about 30% less than bone marrow-derived MSCs, about 60% less than bone marrow-derived MSCs to about 20% less than bone marrow-derived MSCs, about 60% less than bone marrow-derived MSCs to about 10% less than bone marrow-derived MSCs, about 60% less than bone marrow-derived MSCs to about 5% less than bone marrow-derived MSCs, about 60% less than bone marrow-derived MSCs to about 1% less than bone marrow-derived MSCs, about 50% less than bone marrow-derived MSCs to about 40% less than bone marrow-derived MSCs, about 50% less than bone marrow-derived MSCs to about 30% less than bone marrow-derived MSCs, about 50% less than bone marrow-derived MSCs to about 20% less than bone marrow-derived MSCs, about 50% less than bone marrow-derived MSCs to about 10% less than bone marrow-derived MSCs, about 50% less than bone marrow-derived MSCs to about 5% less than bone marrow-derived MSCs, about 50% less than bone marrow-derived MSCs to about 1% less than bone marrow-derived MSCs, about 40% less than bone marrow-derived MSCs to about 30% less than bone marrow-derived MSCs, about 40% less than bone marrow-derived MSCs to about 20% less than bone marrow-derived MSCs, about 40% less than bone marrow-derived MSCs to about 10% less than bone marrow-derived MSCs, about 40% less than bone marrow-derived MSCs to about 5% less than bone marrow-derived MSCs, about 40% less than bone marrow-derived MSCs to about 1% less than bone marrow-derived MSCs, about 30% less than bone marrow-derived MSCs to about 20% less than bone marrow-derived MSCs, about 30% less than bone marrow-derived MSCs to about 10% less than bone marrow-derived MSCs, about 30% less than bone marrow-derived MSCs to about 5% less than bone marrow-derived MSCs, about 30% less than bone marrow-derived MSCs to about 1% less than bone marrow-derived MSCs, about 20% less than bone marrow-derived MSCs to about 10% less than bone marrow-derived MSCs, about 20% less than bone marrow-derived MSCs to about 5% less than bone marrow-derived MSCs, about 20% less than bone marrow-derived MSCs to about 1% less than bone marrow-derived MSCs, about 10% less than bone marrow-derived MSCs to about 5% less than bone marrow-derived MSCs, about 10% less than bone marrow-derived MSCs to about 1% less than bone marrow-derived MSCs, or about 5% less than bone marrow-derived MSCs to about 1% less than bone marrow-derived MSCs. In some embodiments, the vBA-MSC preparations and compositions described herein comprise an amount of cells that express one or more senescent cell markers of about 100% less than bone marrow-derived MSCs, about 90% less than bone marrow-derived MSCs, about 80% less than bone marrow-derived MSCs, about 70% less than bone marrow-derived MSCs, about 60% less than bone marrow-derived MSCs, about 50% less than bone marrow-derived MSCs, about 40% less than bone marrow-derived MSCs, about 30% less than bone marrow-derived MSCs, about 20% less than bone marrow-derived MSCs, about 10% less than bone marrow-derived MSCs, about 5% less than bone marrow-derived MSCs, or about 1% less than bone marrow-derived MSCs. In some embodiments, the vBA-MSC preparations and compositions described herein comprise an amount of cells that express one or more senescent cell markers of at least about 100% less than bone marrow-derived MSCs, about 90% less than bone marrow-derived MSCs, about 80% less than bone marrow-derived MSCs, about 70% less than bone marrow-derived MSCs, about 60% less than bone marrow-derived MSCs, about 50% less than bone marrow-derived MSCs, about 40% less than bone marrow-derived MSCs, about 30% less than bone marrow-derived MSCs, about 20% less than bone marrow-derived MSCs, about 10% less than bone marrow-derived MSCs, or about 5% less than bone marrow-derived MSCs. In some embodiments, the vBA-MSC preparations and compositions described herein comprise an amount of cells that express one or more senescent cell markers of at most about 90% less than bone marrow-derived MSCs, about 80% less than bone marrow-derived MSCs, about 70% less than bone marrow-derived MSCs, about 60% less than bone marrow-derived MSCs, about SO % less than bone marrow-derived MSCs, about 40% less than bone marrow-derived MSCs, about 30% less than bone marrow-derived MSCs, about 20% less than bone marrow-derived MSCs, about 10% less than bone marrow-derived MSCs, about 5% less than bone marrow-derived MSCs, or about 1% less than bone marrow-derived MSCs.

In some embodiments, the preparations and compositions of the present disclosure generate a lessened NK cell-mediated immune response upon administration to a subject comprising mis-matched MHC molecules (e.g., mis-matched human leukocyte antigens when the subject is a human), as compared to administration of a composition comprising bone marrow-derived MSCs. In some embodiments, the preparations and compositions of the present disclosure do not generate a NK cell-mediated immune response upon administration to a subject comprising mis-matched MHC molecules (e.g., mis-matched human leukocyte antigens when the subject is a human).

In some embodiments, the composition of γMSCs and/or MSCs may be comprised of less than about 5% CD45+ cells. In some embodiments, the composition of γMSCs and/or MSCs may be comprised of less than about 90%, 80%, 70%, 60%, 50%, 40%, 30%, 20%, or 10% CD45+ cells. In some embodiments, the composition of γMSCs and/or MSCs may be comprised of less than about 0.5% CD45+ to about 10% CD45+. In some embodiments, the composition of γMSCs and/or MSCs may be comprised of less than about 10% CD45+ to about 9% CD45+, about 10% CD45+ to about 8% CD45+, about 10% CD45+ to about 7% CD45+, about 10% CD45+ to about 6% CD45+, about 10% CD45+ to about 5% CD45+, about 10% CD45+ to about 4% CD45+, about 10% CD45+ to about 3% CD45+, about 10% CD45+ to about 2% CD45+, about 10% CD45+ to about 1% CD45+, about 10% CD45+ to about 0.5% CD45+, about 9% CD45+ to about 8% CD45+, about 9% CD45+ to about 7% CD45+, about 9% CD45+ to about 6% CD45+, about 9% CD45+ to about 5% CD45+, about 9% CD45+ to about 4% CD45+, about 9% CD45+ to about 3% CD45+, about 9% CD45+ to about 2% CD45+, about 9% CD45+ to about 1% CD45+, about 9% CD45+ to about 0.5% CD45+, about 8% CD45+ to about 7% CD45+, about 8% CD45+ to about 6% CD45+, about 8% CD45+ to about 5% CD45+, about 8% CD45+ to about 4% CD45+, about 8% CD45+ to about 3% CD45+, about 8% CD45+ to about 2% CD45+, about 8% CD45+ to about 1% CD45+, about 8% CD45+ to about 0.5% CD45+, about 7% CD45+ to about 6% CD45+, about 7% CD45+ to about 5% CD45+, about 7% CD45+ to about 4% CD45+, about 7% CD45+ to about 3% CD45+, about 7% CD45+ to about 2% CD45+, about 7% CD45+ to about 1% CD45+, about 7% CD45+ to about 0.5% CD45+, about 6% CD45+ to about 5% CD45+, about 6% CD45+ to about 4% CD45+, about 6% CD45+ to about 3% CD45+, about 6% CD45+ to about 2% CD45+, about 6% CD45+ to about 1% CD45+, about 6% CD45+ to about 0.5% CD45+, about 5% CD45+ to about 4% CD45+, about 5% CD45+ to about 3% CD45+, about 5% CD45+ to about 2% CD45+, about 5% CD45+ to about 1% CD45+, about 5% CD45+ to about 0.5% CD45+, about 4% CD45+ to about 3% CD45+, about 4% CD45+ to about 2% CD45+, about 4% CD45+ to about 1% CD45+, about 4% CD45+ to about 0.5% CD45+, about 3% CD45+ to about 2% CD45+, about 3% CD45+ to about 1% CD45+, about 3% CD45+ to about 0.5% CD45+, about 2% CD45+ to about 1% CD45+, about 2% CD45+ to about 0.5% CD45+, or about 1% CD45+ to about 0.5% CD45+. In some embodiments, the composition of γMSCs and/or MSCs may be comprised of less than about 10% CD45+, about 9% CD45+, about 8% CD45+, about 7% CD45+, about 6% CD45+, about 5% CD45+, about 4% CD45+, about 3% CD45+, about 2% CD45+, about 1% CD45+, or about 0.5% CD45+. In some embodiments, the composition of γMSCs and/or MSCs may be comprised of less than at least about 10% CD45+, about 9% CD45+, about 8% CD45+, about 7% CD45+, about 6% CD45+, about 5% CD45+, about 4% CD45+, about 3% CD45+, about 2% CD45+, or about 1% CD45+. In some embodiments, the composition of γMSCs and/or MSCs may be comprised of less than at most about 9% CD45+, about 8% CD45+, about 7% CD45+, about 6% CD45+, about 5% CD45+, about 4% CD45+, about 3% CD45+, about 2% CD45+, about 1% CD45+, or about 0.5% CD45+. In some embodiments, the γMSCs and/or MSCs have an antigen profile of about 0% CD45+ cells.

In some embodiments, the composition of γMSCs and/or MSCs may be comprised of at least about 90% CD105+ cells. In some embodiments, the composition of γMSCs and/or MSCs may be comprised of at least about 10%, 20%, 30%, 40%, 50%, 60%, 70%, or 80% CD105+ cells. In some embodiments, the composition of γMSCs and/or MSCs may be comprised of at least about 70% CD105+ cells to about 100% CD105+ cells. In some embodiments, the composition of γMSCs and/or MSCs may be comprised of at least about 100% CD105+ cells to about 95% CD105+ cells, about 100% CD105+ cells to about 94% CD105+ cells, about 100% CD105+ cells to about 93% CD105+ cells, about 100% CD105+ cells to about 92% CD105+ cells, about 100% CD105+ cells to about 91% CD105+ cells, about 100% CD105+ cells to about 90% CD105+ cells, about 100% CD105+ cells to about 85% CD105+ cells, about 100% CD105+ cells to about 80% CD105+ cells, about 100% CD105+ cells to about 75% CD105+ cells, about 100% CD105+ cells to about 70% CD105+ cells, about 95% CD105+ cells to about 94% CD105+ cells, about 95% CD105+ cells to about 93% CD105+ cells, about 95% CD105+ cells to about 92% CD105+ cells, about 95% CD105+ cells to about 91% CD105+ cells, about 95% CD105+ cells to about 90% CD105+ cells, about 95% CD105+ cells to about 85% CD105+ cells, about 95% CD105+ cells to about 80% CD105+ cells, about 95% CD105+ cells to about 75% CD105+ cells, about 95% CD105+ cells to about 70% CD105+ cells, about 94% CD105+ cells to about 93% CD105+ cells, about 94% CD105+ cells to about 92% CD105+ cells, about 94% CD105+ cells to about 91% CD105+ cells, about 94% CD105+ cells to about 90% CD105+ cells, about 94% CD105+ cells to about 85% CD105+ cells, about 94% CD105+ cells to about 80% CD105+ cells, about 94% CD105+ cells to about 75% CD105+ cells, about 94% CD105+ cells to about 70% CD105+ cells, about 93% CD105+ cells to about 92% CD105+ cells, about 93% CD105+ cells to about 91% CD105+ cells, about 93% CD105+ cells to about 90% CD105+ cells, about 93% CD105+ cells to about 85% CD105+ cells, about 93% CD105+ cells to about 80% CD105+ cells, about 93% CD105+ cells to about 75% CD105+ cells, about 93% CD105+ cells to about 70% CD105+ cells, about 92% CD105+ cells to about 91% CD105+ cells, about 92% CD105+ cells to about 90% CD105+ cells, about 92% CD105+ cells to about 85% CD105+ cells, about 92% CD105+ cells to about 80% CD105+ cells, about 92% CD105+ cells to about 75% CD105+ cells, about 92% CD105+ cells to about 70% CD105+ cells, about 91% CD105+ cells to about 90% CD105+ cells, about 91% CD105+ cells to about 85% CD105+ cells, about 91% CD105+ cells to about 80% CD105+ cells, about 91% CD105+ cells to about 75% CD105+ cells, about 91% CD105+ cells to about 70% CD105+ cells, about 90% CD105+ cells to about 85% CD105+ cells, about 90% CD105+ cells to about 80% CD105+ cells, about 90% CD105+ cells to about 75% CD105+ cells, about 90% CD105+ cells to about 70% CD105+ cells, about 85% CD105+ cells to about 80% CD105+ cells, about 85% CD105+ cells to about 75% CD105+ cells, about 85% CD105+ cells to about 70% CD105+ cells, about 80% CD105+ cells to about 75% CD105+ cells, about 80% CD105+ cells to about 70% CD105+ cells, or about 75% CD105+ cells to about 70% CD105+ cells. In some embodiments, the composition of γMSCs and/or MSCs may be comprised of about 100% CD105+ cells, at least about 99% CD105+ cells, about 98% CD105+ cells, about 97% CD105+ cells, about 96% CD105+ cells, about 95% CD105+ cells, about 94% CD105+ cells, about 93% CD105+ cells, about 92% CD105+ cells, about 91% CD105+ cells, about 90% CD105+ cells, about 85% CD105+ cells, about 80% CD105+ cells, about 75% CD105+ cells, or about 70% CD105+ cells. In some embodiments, the composition of γMSCs and/or MSCs may be comprised of at least at least about 100% CD105+ cells, about 95% CD105+ cells, about 94% CD105+ cells, about 93% CD105+ cells, about 92% CD105+ cells, about 91% CD105+ cells, about 90% CD105+ cells, about 85% CD105+ cells, about 80% CD105+ cells, or about 75% CD105+ cells. In some embodiments, the composition of γMSCs and/or MSCs may be comprised of about 99.9% CD105+ cells, about 99.8% CD105+ cells, about 99.7% CD105+ cells, about 99.6% CD105+ cells, about 99.5% CD105+ cells, about 99.4% CD105+ cells, about 99.3% CD105+ cells, about 99.2% CD105+ cells, about 99.1% CD105+ cells, or about 99.0% CD105+ cells.

In some embodiments, the composition of γMSCs and/or MSCs comprise at least about 90% CD166+ cells. In some embodiments, the composition of γMSCs and/or MSCs comprise at least about 10%, 20%, 30%, 40%, 50%, 60%, 70%, or 80% CD166+ cells. In some embodiments, the composition of γMSCs and/or MSCs maybe comprised of at least about 70% CD166+ cells to about 100% CD166+ cells. In some embodiments, the composition of γMSCs and/or MSCs may be comprised of at least about 100% CD166+ cells to about 95% CD166+ cells, about 100% CD166+ cells to about 94% CD166+ cells, about 100% CD166+ cells to about 93% CD166+ cells, about 100% CD166+ cells to about 92% CD166+ cells, about 100% CD166+ cells to about 91% CD166+ cells, about 100% CD166+ cells to about 90% CD166+ cells, about 100% CD166+ cells to about 85% CD166+ cells, about 100% CD166+ cells to about 80% CD166+ cells, about 100% CD166+ cells to about 75% CD166+ cells, about 100% CD166+ cells to about 70% CD166+ cells, about 95% CD166+ cells to about 94% CD166+ cells, about 95% CD166+ cells to about 93% CD166+ cells, about 95% CD166+ cells to about 92% CD166+ cells, about 95% CD166+ cells to about 91% CD166+ cells, about 95% CD166+ cells to about 90% CD166+ cells, about 95% CD166+ cells to about 85% CD166+ cells, about 95% CD166+ cells to about 80% CD166+ cells, about 95% CD166+ cells to about 75% CD166+ cells, about 95% CD166+ cells to about 70% CD166+ cells, about 94% CD166+ cells to about 93% CD166+ cells, about 94% CD166+ cells to about 92% CD166+ cells, about 94% CD166+ cells to about 91% CD166+ cells, about 94% CD166+ cells to about 90% CD166+ cells, about 94% CD166+ cells to about 85% CD166+ cells, about 94% CD166+ cells to about 80% CD166+ cells, about 94% CD166+ cells to about 75% CD166+ cells, about 94% CD166+ cells to about 70% CD166+ cells, about 93% CD166+ cells to about 92% CD166+ cells, about 93% CD166+ cells to about 91% CD166+ cells, about 93% CD166+ cells to about 90% CD166+ cells, about 93% CD166+ cells to about 85% CD166+ cells, about 93% CD166+ cells to about 80% CD166+ cells, about 93% CD166+ cells to about 75% CD166+ cells, about 93% CD166+ cells to about 70% CD166+ cells, about 92% CD166+ cells to about 91% CD166+ cells, about 92% CD166+ cells to about 90% CD166+ cells, about 92% CD166+ cells to about 85% CD166+ cells, about 92% CD166+ cells to about 80% CD166+ cells, about 92% CD166+ cells to about 75% CD166+ cells, about 92% CD166+ cells to about 70% CD166+ cells, about 91% CD166+ cells to about 90% CD166+ cells, about 91% CD166+ cells to about 85% CD166+ cells, about 91% CD166+ cells to about 80% CD166+ cells, about 91% CD166+ cells to about 75% CD166+ cells, about 91% CD166+ cells to about 70% CD166+ cells, about 90% CD166+ cells to about 85% CD166+ cells, about 90% CD166+ cells to about 80% CD166+ cells, about 90% CD166+ cells to about 75% CD166+ cells, about 90% CD166+ cells to about 70% CD166+ cells, about 85% CD166+ cells to about 80% CD166+ cells, about 85% CD166+ cells to about 75% CD166+ cells, about 85% CD166+ cells to about 70% CD166+ cells, about 80% CD166+ cells to about 75% CD166+ cells, about 80% CD166+ cells to about 70% CD166+ cells, or about 75% CD166+ cells to about 70% CD166+ cells. In some embodiments, the composition of γMSCs and/or MSCs may be comprised of at least about 100% CD166+ cells, about 95% CD166+ cells, about 94% CD166+ cells, about 93% CD166+ cells, about 92% CD166+ cells, about 91% CD166+ cells, about 90% CD166+ cells, about 85% CD166+ cells, about 80% CD166+ cells, about 75% CD166+ cells, or about 70% CD166+ cells. In some embodiments, the composition of γMSCs and/or MSCs may be comprised of at least at least about 100% CD166+ cells, about 95% CD166+ cells, about 94% CD166+ cells, about 93% CD166+ cells, about 92% CD166+ cells, about 91% CD166+ cells, about 90% CD166+ cells, about 85% CD166+ cells, about 80% CD166+ cells, or about 75% CD166+ cells. In some embodiments, the composition of γMSCs and/or MSCs may be comprised of at least at most about 95% CD166+ cells, about 94% CD166+ cells, about 93% CD166+ cells, about 92% CD166+ cells, about 91% CD166+ cells, about 90% CD166+ cells, about 85% CD166+ cells, about 80% CD166+ cells, about 75% CD166+ cells, or about 70% CD166+ cells.

In some embodiments, the γMSCs are obtained from a matched unrelated donor to the subject or an unmatched donor to the subject.

In some embodiments, the composition is formulated to comprise at least about 1×10⁶ γMSCs/kg of ideal body weight or actual body weight of the human subject. In some embodiments, the composition is formulated to comprise at least about 2×10⁶ γMSCs/kg of ideal body weight or actual body weight of the human subject. In some embodiments, the composition is formulated to comprise at least about 5×10⁶γMSCs/kg of ideal body weight or actual body weight of the human subject. In some embodiments, the composition is formulated to comprise at least about 10×10⁶ γMSCs/kg of ideal body weight or actual body weight of the human subject. In some embodiments, the composition is formulated to comprise at least about 3, 4, 6, 7, 8, 9, 11, 12, 13, 14, 15, 16, 17, 18, 19, or 20×10⁶ γMSCs/kg of ideal body weight or actual body weight of the human subject. In some embodiments, the composition is formulated to comprise at least about 1 to about 5×10⁶ γMSCs/kg, about 6 to about 10×10⁶ γMSCs/kg about 11 to about 15×10⁶ γMSCs/kg, or about 16 to about 20×10⁶ γMSCs/kg of ideal body weight or actual bodyweight of the human subject.

Some embodiments include a plurality of compositions. For example, a plurality of compositions may include one composition of γMSCs comprising at least about 2×10⁶ γMSCs/kg of ideal body weight or actual body weight of the human subject and a second composition comprising at least about 5×10⁶ γMSCs/kg of ideal body weight or actual body weight of the human subject. A plurality of compositions may include one composition of γMSCs comprising at least about 2×10⁶ γMSCs/kg of ideal body weight or actual body weight of the human subject and a second composition comprising at least about 10×10⁶γMSCs/kg of ideal body weight or actual body weight of the human subject. A plurality of compositions may include one composition of γMSCs comprising at least about 5×10⁶ γMSCs/kg of ideal body weight or actual body weight of the human subject and a second composition comprising at least about 10×10⁶ γMSCs/kg of ideal body weight or actual body weight of the human subject. A plurality of compositions may include one composition of γMSCs comprising at least about 2×10⁶ γMSCs/kg of ideal body weight or actual body weight of the human subject, a second composition comprising at least about 5×10⁶ γMSCs/kg of ideal body weight or actual body weight of the human subject, and a third composition comprising at least about 10×10⁶ γMSCs/kg of ideal body weight or actual body weight of the human subject. An additional plurality of compositions may include one composition of γMSCs comprising at least about 1×10⁶ γMSCs/kg of ideal body weight or actual body weight of the human subject, a second composition comprising at least about 2×10⁶ γMSCs/kg of ideal body weight or actual body weight of the human subject, a third composition comprising at least about 5×10⁶ γMSCs/kg of ideal body weight or actual body weight of the human subject, and a fourth composition comprising at least about 10×10⁶ γMSCs/kg of ideal body weight or actual bodyweight of the human subject.

Table 5 below shows desirable characteristics of γMSCs compositions for infusion into a patient.

TABLE 5
Desirable characteristics of γMSCs compositions

| | | |
|---|---|---|
| Test | Method | Acceptable Result |

Pre-infusion Data

| | | |
|---|---|---|
| Cell Number | Automated Cell Counter | Required Cell Dose ±15% |
| Cell Viability | Automated Cell Counter | 85 ± 15% |
| Appearance | Automated Cell Counter | No aggregates |
| Sterility (Day −2) | Microscopy | Gram stain Negative |
| | BacT Alert | 48-hour culture negative |
| Sterility (Day 0) | Microscopy | Gram stain Negative |
| Endotoxin | Endosafe | <2.5 EU/ml |
| Mycoplasma | MycoAlert Assay | None Detected |
| Identity | Flow cytometry | CD45 <3% |
| | | CD105 >85% |

Post-Infusion-day Data

| | | |
|---|---|---|
| Sterility (Day −2) | BacT Alert | 14 Day culture Negative |
| Sterility (Day 0) | | 14 Day culture Negative |
| IDO1 Expression | Quantitative PCR | RE >1 |

### Methods of Treating

An aspect of the present disclosure is a method for treating a disease or disorder in a subject in need thereof. The method comprising administering to the subject a therapeutically-effective amount of any herein-disclosed pharmaceutical composition. The pharmaceutical composition comprises any herein disclosed population of isolated MSCs and a pharmaceutically acceptable excipient.

Some embodiments of the present disclosure involve use of any of the compositions disclosed herein for treating of inflammatory conditions, including autoimmune conditions or a symptom thereof.

An aspect of the present disclosure is a method for treating inflammatory conditions, including autoimmune conditions or a symptom thereof and/or reducing the likelihood of inflammatory conditions, including autoimmune conditions attack in a subject. The method comprising administering to the subject an effective amount of interferon γ-primed mesenchymal stromal cells (γMSCs), wherein when the MSCs were being interferon γ-primed, the MSCs were not in a culture in a hypoxic condition.

Another aspect of the present disclosure is a method for treating inflammatory conditions, including autoimmune conditions or a symptom thereof and/or reducing the likelihood of inflammatory conditions, including autoimmune conditions attack thereof in a subject. The method comprising steps of identifying a subject with inflammatory conditions, including autoimmune conditions and/or recurrent of inflammatory conditions, including autoimmune conditions who is minimally treated by a standard of care treatment for the inflammatory conditions, including autoimmune conditions at issue, or has a frequency of recurrent inflammatory conditions, including autoimmune conditions that is not reduced by the standard of care treatment for inflammatory conditions, including autoimmune conditions; and administering to the subject an effective amount of interferon γ-primed mesenchymal stromal cells (γMSCs). In this aspect, when the MSCs were being interferon γ-primed, the MSCs were not in a culture in a hypoxic condition.

Yet another aspect of the present disclosure is a method for treating inflammatory conditions, including autoimmune conditions or a symptom thereof and/or reducing the likelihood of inflammatory conditions, including autoimmune conditions thereof in a subject.

In embodiments, the γMSCs are obtained from a matched unrelated donor to the subject or an unmatched donor to the subject. In some embodiments, the γMSCs are obtained from a deceased donor.

In various embodiments, the amount of γMSCs administered to the subject comprise at least about 1×10⁶ cells/kg of ideal body weight or actual body weight. In embodiments, the subject is administered a plurality of doses of γMSCs in amounts from about 1×10⁶ cells/kg to about 10×10⁶ cells/kg of ideal body weight or actual body weight. In some embodiments, the subject is administered a plurality of doses of γMSCs in amounts from about 2×10⁶ cells/kg to about 5×10⁶ cells/kg of ideal body weight or actual body weight. In various embodiments, the amount of γMSCs administered in a first dose is less than the amount of γMSCs administered in one or more subsequent doses. In embodiments, the first dose is at least about 2×10⁶ cells/kg and the second dose is at least about 5×10⁶ cells/kg of ideal body weight or actual body weight

In some embodiments, the MSCs were expanded in culture prior to being interferon γ-primed. In various embodiments, the MSCs have undergone a primary expansion followed by cryopreservation. In some cases, the cryopreserved MSCs were thawed and then underwent a second expansion. In various cases, the second expansion occurred for about seven days. In some cases, the MSCs were primed with interferon gamma (IFNγ) during the second expansion. In embodiments, during the final day, the final two days, or the final three days of the second expansion, IFNγ was added to the second expansion media. In some embodiments, the IFNγ is present in the second expansion media at a concentration of from about 100 U/ml to about 1000 U/ml or from about Ing/ml to about 30 ng/ml and/or the IFNγ is present in the second expansion media at a concentration of from about 1 ng/ml to about 80 ng/ml. In some cases, the IFNγ is present in the second expansion media at a concentration of about 500 U/ml and/or the IFNγ is present in the second expansion media at a concentration of about 1000 U/ml. In embodiments, when a hypoxic condition is due to the presence of decreased levels of O₂, increased levels of CO₂, and/or a hypoxia mimetic.

In some embodiments, the subject is a human child or a human adult. In various embodiments, the subject has persistent inflammatory conditions, including autoimmune conditions, e.g., the persistent inflammatory conditions, including autoimmune conditions is moderate-to-severe. In embodiments, the γMSCs improve lung function. In some embodiments, the γMSCs reduce the onset of an inflammatory conditions, including autoimmune conditions exacerbation, difficulty in breathing, coughing dyspnea, the number and/or severity of episodes of inflammatory conditions, including autoimmune conditions attacks, shortness of breath, wheezing when exhaling, chest tightness, and/or chest pain experienced by the subject.

In various embodiments, the lung function is characterized by aspirometry, oscillomelly, bronchodilator reversibility test, lung clearance index test, exhaled nitric oxide test, breath condensate collection test, and/or another method for determining lung function.

In embodiments, the γMSC reduces upper airway inflammation, e.g., the upper airway inflammation is determined by analysis of small molecule inflammatory constituents in exhaled breath condensate. In various embodiments, the γMSC reduces circulating cell inflammation, e.g., the circulating cell inflammation is determined by flow cytometric analysis of peripheral blood cells and/or Ab Seq analysis of peripheral blood cells. In some embodiments, the method further comprising administering albuterol sulfate for bronchodilator reversibility test and/or sulfur hexafluoride (SF6) for Lung Clearance Index testing.

In various embodiments, the method further comprises administering a standard of care treatment for inflammatory conditions, including autoimmune conditions, e.g., the standard of care treatment for inflammatory conditions, including autoimmune conditions comprises albuterol, prednisone, and/or prednisolone. In some embodiments, the standard of care treatment for inflammatory conditions, including autoimmune conditions, comprises a biologic, wherein the biologic is selected from the group consisting of reslizumab (anti IL-5 neutralizing antibody), benralizumab (anti IL-5 receptor alpha), dupilumab (anti IL-4 receptor alpha subunit), mepolizumab (anti IL-5 neutralizing antibody), and/or omalizumab (anti-IgE neutralizing antibody). In various embodiments, the standard of care treatment for inflammatory conditions, including autoimmune conditions is a controller therapy for inflammatory conditions, including autoimmune conditions, e.g., inhaled corticosteroids. In some embodiments, the standard of care treatment for inflammatory conditions, including autoimmune conditions comprises analgesics, antihistamines, and/or intranasal corticosteroids.

In various embodiments, the subject is administered diphenhydramine and/or acetaminophen within 60 minutes of cell infusion. In embodiments, the subject is administered the standard of care treatment for inflammatory conditions, including autoimmune conditions before, contemporaneously with, and/or after administration of the γMSC. In some embodiments, the subject is administered the same standard of care treatment for inflammatory conditions, including autoimmune conditions that was administered before administration of the γMSC. In various embodiments, the subject is administered the different standard of care treatment for inflammatory conditions, including autoimmune conditions that was administered before administration of the γMSC.

In embodiments, an anesthetic cream or spray is administered prior to venipuncture or intravenous catheter insertion associated with administering the γMSCs. In some embodiments, the γMSC is administered intravenously, e.g., intravenously infused through a large bore peripheral N. In embodiments, the γMSC is infused by IV push or syringe pump, optionally, infused over 5-15 minutes. In some embodiments, no medications arc administered through a catheter used for the γMSC infusion.

In various embodiments, the MSCs are obtained from one or more vertebral bodies and the MSCs comprise vertebral bone marrow MSCs (vBM-MSCs), vertebral bone-adherent MSCs (vBA-MSCs), or both. In embodiments, the MSCs comprise less than about 5% CD45+ cells (e.g., less than about 5% CD45+ cells, less than about 4% CD45+ cells, less than about 3% CD45+ cells, less than about 2% CD45+ cells, less than about 1% CD45+ cells, or about 0% CD45+ cells), comprises at least about 90% CD105+ cells (e.g., at least about 90% CD105+ cells, at least about 91% CD105+ cells, at least about 92% CD105+ cells, at least about 93% CD105+ cells, at least about 94% CD105+ cells, at least about 95% CD105+ cells, at least about 96% CD105+ cells, at least about 97% CD105+ cells, at least about 98% CD105+ cells, or at least about 99% CD105+ cells), and/or comprises at least about 90% CD166+ cells. In some embodiments, the γMSCs were cultured under a serum starved condition and/or were exposed to a temperature shock during culturing. In various embodiments, the MSCs comprise less than about 10% CD45+ cells, at least about 90% CD105+ cells, and/or at least about 90% CD166+ cells.

In embodiments, the inflammatory conditions, including autoimmune conditions is not treated by the standard of care treatment for inflammatory conditions, including autoimmune conditions.

An infusion dose can be prepared in a 0.5% HSA+PLASMA-LYTE A solution at 4×10⁶ cells/ml and kept at room temperature in an infusion bag or syringe(s) until infused. A sample of the cells, e.g., about 1 to 2×10⁶ γ-MSCs can be collected for detecting microscopic or vegetative contaminating organisms, e.g., bacterial (aerobic & anaerobic) and fungal, endotoxin and mycoplasma presence; the sample can be used for flow cytometry analysis, which may include a determination of viable cell number.

If a clinical product is stored in a syringe, it can be placed on a vertical rotator and maintained at room temperature for not more than 6 hours and if the clinical product is in a bag, it can be placed on a horizontal rotator and maintained at room temperature for not more than 6 hours.

In some embodiments, the amount of γMSCs administered to the subject comprises about 1×10⁶ cells/kg of ideal body weight or actual body weight to about 10×10⁶ cells/kg of ideal body weight or actual body weight. In some embodiments, the amount of γMSCs administered to the subject comprises about 1×10⁶ cells/kg of ideal body weight or actual body weight to about 2×10⁶ cells/kg of ideal body weight or actual body weight, about 1×10⁶ cells/kg of ideal body weight or actual body weight to about 3×10⁶ cells/kg of ideal body weight or actual body weight, about 1×10⁶ cells/kg of ideal body weight or actual body weight to about 4×10⁶ cells/kg of ideal body weight or actual body weight, about 1×10⁶ cells/kg of ideal body weight or actual body weight to about 5×10⁶ cells/kg of ideal body weight or actual body weight, about 1×10⁶ cells/kg of ideal body weight or actual body weight to about 6×10⁶ cells/kg of ideal body weight or actual body weight, about 1×10⁶ cells/kg of ideal body weight or actual body weight to about 7×10⁶ cells/kg of ideal body weight or actual body weight, about 1×10⁶ cells/kg of ideal body weight or actual body weight to about 8×10⁶ cells/kg of ideal body weight or actual body weight, about 1×10⁶ cells/kg of ideal body weight or actual body weight to about 9×10⁶ cells/kg of ideal body weight or actual body weight, about 1×10⁶ cells/kg of ideal body weight or actual body weight to about 10×10⁶ cells/kg of ideal body weight or actual body weight, about 2×10⁶ cells/kg of ideal body weight or actual body weight to about 3×10⁶ cells/kg of ideal body weight or actual body weight, about 2×10⁶ cells/kg of ideal body weight or actual body weight to about 4×10⁶ cells/kg of ideal body weight or actual body weight, about 2×10⁶ cells/kg of ideal body weight or actual body weight to about 5×10⁶ cells/kg of ideal body weight or actual body weight, about 2×10⁶ cells/kg of ideal body weight or actual body weight to about 6×10⁶ cells/kg of ideal body weight or actual body weight, about 2×10⁶ cells/kg of ideal body weight or actual body weight to about 7×10⁶ cells/kg of ideal body weight or actual body weight, about 2×10⁶ cells/kg of ideal body weight or actual body weight to about 8×10⁶ cells/kg of ideal body weight or actual body weight, about 2×10⁶ cells/kg of ideal body weight or actual body weight to about 9×10⁶ cells/kg of ideal body weight or actual body weight, about 2×10⁶ cells/kg of ideal body weight or actual body weight to about 10×10⁶ cells/kg of ideal body weight or actual body weight, about 3×10⁶ cells/kg of ideal body weight or actual body weight to about 4×10⁶ cells/kg of ideal body weight or actual body weight, about 3×10⁶ cells/kg of ideal body weight or actual body weight to about 5×10⁶ cells/kg of ideal body weight or actual body weight, about 3×10⁶ cells/kg of ideal body weight or actual body weight to about 6×10⁶ cells/kg of ideal body weight or actual body weight, about 3×10⁶ cells/kg of ideal body weight or actual body weight to about 7×10⁶ cells/kg of ideal body weight or actual body weight, about 3×10⁶ cells/kg of ideal body weight or actual body weight to about 8×10⁶ cells/kg of ideal body weight or actual body weight, about 3×10⁶ cells/kg of ideal body weight or actual body weight to about 9×10⁶ cells/kg of ideal body weight or actual body weight, about 3×10⁶ cells/kg of ideal body weight or actual body weight to about 10×10⁶ cells/kg of ideal body weight or actual body weight, about 4×10⁶ cells/kg of ideal body weight or actual body weight to about 5×10⁶ cells/kg of ideal body weight or actual body weight, about 4×10⁶ cells/kg of ideal body weight or actual body weight to about 6×10⁶ cells/kg of ideal body weight or actual body weight, about 4×10⁶ cells/kg of ideal body weight or actual body weight to about 7×10⁶ cells/kg of ideal body weight or actual body weight, about 4×10⁶ cells/kg of ideal body weight or actual body weight to about 8×10⁶ cells/kg of ideal body weight or actual body weight, about 4×10⁶ cells/kg of ideal body weight or actual body weight to about 9×10⁶ cells/kg of ideal body weight or actual body weight, about 4×10⁶ cells/kg of ideal body weight or actual body weight to about 10×10⁶ cells/kg of ideal body weight or actual body weight, about 5×10⁶ cells/kg of ideal body weight or actual body weight to about 6×10⁶ cells/kg of ideal body weight or actual body weight, about 5×10⁶ cells/kg of ideal body weight or actual body weight to about 7×10⁶ cells/kg of ideal body weight or actual body weight, about 5×10⁶ cells/kg of ideal body weight or actual body weight to about 8×10⁶ cells/kg of ideal body weight or actual body weight, about 5×10⁶ cells/kg of ideal body weight or actual body weight to about 9×10⁶ cells/kg of ideal body weight or actual body weight, about 5×10⁶ cells/kg of ideal body weight or actual body weight to about 10×10⁶ cells/kg of ideal body weight or actual body weight, about 6×10⁶ cells/kg of ideal body weight or actual body weight to about 7×10⁶ cells/kg of ideal body weight or actual body weight, about 6×10⁶ cells/kg of ideal body weight or actual body weight to about 8×10⁶ cells/kg of ideal body weight or actual body weight, about 6×10⁶ cells/kg of ideal body weight or actual body weight to about 9×10⁶ cells/kg of ideal body weight or actual body weight, about 6×10⁶ cells/kg of ideal body weight or actual body weight to about 10×10⁶ cells/kg of ideal body weight or actual body weight, about 7×10⁶ cells/kg of ideal body weight or actual body weight to about 8×10⁶ cells/kg of ideal body weight or actual body weight, about 7×10⁶ cells/kg of ideal body weight or actual body weight to about 9×10⁶ cells/kg of ideal body weight or actual body weight, about 7×10⁶ cells/kg of ideal body weight or actual body weight to about 10×10⁶ cells/kg of ideal body weight or actual body weight, about 8×10⁶ cells/kg of ideal body weight or actual body weight to about 9×10⁶ cells/kg of ideal body weight or actual body weight, about 8×10⁶ cells/kg of ideal body weight or actual body weight to about 10×10⁶ cells/kg of ideal body weight or actual body weight, or about 9×10⁶ cells/kg of ideal body weight or actual body weight to about 10×10⁶ cells/kg of ideal body weight or actual body weight.

In some embodiments, the amount of γMSCs administered to the subject comprises about 1×10⁶ cells/kg of ideal body weight or actual body weight, about 2×10⁶ cells/kg of ideal body weight or actual body weight, about 3×10⁶ cells/kg of ideal body weight or actual body weight, about 4×10⁶ cells/kg of ideal body weight or actual body weight, about 5×10⁶ cells/kg of ideal body weight or actual body weight, about 6×10⁶ cells/kg of ideal body weight or actual body weight, about 7×10⁶ cells/kg of ideal body weight or actual body weight, about 8×10⁶ cells/kg of ideal body weight or actual body weight, about 9×10⁶ cells/kg of ideal body weight or actual body weight, or about 10×10⁶ cells/kg of ideal body weight or actual body weight. In some embodiments, the amount of γMSCs administered to the subject comprises at least about 1×10⁶ cells/kg of ideal body weight or actual body weight, about 2×10⁶ cells/kg of ideal body weight or actual body weight, about 3×10⁶ cells/kg of ideal body weight or actual body weight, about 4×10⁶ cells/kg of ideal body weight or actual body weight, about 5×10⁶ cells/kg of ideal body weight or actual body weight, about 6×10⁶ cells/kg of ideal body weight or actual body weight, about 7×10⁶ cells/kg of ideal body weight or actual body weight, about 8×10⁶ cells/kg of ideal body weight or actual body weight, or about 9×10⁶ cells/kg of ideal body weight or actual body weight. In some embodiments, the amount of γMSCs administered to the subject comprises at most about 2×10⁶ cells/kg of ideal body weight or actual body weight, about 3×10⁶ cells/kg of ideal body weight or actual body weight, about 4×10⁶ cells/kg of ideal body weight or actual body weight, about 5×10⁶ cells/kg of ideal body weight or actual body weight, about 6×10⁶ cells/kg of ideal body weight or actual body weight, about 7×10⁶ cells/kg of ideal body weight or actual body weight, about 8×10⁶ cells/kg of ideal body weight or actual body weight, about 9×10⁶ cells/kg of ideal body weight or actual body weight, or about 10×10⁶ cells/kg of ideal body weight or actual body weight.

In some embodiments, the subject may also be administered a plurality of doses of γMSCs in amounts from about 1×10⁶ cells/kg to about 10×10⁶ cells/kg of ideal body weight or actual body weight. In some embodiments, the subject is administered a plurality of doses of γMSCs in amounts from about 2×10⁶ cells/kg to about 5×10⁶ cells/kg of ideal body weight or actual body weight. The subject may be administered a plurality of doses of γMSCs in amounts from about 1×10⁶ cells/kg to about 2×10⁶ cells/kg, from about 2×10⁶ cells/kg to about 5×10⁶ cells/kg, or from about 5×10⁶ cells/kg to about 10×10⁶ cells/kg.

In some embodiments, the amount of γMSCs administered in a first dose is less than the amount of γMSCs administered in one or more subsequent doses. In some embodiments, the first dose is at least about 2×10⁶ cells/kg and the second dose is at least about 5×10⁶ cells/kg of ideal body weight or actual body weight.

In some embodiments, the methods described herein comprise a subsequent dose being administered to the subject. The second dose may be administered at an amount greater than the preceding dose. The subject may also receive a plurality of doses comprising at least two doses, three doses, four doses, five doses, six doses, seven doses, eight doses, nine doses, or ten or more doses.

In some embodiments, the MSCs may be expanded in culture as described herein prior to being interferon γ-primed. The MSCs may also undergo a primary expansion followed by cryopreservation, as described herein. The MSCs may also be thawed and undergo a second expansion, as described herein.

In some embodiments, the MSCs have been thawed to at least about 0° C., 1° C., 2° C., 3° C., 4° C., 5° C., 6° C., 7° C., 8° C., 9° C. or 10° C. In some embodiments, the MSCs have been thawed to between about 0° C. to 10° C., 0° C. to 9° C., 0° C. to 8° C., 0° C. to 7° C., 0° C. to 6° C., 0° C. to 5° C., 0° C. to 4° C., 0° C. to 3° C., 0° C. to 2° C. or 0° C. to 1° C. In some embodiments, the MSCs have been thawed at least about 1 hours, 2 hours, 3 hours, 4 hours, 5 hours, 6 hours, 7 hours, 8 hours, 9 hours, 19 hours, 11 hours, 12 hours, 13 hours, 14 hours, 15 hours, 16 hours, 17 hours, 18 hours, 19 hours, 20 hours, 21 hours, 22 hours, 23 or 24 hours prior to administration to the subject. In some embodiments, the MSCs have been thawed for at least about 1 days, 1.5 days, 2 days, 2.5 days, 3 days, 3.5 days, 4 days, 4.5 days, 6 days, 6.5 days, 7 days, or more than 7 days prior to administration to the subject.

In some embodiments the MSCs are warmed to room temperature before administration to a subject using the methods described herein. In some embodiments, the MSCs are warmed to about body temperature before administration to a subject. In some embodiments, the MSCs are warmed to about 10° C., 11° C., 12° C., 13° C., 14° C., 15° C., 16° C., 17° C., 18° C., 19 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., or 38° C. before administration to a subject.

In some embodiments, the second expansion may last for about seven days. The second expansion may occur for about 1 to about 3 days, about 1 to about 5 days, about 1 to about 9 days, about 1 to about 11 days, about 1 to about 13 days, or about 1 to about 15 days. The second expansion may occur for about 1, 2, 3, 4, 5, 6, 7, 8, 9, 10, 11, 12, 13, 14, or 15 days.

In some embodiments, the MSCs are primed with interferon gamma (IFNγ) during the second expansion. The IFNγ may be added to the expansion media during the final day, the final 2 days, or the final 3 days of the second expansion. The IFNγ may also be added to the expansion media during the final 4 days, the final 5 days, the final 6 days, the final 7 days, the final 8 days, the final 9 days, the final 10 days, the final 11 days, the final 12 days, the final 13 days, or the final 14 days of the second expansion.

In some embodiments, the IFNγ may be present in the expansion medium at a concentration of about 100 U/ml to about 1000 U/ml. The IFNγ may be present in the expansion medium at a concentration of about 10 U/ml to about 500 U/ml, about 10 U/ml to about 1000 U/ml, about 10 U/ml to about 1500 U/ml, about 10 U/ml to about 2000 U/ml, about 50 U/ml to about 500 U/ml, about 50 U/ml to about 1000 U/ml, about 50 U/ml to about 1500 U/ml, about 50 U/ml to about 2000 U/ml, about 100 U/ml to about 500 U/ml, about 100 U/ml to about 1500 U/ml, and about 100 U/ml to about 2000 U/ml. The IFNγ may be present in the expansion medium at a concentration of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 1920, 1930, 1940, 1950, 1960, 1970, 1980, 1990, or 2000 U/ml.

In some embodiments, the IFNγ may be present in the expansion medium at a concentration of about 1 ng/ml to about 30 ng/ml. The IFNγ may be present in the expansion medium at a concentration of about 1 ng/ml to about 15 ng/ml, 1 ng/ml to about 45 ng/ml, or 1 ng/ml to about 60 ng/ml. The IFNγ may be present in the expansion medium at a concentration of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 ng/ml.

In any of the herein disclosed aspects or embodiments, rather than cryopreserving MSCs or γMSCs and thawing the MSCs (which are subsequently primed) or thawing the γMSCs prior to use (either immediately or after one or more culturing steps), fresh MSCs or fresh γMSCs may be used.

In some cases, when the MSCs were being interferon γ-primed, the culture was not in a hypoxic condition. As examples, the culture did not have decreased levels of O₂, did not have increased levels of CO₂, and/or lacked the presence of a hypoxia mimetic. Decreased levels of O₂ may be from about 1% O₂ to about 5% 02. Increased levels of CO₂ can be about 5% CO₂ or higher. Illustrative hypoxia mimetics include desferoxamine, cobalt chloride, hydralazine, nickel chloride, diazoxide, and dimethyloxalyglycine.

In some embodiments, the subject is a human. In some embodiments, the human may be a child or an adult. In some embodiments, the subject has persistent inflammatory conditions, including autoimmune conditions. In some embodiments, the subject has persistent atopic inflammatory conditions, including autoimmune conditions. In some embodiments, the inflammatory conditions, including autoimmune conditions is moderate-to-severe.

In some embodiments, the γMSCs improve lung function. In some embodiments, the γMSCs reduce the onset of an inflammatory conditions, including autoimmune conditions exacerbation, difficulty in breathing, coughing, dyspnea, the number and/or severity of episodes of inflammatory conditions, including autoimmune conditions attacks, shortness of breath, wheezing when exhaling chest tightness, and/or chest pain experienced by the subject. In some embodiments, the lung function is characterized by aspirometry, oscillometry, bronchodilator reversibility test, lung clearance index test, exhaled nitric oxide test, breath condensate collection test, and/or another method for determining lung function.

In some embodiments, the γMSC reduces upper airway inflammation. In some embodiments, the upper airway inflammation is determined by analysis of small molecule inflammatory constituents in exhaled breath condensate. In some embodiments, the γMSC reduces circulating cell inflammation. In some embodiments, the circulating cell inflammation is determined by flowcytometric analysis of peripheral blood cells and/or Ab Seq analysis of peripheral blood cells.

In some embodiments, the method for treating inflammatory conditions, including autoimmune conditions or a symptom thereof and/or reducing the likelihood of inflammatory conditions, including autoimmune conditions attack in a subject further comprises administering albuterol sulfate for bronchodilator reversibility test and/or sulfur hexafluoride (SF6) for Lung Clearance Index testing. In some embodiments, the method further comprises administering a standard of care treatment for inflammatory conditions, including autoimmune conditions. In some embodiments, the standard of care treatment for inflammatory conditions, including autoimmune conditions comprises albuterol, prednisone, and/or prednisolone. In some embodiments, the standard of care treatment for inflammatory conditions, including autoimmune conditions, comprises a biologic, wherein the biologic is selected from the group consisting of reslizumab (anti IL-5 neutralizing antibody), benralizumab (anti IL-5 receptor alpha), dupilumab (anti IL-4 receptor alpha subunit), mepolizumab (anti IL-5 neutralizing antibody), and/or omalizumab (anti-IgE neutralizing antibody). In some embodiments, the standard of care treatment for inflammatory conditions, including autoimmune conditions is a controller therapy for inflammatory conditions, including autoimmune conditions. In some embodiments, the controller therapy for inflammatory conditions, including autoimmune conditions comprises inhaled corticosteroids. In some embodiments, the standard of care treatment for inflammatory conditions, including autoimmune conditions comprises analgesics, antihistamines, and/or intranasal corticosteroids. In some embodiments, the subject is administered diphenhydramine and/or acetaminophen within 60 minutes of cell infusion.

In some embodiments, the subject is administered the standard of care treatment for inflammatory conditions, including autoimmune conditions before, contemporaneously with, and/or after administration of the γMSC. In some embodiments, the subject is administered the same standard of care treatment for inflammatory conditions, including autoimmune conditions that was administered before administration of the γMSC. In some embodiments, the subject is administered the different standard of care treatment for inflammatory conditions, including autoimmune conditions that was administered before administration of the γMSC. In some embodiments, an anesthetic cream or spray is administered prior to venipuncture or intravenous catheter insertion associated with administering the γMSCs. In some embodiments, the inflammatory conditions, including autoimmune conditions is not treated by the standard of care treatment for inflammatory conditions, including autoimmune conditions.

In some embodiments, the γMSC is administered intravenously. In some embodiments, the γMSC is intravenously infused through a large bore peripheral IV. In some embodiments, the γMSC is infused by IV push or syringe pump, optionally, infused over 5-15 minutes. In some embodiments, no medications are administered through a catheter used for the γMSC infusion.

In some embodiments, the MSCs are obtained from one or more vertebral bodies and the MSCs comprise vertebral bone marrow MSCs (vBM-MSCs), vertebral bone-adherent MSCs (vBA-MSCs), or both.

In some embodiments, the methods comprise obtaining the MSCs from one or more vertebral bodies. The MSCs may comprise vertebral bone marrow MSCs (vBM-MSCs), vertebral bone-adherent MSCs (vBA-MSCs), or both. The MSCs may comprise less than about 5% CD45+ cells (e.g., less than less than about 5% CD45+ cells, less than less than about 4% CD45+ cells, less than about 3% CD45+ cells, less than about 2% CD45+ cells, less than about 1% CD45+ cells, or about 0% CD45+ cells), comprises at least about 90% CD105+ cells (e.g., about 90% CD105+ cells, about 91% CD105+ cells, about 92% CD105+ cells, about 93% CD105+ cells, about 94% CD105+ cells, about 95% CD105+ cells, about 96% CD105+ cells, about 97% CD105+ cells, about 98% CD105+ cells, or at least about 99% CD105+ cells), and/or comprises at least about 90% CD166+ cells. In some embodiments, the MSCs comprise less than about 10% CD45+ cells, at least about 90% CD105+ cells, and/or at least about 90% CD166+ cells. In some embodiments, the γMSCs were cultured under a serum starved condition and/or were exposed to a temperature shock during culturing.

In one aspect, the present disclosure provides a method for treating inflammatory conditions, including autoimmune conditions or a symptom thereof and/or reducing the likelihood of inflammatory conditions, including autoimmune conditions attack thereof in a subject, the method comprising identifying a subject with inflammatory conditions, including autoimmune conditions and/or recurrent of inflammatory conditions, including autoimmune conditions attack who is minimally treated by a standard of care treatment for inflammatory conditions, including autoimmune conditions or has a frequency of recurrent inflammatory conditions, including autoimmune conditions attack that is not reduced by the standard of care treatment for inflammatory conditions, including autoimmune conditions; and administering to the subject an effective amount of interferon γ-primed mesenchymal stromal cells (γMSCs), wherein when the MSCs were being interferon γ-primed, the MSCs were not in a culture in a hypoxic condition. In another aspect, the present disclosure provides a method for treating inflammatory conditions, including autoimmune conditions or a symptom thereof and/or reducing the likelihood of inflammatory conditions, including autoimmune conditions attack thereof in a subject, the method comprising identifying a subject with moderate-to-severe persistent inflammatory conditions, including autoimmune conditions; and administering to the subject an effective amount of interferon γ-primed mesenchymal stromal cells (γMSCs), wherein when the MSCs were being interferon γ-primed, the MSCs were not in a culture in a hypoxic condition.

In an aspect, the present disclosure provides a use of any herein-disclosed composition in a method for preventing or reducing the likelihood of inflammatory conditions, including autoimmune conditions or a symptom thereof.

The present disclosure should be considered as illustrative and not restrictive in character. It is understood that only certain embodiments have been presented and that all changes, modifications and further applications that come within the spirit of the disclosure are desired to be protected.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. Various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

Methods for Obtaining Cells from Cadaver Bones

Methods for obtaining bone marrow cells, comprising one or more of HSCs and MSCs (including vBM-MSC and vBA-MSCs) are described elsewhere, e.g., WO2022226356A1, WO2022221672A1, WO2022159824A1, WO2022140296A1, WO2022140613A1, WO2022133282A1, WO2022081909A1, and WO2022081896A1, the contents of which are herein incorporated by reference to the same extent as if each individual patent application was specifically and individually indicated to be incorporated by reference.

### Preparing the Donor Bone

In various embodiments, an Organ Procurement Organization (OPOs) performs evaluation and donor recoveries according to 21 CFR § 1271. A donor screening and monitoring process may include completion and assessment of a Uniform Donor Risk Assessment Interview (UDRAI). The UDRAI comprises of flowcharts, guidance documents, and questionnaires which are used to screen potential donors related to medical history, behavioral history, travel history and social history. Serology testing may be completed in a CLIA/CMS-approved laboratory using FDA-cleared test kits to rule out viral pathogens. Donors should be negative or non-reactive for HIV-I, HIV-2, hepatitis B virus (HBV, surface and core antigen), hepatitis C virus (HCV), Syphilis (Treponema pallidum), human T-lymphotropic virus types 1 and 2 (HTLV-1, HTLV-2), West Nile Virus, Chagas (Trypanosoma cruzi), Toxoplasmosis, and Epstein-Barr Virus (EBV). Donors may be screened for Cytomegalovirus (CMV). In some embodiments, a donor is under 30 years old, non-smoker, and confirmed CMV negative.

In some embodiments, the donor bone is vertebral bodies. However, it is understood that the methods described herein can be used on the ilium, a combination of the vertebral bodies and ilium, or other bones suitable for extraction of MSCs, even donor bones with lower expected yields.

It is understood that the donor bones can be procured according to fixed protocols for clinical recovery. Bones can be recovered by surgeons or by personnel at a trained OPO (organ procurement organization) using an osteotome and mallet from consented organ and tissue donors. Unprocessed bones are preferably wrapped in sponges and towels soaked in saline to ensure moisture retention during hypothermic shipment on wet ice at a temperature of 0 to 10° F. to a processing facility.

The process for preparing the donor bone can occur soon after the bone is obtained from the deceased donor or can occur after the donor bone has been shipped in a hypothermic environment to a processing facility. Since the donor bone can experience prolonged periods of ischemia during recovery and shipment to the processing facility, care must be taken to track the length and type of ischemia—i.e., warm ischemia and cold ischemia. As described in more detail herein, bone subject to predetermined periods of warm and/or cold ischemia are suitable for obtaining meaningful quantities of viable bone marrow cells.

During the processing of the donor bone, the bone is debrided in an ISO-5 (class 100) environment (biosafety cabinet) with an ISO-7 (class 10,000) background (clean room), with special care taken to sterilize the bag containing the donor bone, such as by spraying with 70% isopropanol. In one embodiment, the debridement is conducted manually using scalpels, osteotomes and gouges. In processing vertebrae, typically a spinal segment including multiple vertebral levels will be provided. In a typical case, the spine segment runs from T8 to LS, for ten vertebral bodies. During initial debridement of the spinal segment, when enough soft tissue has been removed to visualize the pedicles, the pedicles are removed using either a tissue processing band saw or a bone saw, such as the Stryker System 6 Saw (Stryker, Kalamazoo, MI). Special care is taken to avoid breaching the cortical bone which would expose the cancellous bone, to ensure that the hypoxic cancellous bone marrow remains protected throughout the entire debriding process. The anterior element of the vertebral bodies remains, while the pedicles and posterior elements are discarded.

Using a boning knife or tissue processing band saw, the vertebral bodies are separated at the intervertebral discs. The intervertebral disc and soft tissue remaining on each vertebral body is removed with a scalpel, scissors and/or osteotomes, leaving clean, separated VBs. In the case of donor ilium, the soft tissue can be removed with gouges and a scalpel, with special care again taken to ensure that the cortical bone is not breached. Any anatomical pathologies or injuries of the bone are noted and recorded as part of the batch record for the marrow ultimately obtained from the bones. Bones damaged during the recovery process are discarded.

The VBs are placed into a sterile bag and submerged in a 10% bleach solution, yielding a concentration of 5,000 ppm free chlorine, for a predetermined period, typically 5 or more minutes. Bleach has a broad spectrum of anti-microbial activity, does not leave a toxic residue, is unaffected by water hardness and is fast acting. At the end of the period, the bones are transferred to another sterile bag and submerged in a 3% hydrogen peroxide (H₂O₂) solution. The bag is closed and shaken briefly to ensure that the entire surf ace of the bone is in contact with the solution. Most living cells include catalase, which is an enzyme that catalyzes the breakdown of H₂O₂ into H₂O and O₂. This breakdown manifests as foam or froth when the H₂O₂ solution contacts soft tissue but not bone. The foam level can be observed as an indication of the amount of soft tissue remaining on the bone. This observation can be performed manually by a human processor or, in another embodiment, by an automated processor. The automated processor incorporates a visualization device, such as a camera, and object recognition software that can determine foam levels within the bag. The addition of an inert contrast dye can help the human or automated processor detect the foam level. If any foam or froth is observed, the bone is returned for further processing to remove all of the remaining soft tissue from the bone. Once the VBs or ilium has been cleaned of all soft tissue, the bones are transferred to a new sterile bag. The bag is filled with 1 L of PLASMA-LYTE™ (multiple electrolytes injection obtained from Baxter Healthcare, Ltd.), or other suitable sterile, nonpyrogenic isotonic solution. The bag is closed and shaken briefly to ensure that the entire bone is contacted with the PLASMA-LYTE™.

Bone marrow from each group of VBs processed at different duration of bleach treatment can be tested by flow cytometry to assess the viability of the cells isolated from the bone marrow (Table 6). As seen from Table, soaking the VBs for more than 10 minutes yields no significant difference in cell viability compared to when the VBs are soaked for up to 25 minutes.

TABLE 6
Bleach Soak of Vertebral Bodies

| Time Point | CD45+ | CD34+ | CD3+ |
|---|---|---|---|
| (min) | % Viability | % Viability | % Viability |
| 10 | 88.59 | 95.92 | 80.50 |
| 15 | 88.78 | 97.95 | 75.61 |
| 20 | 87.12 | 96.85 | 71.14 |
| 25 | 86.81 | 96.75 | 71.67 |
| Avg. | 87.825 | 96.8675 | 74.73 |
| SD | 0.869554 | 0.721747 | 3.752299 |

In some embodiments, the bleach treatment comprises using 1%, 2%, 3%, 4%, 5%, 6%, 7%, 8%, 9%, 10%, 11%, 12%, 13%, 14%, 15%, 20%, 25%, 30%, 35%, 40%, 45%, 50%, or higher percentage of bleach. In some embodiments, the bleach treatment comprises contacting the VBs with bleach for at least 1 minute, 2 minutes, 3 minutes, 4 minutes, 5 minutes, 6 minutes, 7 minutes, 8 minutes, 9 minutes, 10 minutes, 11, minutes, 12 minutes, 13 minutes, 14 minutes, 15 minutes, 20 minutes, 25 minutes, 30 minutes, 35 minutes, 40 minutes, 45 minutes, 50 minutes, or longer duration. In some embodiments, the viability of the bone marrow cells isolated from the VBs treated with the bleach treatment is not significantly decreased at any duration of bleach treatment described herein compared to bone marrow cells isolated from the VBs without the bleach treatment. In some embodiments, the viability of the bone marrow cells isolated from the VBs treated with 11 minutes, 12 minutes, 13 minutes, 14 minutes, 15 minutes, 20 minutes, 25 minutes, 30 minutes, 35 minutes, 40 minutes, 45 minutes, 50 minutes, or longer duration of the bleach treatment is not decreased or is decreased by less than 3% compared to the viability of the bone marrow cells isolated from the VBs treated with the 10 minutes bleach treatment. In some embodiments, the viability of the bone marrow cells isolated from the VBs treated with more than 10 minutes decreased by less than 2% compared to the viability of the bone marrow cells isolated from the VBs treated with the 10 minutes bleach treatment. In some embodiments, the viability of the bone marrow cells isolated from the VBs treated with more than 10 minutes decreased by less than 1% compared to the viability of the bone marrow cells isolated from the VBs treated with the 10 minutes bleach treatment.

The bone is removed from the bag and from the PLASMA-LYTE™, and a sterile gauze or sponge is used to absorb any liquid remaining on the VBs. In one approach, a saw and/or anvil shears are used to cut the VBs are cut into smaller pieces, such as 1.5 cm 2 pieces, that are small enough for fragmenting with a bone grinder. To simplify the process and for increased safety to the processing personnel, a custom bone cutting tool as described in WO2020/214400A1, which is hereby incorporated by reference in its entirety, is provided is used to cut the VBs into the smaller pieces.

Methods of Deriving Hematopoietic Stem Cells from Bone Marrow

A fresh cadaver vertebral body (VB) or a warmed, previously cryopreserved VB is prepared for grinding.

In one approach, a saw and/or anvil shears are used to cut the VBs are cut into smaller pieces, such as 1.5 cm² pieces, that are small enough for fragmenting with a bone grinder. In order to simplify the process and for increased safety to the processing personnel, a custom bone cutting tool as described in US2019/0343112, which is hereby incorporated by reference in its entirety, is provided is used to cut the VBs into the smaller pieces. Another custom bone cutting tool can be used in combination, or in lieu of the custom bone cutting tool as described in US2019/0343112. The additional bone cutting tool is described in US2020/0325451, which is hereby incorporated by reference in its entirety.

The elements of the bone cutting tool are formed of medical grade stainless steel. The steel is preferably hardened steel capable of withstanding the forces required to cut through bone. In the cleaning process, the tool is subjected to steam sterilization, which can be deleterious to the steel. Thus, in one feature of the present disclosure, the surf aces of the stainless-steel elements are passivated to prevent oxidation of the steel elements during sterilization.

The pieces produced by the bone cutting tool are immediately placed into a sterile pitcher and submerged in 300-500 ml of a grind media. In one aspect of the present system and method, the grind media uses PLASMA-LYTE™-A as a base with heparin, human serum albumin (HSA), and a nuclease (Merck KGAA Corporation). Heparin is used as an anticoagulant. Other anticoagulants at various quantities can also be used. HSA provides a protein source to prevent cell adherence and adsorption to surfaces, as well as reactive oxygen scavenging. It is noted that conventional grind media utilizes DNase, but for the present disclosure Benzonase® or Denarase® reagent is substituted for DNase™ reagent (Qiagen Sciences LLC). Whereas DNase works only on DNA, modem pharmaceutical biotechnology processing relies on enzymes that can cleave all forms of DNA and RNA, and can reduce the viscosity of the solution in which the cells are suspended. It is noted that IMDM (Iscove's Modified Dulbecco's Media) can substitute for the PLASMA-LYTE™-A, since IMDM is suitable for rapidly proliferating high-density cell cultures and ideal for supporting T- and B-lymphocytes. It is further noted that Denarase reagent (C-Lecta GmbH) is equivalent to Benzonase reagent in the same quantity in the present process.

In some embodiments, the amount of heparin in the grind media is about 5 U/ml to about 15 U/ml. In some embodiments, the amount of heparin in the grind media is about 5 U/ml to about 6 U/ml, about 5 U/ml to about 7 U/ml, about 5 U/ml to about 8 U/ml, about 5 U/ml to about 9 U/ml, about s U/ml to about 10 U/ml, about s U/ml to about 11 U/ml, about s U/ml to about 12 U/ml, about 5 U/ml to about 13 U/ml, about 5 U/ml to about 14 U/ml, about 5 U/ml to about 15 U/ml, about 6 U/ml to about 7 U/ml, about 6 U/ml to about 8 U/ml, about 6 U/ml to about 9 U/ml, about 6 U/ml to about 10 U/ml, about 6 U/ml to about 11 U/ml, about 6 U/ml to about 12 U/ml, about 6 U/ml to about 13 U/ml, about 6 U/ml to about 14 U/ml, about 6 U/ml to about 15 U/ml, about 7 U/ml to about 8 U/ml, about 7 U/ml to about 9 U/ml, about 7 U/ml to about 10 U/ml, about 7 U/ml to about 11 U/ml, about 7 U/ml to about 12 U/ml, about 7 U/ml to about 13 U/ml, about 7 U/ml to about 14 U/ml, about 7 U/ml to about 15 U/ml, about 8 U/ml to about 9 U/ml, about 8 U/ml to about 10 U/ml, about 8 U/ml to about 11 U/ml, about 8 U/ml to about 12 U/ml, about 8 U/ml to about 13 U/ml, about 8 U/ml to about 14 U/ml, about 8 U/ml to about 15 U/ml, about 9 U/ml to about 10 U/ml, about 9 U/ml to about 11 U/ml, about 9 U/ml to about 12 U/ml, about 9 U/ml to about 13 U/ml, about 9 U/ml to about 14 U/ml, about 9 U/ml to about 15 U/ml, about 10 U/ml to about 11 U/ml, about 10 U/ml to about 12 U/ml, about 10 U/ml to about 13 U/ml, about 10 U/ml to about 14 U/ml, about 10 U/ml to about 15 U/ml, about 11 U/ml to about 12 U/ml, about 11 U/ml to about 13 U/ml, about 11 U/ml to about 14 U/ml, about 11 U/ml to about 15 U/ml, about 12 U/ml to about 13 U/ml, about 12 U/ml to about 14 U/ml, about 12 U/ml to about 15 U/ml, about 13 U/ml to about 14 U/ml, about 13 U/ml to about 15 U/ml, or about 14 U/ml to about 15 U/ml. In some embodiments, the amount of heparin in the grind media is about 5 U/ml, about 6 U/ml, about 7 U/ml, about 8 U/ml, about 9 U/ml, about 10 U/ml, about 11 U/ml, about 12 U/ml, about 13 U/ml, about 14 U/ml, or about 15 U/ml. In some embodiments, the amount of heparin in the grind media is at least about 5 U/ml, about 6 U/ml, about 7 U/ml, about 8 U/ml, about 9 U/ml, about 10 U/ml, about 11 U/ml, about 12 U/ml, about 13 U/ml, or about 14 U/ml. In some embodiments, the amount of heparin in the grind media is at most about 6 U/ml, about 7 U/ml, about 8 U/ml, about 9 U/ml, about 10 U/ml, about 11 U/ml, about 12 U/ml, about 13 U/ml, about 14 U/ml, or about 15 U/ml. In some embodiments, the amount of Benzonase® or Denarase® in the grind media is about 11 U/ml to about 55 U/ml. In some embodiments, the amount of Benzonase in the grind media is about 11 U/ml to about 15 U/ml, about 11 U/ml to about 20 U/ml, about 11 U/ml to about 25 U/ml, about 11 U/ml to about 30 U/ml, about 11 U/ml to about 35 U/ml, about 11 U/ml to about 40 U/ml, about 11 U/ml to about 45 U/ml, about 11 U/ml to about 50 U/ml, about 11 U/ml to about 55 U/ml, about 15 U/ml to about 20 U/ml, about 15 U/ml to about 25 U/ml, about 15 U/ml to about 30 U/ml, about 15 U/ml to about 35 U/ml, about 15 U/ml to about 40 U/ml, about 15 U/ml to about 45 U/ml, about 15 U/ml to about 50 U/ml, about 15 U/ml to about 55 U/ml, about 20 U/ml to about 25 U/ml, about 20 U/ml to about 30 U/ml, about 20 U/ml to about 35 U/ml, about 20 U/ml to about 40 U/ml, about 20 U/ml to about 45 U/ml, about 20 U/ml to about 50 U/ml, about 20 U/ml to about 55 U/ml, about 25 U/ml to about 30 U/ml, about 25 U/ml to about 35 U/ml, about 25 U/ml to about 40 U/ml, about 25 U/ml to about 45 U/ml, about 25 U/ml to about 50 U/ml, about 25 U/ml to about 55 U/ml, about 30 U/ml to about 35 U/ml, about 30 U/ml to about 40 U/ml, about 30 U/ml to about 45 U/ml, about 30 U/ml to about 50 U/ml, about 30 U/ml to about 55 U/ml, about 35 U/ml to about 40 U/ml, about 35 U/ml to about 45 U/ml, about 35 U/ml to about 50 U/ml, about 35 U/ml to about 55 U/ml, about 40 U/ml to about 45 U/ml, about 40 U/ml to about 50 U/ml, about 40 U/ml to about 55 U/ml, about 45 U/ml to about SOU/ml, about 45 U/ml to about 55 U/ml, or about 50 U/ml to about 55 U/ml. In some embodiments, the amount of Benzonase in the grind media is about 11 U/ml, about 15 U/ml, about 20 U/ml, about 25 U/ml, about 30 U/ml, about 35 U/ml, about 40 U/ml, about 45 U/ml, about 50 U/ml, or about 55 U/ml. In some embodiments, the amount of Benzonase in the grind media is at least about 11 U/ml, about 15 U/ml, about 20 U/ml, about 25 U/ml, about 30 U/ml, about 35 U/ml, about 40 U/ml, about 45 U/ml, or about 50 U/ml. In some embodiments, the amount of Benzonase in the grind media is at most about 15 U/ml, about 20 U/ml, about 25 U/ml, about 30 U/ml, about 35 U/ml, about 40 U/ml, about 45 U/ml, about 50 U/ml, or about 55 U/ml.

In some embodiments, the amount of Benzonase® in the grind media is about 1 U/ml to about 10 U/ml. In some embodiments, the amount of Benzonase in the grind media is about 1 U/ml to about 2 U/ml, about 1 U/ml to about 3 U/ml, about 1 U/ml to about 4 U/ml, about 1 U/ml to about 5 U/ml, about 1 U/ml to about 6 U/ml, about 1 U/ml to about 7 U/ml, about 1 U/ml to about 8 U/ml, about 1 U/ml to about 9 U/ml, about 1 U/ml to about 10 U/ml, about 2 U/ml to about 3 U/ml, about 2 U/ml to about 4 U/ml, about 2 U/ml to about 5 U/ml, about 2 U/ml to about 6 U/ml, about 2 U/ml to about 7 U/ml, about 2 U/ml to about 8 U/ml, about 2 U/ml to about 9 U/ml, about 2 U/ml to about 10 U/ml, about 3 U/ml to about 4 U/ml, about 3 U/ml to about 5 U/ml, about 3 U/ml to about 6 U/ml, about 3 U/ml to about 7 U/ml, about 3 U/ml to about 8 U/ml, about 3 U/ml to about 9 U/ml, about 3 U/ml to about 10 U/ml, about 4 U/ml to about 5 U/ml, about 4 U/ml to about 6 U/ml, about 4 U/ml to about 7 U/ml, about 4 U/ml to about 8 U/ml, about 4 U/ml to about 9 U/ml, about 4 U/ml to about 10 U/ml, about 5 U/ml to about 6 U/ml, about 5 U/ml to about 7 U/ml, about 5 U/ml to about 8 U/ml, about 5 U/ml to about 9 U/ml, about 5 U/ml to about 10 U/ml, about 6 U/ml to about 7 U/ml, about 6 U/ml to about 8 U/ml, about 6 U/ml to about 9 U/ml, about 6 U/ml to about 10 U/ml, about 7 U/ml to about 8 U/ml, about 7 U/ml to about 9 U/ml, about 7 U/ml to about 10 U/ml, about 8 U/ml to about 9 U/ml, about 8 U/ml to about 10 U/ml, or about 9 U/ml to about 10 U/ml. In some embodiments, the amount of Benzonase in the grind media is about 1 U/ml, about 2 U/ml, about 3 U/ml, about 4 U/ml, about 5 U/ml, about 6 U/ml, about 7 U/ml, about 8 U/ml, ab out 9 U/ml, or about 10 U/ml. In some embodiments, the amount of Benzonase in the grind media is at least about 1 U/ml, about 2 U/ml, about 3 U/ml, about 4 U/ml, about 5 U/ml, about 6 U/ml, about 7 U/ml, about 8 U/ml, or about 9 U/ml. In some embodiments, the amount of Benzonase in the grind media is at most about 2 U/ml, about 3 U/ml, about 4 U/ml, about 5 U/ml, about 6 U/ml, about 7 U/ml, about 8 U/ml, about 9 U/ml, or about 10 U/ml.

In some embodiments, HSA is present in the grind media at about 0.5% to about 5%. In some embodiments, HSA is present in the grind media at about 0.5% to about 1%, about 0.5% to about 1.5%, about 0.5% to about 2%, about 0.5% to about 2.5%, about 0.5% to about 3%, about 0.5% to about 3.5%, about 0.5% to about 4%, about 0.5% to about 4.5%, about 0.5% to about 5%, about 1% to about 1.5%, about 1% to about 2%, about 1% to about 2.5%, about 1% to about 3%, about 1% to about 3.5%, about 1% to about 4%, about 1% to about 4.5%, about 1% to about 5%, about 1.5% to about 2%, about 1.5% to about 2.5%, about 1.5% to about 3%, about 1.5% to about 3.5%, about 1.5% to about 4%, about 1.5% to about 4.5%, about 1.5% to about 5%, about 2% to about 2.5%, about 2% to about 3%, about 2% to about 3.5%, about 2% to about 4%, about 2% to about 4.5%, about 2% to about 5%, about 2.5% to about 3%, about 2.5% to about 3.5%, about 2.5% to about 4%, about 2.5% to about 4.5%, about 2.5% to about 5%, about 3% to about 3.5%, about 3% to about 4%, about 3% to about 4.5%, about 3% to about 5%, about 3.5% to about 4%, about 3.5% to about 4.5%, about 3.5% to about s %, about 4% to about 4.5%, about 4% to about s %, or about 4.5% to about s %. In some embodiments, HAS is present in the grind media at about 0.5%, about 1%, about 1.5%, about 2%, about 2.5%, about 3%, about 3.5%, about 4%, about 4.5%, or about 5%. In some embodiments, HSA is present in the grind media at least about 0.5%, about 1%, about 1.5%, about 2%, about 2.5%, about 3%, about 3.5%, about 4%, or about 4.5%. In some embodiments, HSA is present in the grind media at most about 1%, about 1.5%, about 2%, about 2.5%, about 3%, about 3.5%, about 4%, about 4.5%, or about 5%.

Another pitcher of 300-500 ml of grind media is retained for collecting the bone fragments after grinding, and another supply of about 100 ml of the grind media is retained for rinsing through the grinder during the grinding process to prevent bone fragments from sticking to the surface of the pitcher of the grinding components. In some embodiments, the additional grind media may have different quantities of heparin, HSA, and Benzonase as compared to the initial grind media.

An electric bone grinder or a purpose-built bone grinder, such as the grinder of Biorep Technologies Inc, (Miami, FL) can be used in an ISO-5 environment within an ISO-7 clean room. Bone types are kept separate if both VB and ilium from the same donor are being processed. The bone is kept submerged in grind media at all times during and after the grinding process. Once all of the donor bone pieces are ground, the chamber of the bone grinder is thoroughly rinsed with fresh processing media. The bone fragments are discharged from the grinder into the pitcher containing grind media.

The contents of the pitcher are transferred to sterile bags. Next, the contents of the sterile bags are filtered to extract the solid components. In one embodiment, the contents of each bag are passed through a series of stainless-steel sieves. In this embodiment, a No. 40 (425 μm) sieve is stacked on top of a No. 80 (177 μm) sieve, which is seated over a catch-pan to receive the liquid filter contents. The sterile bags containing the output from the grinder is swirled and then poured evenly over the sieve stack or filtration sets. The filtering process is observed to ensure that excessive clumping is not occurring, which can signal the presence of soft tissue or other contaminants. Bone fragments retained on the surface of the sieves are distributed evenly on the sieves and rinsed with 250 ml of fresh processing medium. In one embodiment, the processing medium used for rinsing is the grind media described above or PLASMA-LYTE™ with 2.5% HSA. The sieved bone marrow product, which can be approximately 1000 ml in a well-performed process, is transferred to sterile packs for subsequent processing and analysis. The contents of each bag are visually inspected to confirm that the contents do not include any visible bone fragments or soft tissue.

In some embodiments, the rinse media can contain the various amounts of HSA as described for the grind media. In some embodiments, the rinse media can contain, additionally, heparin and/or Benzonase.

In another embodiment, the contents of each bag are passed through bone marrow filtration units. In this embodiment, the system includes a stand configured to support a sterile collection bag which contains the bone fragments and media from the grinding operation described above. The stand includes a container hanger configured to engage the cap of the sterile bag to suspend the container. The bottom of the bag includes a discharge assembly that includes a pre-filter projecting into the body of the collection bag. In one specific embodiment the pre-filter is an 850 μm filter. In some embodiments, the bone marrow passes first through an 800 μm pre-filter. The filter is connected to an output tube that is connected by a container claim to the input line of a first in-line filter. In the specific embodiment, the first in-line filter is a 200 μm or a 500 μm filter. The output line of the first in-line filter is connected to the input line of a second in-line filter. The second in-line filter is a 200 μm or a 500 μm filter. The two in-line filters are both initially 500 μm for a first pass through the filter system. A second rinse is then performed on the grindings with the two in-line filters being 200 μm. This double-pass filtration results in a cleaner suspension and enhances removal of fat from the suspension. The second in-line filter has an output line that can be engaged to a sterile bag, such as bag for the second filtration pass. On the second pass through the system, the output line of the second in-line filter can be engaged to a container clamp of a transfer pack container. The transfer pack container can be a 600-2000 ml bag to accommodate the filtered bone marrow product, which can be approximately 1000 ml in a well-performed process.

Agitation of Bone Grindings and/or Bone Grinding Filtrate

Described herein, in some embodiments, is a method for processing bone marrow or derivatives thereof, the method comprises mechanically agitating the bone grindings and/or bone grinding filtrate during the grinding and filtration portion of the processing of the bone marrow. In some instances, the bone marrow can be obtained from a deceased donor. In some cases, the bone marrow can be obtained from a sample (e.g., bone or VB) that was previously chilled. In some cases, the bone marrow can be obtained from a sample (e.g., bone or VB) that was previously chilled but not frozen. In some cases, the bone marrow can be obtained from a sample (e.g., bone or VB) that is thawed. In some cases, the bone marrow can be processed for obtaining bone marrow cells. In some embodiments, the bone marrow cells can be hematopoietic stem cells (HSCs). In some embodiments, the bone marrow cells can be mesenchymal stem cells (MSCs).

Aspect disclosed in the present disclosure comprises a method for processing bone marrow or a derivative thereof, wherein the bone marrow or the derivative thereof is derived from a deceased donor, the method comprising: obtaining a bone or bone fragment from a deceased donor, optionally, processing the bone into bone fragments; mechanically grinding the bone or bone fragment in the presence of a grinding solution to generate a plurality of bone grindings; placing the plurality of bone grindings on a shaker at about 100 to about 200 rounds per minute (“RPM”) for about 1 to about 20 minutes; and removing the solution from the shaker, wherein the solution comprises the bone marrow or the derivative thereof and wherein the bone marrow or the derivative thereof comprises at least about 1,500,000 CD34+ cells/ml of the bone marrow or the derivative thereof. In some embodiments, the method further comprises contacting the solution with a rinse media and repeating the placing of the bone grindings on the shaker and then removing the solution from the shaker. In some embodiments, the method further comprises repeating step placing the bone grinding on the shaker and then removing the solution from the shaker one or more times. In some embodiments, the at least about 1,500,000 CD34+ cells/ml of the bone marrow or the derivative thereof comprises at least 85% viable CD34+ cells. In some embodiments, the method further comprises the at least about 1,500,000 CD34+ cells/ml of the bone marrow or the derivative thereof comprises at least 90% viable CD34+ cells.

The mechanical agitation can comprise agitating the bone grindings in a linear fashion. In some embodiments, the mechanical agitation can comprise agitating the bone grindings in a three-dimensional fashion. In some cases, the mechanical agitation of the bone grindings can comprise orbital shaking (via an orbital shaker) such as placing the bone grinding on a shaker. In some cases, the bone grindings can be mechanically agitated by the shaker at a rate at least about 10 rounds per minute (RPM), 20 RPM, 30 RPM, 40 RPM, 50 RPM, 60 RPM, 70 RPM, 80 RPM, 90 RPM, 100 RPM, 110 RPM, 120 RPM, 130 RPM, 140 RPM, 150 RPM, 160 RPM, 170 RPM, 180 RPM, 190 RPM, 200 RPM, 210 RPM, 220 RPM, 230 RPM, 240 RPM, 250 RPM, or more. In some cases, the bone grindings can be mechanically agitated by centrifugation (e.g., spinning). In some embodiments, the bone grindings can be spun at least 10 RPM, 20 RPM, 30 RPM, 40 RPM, 50 RPM, 60 RPM, 70 RPM, 80 RPM, 90 RPM, 100 RPM, 110 RPM, 120 RPM, 130 RPM, 140 RPM, 150 RPM, 160 RPM, 170 RPM, 180 RPM, 190 RPM, 200 RPM, 210 RPM, 220 RPM, 230 RPM, 240 RPM, 250 RPM, or more. In some embodiments, the bone grindings can be spun at least 300 RPM, 400 RPM, 500 RPM, 600 RPM, or more. In some embodiments, the bone grindings can be mechanically agitated by both shaking and spinning. In some embodiments, the mechanical agitation of the bone grindings can be for at least 1 minute, 2 minutes, 3 minutes, 4 minutes, 5 minutes, 10 minutes, 15 minutes, 20 minutes, 25 minutes, 30 minutes, or longer.

In some embodiments, the mechanical agitation of the bone grindings increases the yield of the bone marrow cells obtained. In some instances, the yield of the bone marrow cells obtained by mechanical agitation of the bone grindings is increased by at least about 10%, 20%, 30%, 40%, 50%, 60%, 70%, 80%, 90%, 100%, 2-fold, 3-fold, 4-fold, 5-fold, 10-fold, 20-fold, 50-fold, or more compared to yield of bone marrow cells obtained without the mechanical agitation.

In some embodiments, the mechanical agitation of the bone grindings increases the viability of the bone marrow cells obtained. In some instances, the viability of the bone marrow cells obtained by mechanical agitation of the bone grindings is increased by at least about 10%, 20%, 30%, 40%, 50%, 60%, 70%, 80%, 90%, 100%, 2-fold, 3-fold, 4-fold, 5-fold, 10-fold, 20-fold, 50-fold, or more compared to the viability of bone marrow cells obtained without the mechanical agitation.

In some embodiments, the mechanical agitation of the bone grindings increases the number of CD34 expressing bone marrow cells obtained. In some instances, the number of CD34 expressing bone marrow cells obtained by mechanical agitation of the bone grindings is increased by at least about 10%, 20%, 30%, 40%, 50%, 60%, 70%, 80%, 90%, 100%, 2-fold, 3-fold, 4-fold, 5-fold, 10-fold, 20-fold, 50-fold, or more compared to the number of CD34 expressing bone marrow cells obtained without the mechanical agitation.

In some embodiments, the mechanical agitation of the bone grindings increases the number of CD45 expressing bone marrow cells obtained by the methods described herein. In some instances, the number of CD45 expressing bone marrow cells obtained by mechanical agitation of the bone grindings is increased by at least about 10%, 20%, 30%, 40%, 50%, 60%, 70%, 80%, 90%, 100%, 2-fold, 3-fold, 4-fold, 5-fold, 10-fold, 20-fold, 50-fold, or more compared to the number of CD45 expressing bone marrow cells obtained without the mechanical agitation.

In some embodiments, the amount of CD34+ cells/ml of the bone marrow or the derivative thereof obtained is at least about 1,500,000 CD34+ cells/ml to about 2,000,000 CD34+ cells/ml. In some embodiments, the amount of CD34+ cells/ml of the bone marrow or the derivative thereof obtained is at least about 1,500,000 CD34+ cells/ml to about 1,750,000 CD34+ cells/ml, about 1,500,000 CD34+ cells/ml to about 2,000,000 CD34+ cells/ml, or about 1,750,000 CD34+ cells/ml to about 2,000,000 CD34+ cells/ml. In some embodiments, the amount of CD34+ cells/ml of the bone marrow or the derivative thereof obtained is at least about 1,500,000 CD34+ cells/ml, about 1,750,000 CD34+ cells/ml, or about 2,000,000 CD34+ cells/ml. In some embodiments, the amount of CD34+ cells/ml of the bone marrow or the derivative thereof obtained is at least at least about 1,500,000 CD34+ cells/ml, or about 1,750,000 CD34+ cells/ml. In some embodiments, the amount of CD34+ cells/ml of the bone marrow or the derivative thereof obtained is at least at most about 1,750,000 CD34+ cells/ml, or about 2,000,000 CD34+ cells/ml.

In some embodiments, the viability of the CD34+ cells is at least about 70% to about 95%. In some embodiments, the viability of the CD34+ cells is at least about 70% to about 75%, about 70% to about 80%, about 70% to about 85%, about 70% to about 90%, about 70% to about 95%, about 75% to about 80%, about 75% to about 85%, about 75% to about 90%, about 75% to about 95%, about 80% to about 85%, about 80% to about 90%, about 80% to about 95%, about 85% to about 90%, about 85% to about 95%, or about 90% to about 95%. In some embodiments, the viability of the CD34+ cells is at least about 70%, about 75%, about 80%, about 85%, about 90%, or about 95%. In some embodiments, the viability of the CD34+ cells is at least at least about 70%, about 75%, about 80%, about 85%, or about 90%. In some embodiments, the viability of the CD34+ cells is at least at most about 75%, about 80%, about 85%, about 90%, or about 95%.

For quality control, a small quantity of bone marrow, such as 0.3 mL, is extracted from the sterile pack using a syringe at an injection site and conducting inversion mixing before pulling the sample. The sample can be tested by a hematology analyzer, such as a Sysmex Hematology Analyzer, to determine the total nucleated cell (TNC) content of the sample, as an indicator of the TNC content of the bone marrow being subsequently processed.

### Fat Removal and Concentration

The bone marrow product collected from the filtering is essentially a fatty emulsion. The fat content of the suspension obtained from the sieve filtering approach disclosed above is greater than the fat content of the suspension obtained from the double-pass filtration system. However, in both cases, there is a need to remove the fat content from the suspension. The suspension obtained from the filtering is recovered into 250 ml bags which are hermetically sealed with tube welders. Pairs of sterile bags and taring sticks are mounted within a centrifuge with bag ports facing down, and balanced. Volume compensating plates are used to prevent creasing of the bags during centrifugation. In one embodiment, the bags are centrifuged at 500×g for 15 minutes at room temperature to concentrate the cells, preferably to 2-3×10⁸/ml. After centrifugation is complete, each bag is individually hung on a ring stand. The distinct layers within the bag are visible, with the fat layer clearly delineated on top of the supernatant with the bone marrow pellet at the bottom. A new sterile bag is welded to the bag removed from the centrifuge. A bag clamp or clip is placed on the bag just below the fat layer, to clamp off or squeeze the bag closed beneath the fat layer. The pellet is then drained from the centrifuge bag into the new sterile bag, with the bag clip preventing passage of the fat layer. The pellet is agitated as it is drained to resuspend all of the pellet. After about half of the pellet has drained into the new bag, the tubing is closed with a hemostat or tube sealer. The second centrifuge bag is then welded to the new bag containing the pellet, and the contents of this second centrifuge bag are drained into the new bag.

The result is new sterile bags containing the bone marrow centrifuged to remove the fat. These bags of de-fatted bone marrow are then centrifuged at 500×g for 15 minutes at room temperature, with volume compensating plates to prevent creasing of the bags. Each bag is removed and suspended on a ring stand and a waste bag is welded to the bag, and a plasma extractor is used to remove the supernatant into the waste bag. The tubing is clamped with a hemostat when the pellet rises or breaks. The tubing is then sealed and severed to remove the pellet-containing bag from the waste bag, which is discarded. A Luer connection is welded to the pellet-containing bag. The pellets from each bag are combined into a bulk bag using a large syringe. The pellet-containing bags are rinsed into the bulk bag using a rinse media. The bulk bag is inverted several times to ensure that all of the pellet is resuspended. A small quantity of the processed BM, such as 0.5 mL, can be removed for quality control testing for density and cell count. The test sample can also be evaluated for human leukocyte antigens, CCR5delta 32 mutation and apolipoprotein (APOE), among other things.

In some embodiments, the centrifuge settings at one or more steps can be increased. In some embodiments, the centrifuge is spun at about 400 g to about 650 g. In some embodiments, the centrifuge is spun at about 400 g to about 450 g, about 400 g to about 500 g, about 400 g to about 550 g, about 400 g to about 600 g, about 400 g to about 650 g, about 450 g to about 500 g, about 450 g to about 550 g, about 450 g to about 600 g, about 450 g to about 650 g, about 500 g to about 550 g, about 500 g to about 600 g, about 500 g to about 650 g, about 550 g to about 600 g, about 550 g to about 650 g, or about 600 g to about 650 g. In some embodiments, the centrifuge is spun at about 400 g, about 450 g, about 500 g, about 550 g, about 600 g, or about 650 g. In some embodiments, the centrifuge is spun at least about 400 g, about 450 g, about 500 g, about 550 g, or about 600 g. In some embodiments, the centrifuge is spun at most about 450 g, about 500 g, about 550 g, about 600 g, or about 650 g. In some embodiments, the centrifuge is spun for about 10 minutes to about 40 minutes. In some embodiments, the centrifuge is spun for about 10 minutes to about 15 minutes, about 10 minutes to about 20 minutes, about 10 minutes to about 25 minutes, about 10 minutes to about 30 minutes, about 10 minutes to about 35 minutes, about 10 minutes to about 40 minutes, about 15 minutes to about 20 minutes, about 15 minutes to about 25 minutes, about 15 minutes to about 30 minutes, about 15 minutes to about 35 minutes, about 15 minutes to about 40 minutes, about 20 minutes to about 25 minutes, about 20 minutes to about 30 minutes, about 20 minutes to about 35 minutes, about 20 minutes to about 40 minutes, about 25 minutes to about 30 minutes, about 25 minutes to about 35 minutes, about 25 minutes to about 40 minutes, about 30 minutes to about 35 minutes, about 30 minutes to about 40 minutes, or about 35 minutes to about 40 minutes. In some embodiments, the centrifuge is spun for about 10 minutes, about 15 minutes, about 20 minutes, about 25 minutes, about 30 minutes, about 35 minutes, or about 40 minutes. In some embodiments, the centrifuge is spun for at least about 10 minutes, about 15 minutes, about 20 minutes, about 25 minutes, about 30 minutes, or about 35 minutes. In some embodiments, the centrifuge is spun for at most about 15 minutes, about 20 minutes, about 25 minutes, about 30 minutes, about 35 minutes, or about 40 minutes. In some embodiments, the centrifuge is stopped without the use of a brake. In some embodiments, the centrifuge is stopped with a brake. In some embodiments, the centrifuge brake is set at about 25% to about 100%. In some embodiments, the centrifuge brake is set at about 25% to about 50%, about 25% to about 75%, about 25% to about 100%, about 50% to about 75%, about 50% to about 100%, or about 75% to about 100%. In some embodiments, the centrifuge brake is set at about 25%, about 50%, about 75%, or about 100%. In some embodiments, the centrifuge brake is set at least about 25%, about 50%, or about 75%. In some embodiments, the centrifuge brake is set at most about 50%, about 75%, or about 100%.

### Isolation of CD34+ Cells

Described herein, in some aspects, is a method for processing (e.g., isolating) CD34+ cells obtained from bone marrow or bone marrow derivative. In some cases, the bone marrow or bone marrow derivative can be fresh (e.g., never frozen) or thawed from being previously frozen. In some embodiments, the bone marrow or bone marrow derivative can be ground by the methods and systems described herein. In some embodiments, ground bone marrow or bone marrow cells can be contacted with the stabilization buffer described herein. In some embodiments, the stabilization prevents formation of aggregates of the bone marrow cells. In some instances, the bone marrow cells contacted and suspended in the stabilization buffer can be isolated by attaching to antibody such as a conjugated antibody. For example, bone marrow cells expressing CD34+ can be isolated and enriched by contacting the bone marrow cells with the CD34 antibody conjugated with iron, where the bone marrow cells expressing CD34 are then trapped a magnetic separation column (e.g., “CliniMACS®”). The bone marrow cells not expressing CD34 can be washed away. The trapped CD34+ bone marrow cells can be harvested by removing the magnetic field and eluting the targeted CD34+ bone marrow cells. Such approach does not require isolating the bone marrow cells with a Ficoll gradient.

Aspect described in the present disclosure comprises a method for processing a population of CD34+ cells obtained from bone marrow or a derivative thereof, wherein the bone marrow or the derivative thereof is derived from a deceased donor, the method comprising: obtaining a bone or bone fragment from a deceased donor, optionally, processing the bone into bone fragments; extracting the bone marrow or derivative thereof from the bone or bone fragment; and contacting the bone marrow or derivative thereof with a stabilization buffer, wherein the stabilization buffer comprises more than about 3 U/ml of a nuclease; performing a CD34+ cell isolation assay to generate a cellular composition comprising the population of CD34+ cells, wherein the composition comprising the population of CD34+ cells comprises at least about 80,000 CD34+ cells/750 μl of the bone marrow or the derivative thereof contacted with the stabilization buff er. In some embodiments, the at least about 80,000 CD34+ cells/750 μl of the bone marrow or the derivative thereof contacted with the stabilization buffer comprise at least 70% viable CD34+ cells. In some embodiments, the at least about 80,000 CD34+ cells/750 μl of the bone marrow or the derivative thereof contacted with the stabilization buffer comprise at least 80% viable CD34+ cells. In some embodiments, the at least about 80,000 CD34+ cells/750 μl of the bone marrow or the derivative thereof contacted with the stabilization buffer comprise at least 90% viable CD34+ cells.

Another aspect of the present disclosure comprises a stabilization buffer comprising at least 5 U/ml of an anticoagulant; and more than 3 U/ml of a nuclease. In some embodiments, stabilization buff er comprises more than about 5 U/ml of a nuclease. In some embodiments, the stabilization buffer comprises more than about 10 U/ml of a nuclease. In some embodiments, the stabilization buffer comprises more than about 15 U/ml of a nuclease. In some embodiments, the stabilization buffer comprises more than about 20 U/ml of a nuclease. In some embodiments, the stabilization buffer comprises about 20 U/ml of a nuclease. In some embodiments, the nuclease is Benzonase® or Denarase®. In some embodiments, the stabilization buffer further comprises more than about 10 U/ml of an anticoagulant. In some embodiments, the stabilization buffer further comprises about 10 U/ml of an anticoagulant. In some embodiments, the anticoagulant is heparin. In some embodiments, the stabilization buffer further comprises human serum albumin (HSA). In some embodiments, the stabilization buffer comprises 0.5% HSA.

In some embodiments, the stabilization buffer comprises nuclease. In some embodiments, the nuclease is Benzonase® or Denarase®. In some embodiments, the stabilization buffer comprises nuclease at about 3 U/ml, 4 U/ml, 5 U/ml, 6 U/ml, 7 U/ml, 8 U/ml, 9 U/ml, 10 U/ml, 11 U/ml, 12 U/ml, 13 U/ml, 14 U/ml, 15 U/ml, 16 U/ml, 17 U/ml, 18 U/ml, 19 U/ml, 20 U/ml, 21 U/ml, 22 U/ml, 23 U/ml, 24 U/ml, 25 U/ml, 26 U/ml, 27 U/ml, 28 U/ml, 29 U/ml, 30 U/ml, 50 U/ml, 100 U/ml, 200 U/ml, or more U/ml. In some embodiments, the stabilization buffer comprises an anticoagulant. In some cases, the anticoagulant is Heparin. In some instances, the stabilization buffer comprises anticoagulant at about 0.1 U/ml, 0.2 U/ml, 0.3 U/ml, 0.4 U/ml, 0.5 U/ml, 0.6 U/ml, 0.7 U/ml, 0.8 U/ml, 0.9 U/ml, 1.0 U/ml, 2.0 U/ml, 3.0 U/ml, 4.0 U/ml, 5.0 U/ml, 6.0 U/ml, 7.0 U/ml, 8.0 U/ml, 9.0 U/ml, 10 U/ml, 11 U/ml, 12 U/ml, 13 U/ml, 14 U/ml, 15 U/ml, 16 U/ml, 17 U/ml, 18 U/ml, 19 U/ml, 20 U/ml, 21 U/ml, 22 U/ml, 23 U/ml, 24 U/ml, 25 U/ml, 26 U/ml, 27 U/ml, 28 U/ml, 29 U/ml, 30 U/ml, 50 U/ml, 100 U/ml, 200 U/ml, or more U/ml.

In some embodiments, the stabilization buffer comprises about 0.001%, 0.002%, 0.003%, 0.004%, 0.005%, 0.01%, 0.02%, 0.03%, 0.04%, 0.05% HSA, 0.1% HSA, 0.2% HSA, 0.3% HSA, 0.4% HSA, 0.5% HSA, 0.6% HSA, 0.7% HSA, 0.8% HSA, 0.9% HSA, 1.0% HSA, 1.5% HSA, 2% HSA, 2.5% HSA, 5% HSA, 10% HSA, 20% HSA, or more HSA.

Described herein, in some embodiments, is a method of processing bone marrow to obtain bone marrow cells. In some embodiments, the method comprises contacting the bone marrow or the bone marrow cells with the stabilization buffer described herein.

Another aspect of the present disclosure comprises a method for processing a population of CD34+ cells comprised in bone marrow or a derivative thereof, wherein the bone marrow or the derivative thereof is derived from a deceased donor, the method comprising: obtaining a bone or bone fragment from a deceased donor, optionally, processing the bone into bone fragments; extracting the bone marrow or derivative thereof from the bone or bone fragment; and contacting the bone marrow or derivative thereof with a stabilization buffer, wherein the stabilization buffer comprises more than about 3 U/ml of a nuclease; performing a CD34+ cell isolation assay to generate a cellular composition comprising the population of CD34+ cells, wherein the composition comprising the population of CD34+ cells comprises at least about 80,000 CD34+ cells/750 μl of the bone marrow or the derivative thereof contacted with the stabilization buffer.

In some embodiments, processing or contacting the bone marrow or bone marrow cells described herein with the stabilization buff er increases the yield of the bone marrow cells obtained from the methods described herein compared to the yield of the bone marrow cells processed in the absence of the stabilization buff er. In some instances, processing or contacting the bone marrow or bone marrow cells described herein with the stabilization buff er increases the yield of the bone marrow cells by at least about 10%, 20%, 30%, 40%, 50%, 60%, 70%, 80%, 90%, 100%, 2-fold, 3-fold, 4-fold, 5-fold, 10-fold, 20-fold, 50-fold, or more compared to yield of bone marrow cells processed in the absence of the stabilization buffer. In some embodiments, processing or contacting the bone marrow or bone marrow cells described herein with the stabilization buffer increases the viability of the bone marrow cells obtained from the methods described herein compared to the viability of the bone marrow cells processed in the absence of the stabilization buff er. In some instances, processing or contacting the bone marrow or bone marrow cells described herein with the stabilization buffer increases the viability of the bone marrow cells by at least about 10%, 20%, 30%, 40%, 50%, 60%, 70%, 80%, 90%, 100%, 2-fold, 3-fold, 4-fold, 5-fold, 10-fold, 20-fold, 50-fold, or more compared to viability of bone marrow cells processed in the absence of the stabilization buffer.

In some embodiments, processing or contacting the bone marrow or bone marrow cells described herein with the stabilization buffer increases the number of CD34+ bone marrow cells compared to the number of CD34+ bone marrow cells processed in the absence of the stabilization buffer. In some cases, the number of CD34+ bone marrow obtained from processing with the stabilization buffer is increased by at least about 10%, 20%, 30%, 40%, 50%, 60%, 70%, 80%, 90%, 100%, 2-fold, 3-fold, 4-fold, 5-fold, 10-fold, 20-fold, 50-fold, or more compared to the number of CD34+ bone marrow obtained from processing in the absence of stabilization buffer. In some embodiments, processing or contacting the bone marrow or bone marrow cells described herein with the stabilization buffer increases the number of CD45+ bone marrow cells compare to the number of CD45+ bone marrow cells processed in the absence of the stabilization buffer. In some cases, the number of CD45+ bone marrow obtained from processing with the stabilization buffer is increased by at least about 10%, 20%, 30%, 40%, 50%, 60%, 70%, 80%, 90%, 100%, 2-fold, 3-fold, 4-fold, 5-fold, 10-fold, 20-fold, 50-fold, or more compared to the number of CD45+ bone marrow obtained from processing in the absence of stabilization buffer.

In some embodiments, cellular compositions comprising CD34+ cells derived from bone marrow samples processed with the stabilization buffers described herein have an increased amount of CD34+ cells, as compared to cellular compositions generated from known CD34+ isolation methods. In some embodiments. The amount of CD34+ cells isolated from the bone marrow samples contacted with the stabilization buffers described herein is at least about 70,000 CD34+ cells/750 μl of bone marrow or a derivative thereof contacted with the stabilization buffers described herein. In some embodiments, the amount of CD34+ cells isolated from the bone marrow samples contacted with the stabilization buffers described herein is at least about 70,000 cells/750 μl to about 100,000 cells/750 μl. In some embodiments, the amount of CD34+ cells isolated from the bone marrow samples contacted with the stabilization buffers described herein is at least about 70,000 cells/750 μl to about 75,000 cells/750 μl, about 70,000 cells/750 μl to about 80,000 cells/750 μl, about 70,000 cells/750 μl to about 85,000 cells/750 μl, about 70,000 cells/750 μl to about 90,000 cells/750 μl, about 70,000 cells/750 μl to about 95,000 cells/750 μl, about 70,000 cells/750 μl to about 100,000 cells/750 μl, about 75,000 cells/750 μl to about 80,000 cells/750 μl, about 75,000 cells/750 μl to about 85,000 cells/750 μl, about 75,000 cells/750 μl to about 90,000 cells/750 μl, about 75,000 cells/750 μl to about 95,000 cells/750 μl, about 75,000 cells/750 μl to about 100,000 cells/750μ, about 80,000 cells/750 μl to about 85,000 cells/750μ, about 80,000 cells/750 μl to about 90,000 cells/750 μl, about 80,000 cells/750 μl to about 95,000 cells/750 μl, about 80,000 cells/750 μl to about 100,000 cells/750 μl, about 85,000 cells/750μ 1 to about 90,000 cells/750μ, about 85,000 cells/750μ 1 to about 95,000 cells/750μ, about 85,000 cells/750 μl to about 100,000 cells/750 μl, about 90,000 cells/750 μl to about 95,000 cells/750μ, about 90,000 cells/750 μl to about 100,000 cells/750 μl, or about 95,000 cells/750 μl to about 100,000 cells/750 μl. In some embodiments, the amount of CD34+ cells isolated from the bone marrow samples contacted with the stabilization buffers described herein is at least about 70,000 cells/750 μl, about 75,000 cells/750 μl, about 80,000 cells/750μ, about 85,000 cells/750μ, about 90,000 cells/750μ, about 95,000 cells/750μ, or about 100,000 cells/750 μl. In some embodiments, the amount of CD34+ cells isolated from the bone marrow samples contacted with the stabilization buffers described herein is at least at least about 70,000 cells/750 μl, about 75,000 cells/750 μl, about 80,000 cells/750 μl, about 85,000 cells/750 μl, about 90,000 cells/750 μl, or about 95,000 cells/750 μl. In some embodiments, the amount of CD34+ cells isolated from the bone marrow samples contacted with the stabilization buffers described herein is at least at most about 75,000 cells/750 μl, about 80,000 cells/750 μl, about 85,000 cells/750 μl, about 90,000 cells/750 μl, about 95,000 cells/750 μl, or about 100,000 cells/750 μl.

In some embodiments, the CD34+ cells derived from bone marrow samples processed with the stabilization buffers described herein also exhibit higher viability as compared to cellular compositions generated from known CD34+ isolation methods.

In some embodiments, the amount of CD34+ cells isolated from the bone marrow samples contacted with the stabilization buffers described herein comprise a percent viability of at least about 70% to about 95%. In some embodiments, the amount of CD34+ cells isolated from the bone marrow samples contacted with the stabilization buffers described herein comprise a percent viability of at least about 70% to about 95%. In some embodiments, the amount of CD34+ cells isolated from the bone marrow samples contacted with the stabilization buffers described herein comprise a percent viability of at least about 70% to about 75%, about 70% to about 80%, about 70% to about 85%, about 70% to about 90%, about 70% to about 95%, about 75% to about 80%, about 75% to about 85%, about 75% to about 90%, about 75% to about 95%, about 80% to about 85%, about 80% to about 90%, about 80% to about 95%, about 85% to about 90%, about 85% to about 95%, or about 90% to about 95%. In some embodiments, the amount of CD34+ cells isolated from the bone marrow samples contacted with the stabilization buffers described herein comprise a percent viability of at least about 70%, about 75%, about 80%, about 85%, about 90%, or about 95%. In some embodiments, the amount of CD34+ cells isolated from the bone marrow samples contacted with the stabilization buffers described herein comprise a percent viability of at least at least about 70%, about 75%, about 80%, about 85%, or about 90%. In some embodiments, the amount of CD34+ cells isolated from the bone marrow samples contacted with the stabilization buffers described herein comprise a percent viability of at least at most about 75%, about 80%, about 85%, about 90%, or about 95%.

In an aspect of the present disclosure, a method is provided for selecting CD34 expressing (CD34+) cells from deceased donor bone marrow using density reduced Ficoll and an immunomagnetic CD34+ cell isolation kit. Surprisingly, it has been found that cell isolation using density reduced Ficoll prior to CD34 selection is beneficial to obtain high purity and viability CD45/CD34+ cells from freshly prepared deceased donor bone marrow. On the other hand, Ficoll at conventional density has been found to be optimal for CD45/CD34+ cell selection from thawed cryopreserved deceased donor bone marrow.

Vertebral sections obtained from a recently deceased donor were processed as described above. Thus, in one embodiment, the bone is cleaned of all soft tissue and then cut into small pieces that were immediately submerged into 500 ml of grinding media. The grinding media can be PLASMA-LYTE™ A injection pH 7.4, multiple electrolytes, injection type 1 USP (PLASMA-LYTE™) containing 2.5% human serum albumin (HSA), 3 U/ml Denarase, and 10 U/ml heparin. The sectioned VB are ground using a bone grinder, filtered and rinsed with rinse media (such as PLASMA-LYTE™ with 2.5% HSA). The entire cell suspension is centrifuged to concentrate cells to 2-3×10⁸/ml and the cell concentration is extracted. A portion or all of the resulting BM preparation can be used immediately for CD34 selection, while the remainder can be prepared for cryopreservation. The cryopreserved portion involves adding a final concentration of 10% DMSO and 5% HSA to the BM cells and bringing the preparation to −86° C., either by passive cooling or by controlled cooling at a rate of approximately −1° C./min, after which the cryopreserved portion is plunged into liquid nitrogen.

For selection of CD34+ cells, either the newly processed BM preparation is used, or a previously cryopreserved portion is thawed for use. Ficoll-Paque PLUS is added to the BM preparation to separate the desired CD34+ cell component of the bone marrow. It has been found for cell selection from cryopreserved bone marrow that the conventional density for the Ficoll of 1.077 g/ml produces acceptable results. However, in one aspect of the present disclosure, for cell selection from freshly prepared deceased donor bone marrow the Ficoll density is reduced from the conventional density. In particular, the density is reduced by mixing Ficoll-Paque PLUS (density 1.077 g/ml, GE Company) with Plasma Lyte-A Injection pH 7.4 (Baxter Healthcare 2B2544 X) in specific proportions to obtain an overall density of less than 1.077 g/ml, particularly 1.063-1.052 g/ml. In one specific embodiment, the density of 1.063 g/ml was found to be optimal for isolation of CD34+ cells, taking into account quantity, viability and purity of the CD34+ cells.

In one embodiment, 5 ml of the 1.063 g/ml density Ficoll solutions is pipetted into 15-ml centrifuge tubes, and the BM solution generated from VBs of deceased donors is carefully layered over the Ficoll gradient. The tubes are centrifuged for 30 min at 400 g without break at room temperature. After centrifugation, buffy coat cells are harvested carefully, and the cells are washed in phosphate-buffered saline (PBS) containing 0.5% HSA and 2 mM Ethylenediaminetetraacetic acid (EDTA) (MACS buffer, Miltenyi). In one specific embodiment, centrifugation is performed for 5 min at 400 g, and the resulting cell pellets are resuspended in 10 ml PBS, followed by a second centrifugation for 5 min at 400 g.

Nucleated cells in the isolated buffy coat can be counted using a Sysmex XP-300. A Cellometer Vision (Nexcellom) or flow cytometer can be used to determine cell counts of purified CD34 cells. 20 microliters of AOPI can be added to 20 microliters of cells and after mixing total viable cells can be determined. The CD34+ cells can be selected by a positive immune separation method using a CliniMAX system (Miltenyi, Bergisch Gladbach, Germany) or an EasySep CD34 kit (Stemcell Technologies, Vancouver, BC, Canada) in accordance with the protocol of the manufacturer. From testing at various Ficoll densities it has been surprisingly determined that the lower Ficoll density contemplated in the present disclosure (i.e., 1.063-1.052 gm/ml vs. the conventional 1.077 gm/ml density) leads to more optimum cell recovery. Optimization is based on purity, viability and yield of selected CD34 cells. A target of >90% purity and >90% viable CD34+ cells is preferred. While lower Ficoll densities resulted in greater purity and fewer dead cells, it was surprisingly found that a greater portion of the CD34+ cells present in the deceased donor whole bone marrow before selection are lost using the lower Ficoll densities to prepare buffy coat. Thus, the high viability and purity of CD45/CD34+ cells achieved at the conventional Ficoll density gradient also leads to a large loss in yield (approximately 60% loss of input CD34+ cells).

Thus, in accordance with one aspect of the present disclosure, for freshly prepared the optimal density of Ficoll for selection of CD45/CD34+ cells at >90% purity and viability is less than 1.077 and particularly 1.063-1.052. This Ficoll density provides a higher yield of CD45/CD34+ cells with similar purity and cell viability to the conventional Ficoll density approach.

In another aspect of the present disclosure, the CD34+ cells can be initially acquired from a freshly prepared deceased donor bone marrow using the reduced density Ficoll-Paque described above. The BM can be cryogenically frozen and then the CD34+ cells can be acquired later using conventional density Ficoll-Paque. This approach essentially allows selective recovery of cells from deceased donor bone marrow-either before freezing using the modified Ficoll density or after freezing and thawing using conventional Ficoll density.

Once CD34+ have been isolated from bone marrow product, it could be said that the remaining cell product is enriched for MSCs, and especially vBM-MSCs.

Recovery of MSCs from Processed Bone

In another feature of the systems and methods disclosed herein, a method is provided for preparing a composition of cadaveric human MSCs from bone. In some embodiments, the preparation may include providing a bone derived from a deceased donor, grinding the bone into one or more ground bone segments, filtering the one or more ground bone segments and extracting the cadaveric human MSCs from the one or more ground bone segments. In some embodiments, the MSCs may be recovered from thawed or cryopreserved vertebral body bone fragments. In some embodiments, the extracted cadaveric human MSCs may be vertebral bone marrow MSCs (vBM-MSCs), adherent vertebral body mesenchymal stem cells (vBA-MSCs), or both. In some embodiments, the extracted cadaveric human MSCs are derived from a bone or fragments thereof that has already been processed to remove bone marrow or derivates thereof associated with the bone or fragment thereof (e.g., bone marrow derived cells, hematopoietic stem cells). In some embodiments, the extracted cadaveric human MSCs are derived from a bone or fragments thereof that has been processed for bone marrow and/or bone marrow-derived cells (e.g., hematopoietic stem cells) as described herein. In some embodiments, the extracted cadaveric human MSCs are derived from the bone grindings and/or segments described herein following filtration and/or extraction and/or isolation of bone marrow and/or bone marrow-derived cells as described herein. The processing and extraction of viable vBA-MSCs from the bone and/or derivates thereof (e.g., bone grindings described herein, bone segments described herein) results in significant improvements in cell yield, especially with respect to total cell yield (vBA-MSCs and hematopoietic stem cells) per weight of bone derived from a donor, and viability of cells with respect to the state of the art. In some embodiments, the vBA-MSCs described herein can be combined with bone marrow-derived MSCs (vBM-MSCs) isolated from bone marrow isolated and processed as described herein.

In some embodiments, the extraction of cadaveric human vBA-MSCs may include contacting the bone or derivatives thereof with a digestion solution. In some embodiments, the digestion solution may include one or more distinct enzymes. In some embodiments, the one or more distinct enzymes may include one or more collagenases and neutral proteases. In some embodiments, the digestion solution may be present at a ratio of volume to weight of the one or more ground bone segments and enzymatic solution of about 1:1 to about 15:1. In some embodiments, the ratio may be 1:1, 2.5:1, 5:1, 7.5:1, 10:1 and 15:1 (volume:weight). In some embodiments, the combination of one or more collagenases and neutral proteases is used to obtain the highest possible yields of vBA-MSC.

In some embodiments, a collagenase may include Clostridium histolyticum further comprising two active isoforms, C1 and C2. In some embodiments, one or more collagenases comprising isoforms C1 and C2 may be present in the digestion solution at a ratio comprising more collagenase isoform C1 than collagenase isoform C2. In some embodiments, the ratio of collagenase isoform C1 to collagenase isoform C2 may be about 30 to about 70: about 10 to about 29. In some embodiments, the ratio of collagenase isoform C1 to collagenase C2 may be 35:15. In some embodiments, the mass ratio of C1 and C2 for each concentration may be 70:30, 54:46, 37:63, 82:18, 54:46, and 90:10.

In some embodiments, the neutral protease may be Paneibacillus polymyxa neutral protease. In some embodiments, the neutral protease concentration may be about 2 U/ml to about 21 U/ml. In some embodiments, the neutral protease concentration may be about 2 U/ml to about 7 U/ml, about 2 U/ml to about 12 U/ml, about 2 U/ml to about 17 U/ml, about 2 U/ml to about 21 U/ml, about 7 U/ml to about 12 U/ml, about 7 U/ml to about 17 U/ml, about 7 U/ml to about 21 U/ml, about 12 U/ml to about 17 U/ml, about 12 U/ml to about 21 U/ml, or about 17 U/ml to about 21 U/ml. In some embodiments, the neutral protease concentration may be about 2 U/ml, about 7 U/ml, about 12 U/ml, about 17 U/ml, or about 21 U/ml. In some embodiments, the neutral protease concentration may be at least about 2 U/ml, about 7 U/ml, about 12 U/ml, or about 17 U/ml. In some embodiments, the neutral protease concentration may be at most about 7 U/ml, about 12 U/ml, about 17 U/ml, or about 21 U/ml. In some embodiments, the digestion solution may comprise the neutral protease at an activity of about 19.6 U/ml.

In some embodiments, the collagenase concentration is about 0.05 U/ml to about 1.6 U/ml. In some embodiments, the collagenase concentration is about 0.05 U/ml to about 0.1 U/ml, about 0.05 U/ml to about 0.15 U/ml, about 0.05 U/ml to about 0.2 U/ml, about 0.05 U/ml to about 0.25 U/ml, about 0.05 U/ml to about 0.3 U/ml, about 0.05 U/ml to about 0.35 U/ml, about 0.05 U/ml to about 0.4 U/ml, about 0.05 U/ml to about 0.8 U/ml, about 0.05 U/ml to about 1.2 U/ml, about 0.05 U/ml to about 1.6 U/ml, about 0.1 U/ml to about 0.15 U/ml, about 0.1 U/ml to about 0.2 U/ml, about 0.1 U/ml to about 0.25 U/ml, about 0.1 U/ml to about 0.3 U/ml, about 0.1 U/ml to about 0.35 U/ml, about 0.1 U/ml to about 0.4 U/ml, about 0.1 U/ml to about 0.8 U/ml, about 0.1 U/ml to about 1.2 U/ml, about 0.1 U/ml to about 1.6 U/ml, about 0.15 U/ml to about 0.2 U/ml, about 0.15 U/ml to about 0.25 U/ml, about 0.15 U/ml to about 0.3 U/ml, about 0.15 U/ml to about 0.35 U/ml, about 0.15 U/ml to about 0.4 U/ml, about 0.15 U/ml to about 0.8 U/ml, about 0.15 U/ml to about 1.2 U/ml, about 0.15 U/ml to about 1.6 U/ml, about 0.2 U/ml to about 0.25 U/ml, about 0.2 U/ml to about 0.3 U/ml, about 0.2 U/ml to about 0.35 U/ml, about 0.2 U/ml to about 0.4 U/ml, about 0.2 U/ml to about 0.8 U/ml, about 0.2 U/ml to about 1.2 U/ml, about 0.2 U/ml to about 1.6 U/ml, about 0.25 U/ml to about 0.3 U/ml, about 0.25 U/ml to about 0.35 U/ml, about 0.25 U/ml to about 0.4 U/ml, about 0.25 U/ml to about 0.8 U/ml, about 0.25 U/ml to about 1.2 U/ml, about 0.25 U/ml to about 1.6 U/ml, about 0.3 U/ml to about 0.35 U/ml, about 0.3 U/ml to about 0.4 U/ml, about 0.3 U/ml to about 0.8 U/ml, about 0.3 U/ml to about 1.2 U/ml, about 0.3 U/ml to about 1.6 U/ml, about 0.35 U/ml to about 0.4 U/ml, about 0.35 U/ml to about 0.8 U/ml, about 0.35 U/ml to about 1.2 U/ml, about 0.35 U/ml to about 1.6 U/ml, about 0.4 U/ml to about 0.8 U/ml, about 0.4 U/ml to about 1.2 U/ml, about 0.4 U/ml to about 1.6 U/ml, about 0.8 U/ml to about 1.2 U/ml, about 0.8 U/ml to about 1.6 U/ml, or about 1.2 U/ml to about 1.6 U/ml. In some embodiments, the collagenase concentration is about 0.05 U/ml, about 0.1 U/ml, about 0.15 U/ml, about 0.2 U/ml, about 0.25 U/ml, about 0.3 U/ml, about 0.35 U/ml, about 0.4 U/ml, about 0.8 U/ml, about 1.2 U/ml, or about 1.6 U/ml. In some embodiments, the collagenase concentration is at least about 0.05 U/ml, about 0.1 U/ml, about 0.15 U/ml, about 0.2 U/ml, about 0.25 U/ml, about 0.3 U/ml, about 0.35 U/ml, about 0.4 U/ml, about 0.8 U/ml, or about 1.2 U/ml. In some embodiments, the collagenase concentration is at most about 0.1 U/ml, about 0.15 U/ml, about 0.2 U/ml, about 0.25 U/ml, about 0.3 U/ml, about 0.35 U/ml, about 0.4 U/ml, about 0.8 U/ml, about 1.2 U/ml, or about 1.6 U/ml.

In accordance with one aspect of the disclosure, neutral protease concentration and collagenase concentrations (C1 and C2 collagenase) and ratio of solution volume (mls) to bone fragment weight (mgs) are determined.

In some embodiments, the total collagenase concentrations (C1 and C2 collagenase) are about 25 μg/ml to about 100 μg/ml. In some embodiments, the total collagenase concentrations are about 25 μg/ml to about 32.5 μg/ml, about 25 μg/ml to about 47.5 μg/ml, about 25 μg/ml to about 42.5 μg/ml, about 25 μg/ml to about 50 μg/ml, about 25 μg/ml to about 65 μg/ml, about 25 μg/ml to about 77.5 μg/ml, about 25 μg/ml to about 85 μg/ml, about 25 μg/ml to about 100 μg/ml, about 32.5 μg/ml to about 47.5 μg/ml, about 32.5 μg/ml to about 42.5 μg/ml, about 32.5 μg/ml to about 50 μg/ml, about 32.5 μg/ml to about 65 μg/ml, about 32.5 μg/ml to about 77.5 μg/ml, about 32.5 μg/ml to about 85 μg/ml, about 32.5 μg/ml to about 100 μg/ml, about 47.5 μg/ml to about 42.5 μg/ml, about 47.5 μg/ml to about 50 μg/ml, about 47.5 μg/ml to about 65 μg/ml, about 47.5 μg/ml to about 77.5 μg/ml, about 47.5 g/ml to about 85 μg/ml, about 47.5 μg/ml to about 100 μg/ml, about 42.5 μg/ml to about 50 μg/ml, about 42.5 μg/ml to about 65 μg/ml, about 42.5 μg/ml to about 77.5 μg/ml, about 42.5 μg/ml to about 85 μg/ml, about 42.5 μg/ml to about 100 μg/ml, about 50 μg/ml to about 65 μg/ml, about 50 μg/ml to about 77.5 μg/ml, about 50 μg/ml to about 85 μg/ml, about 50 μg/ml to about 100 μg/ml, about 65 μg/ml to about 77.5 μg/ml, about 65 μg/ml to about 85 μg/ml, about 65 μg/ml to about 100 μg/ml, about 77.5 μg/ml to about 85 μg/ml, about 77.5 μg/ml to about 100 μg/ml, or about 85 μg/ml to about 100 μg/ml. In some embodiments, the total collagenase concentrations are about 25 μg/ml, about 32.5 μg/ml, about 47.5 μg/ml, about 42.5 μg/ml, about 50 μg/ml, about 65 μg/ml, about 77.5 μg/ml, about 85 μg/ml, or about 100 μg/ml. In some embodiments, the total collagenase concentrations are at least about 25 μg/ml, about 32.5 μg/ml, about 47.5 μg/ml, about 42.5 μg/ml, about 50 μg/ml, about 65 μg/ml, about 77.5 μg/ml, or about 85 μg/ml. In some embodiments, the total collagenase concentrations are at most about 32.5 μg/ml, about 47.5 μg/ml, about 42.5 μg/ml, about 50 μg/ml, about 65 μg/ml, about 77.5 μg/ml, about 85 μg/ml, or about 100 μg/ml.

In some embodiments, the mass ratio of C1 and C2 for each concentration are 70:30, 54:46, 37:63, 82:18 and 90:10, respectively.

According to the process, fragments of VB bone are placed in cryoprotectant solution comprised of PLASMA-LYTE™, 2.5% human serum albumin and 10% dimethyl sulfoxide (DMSO) and incubated for 1 hour at 4° C. In some embodiments, the incubation period is about 1 hour to about 3 hours. In some embodiments, the incubation period is about 1 hour to about 1.5 hours, about 1 hour to about 2 hours, about 1 hour to about 2.5 hours, about 1 hour to about 3 hours, about 1.5 hours to about 2 hours, about 1.5 hours to about 2.5 hours, about 1.5 hours to about 3 hours, about 2 hours to about 2.5 hours, about 2 hours to about 3 hours, or about 2.5 hours to about 3 hours. In some embodiments, the incubation period is about 1 hour, about 1.5 hours, about 2 hours, about 2.5 hours, or about 3 hours. In some embodiments, the incubation period is at least about 1 hour, about 1.5 hours, about 2 hours, or about 2.5 hours. In some embodiments, the incubation period is at most about 1.5 hours, about 2 hours, about 2.5 hours, or about 3 hours. The solution is removed, and the bone fragments cooled at a rate of ˜1 °/min to −86° C. and then plunged into liquid nitrogen. After 24-48 hours in liquid nitrogen, the bone fragments are thawed rapidly in a water bath set at 37° C. and then washed in saline and digested using the collagenase/protease solution described above.

In some embodiments, the volume-to-weight ratio was 5:1 at an incubation time of 2.5 hours. In some embodiments, the protease produced neutral protease activity of 19.6 U/ml.

The mixture of cells liberated by digesting VB bone fragment is cultured on tissue-coated plastic in the presence of Mesencult medium to select proliferative vBA-MSC. Freshly digested preparations as well as different passages of VB-MSC can be characterized by flow cytometry, colony forming unit-fibroblast (CFU-F) potential, population doubling time (PDT) and trilineage (adipogenic, chondrogenic, and osteogenic) differentiation in vitro.

In some embodiments, the method of cadaveric human MSC extraction dis closed herein may be capable of extracting quantities of about 10 million to about 10 billion. In some embodiments, cadaveric human MSCs may be administered in quantities of about 10 million to about 100 million, about 10 million to about 1 billion, about 10 million to about 10 billion, about 100 million to about 1 billion, about 100 million to about 10 billion, or about 1 billion to about 10 billion. In some embodiments, cadaveric human MSCs may be administered in quantities of about 10 million, about 100 million, about 1 billion, or about 10 billion. In some embodiments, cadaveric human MSCs may be administered in quantities of at least about 10 million, about 100 million, or about 1 billion. In some embodiments, cadaveric human MSCs may be administered in quantities of at most about 100 million, about 1 billion, or about 10 billion.

### Advantages of the Present Disclosure

The demonstrated potential of mesenchymal stem/stromal cells (MSC) to address intractable diseases has been slow to materialize into approved therapies. The MSC development field has been hindered by a lack of clear efficacy in late-stage, multi-center, randomized controlled trials, even though preclinical and early-stage trials have appeared exceptionally positive. The disconnect between promising early data and demonstrating unequivocal clinical efficacy for other than a handful of diseases has been broadly attributed to batch-to-batch variation of critical quality attributes caused by non-optimal manufacturing practices. Advanced manufacturing methods and strong quality assurance systems can prevent release of nonconforming MSC lots; however, an adequate supply of primary cell seedstocks are necessary to economically manufacture cells without overexpansion, which induces senescence and loss of potency.

Other sources of variability are injury due to cryopreservation of intermediate cell stocks used in manufacturing and final products as well as improper handling of the cells during thawing for administration. Cryopreservation is an essential process for maintaining viability during manufacturing and subsequent storage and shipment of cellular therapies. However, substantial loss of function after thawing is commonly observed and is a major hurdle impeding full realization of the broad clinical potential of all cellular therapeutics, including MSC. Thus, establishing procedures that overcome cryo-injury are of paramount importance for enhancing clinical successes with MSC therapies. One presently employed solution is to briefly (18 to 72 hr) culture MSC after thawing so that the cells have time to recover (ideally without proliferating) prior to infusion into patients. Besides adding to costs for treatment by requiring personnel time and material resources, cryorecovery adds logistical complexity to the treatment given the lability of cells during shipment and the short product expiration window. Developing a procedure that enables thawing and infusing functional cells at the patient's bedside would be much more advantageous.

It has been suggested that so called “cryoinjury” and related thawing injury were major contributors to high profile failures of MSC therapies in advanced phase clinical trials. In addition to the immediately observable lower viability upon thaw, cryoinjured MSC display diminished immunomodulatory activity and an increased susceptibility to clearance in vivo. The causes of cryoinjury are multifaceted and include osmotic and thermal shock, cryoprotectant toxicity, and intracellular ice crystal formation. Each of these insults can lead to a sequalae of physiological responses, further enhancing cell injury during recovery. These delayed effects, which manifest over hours and days, are more accurate predictors of post-thaw cellular yield and function compared to immediate post-thaw viability assessments. Thus, sampling at intervals rather than at a discrete timepoint immediately post-thaw is required; however, it is not practical to collect this data prior to administering MSC in the clinical setting.

Strategies to prevent or at least limit cellular injury during cryopreservation and thawing include, to name a few, developing cryopreservation solutions which mirror intracellular ion species and concentrations, controlled rate freezing apparatuses, novel cryoprotectant agents, inhibitors of cell injury processes, and ice recrystallization inhibitors. Although promising results have been attained, it is likely that a single solution that is effective across different cell types, having vastly different physiological characteristics might not be achievable.

Even for a homologous population, cell expansion in nutrient rich media generates a spectrum of physiological states due to asynchronous progression of the population through the cell cycle. Besides a gradual increase in volume which occurs as daughter cells expand and replicate DNA, mitotic phase cells rapidly swell by up to 30% due to a rapid influx of water, resulting in reduced density and osmolality of the cytoplasm. Thus, cells in different phases of the cell cycle possess different intracellular compositions and volumes. Furthermore, and in contrast to a generalized view of populations of proliferating cells, on the individual cell level, mass and volume oscillates nonparametrically in response to a multitude of extrinsic and intrinsic modulators and somewhat autonomously with respect to the stage in the cell cycle. For instance, checkpoint regulation due to DNA strand breaks will either stop or slow cell cycle progression, depending on the phase of the cell cycle, until repairs are complete. If repairs cannot be completed due to the severity of DNA damage, exit from the cell cycle will be triggered to prevent passing on damaged DNA to daughter cells.

The differences m size and internal composition of asynchronously proliferating monocultures produces a spectrum of physiological states with a commensurate range of responses to osmotic imbalances, hypothermia and phase transitions during freezing and warming. The ability of cells to tolerate the rather harsh conditions experienced during cryopreservation depends on cell size (surface to volume ratio), membrane permeability and osmotic constraints. Cell volume is governed through an equilibrium between internal hydrostatic pressure, osmolarity, and membrane tension with each fluctuating due to active transport and passive diffusion of water, small solutes and ions across the membrane over timescales of minutes to hours. Thus, hydrodynamic forces acting on individual cells within a population are constantly in flux leading to a distribution of responses to sudden changes in external osmolarity that occur during addition or removal of molar concentrations of commonly used cryoprotectants such as dimethyl sulfoxide (Me₂SO₄). Relatedly, water content will influence the size of ice crystal formation and the concentrations of monomeric precursors (e.g., amino acids, nucleosides and monosaccharides) versus polymeric structures influence internal osmolality. Thus, dynamic fluctuations in cell size and composition throughout the cell cycle will influence tolerance limits to the cryopreservation and thawing processes.

Given this potential for high variability with cultures, the effects of cell cycle status on susceptibility of MSC to injury due to cryopreservation and subsequent thawing was examined. In the experiments disclosed herein, it was discovered that S phase MSCs are exquisitely sensitive to cryoinjury, demonstrating heightened levels of apoptosis post-thaw and reduced immunomodulatory function. Synchronizing MSC cultures by growth factor deprivation (also known as serum starvation) to block progression through G1 phase prior to cryopreservation greatly reduced post-thaw dysfunction of the cells by preventing DNA double strand breaks (DSB) during replication and subsequent triggering of apoptosis. Thus, the present disclosure provides a robust and effective strategy to enhance post-thaw recovery of therapeutic MSC which may have benefits for other cellular therapies. Similarly important is that this process makes MSC therapies safer by preventing genetic instability in post-thaw cells; thereby, reducing the potential for administering cells harboring mutations.

### Definitions

As used herein, IFNγ-primed MSCs are referred to as “γMSCs”.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as illustrative.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, Band C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to +10% of a stated number or value. In some instances, the term “about” refers to one standard deviation greater or less than the stated number or value.

The term “from” as in “from 1 to 10” includes the initial and final number recited. Therefore, “from 1 to 10” includes the whole numbers 1, 2, 3, 4, 5, 6, 7, 8, 9, and 10 and includes fractions thereof, (e.g., about 0.1, 0.2, 0.3, 0.4, 0.5, 0.6, 0.7, 0.8, and about 0.9).

The terms “increased”, “increasing”, or “increase” are used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least ab out 50%, or at least about 60%, or at least about 70%, or at least about 80%, or at least about 90% or up to and including a 100% increase or any increase from about 10 to about 100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease from about 10 to about 100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

### EXAMPLES

### Example 1: Manufacturing γMSCs

This example illustrates a two-step MSC manufacturing process to maximize the immune modularity and tissue regenerative potential of MSCs.

A practical barrier to successful MSC therapy Is that freshly thawed, previously cryopreserved cells seem to lack much of the immune modulatory and tissue regenerative potential of freshly expanded cells. The infusion of freshly expanded cells could counteract this. However, the growth kinetics of MSCs in vitro is highly variable; therefore, preparing MSCs to be infused on a preplanned day is challenging.

A two-step MSC manufacturing process is used to overcome this obstacle. MSCs are isolated and undergo a primary expansion. The resulting cells are cryopreserved to generate a stock of MSCs. At the appropriate time, the indicated number of cells are thawed and returned to tissue culture for a secondary expansion over a predefined interval (e.g., 7 days). During this secondary expansion, MSCs are primed with IFNγ to reliably yield freshly expanded γMSCs for infusion on the indicated day. This manufacturing process shows that MSCs and γMSCs prepared according to this protocol have the expected characteristics suggesting that these γMSCs do not pose a greater risk to subjects than conventionally prepared cells.

Notably, in some embodiments, when the MSCs were being interferon γ-primed, the culture was not in a hypoxic condition. As examples, the culture did not have decreased levels of 02, did not have increased levels of CO₂, and/or lacked the presence of a hypoxia mimetic. Decreased levels of O₂ may be from about 1% O₂ to about 5% O₂. Increased levels of CO₂ can be about 5% CO₂ or higher. Illustrative hypoxia mimetics include desferoxamine, cobalt chloride, hydralazine, nickel chloride, diazoxide, and dimethyloxalyglycine.

Bone marrow was obtained from cadaver bones and cryopreserved as described elsewhere in the present disclosure.

Passage 0: One-half bag of the cryopreserved bone marrow is utilized for primary cell culture. The cryopreserved bag was taken out of inventory and the unit was thawed as per the batch record instruction. Approximately 3.9 billion mononuclear cells were plated in six CellBIND® 10-chamber CellSTACKS® and cultured for ˜14 days in MSC Culture Media. These Passage 0 (PO) cells were cultured for-14 days with fresh media changes every 3-4 days.

Passage 1: At >75% confluence, P0 cells were detached using TrypLE™ Select, and ˜410M cells are immediately replated in MSC Culture Media in sixteen CellBIND® 10-chamber CellSTACKS®. These Passage 1 (P1) cells were incubated for 4-5 days and once the flasks were >75% confluent, cells were detached using TrypLE™ Select. P1 cells were resuspended in PLASMA-LYTE A+2.5% HSA+5% DMSO at 13M cells/ml and packaged in ˜220×2 mL cryovials at 2 mL per cryovial using an automatic filler for the CellSeal vials. Cells were then cryopreserved and placed into the vapor phase above LN2 in alarm monitored cryogenic tanks for storage at ≤−140° C. The complete P1 cells testing criteria are presented elsewhere in this disclosure at Table 2.

Passage 2/3: Next, one P1 vial was thawed and plated in one CellBIND® 10-chamber CellSTACK® in MSC Culture Media. These cells became Passage 2 (P2) cells. The P2 cells were cultured for 4-5 days and once the flasks were >75% confluent, cells were detached using TrypIB™ Select. ˜410 million cells were immediately replated in MSC Culture Media in sixteen CellBIND® 10-chamber CellSTACKS®. These then became Passage 3 (P3) cells.

The P3 cells were cultured for 4-5 days and once the flasks were >75% confluent, cells were detached using TrypLE™ Select. P3 cells were resuspended in PLASMA-LYTE A+2.5% HSA+5% DMSO at 20M cells/ml and packaged in 5 mL CellScal closed-system cryovials at 5 mL per cryovial using an automatic filler. Cells were then cryopreserved, which consisted of precooling or equilibration at 4° C. followed by passive cryopreservation at a rate of −1° C./minute to <−80° C. and then moving into vapor phase above LN2 for storage at ≤−140° C. The complete P3 cells testing criteria were presented in Table 3. The P3 cells constitute the cells which were used for manufacture and final product formulation.

Process Timing and Intermediate Storage: The time elapsed through each culture step was variable, with >75% confluence generally was achieved in 4-5 days. Time elapsed from a culture harvest to the start of the cryopreservation was <6 hours. Upon cryopreservation cells are theoretically stable indefinitely, however, real-time stability studies are ongoing.

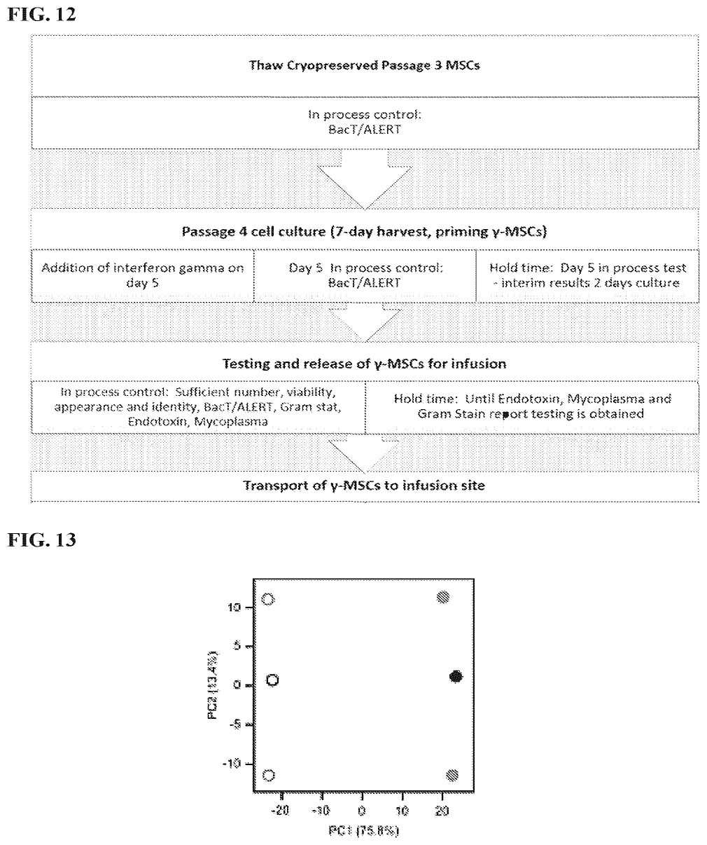

Final Product Manufacturing Process (Secondary Expansion): Processing of the final product for infusion included thawing of the cryopreserved P3 MSCs from Ossium, priming with interferon gamma, and then final formulation. A flow diagram illustrating the process is provided in FIG. 12.

When shipped to a distant site, the vial(s) were shipped in a Cryoport EXP-6 dry vapor shipper (holding temperature validated for up to 10 days). The Cryoport dry vapor shipper was boxed and sent overnight to the distant site. The dry vapor shipper was continuously monitored via Cryoport's Smartpak II® monitoring system. Once at the distant site, the vials were promptly removed from the dry vapor shipper and placed in a liquid nitrogen freezer.

The next step was to remove a vial of the cryopreserved P3 cells and place it into a 37° C. water bath. The vial was kept in the water bath until approximately 80% of ice has melted (approximately 2-3 minutes). Vial was then sprayed with sterile 70% ethanol (EtOH), wiped with sterile wipes and transferred into the biosafety cabinet. The thawed sample was then transferred to a sterile conical tube (50 mL) using a Sexton CellSeal Vial Adapter and syringe. Once all P3 cells had been transferred, the cells were diluted with 2 volumes of PLASMA-LYTE A+0.5% HSA. The cells were then centrifuged at 500 RCF for 10 minutes at room temperature to form a cell pellet. The supernatant was then removed, and the pellet was resuspended in MSC Culture Media. Cell viability and count were then determined using Trypan Blue Exclusion via Manual Counting (or using AO/PI Method via automatic Cellometer). MSCs were then be seeded at 3,000-4,000/cm² onto eight CellBIND® 5-chamber CellSTACKs® and placed into a 5% CO₂, 37° C., humidified tissue culture incubator. The plating step is considered Day 0 and this cell culture step is considered Passage 4 (P4).

At Day 3, the MSC culture underwent a complete media change to remove expended media and non-adherent cells. MSCs were then returned to the tissue culture incubator for two additional days.

At Day 5, half of the media from each flask was removed and a sample for BACT/Alert was collected from this expended media from each flask. An aliquot (equating to the half of expended media that was removed) of culture media+50 ng/ml γ-IFN (R&D Systems, Minneapolis, USA) was made and added to each flask according to a standard operating procedure. The interferon gamma primed P4 cells were once again returned to the tissue culture incubator for 2 additional days.

At Day 7, γ-MSCs cell density and morphology were inspected using a bright-field inverted microscope. γ-MSCs were harvested from culture via TrypLE™ Select, washed three times with phosphate buffered saline (PBS), and assessed for cell viability and count. Final product met the preferable features as defined in Table 4.

An infusion dose was prepared in a 0.5% HSA+PLASMA-LYTE A solution at 4×10⁶ cells/ml and kept at room temperature in an infusion bag or syringe(s) until infused. Prior to release, approximately 1-2×10⁶ γ-MSCs were taken for a stat Gram Stain, which was examined for any microscopic or vegetative organisms and determination of viable cell number. Testing was initiated for bacterial (acrobic & anaerobic) and fungal, endotoxin and mycoplasma presence, and flow cytometry analysis.

Several studies have been performed to characterize and evaluate the potential effects of the use of γ-IFN for priming MSCs. Residual levels of γ-IFN, product variability, karyotype, phenotype and potential for in vitro differentiation were evaluated for γ-IFN primed MSCs compared to non-primed MSCs.

To determine whether γ-IFN priming of MSCs imparts the potential for residual activity to the product, residual γ-IFN in the clinical product was examined An ELISA detected the anticipated concentration of γ-IFN in the culture media but no cytokine was detected in the wash media nor in the cell product suspended in infusion media (linear sensitivity, 15 pg/ml). Thus, subjects should not incur any risk of γ-IFN mediated toxicity as relevant amounts will not be infused into patient (Table 7). γ-IFN was measured by ELISA and reported as pg/ml.

TABLE 7
Residual γ-IFN cells in washing and infusion medium

| | Expanded | Expanded MSCs with |
|---|---|---|
| Test Media | MSCs | (48 h) γ-IFN Priming |
| Fresh Media + γ-IFN, | NA | 5210 |
| 500 U/ml (control) | | |
| Final Culture Medium | 7 | 5239 |
| PBS Plate Rinse | <1 | 86 |
| Wash #1 | <1 | <1 |
| Wash #2 | <1 | <1 |
| Infusion medium (0 h) | <1 | <1 |
| Infusion medium (6 h) | <1 | <1 |

To determine if γ-IFN of MSCs introduced additional variability in the products compared to the intrinsic variability between donors, which has not shown to be problematic, three different preparations of γ-MSCs were assayed with RNA Seq.

Principal component analysis (PCA) using the normalized expression values for the 500 genes with the highest variance, showed distinct clustering of the three γ-MSC samples from MSCs, with the first principal component accounting for 86% of the variance. This analysis also showed a separation of the three donor samples, but this only accounted for 8% of the variance. While the preponderance of variation was due to γ-IFN priming, the cytokine produces a similar variance in each of the patients; thus, γ-IFN priming does not introduce additional inter-donor variation other than what intrinsically exists in MSCs. FIG. 13 demonstrates differential gene expression of MSCs (open shapes) and γ-MSC (filled shapes) from each donor (same color and similar verticality).

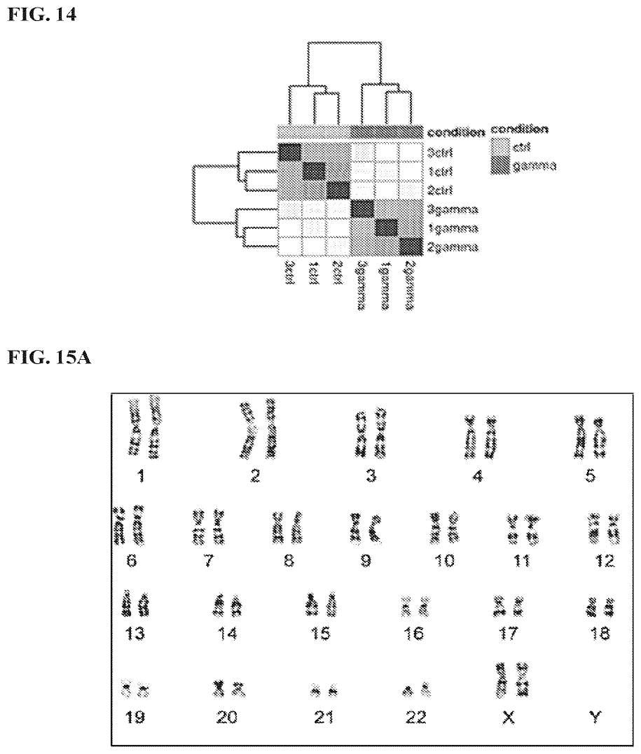

Like PCA, Euclidean distance was calculated to assess the overall similarity between samples. This analysis revealed tight hierarchical clustering of the three treated or untreated samples and distinct separation was clearly visible between the two groups suggesting that γ-IFN priming has a similar impact among the donors. FIG. 14 demonstrates overall similarity among the samples. The darker blue indicating a shorter distance, i.e., more similar; lighter blue indicating a greater distance (less similar).

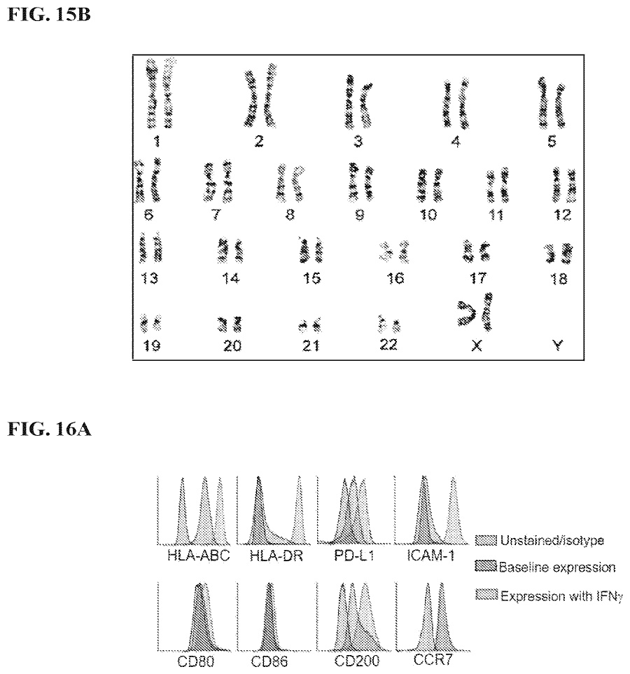

The final MSC product that was to be infused in a patient was assessed for the integrity of the chromosomes (karyotype). Twenty cells in metaphase from the standard MSC preparation and γ-IFN-primed MSCs were examined. All exhibited modal number of 46 chromosomes including two sex chromosomes. No consistent structural or numerical abnormalities were detected. All five cells assessed for G-banding from each preparation displayed a normal pattern. FIG. 15A and FIG. 15B show representative karyotypes of (FIG. 15A) MSCs and (FIG. 15B) γ-IFN-primed MSCs from cells that have undergone primary expansion, cryopreservation, and secondary expansion, and are appropriate to infuse into a subject.

The surface phenotype of the cell products was determined by flowcytometry. γ-IFN-primed MSCs express HLA Class I and II molecules, as well as PD-L1 and PD-L2, but not CD80 or CD86 as expected. MSCs and γ-MSCs underwent in vitro differentiation to osteoblasts, adipocytes, and chondroblasts. Both cell preparations differentiated similarly as determined by Alizarin Red S, Oil Red 0, and Alcian Blue histochemical staining, respectively.

Potency of PI and P3 cells was evaluated through colony forming unit-fibroblast (CFU-F) analysis (for information/characterization only at P1). For this assay, cells were resuspended in medium and counted using a Cellometer or via Trypan Blue Manual Counting. Next, 50 cells were each pipetted into 2 wells of a 6 well tissue culture treated plate. The plate was incubated for 10-14 days or until colonies are at 80% confluence, refeeding with prewarmed medium every 3-4 days. Once confluent, medium was aspirated, the plate was washed twice with phosphate buffered saline (PBS), and 5 mL of Methanol was added to each well. The cells were allowed to sit for 5 minutes in methanol to fix the cells, then the methanol was removed, and the wells were stained with Crystal Violet for 20 minutes. Finally, the Crystal Violet was removed, and the plates were washed 3 times with DI water, and colonies were counted (a colony was defined as having 50 or more cells). An average of the two wells was taken and the average was used to calculate CFU-F colonies per 1M cells plated.

Table 8 (below) lists the release testing of MSCs has been completed and the acceptable results is presented below.

TABLE 8
Release Testing: Validation Expansions

| Test | Method | Acceptable Result | Measured Result |
|---|---|---|---|
| Cell Number | Automated Cell | Required Cell | 12.2-16-fold expansion |
| | Counter | Dose ±l5% | Vial 1 = 1.15 × 10⁹ viable |
| Cell Viability | Automated Cell | 85 ± 15% | 95.80% |
| | Counter | | 95.10% |
| | | | 90.60% |
| Appearance | Automated Cell | No aggregates | No aggregates |
| | Counter | | |
| Sterility (Day −2) | Microscopy | Gram stain | Negative for all three |
| | | Negative | expansions |
| | BacT Alert | 48-hour culture | Negative for all three |
| | | negative | expansions |
| Sterility (Day 0) | Microscopy | Gram stain | Negative for all three |
| | | Negative | expansions |
| Endotoxin | Endosafe | <2.5 EU/ml | <2.5 EU/ml for all three |
| | | | expansions |
| Mycoplasma | MycoAlert Assay | None Detected | None detected for all three |
| | | | expansions |
| Identity | Flow cytometry | CD45 <3% | 0.0% for all three expansions |
| | | CD105 >85% | 99.8% for all three |
| | | | expansions |

Post-Infusion-day Data

| | | | |
|---|---|---|---|
| Sterility (Day −2) | BacT Alert | 14 Day culture | Negative for all three |
| | | Negative | expansions |
| Sterility (Day 0) | | 14 Day culture | Negative for all three |
| | | Negative | expansions |
| IDO1 Expression | Quantitative PCR | RE >1 | RE = 11.5 |
| | | | RE = 26.0 |
| | | | RE = 33.7 |

The method of this example generally follows the process shown in FIG. 11A. However, the steps and ordering thereof as disclosed in this example can be adjusted to follow the processes shown in any of FIG. 11B, FIG. 11C, or FIG. 11D.

### Example 2: Phenotype Analysis of MSC after Exposure to IFNγ

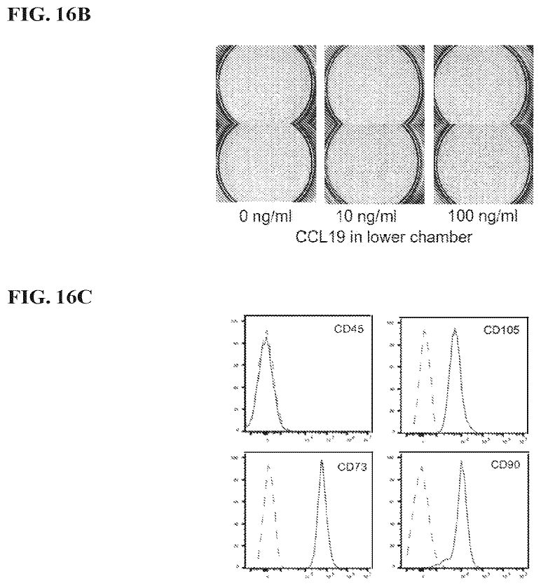

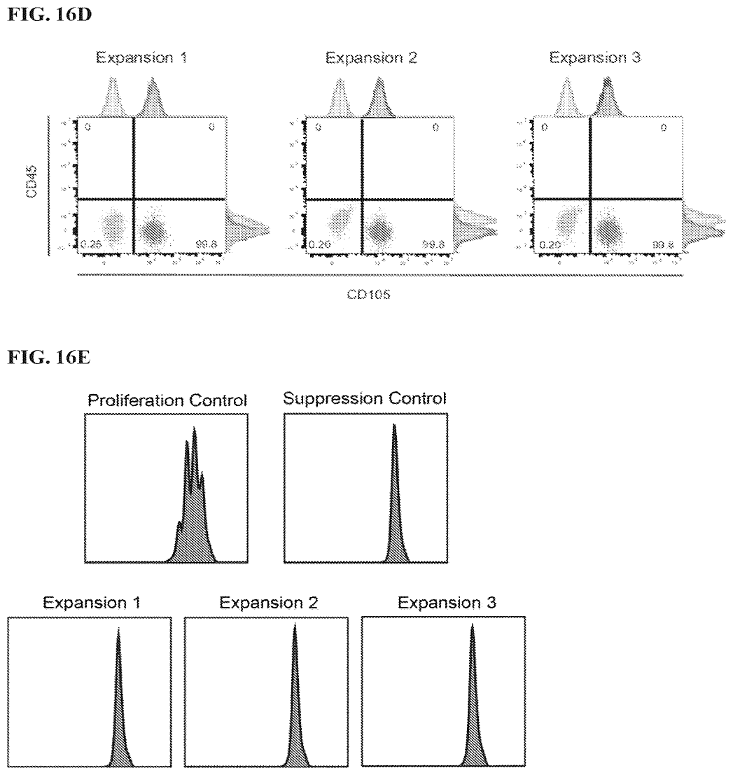

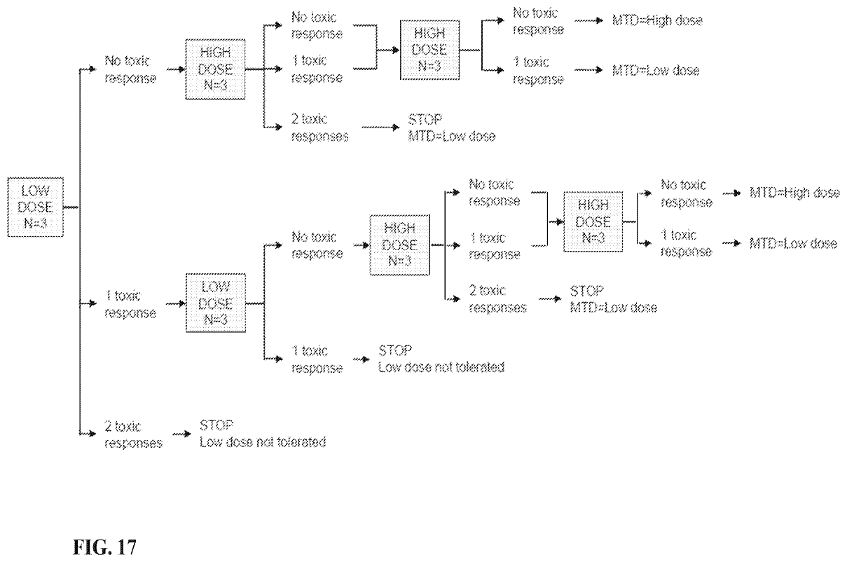

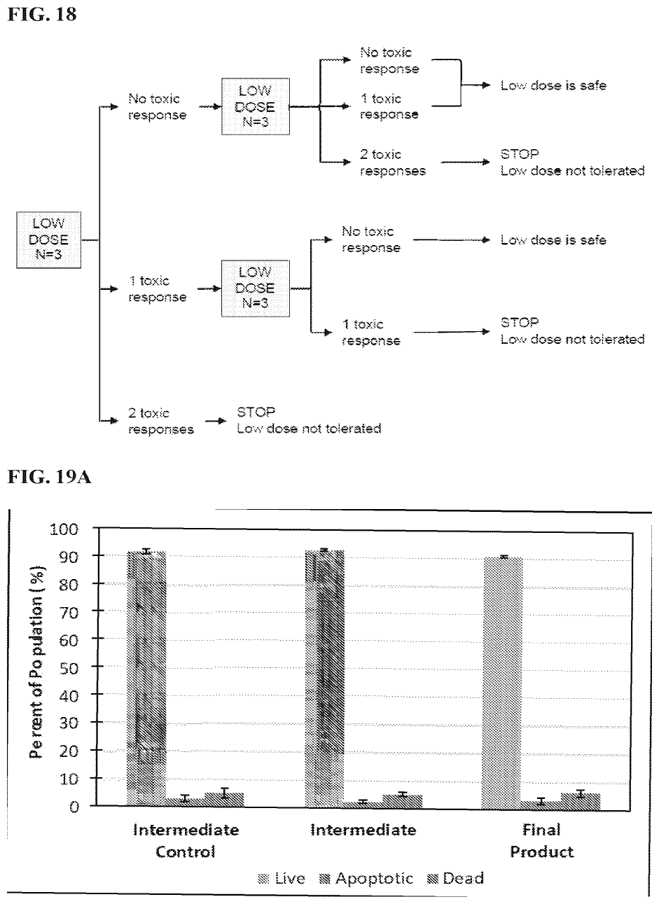

The phenotypes of vBM-MSC and/or vBA-MSCs (collectively MSCs) recovered by the methods described herein, after exposure to IFNγ, were characterized (see FIG. 16A). The observed phenotypes were consistent with the phenotypes previously reported for bone marrow derived MSCs exposed to IFNγ. Moreover, vBA-MSC express low levels of CCR7 which was sufficient to induce migration towards CCL19 in a transwell assay (see FIG. 16B). A flow cytometric analysis of ex vivo expanded, interferon γ primed BM MSCs shows the widely accepted defining antigen expression (FIG. 16C), which shows histograms of flow cytometric analyses of interferon γ primed BM MSCs for the indicated antigens (blue solid line) or isotype control (red dashed line). As shown in FIG. 16C, the γMSCs lacked expression of CD45 indicating the absence of hematopoietic contamination, and highly express CD105, CD73, and CD90 characteristic of the MSC phenotype. Scatter plots of three γ primed BM-MSCs expansions showed percentages of CD45+ cells of less than 0.25% and percentages of CD105+ cells of 99.8% (See, FIG. 16D). A 10.5-fold expansion over seven days (3.4 population doubling, 48.3 h doubling time), with >99% viability (n=2) was observed with these cells.

The interferon γ primed BM MSCs were able to suppress T cells in vitro. Freshly isolated human T cells were labeled with Cell Trace Far Red (membrane tracking dye, Thermo Fisher) and activated with CD3CD28 Dynabeads® (Thermo Fisher) according to a standard laboratory protocol. The positive control was activated in fresh media while the suppression control was in locally produced (suppressive) γMSC conditioned media. From each validation, MSC expansion and priming, conditioned media was prepared and tested for T cell suppression activity. The cell product for each validation cell expansion suppressed T cell proliferation in response to CD3CD28 bead activation similar to the suppression control (See, FIG. 16E). FIG. 16E demonstrates the dilution of cell trace far red membrane dye (to the left) with expansion compared with a uniform bright signal (to the right) of nondividing cells.

Expression of IDO1 after interferon γ priming was determined and comparison to DDIT4. The expression of DDIT4 is unchanged by interferon γ. The ΔCt was determined by normalizing the cycle threshold cycle (Ct) for IDO1 and DDIT4 to ACTGI which is consistently expressed in MSCs and γMSCs. The ΔΔCt for IDO1 to DDIT4 and the Relative Expression was determined according to the formula RE=2^{-ΔΔCt}. IDO1 expression was undetectable in MSCs as expected. Any value RE>1 was deemed acceptable. Data is shown below:

| | ΔΔCt | Relative Expression |
|---|---|---|
| Sample 1 of MSCs | −2.279 | 2.8 |
| Sample 2 of MSCs | −1.505 | 4.9 |

Results show equivalence in morphology, phenotype, growth rate and viability, T cell suppression in vitro, and IDO1 expression before and after interferon γ priming of the MSCs. Thus, the γMSC of the present disclosure are suitable for infusion into human patients and for treating various disorders of an asthma exacerbation, difficulty in breathing, coughing, dyspnea, episodes of asthma attacks, shortness of breath, wheezing when exhaling, or chest tightness or pain experienced by the subject.

In this example, the subject is a human child or a human adult.

In this example, the subject is further administered a standard of care treatment for asthma, e.g., administered albuterol, prednisone, and/or prednisolone). The subject may be administered a biologic, e.g., reslizumab (anti IL-5 neutralizing antibody), benralizumab (anti IL-5 receptor alpha), dupilumab (anti IL-4 receptor alpha subunit), mepolizumab (anti IL-5 neutralizing antibody), and/or omalizumab (anti-IgE neutralizing antibody). In this example, the subject may use a controller therapy for asthma, e.g., inhaled corticosteroids. The subject may also use analgesics, antihistamines, and/or intranasal corticosteroids.

### Example 3: Up to ˜40% of MSCs do not Survive Over the Next ˜24 Hours in Culture after Thawing

While MSCs exhibit high viability post thaw based on membrane integrity, up to ˜40% do not survive over the next ˜24 hours in culture (see Table 9). In vivo studies have observed that the bulk of infused MSCs persist for ˜2 days. However, it was observed that when using cryopreserved cells thawed and immediately infused, a significant number of them become necrotic following apoptosis during this brief window during which their primary effect must be realized. Additional analysis evaluating culture periods ranging from 12 to 48 hours confirmed this result and indicated that surviving cells begin cycling again as expected, with doubling beginning at approximately 30 hours (Table 10 A, B).

Table 9. Recovery of cells following cryopreservation and thaw, evaluated for membrane integrity via 7-AAD using flow cytometry immediately post thaw, cell count as a percentage of cryopreserved, and percent of cells recovered based on the number initially plated after the stated culture period (n=1 donor, 11 replicates performed on different days).

| | | Post Thaw | Time in | |
|---|---|---|---|---|
| Donor: | Post Thaw | Recovery | Culture | % of Recovered |
| W-20-043 | Viability | (yield) | (hours) | Cells Harvested |
| Mean | 92.27% | 90.20% | 24.88 | 57.46% |
| Minimum | 88.72% | 62.10% | 23.12 | 25.96% |
| Maximum | 97.58% | 142.50% | 26.25 | 80.81% |

Table 10. Recovery of cells at discrete culture periods post-thaw, comparing viability as assessed through 7-AAD using flow cytometry immediately post thaw and at each time point as well as cell yield at each time point represented as the percentage of cells initially plated.

A

Percent Viability (Flow Cytometry, 7-AAD)

Cryo-Recovery

| Donor | Pre-Freeze | Post-Thaw | 12 hr | 24 hr | 36 hr | 48 hr |
|---|---|---|---|---|---|---|
| W-20-043 | 99% | 87% | 92% | 95% | 86% | 94% |
| W-21-206 | 86% | 88% | 78% | 79% | 85% | 89% |
| W-21-298 | 83% | 85% | 79% | 77% | 85% | 88% |

B

| | Live Cells per T75 | | | | | Percent Recovery (Lifted/Plated) | | | |
|---|---|---|---|---|---|---|---|---|---|
| | Cryo-Recovery | | | | | Cryo-Recovery | | | |
| Donor | 12 hr | 24 hr | 36 hr | 48 hr | Donor | 12 hr | 24 hr | 36 hr | 48 hr |
| W-20-043 | 8.8E+0S | 1.2E+06 | 1.SE+06 | 2.2E+06 | W-20-043 | 37% | 52% | 64% | 91% |
| W-21-206 | 1.SE+06 | 1.6E+06 | 1.9E+06 | 3.9E+06 | W-21-206 | 63% | 69% | 77% | 164% |
| W-21-298 | 9.6E+0S | 1.4E+06 | 1.6E+06 | 2.1E+06 | W-21-298 | 40% | 60% | 65% | 87% |

Furthermore, it was observed that, of the cells that do survive cryopreservation, immediately thawed MSCs have a diminished ability to suppress T cell proliferation (an important indicator of immunomodulatory fitness) until normal cell cycle is resumed. This is consistent with other studies that have also noted introduction of interferon gamma (IFNγ), a potent cytokine that deploys MSC immunosuppressive properties, prior to cryopreservation to “pre-license” or “pre-prime” MSCs can enhance this fitness post thaw.

### Example 4: Cells in S Phase are More Susceptible to Harm During Cryopreservation

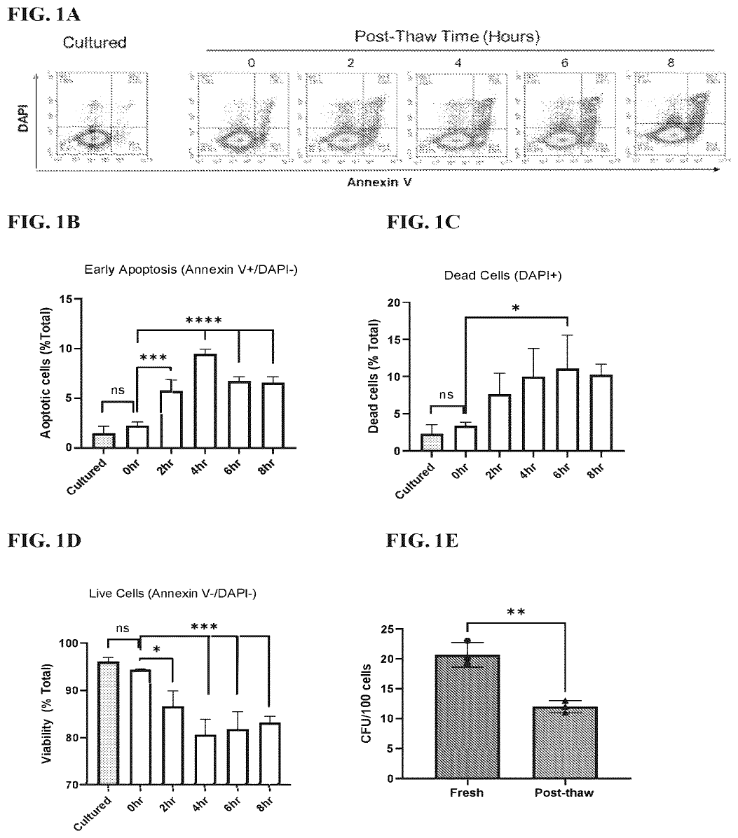

As shown in FIG. 1A to FIG. 1E, cryopreservation of MSCs induces cell death during post-thaw recovery in culture. Apoptosis of freshly cultured (i.e., non-cryopreserved) and thawed cryopreserved MSC was determined by Annexin VandDAPI staining (FIG. 1A). Immediately after thawing there was no difference in apoptosis and viability of cryopreserved cells compared to cells in culture (FIG. 1B and FIG. 1C; P>0.05). The percentage of Annexin v⁺/DAPI· apoptotic and DAPI+ dead cells increased overtime in culture, peaking by approximately 4 hr at 9.5±0.48% (mean±standard deviation) and 10.0±3.8%, respectively, which were both significantly higher than immediately post-thaw (P<0.05). By 4 hr, viable cells decreased to 81±3.3% from an initial viability of 94±0.12% (FIG. 1D). Additionally, the frequency of colony forming unit-fibroblast cells (CFU-F), which is an indication of clonogenicity, was severely compromised in cryopreserved MSC (FIG. 1E). Thus, immediate post-thaw analysis is not a reliable predictor of cell health, which decreases substantially over time even when cells are cultured in optimized nutrient rich conditions.

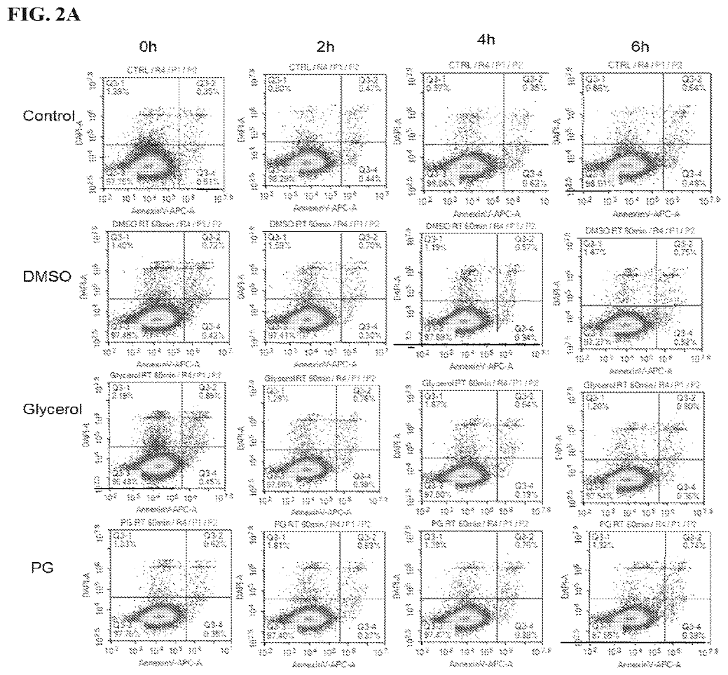

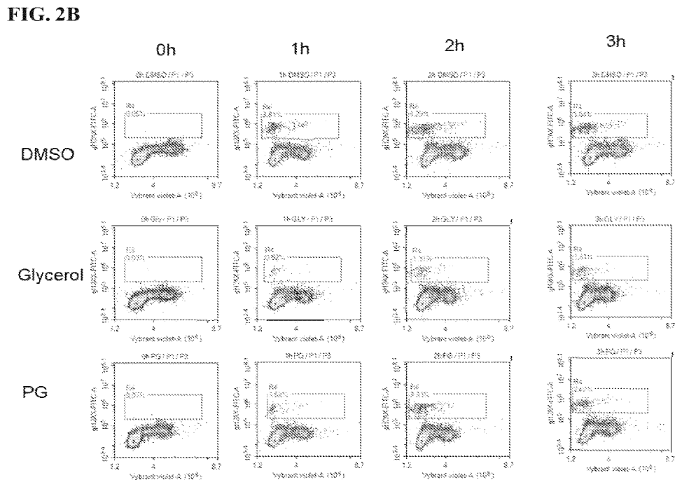

Delayed apoptosis and death were confined to a small percentage of thawed cells, suggesting that catastrophic cryoinjuries, such as Me₂SO₄-induced toxicity and osmotic stress, were not the primary causes of progressive viability loss after 2 hr in culture, which was subsequently confirmed (FIG. 2A and FIG. 2B). FIG. 2A shows MSCs that were suspended in culture medium (control) or cryoprotection medium containing either 5% Me₂SO₄, 5% glycerol or 5% propylene glycol (PG) for 60 min, then washed and placed in culture medium before evaluating apoptosis at 0, 2, 4 and 6 hr. FIG. 2B shows that a portion of the MSC in the different cryoprotectants were passively frozen to −80° C. and then placed in LN2 for 1 week. The cells were rapidly thawed and evaluated for DSB formation in relation to cell cycle using a gH2AX antibody and a DNA stain (Vybrant violet).

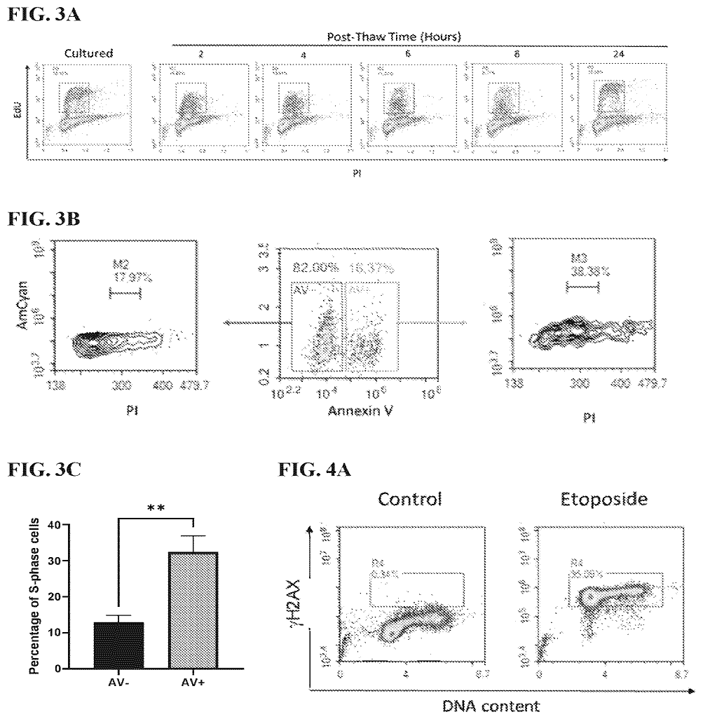

It has been reported that cryopreservation induces breaks in single-stranded DNA during S phase replication. It has been demonstrated that single stranded lesions were initially formed, followed by double-stranded breaks (DSB) and disruptions of chromatin higher order. Thus, apoptosis in relation to cell cycle status and correlated this to DNA synthesis was investigated (FIG. 3A to FIG. 3C). Compared to continuously culturing or post-thaw recovery for 24 hr, the fluorescence intensity of EdU staining was reduced, indicating compromised DNA synthesis in thawed MSC (FIG. 3A). This decline suggested that S phase cells were impacted by freezing and thawing. Analysis of apoptosis indicated that S phase cells were disproportionately represented in the population of dying cells (32.4±4.6%) compared to the Annexin V. population (12.9±1.9%) of S phase cells, indicating that MSC in this phase were more labile to cryoinjury (FIG. 3B and FIG. 3C).

The presence of DNA DSB in thawed MSC was detected by labeling with an antibody to H2AX subunits surrounding the breaks which are phosphorylated (termed γH2AX) within minutes of DNA damage (FIG. 4A to FIG. 4D). It was first confirmed that γH2AX could be measured in the assay conditions employed in this study, using the topoisomerase II inhibitor etoposide as a positive control which induced extensive DSB (FIG. 4A). Cryopreserved MSC from multiple donors were then evaluated for DSB at increasing time intervals after thawing. Very few γH2AX+ cells were detected immediately after thawing (FIG. 4B and FIG. 4C). Double-stranded breaks were observed at 1 hr post-thaw in MSC from three donor sources and increased thereafter until 3 hr, the longest period measured (FIG. 4D).

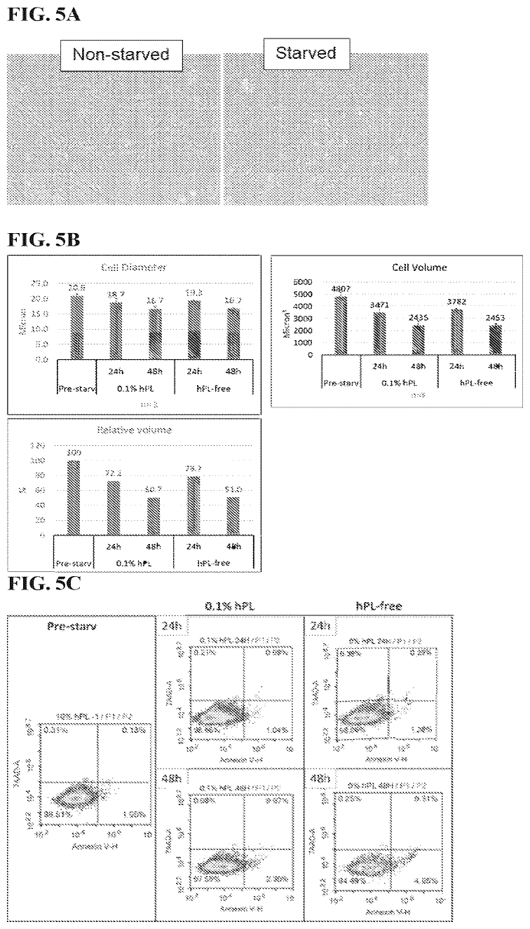

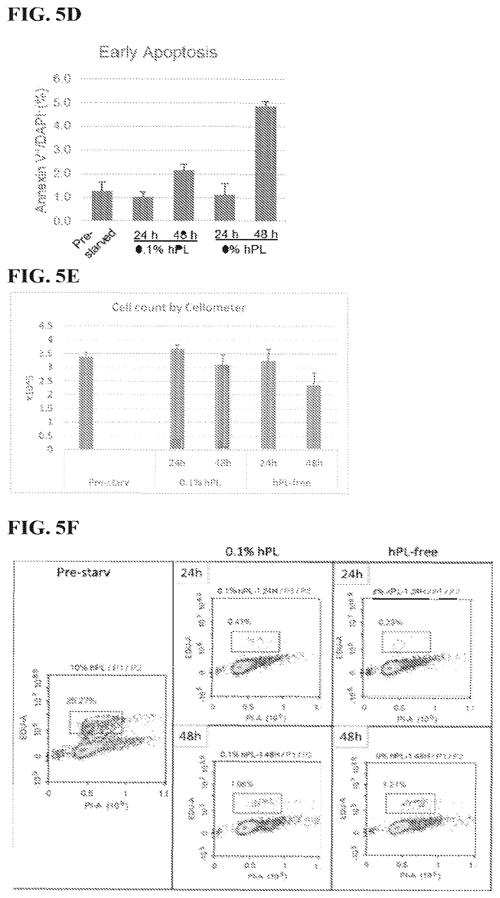

The association between increased apoptosis and S phase cells harboring DSB led to an examination of whether blocking progression to the DNA replication phase, and thereby preventing formation of labile single-stranded DNA, would protect cryopreserved cells. Serum starvation to deprive cells of mitogenic growth factors is a well-established method for synchronizing cells through inducing quiescence at G0, thus, blocking entry to S phase. Experiments were performed to determine appropriate levels of “serum starvation”, in this case deprivation of human platelet lysate (hPL). Cells were transferred from nutrient rich medium containing 10% hPL to base medium either totally devoid of hPL or containing a very low level (0.1%) of hPL and cultured for 24 and 48 hr. The cell morphology was unchanged by starvation; however, there was a diminution in size (FIG. 5A and FIG. 5B). The smaller cell diameter may help the cells distribute more readily in a patient, for example, get past the lungs easier. Also, the smaller volume means less water so potentially less intracellular ice potential during cryopreservation. The percentage of cells undergoing apoptosis for both starvation conditions was similar to non-starved cells at 24 hr (FIG. 5C and FIG. 5D). Conversely, MSC were adversely impacted by hPL starvation for longer periods as indicated by a more than doubling of the percentage of apoptotic cells at 48 hr, which was reflected in lower cell counts at this time (FIG. 5E). DNA synthesis was barely detectable by 24 hr, indicating that the MSC exited the cell cycle during the starvation period (FIG. 5F).

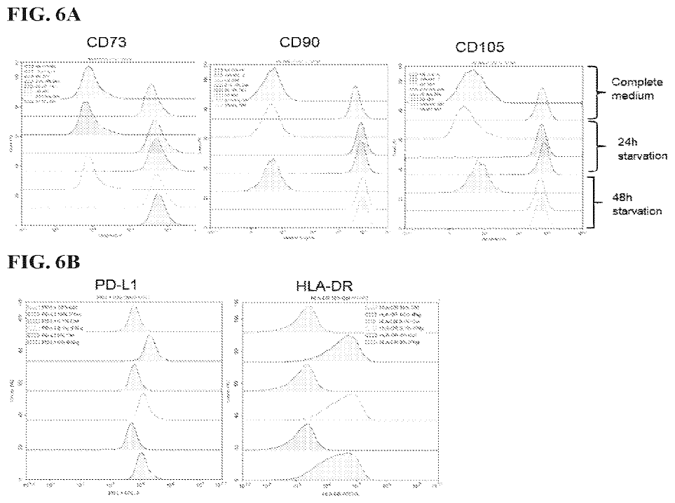

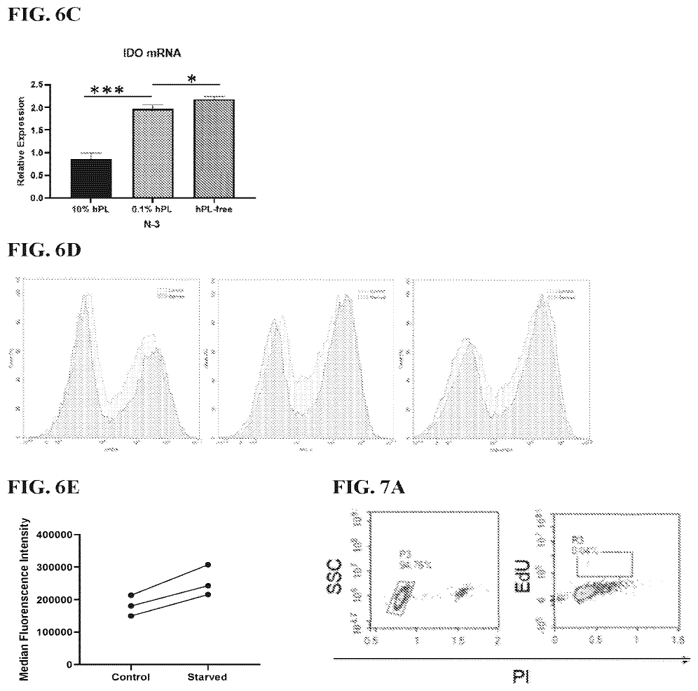

The impact of starvation on classical properties of MSC was next examined. Starving the cells did not alter expression of surface markers commonly associated with MSC (FIG. 6A). Additionally, starved MSC cultured in complete growth medium were responsive to interferon-g (IFN g) priming, as evidenced by increased surface expression of Class II human leukocyte antigen (HLA) DR and programmed death ligand 1 (PD-L1) (FIG. 6B). Interestingly, gene expression of the immunoregulatory protein, indoleamine 2,3-dioxygenase-1 (IDO-1), increased with starvation (FIG. 6C). There was also a slight increase in IDO-1 protein expression (FIG. 6D and FIG. 6E). These experiments indicated that culturing MSC for 24 hr under conditions of hPL deprivation was sufficient to block progression to S phase and didn't promote any obvious deleterious effects. Furthermore, starvation of MSC did not fundamentally alter the phenotype or responses to environmental stimuli.

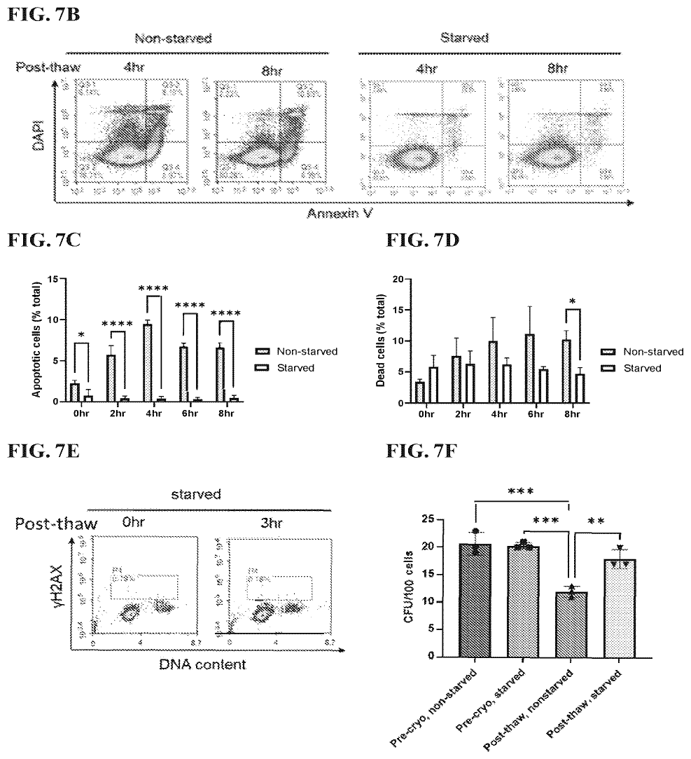

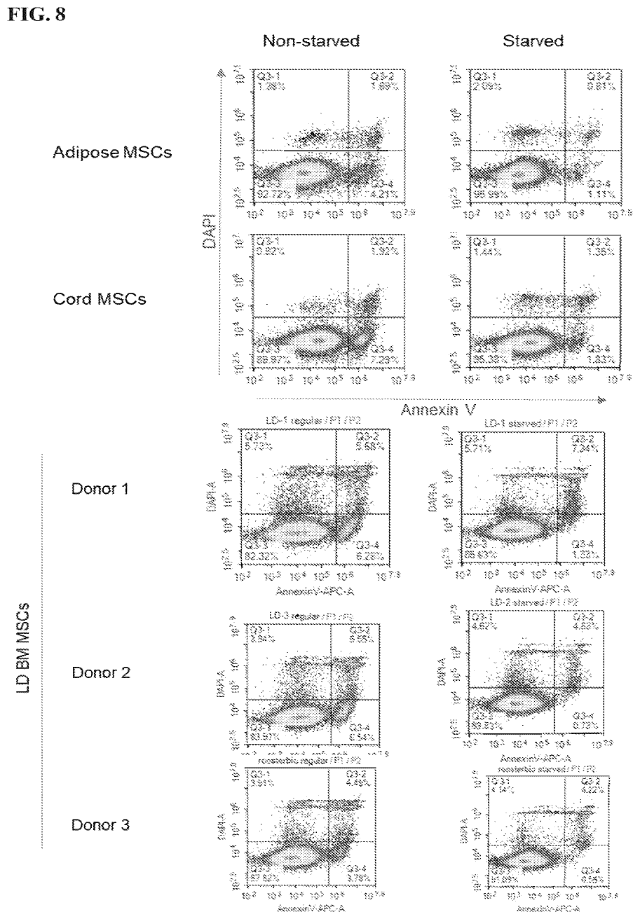

To determine the impact of growth factor starvation on post-thaw recovery of MSCs, cells were transferred from complete medium to basal medium without hPL and cultured for 24 hr. The majority of cells were in G0/G1 as indicated by the absence of DNA synthesis (FIG. 7A). Apoptosis of both starved and non-starved MSC was analyzed prior to cryopreservation and following thawing and culturing in complete medium for 4 and 8 hr. Starving cells prior to cryopreservation greatly reduced the number of apoptotic cells (annexin v⁺/DAPI⁻) after thawing (FIG. 7B and FIG. 7C). This effect was observed at all timepoints measured (0 to 8 hr). Whereas the percentage of apoptotic non-starved cells increased until 4 hr, apoptosis in starved cells remained low throughout the experimental period. Similarly, the percentage of dead (DAPI+) cells increased with time in culture for non-starved cells (FIG. 7D). The percentage of dead cells in starved MSC held steady during the 8 hr and were significantly lower than non-starved cells at 8 hr.

Importantly, starvation completely prevented the occurrence of DSB breaks in thawed cells by blocking DNA synthesis prior to cryopreserving MSC (FIG. 7E). In contrast to cells that were not starved prior to cryopreservation (FIG. 4C), the percentage of γH2AX-positive cells did not increase over background levels after thawing (FIG. 7E). The very low level of γH2AX staining in starved compared to non-starved cells was confirmed by immunofluorescence staining (FIG. 7E).

The impact of growth factor deprivation on the CFU-F potential of cryopreserved MSC was determined. Cells grown in complete medium prior to cryopreservation exhibited impaired (P<0.01, unpaired T test) CFU-F potential compared to GO-arrested MSC (FIG. 7F). In fact blocking mitogenesis prior to cryopreservation resulted in CFU-F potentials that were indistinguishable from non-cryopreserved MSC.

This phenomenon was shown to be associated with the process of cryopreservation and thawing and not caused by Me₂SO₄ as DSB were not detected prior to cryopreservation after incubating cells for 1 hr in Me₂SO₄ or the alternative cryoprotectant agents, propylene glycol and glycerol (FIG. 2A and FIG. 2B). The protective effect of growth factor starvation on cryopreserved MSC was also observed with MSC obtained from living donor iliac crest BM and MSC derived from other tissues (umbilical cord and adipose) (FIG. 8).

In total, these data are highly supportive of DNA double-stranded breakage being a major factor contributing to post-thaw loss of MSC viability and function and blocking S phase entry prior to cryopreservation prevents these occurrences.

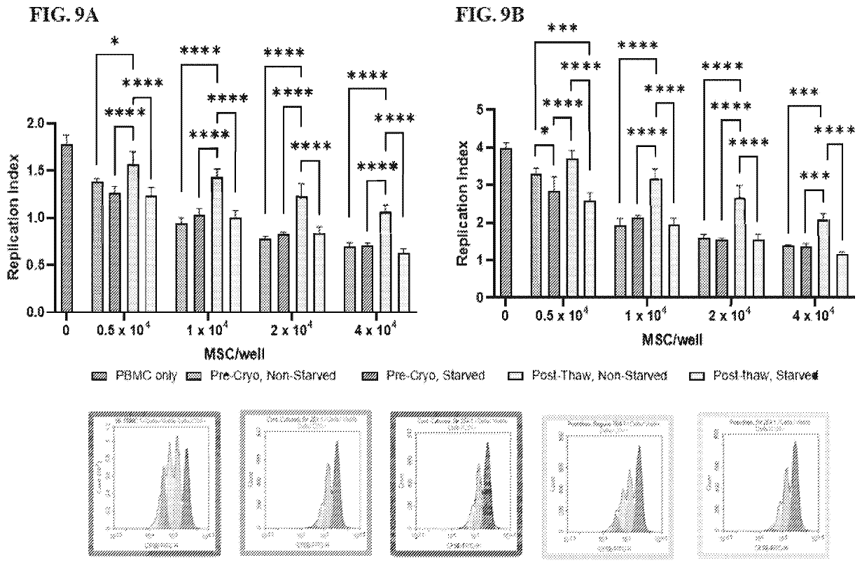

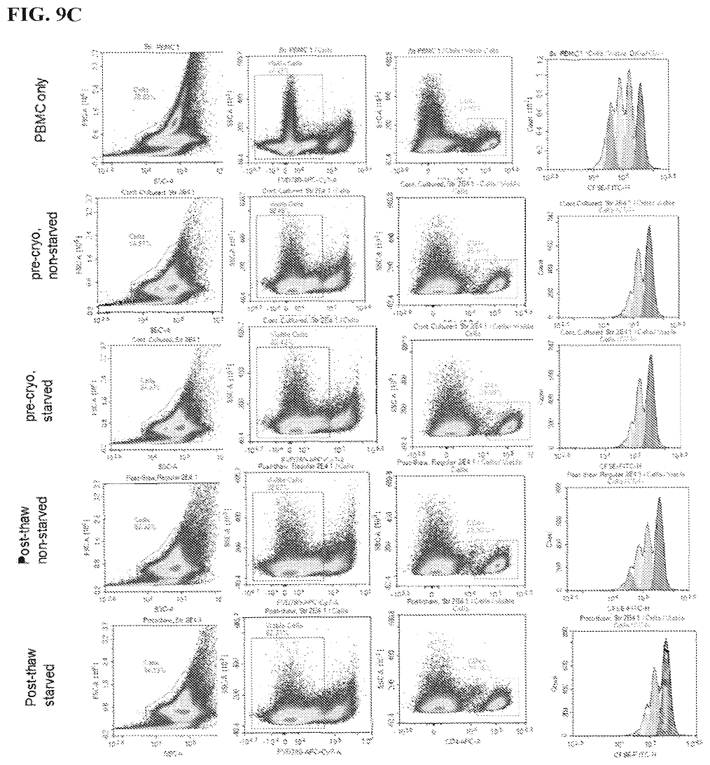

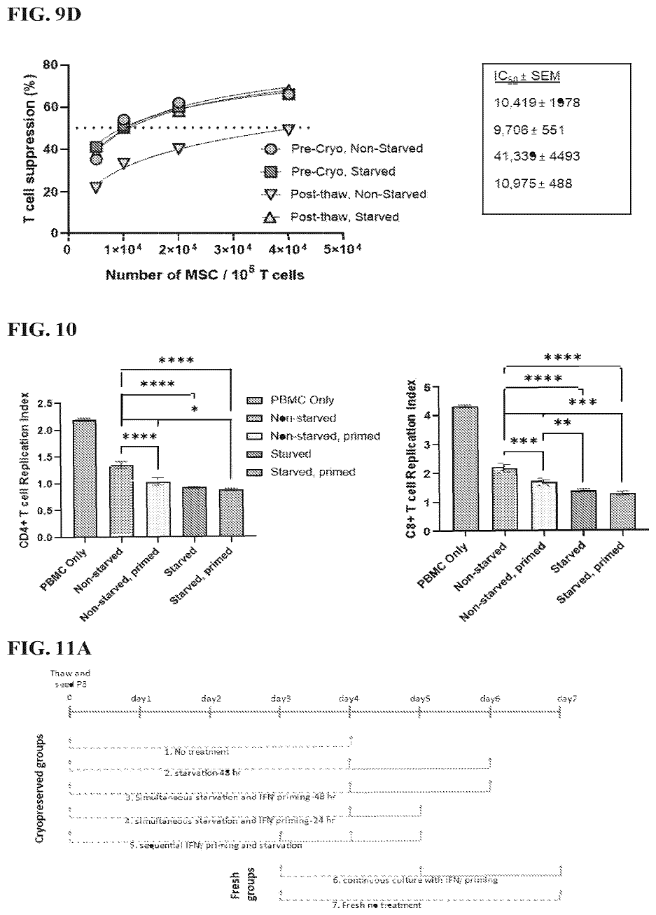

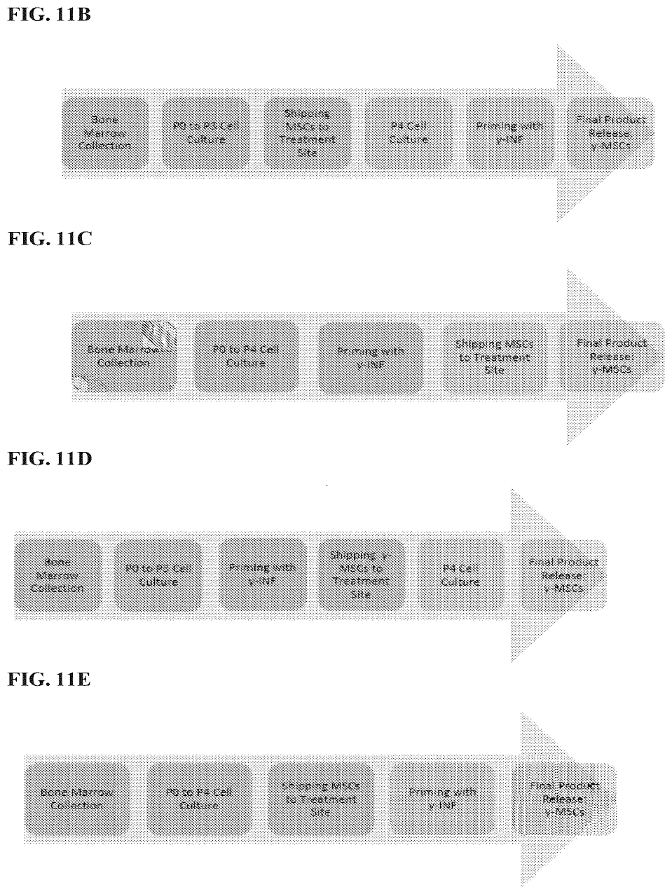

The effect of growth arrest on MSC function was further evaluated. Cryopreserved MSC have been reported to be impaired for immunosuppressive function post-thaw; therefore, starved and non-starved cells were tested prior cryopreservation or after thawing in T cell suppression assays. Non-starved cells demonstrated the least suppression immediately post-thaw, whereas there was no difference in the CD4+ and CD8⁺ T cell suppressive activity between the other groups at the highest concentrations of MSC (FIG. 9A and FIG. 9C). At the lowest ratio of 1:80 MSC: T cells, which comprised approximately 50% of PBMC, post-thaw, starved MSC were significantly (p<0.05) more suppressive than non-starved, freshly cultured MSC (FIG. 9B). The ratio of thawed MSC required to suppress activated T cell proliferation by 50% (IC₅₀) was 8-fold less (1:80) without starvation than with starvation (1:10) (FIG. 9D). The IC₅₀ for post-thaw starved MSC was no different than MSC prior to cryopreservation. These data suggest that growth arrested MSC retain pre-cryopreservation levels of immunosuppressive function.

It was previously demonstrated that priming BM-MSC with IFNγ prior to cryopreservation partially rescued post-thaw viability and function. Therefore, whether IFNγ-licensing could further enhance growth arrested MSC function post-thaw was investigated. Both starvation alone and starvation with IFNγ priming significantly (p<0.001) enhanced post-thaw T cell suppression activity compared to MSC cryopreserved directly after culturing in complete medium (FIG. 10). Simultaneously starving and priming MSC enhanced T cell suppression activity over priming alone (p<0.05 and p<0.001, CD4⁺ and CD8⁺ T cells, respectively).

This Examples discloses method for enhancing recovery of cryopreserved MSC to yield more viable and functional cells compared to traditional cryopreservation methods. Subjecting to mitogenic factor deprivation for 24 hours prior to cryopreservation induces synchronization of the entire cell population by forcing exit from the cell cycle at early G1 phase, thus, preventing entry into S phase which avoids formation of stalled single-stranded DNA replication forks that are susceptible to freeze-thaw damage. Without wishing to be bound by theory, cell death due to cryopreservation induced DSB is a delayed effect which manifests with extended periods of culture in vitro, which presumably models kinetics of cell viability in vivo. Thus, viability assessments that rely on a single timepoint at an interval close to the time of thawing cells for infusion may not be indicative of the actual physiological status of cells used for therapeutic applications.

Previous reports describe the functional impairment of cryopreserved MSC. The experimental data disclosed herein further provides a mechanistic explanation, which directly led to the practical solutions for overcoming this serious impediment to clinical translation of MSC therapies. Furthermore, growth factor starvation can be combined with other strategies to further enhance post-thaw viability and function. For instance, as disclosed herein, combining starvation with IFNγ priming (also known as IFNγ licensing) has an additive effect on functional recovery of cryopreserved MSC (FIG. 10).

It is interesting to note that the enhanced survival afforded by preventing 15-20% cells in a non-synchronized culture from entering S phase (FIG. 3A to FIG. 3C) led to a 4-fold enhancement in T cell suppression activity (FIG. 9A to FIG. 9D). This observation indicates that synchronizing MSC to G0/G1 phase prior to cryopreservation has additional benefits in addition to enhancing post-thaw survival. A slight increase in IDO-1 mRNA and protein steady-state levels was observed in growth factor starved cells (FIG. 6A to FIG. 6E). The increase may contribute to the effect of starvation on enhanced T cell suppression and/or it might indicate that growth factor deprivation confers a metabolic state that is receptive to inflammatory factor-mediated stimulation of MSC suppression. Growth factor deprivation may also enhance expression of other immunomodulatory factors. Culturing MSC under conditions that induce metabolic reprogramming has been shown to enhance anti-inflammatory cytokine expression. It is also possible that starvation induces a quiescent state, which promotes greatly reduced metabolic activity, with many cell types transitioning from oxidative phosphorylation to glycolysis or fatty acid oxidation to meet basal metabolic needs. A transition to glycolysis in response to inflammatory stimulus enhances the immunosuppressive phenotype of MSC, which may explain the results obtained here.

Prolonged growth factor starvation induces reversable exit from the cell cycle and adoption of a quiescent state until growth conditions are favorable, at which time the cell will re-enter the cycle. Quiescence is a property of stem cells as well as many lineage-restricted and differentiated cell types and is associated with increased resistance to stresses and injury as well as epigenetic changes that maintain a state of readiness for quickly responding to changes in environmental states. Starvation-induced quiescence of MSC produces cells that are better able than actively proliferating cells to withstand heat shock through faster DNA repair, more rapid resumption of proliferation and preventing triggering of the senescence response. Quiescent cells can exist in a primed state which was first described in skeletal satellite cells, skeletal muscle resident MSC and hematopoietic stem cells and termed GALERT-Cells in the GALERT state are characterized by increased transcriptional and metabolic activity compared to quiescent (i.e., classical G0) cells and, thereby, poised to respond quickly to paracrine signals. This state has been described as “idling”. It is possible that starved MSC adopt a state that promotes accelerated re-entry into the cell cycle combined with a heightened response to inflammatory signals when transferred to growth-permissive conditions.

Another probable benefit of growth factor starvation prior to cryopreservation is the −25% reduction in the size of cells. The reduced size of starved cells would be expected due to growth factor-responsive restriction point control of the cell cycle which blocks progression early in G1 and causes exit from the cycle prior to completing G1 and G2 growth phases. Mitogenic factor withdrawal also induces autophagy which could further contribute to reducing cell size. Smaller size reduces the surface to volume ratio and intracellular water concentration, which is expected to enhance exchange of water and cryoprotectant. Additionally, the small size after thawing could lead to better localization to targeted tissues. It has been frequently observed in animal models that the vast bulk of intravenously administered MSC trap in the lungs and are quickly cleared by lung resident macrophages. The smaller cell size of G0/G1 synchronized cells could promote transit of lung microvasculature; thereby, enhancing the probability that systemically delivered MSC will reach the injured or diseased target tissues.

Beyond enhancing function, starved, cryopreserved MSC are likely to be safer due to preventing infusion of cells with extensively damaged DNA into patients. Damage to DNA is not uncommon in actively dividing cells, occurring at a rate of approximately 5000 single-stranded lesions/cell cycle in a typical human cell. Most errors are trivial and can be repaired by pausing or decelerating the cell cycle. However, DSB repair is much more complex and may require temporary exit from the cell cycle to provide sufficient time to perform repairs. A cell harboring irreparable DNA lesions will almost always undergo cither apoptosis, senescence or terminal differentiation to prevent genetic instability due to mutant genomes being passed along to progeny. It has been observed, however, that cells harboring DSB involving large regions of the genome will, at some low frequency, undergo chromosomal recombination, which allows the cell to remain in the cell cycle, although, at the expense of genomic instability. Genetic rearrangements are the hallmarks of many cancer cells. Thus, synchronizing cells to G0/G1, using serum starvation or by other means, prevents potential transformation of MSC; thus, enhancing safety by preventing infusion of potentially tumorigenic cells.

In summary, the experimental results disclosed herein provide a simple GMP-compliant manufacturing step for enhancing post-thaw recovery of cryopreserved MSC. This process should remove barriers to clinical development and ultimately post-approval marketing caused by the logistical challenges due to poor effectiveness of cryopreserved cells. Furthermore, the higher activity, combined with smaller size, is likely to enhance the pharmacokinetic and pharmacodynamic properties of thawed MSC, which should in turn increase effectiveness over normally cultured and cryopreserved cells. Assuming greater potency is observed in clinical studies, the cost per dose of starved cells should be significantly lower than that for non-starved cells. Finally, cryopreserved starved cells are expected to be safer than non-starved MSC due to the reduced risk of accumulating tumorigenic chromosomal translocations caused by DSB.

### Example 5: Illustrative Methods

In these Examples, the following illustrative Methods and Materials were employed.

MSC culture, synchronization by starvation and cryopreservation. Deceased donor derived vertebral bone marrow MSC were isolated, cultured and expanded utilizing our published methods. Briefly, eluted BM from deceased donor vertebral bodies was seeded in CellBIND T-225 flasks (Corning, MA, USA) at a density of 2.5×10⁴ viable cells/cm² with a MEM (Biologos, Montgomery, IL USA) containing 10% human platelet lysate (hPL; Stemulate®, Sexton, Indianapolis, IN, USA), 2 ng/ml each of recombinant human epidermal growth factor (rhuEGF; R&D Systems, Minneapolis, MN, USA) and recombinant human basic fibroblast growth factor (bFGF; R&D Systems) and maintained at 5% humidified CO₂ and 37° C. At 70% confluence cells were detached using 1×TrypIB (Thermo Fisher Scientific, Waltham, MA USA) and passaged a density of 4000 cells/cm² until passage 4 for use in experiments. Umbilical cord and adipose MSC were purchased from Lonza (Morristown, NJ USA) and live donor BM-MSC were purchased from RoosterBio (Frederick, MD USA).

Synchronization to G0/G1 by hPL starvation: When passage 4 MSC in T175 flasks attained 70-80% confluency, the cells were washed once with 20 mL a MEM and then cultured in a MEM or a MEM containing 0.1% hPL for another 24 to 48 hr. Where indicated, IFNγ (R&D Systems; 25 mg/ml) was also added to the medium during starvation.

To cryopreserve MSCs, cells were harvested from cultures using TrypLE, pelleted and resuspended in pre-chilled homemade freezing solution (Plasma-Lyte; Baxter, Deerfield, IL USA; containing 2.5% human serum albumin; Octapharma, Paramas, NJ USA; supplemented with 5% Me₂SO₄; Biolife Solutions, Bothell, WA USA) at a density of 1×10⁶ cells/mL and aliquoted into cryovials. Vials were placed in a CoolCell (Corning Life Sciences, Durham, NC USA) and frozen in a −80° C. freezer overnight before storage in liquid nitrogen vapor.

For thawing, all vials were removed from liquid nitrogen and quickly placed in a 37° C. water bath until a small amount of ice remained. Cells were then gently transferred into 15 ml conical tubes containing 10 ml pre-warmed media, centrifuged at 500×g for 5 min, and resuspended in 1 mL pre-warmed media for use in downstream applications.

For colony forming unit-fibroblast (CFU-F) assay, 100 viable continuously cultured or thawed MSC were plated in triplicate wells of 6-well plates in MethoCult™ medium (StemCell Technologies, Vancouver, BC Canada). After 11 days, the cultures were washed with PBS and fixed with methanol, followed by 0.5% crystal violet staining. A cell cluster that had more than 50 cells was counted as a colony under microscopy.

Apoptosis assay. Post-thaw apoptosis of MSC was assessed by staining the cells at different time points with Annexin V-APC (Biolegend, San Diego, CA USA) and DAPI (Biolegend). Briefly, thawed cells were plated in 6-well plates at 2×10⁵ per well and recovered by being placed in a 37° C. incubator. Cells immediately after thawing served as the 0 hr time point. At various time points (2, 4, 6, and 8 hr), cells (both floating and attached) were harvested and stained with Annexin V-APC and DAPI (0.5 μg/mL) in Annexin V binding buffer (Thermo Fisher Scientific) according to manufacturer's instruction. A NovoCyte flow cytometer (Agilent Technologies, Santa Clara, CA USA) was used for data collection, and data were analyzed using NovoExpress software (Agilent). To determine DNA content in apoptotic population, Annexin V stained MSC were fixed with 3.7% formaldehyde solution by diluting the 37% formaldehyde stock (Sigma-Aldrich, Saint Louis, MO USA) and followed by permeabilization with 0.5% triton X-100. Then cells were treated with 10 mg/mL RNase (Sigma-Aldrich) to remove RNA and subsequently stained with propidium iodide (Biotium, Fremont, CA USA) and analyzed by flow cytometry. In the whole procedure the annexin V binding buffer was used to ensure the binding of Annexin V-APC on the surface of the cells.

Ethylnyl-2′-deoxyuridine (EdU) incorporation assay. To determine the DNA incorporation efficiency of S phase cells during post-thaw recovery, the EdU incorporation assay was performed using a Click-iT Plus EdU Alexa Fluor 647 Flow Cytometry Assay Kit (Thermo Fisher Scientific), according to the manufacturer's instructions. Briefly, 1 hr prior to each indicated time point, 10 μm final concentration of EdU was added to each well containing post-thaw MSCs. After 1 hr incubation, cells were fixed, permeabilized and the incorporated EdU was detected using Alexa Fluor 647 azide. After wash cells were incubated in PBS buffer containing 10 mg/mL RNase for 1 hr at room temperature and subsequently stain with propidium iodide and analyzed by flow cytometry.

Detection of DNA DSB by gH2AX staining. g-H2AX detection was performed as described by Muslimovic et al. Briefly, at indicated time point, at least 105 cells in 50 mL (PBS supplemented 1% BSA) were added to 150 uL anti-H2AXS139ph FITC conjugate (0.6 μg/mL, EMD Millipore, Burlington, MA USA) supplemented Block-9 staining buffer (PBS, 1 g/l BSA, 8% mouse serum, 0.1 g/l RNaseA, 0.25 g/l herring sperm DNA, 0.1% Triton X-100, 5 mM EDTA, and phosphatase inhibitors including 10 mMNaF, 1 mM Na₂MoO₄, 1 mM NaVO₃. Staining was performed on ice in dark for 3 hr. Cell cycle distribution was monitored by addition of 5 μm Vybrant dye cycle violet stain (Thermo Fisher Scientific) during the last hour of staining. Then the cells were subjected to flow analysis. MSC treated with 100 μM Etoposide (Sigma-Aldrich) for 30 min were used as positive control.

Immunofluorescence microscopy. Continuously cultured or post-thaw MSC were seeded into 6-well plates at a density of 2×10⁵ per well and incubated for 2 hr in the presence of 10 μm EdU in a 37° C. incubator. Then cells, both floating and attached, were collected and resuspended in PBS at 5×10⁵/mL and cell suspensions (75 μL/spot) was spun onto slides using a cyto-centrifuge (5 min at 1100 rpm, Cytofuge 2, Statspin, Norwood, MA USA). Then the cells were fixed with 3.7% formaldehyde for 15 min at room temperature. After being washed with PBS, cells were permeabilized by cold methanol at −20° C. for 10 min. Next incorporated EdU was detected using Click-&-Go® EdU 594 Cell Proliferation Assay Kit (Click Chemistry Tools, Scottsdale AZ, USA) according to manufacturer's instruction. Cells were then blocked using 5% FBS in PBS for 1 hr at room temperature followed by the addition and incubation of the following primary antibodies overnight at 4° C.: mouse anti-phospho-Histone H2A.X (Ser139) antibody (1:300, Millipore), rabbit anti-Cleaved Caspase-3 (Aspl 75) (1:400, Cell Signaling Technology, Danvers, MA USA). After three washes with 0.5% Tween in PBS, bound antibodies were detected with FITC conjugated goat anti-mouse IgG (1:300), FITC conjugated goat anti-rabbit IgG (1:300), and DyLight 594 conjugated goat anti-rabbit IgG (1:300) at room temperature for 2 hr. All secondary antibodies were purchased from Boster Biological Technology (Pleasanton, CA USA). After three washes, 5 μg/mL Hoechst 33342 PBS solution was used for DNA stain and fluorescent images were obtained by Leica DM4B system (Leica, Wetzlar Germany).

T cell suppression assay. Fresh whole blood was purchased from Versiti Blood Center in Indianapolis. Peripheral blood mononuclear cells (PBMCs) were isolated using Ficoll-Paque® (Cytiva, Marlborough, MA USA) density gradient centrifugation. The isolated PBMCs were labeled with carboxy fluorescein succinimidyl ester (CFSE; BD Bioscience) at a concentration of 5 mM per 20×10⁶ cells at room temperature for 10 min, followed by cryopreservation in 90% FBS/10% Me₂SO₄. Thawed MSC were seeded in RPMi with 10% FBS at a density of 1.2×10⁵ or 2.4×10⁵ cells per well of a 24-well plate and cultured for 24 hr. The supernatants were carefully removed and 1.2×10⁶ CFSE-labeled PBMCs in RPMI with 10% FBS were added into each well. The PBMCs were activated using a CD3c antibody (Bio X Cell, Lebanon, NH USA) at a concentration of 150 ng/ml. The MSC were co-cultured with the PBMCs at 37° C. for 4 days. Cell proliferation was determined by monitoring CFSE dilution in CD4⁺ and CD8⁺ T cells on Flow Cytometry.

Evaluation of cryoprotectant effects. Glycerol and Propylene Glycol (both purchased from Sigma, MO USA) were used as alternative CPAs. Alternative freezing solutions contain 2.5% HSA supplemented with 5% Glycerol or PG in Plasma-Lyte. To test the CPA effect on cells without cryopreservation, P4 MSCs were harvested and resuspended in freezing solutions made of Me₂SO₄, Glycerol or PG at a density of 1×10⁶ cells/mL and left in room temperature for 1 hr. The cells suspended in CPA free solution were used as control. Then the cells were treated as thawed cells for downstream experiment.

RNA extraction and real-time PCR. According to the manufacturer's instructions, total RNA was extracted from MSC using The Aurum total RNA mini kit (Bio-Rad, Hercules, CA, USA). One microgram of RNA was used for reverse transcription into cDNA using the high-capacity cDNA reverse transcription kit (Thermo Fisher Scientific, USA). Complementary DNA was then diluted and used for real-time PCR with GAPDH and IDO-1 probes (Applied Biosystems, UK) using TaqMan™ Universal PCR Master Mix (Thermo Fisher Scientific, USA) in a Mic qPCR Cycler system (Bio Molecular Systems, Queensland, Australia). Triplicate CT values were analyzed in Microsoft Excel using the comparative CT (L1L1CT) method and the amount of target (2-L1L1CT) was obtained by normalization to an endogenous reference GAPDH and relative to a calibrator.

Surface and intracellular protein analysis via flow cytometry. For surface marker analysis, MSC were re-suspended in PBS to a density of 2×10⁵ cells/ml, and labelled with corresponding anti-human monoclonal antibodies purchased from Biolegend, including CD73-FITC, CD90-APC-Cy7, CD105-APC, PD-L1-APC and HLA-DR-APC. DAPI was used to exclude dead cells from flow cytometry analysis.

For intracellular IDO-1 assessment, MSC were fixed and permeabilized using eBioscience™ Intracellular Fixation & Permeabilization Buffer Set (Thermo Fisher Scientific, USA) and stained with APC conjugated anti-IDO-1 antibody according to manufacturer's instruction. Labelled cells were analyzed via flow cytometry.

Statistical analyses. Results are expressed as the mean±SD. All statistical comparisons were made using Prism 9 (GraphPad Software Inc, La Jolla, CA USA).

### Example 6: Supplements to Priming MSCs with IFNγ

This example illustrates supplements to priming MSCs with IFNγ.

Following the steps described in Example 1, during the secondary expansion, MSCs are primed with IFNγ and serum starved and/or temperature shocked. More specifically, to enact serum starvation, during the last two days or the last day of culturing, human plateletlysate (hPL) is omitted from the culturing medium. Alternately, or additionally, during the final two hours or final hour of culturing, the temperature of the cell culture is raised to about 40° C., to enact temperature shock.

### Example 7: Alternates to Priming MSCs with IFNγ

This example illustrates alternates to priming MSCs with IFNγ.

Following the steps described in Example 1, during the secondary expansion, rather than priming MSCs with IFNγ, the MSCs are serum starved and/or temperature shocked. More specifically, to enact serum starvation, during the last two days or the last day of culturing, human platelet lysate (hPL) is omitted from the culturing medium. Alternately, or additionally, during the final two hours or final hour of culturing, the temperature of the cell culture is raised to about 40° C., to enact temperature shock.

### Example 8: Treating an Inflammatory Condition, Including Auto-Immune Conditions with IFNγ-Primed MSC (γMSCs)

In this example, a composition comprising an effective amount of interferon γ-primed mesenchymal stromal cells (γMSCs) is administered to a subject for treating an inflammatory condition, including an autoimmune condition, or a symptom thereof and/or reducing the likelihood of asthma attack thereof.

In this example, the subject has asthma or symptoms related to inflammatory condition, including an autoimmune condition, e.g., difficulty in T-cell activity. The subject may also experience shortness of breath or chess tightness or pain. In this example, the subject may have moderate-to-severe persistent inflammatory condition, including an autoimmune condition. In this example, the inflammatory condition, including an autoimmune condition is minimally treated by the standard of care treatment for inflammatory condition, including an autoimmune condition, or a frequency of recurrent inflammatory condition, including an autoimmune condition is not reduced by the standard of care treatment for inflammatory condition, including an autoimmune condition.

The composition comprising γMSCs (as described in Example 1 or 5) is administered via intravenously infusion with a dose of γMSCs in the amounts from about 2×10⁶ cells/kg to about 5×10⁶ cells/kg of ideal body weight or actual body weight. In this example, the subject may be administered diphenhydramine and/or acetaminophen within 60 minutes of cell infusion.

In some embodiments, the γMSC treats the inflammatory condition, including an autoimmune condition. Additionally, the γMSC reduces inflammation and circulating cell inflammation. The γMSC reduces inflammatory condition, including an autoimmune condition or symptoms related to the inflammatory condition, including an autoimmune condition, e.g., onset of an asthma exacerbation, difficulty in breathing, coughing, dyspnea, episodes of asthma attacks, shortness of breath, wheezing when exhaling, or chest tightness or pain experienced by the subject.

In this example, the subject is a human child or a human adult.

In this example, the subject is further administered a standard of care treatment for asthma, e.g., administered albuterol, prednisone, and/or prednisolone). The subject may be administered a biologic, e.g., reslizumab (anti IL-5 neutralizing antibody), benralizumab (anti IL-5 receptor alpha), dupilumab (anti IL-4 receptor alpha subunit), mepolizumab (anti IL-5 neutralizing antibody), and/or omalizumab (anti-IgE neutralizing antibody). In this example, the subject may use a controller therapy for asthma, e.g., inhaled corticosteroids. The subject may also use analgesics, antihistamines, and/or intranasal corticosteroids.

### Example 9: Fresh, not Previously Frozen IFNγ Primed MSC

In any of the above examples, rather than cryopreserving MSCs or γMSCs and thawing the MSCs (which are subsequently primed) or thawing the γMSCs prior to use (either immediately or after one or more culturing steps), fresh MSCs or fresh γMSCs may be used.

### Example 10: Re-Frozen Synchronized Cells can be Repeatedly Cryopreserved and Thawed without Substantial Loss of Potency

Cells that have been synchronized according to the herein-disclosed methods are resistant to damage due to cryopreservation. As such, synchronized cells may be frozen and thawed multiple times. Moreover, once finally thawed, an additional culturing step may not be required. Therefore, in some embodiments, methods of the present disclosure allow frozen cellular products to be shipped to a treatment site, thawed, and immediately administered to a subject in need.

In this example P3 MSCs were thawed and plated in a 5-chamber CellSTACK (Corning cat no. 3311) with MSC Culture Medium (MSC Growth Media with 10% human plateletlysate, 2 ng/mL EGF, and 2 ng/mL FGF2). The MSC were cultured for 5 days to yield synchronized P4 MSCs. On day 3, the MSC were refed with MSC Culture Medium. On day 4, the MSC were synchronized with MSC Growth Media (i.e., un-supplemented base medium).

On day 5, the MSC (75% confluent) were lifted and cryopreserved in MSC Growth Media with 10% human platelet lysate and 5% DMSO (“MSC Medium”), yielding P4 MSCs “Intermediate”. To serve as a control (“Intermediate Control”), some of the lifted MSC were cryopreserved in PLASMA-LYTE A with 2.5% human serum albumin and 5% DMSO (“Freeze Medium”). Both the Intermediate and Intermediate Control MSC were cryopreserved at a concentration of 20×10⁶ cells in 1 mL.

Following cryopreservation, a vial of Intermediate MSC was thawed, washed with PBS (IX), and then cryopreserved again but in Freeze Medium at a concentration of 10×10⁶ cells in 1 mL, yielding P4 MSCs “Final Product”.

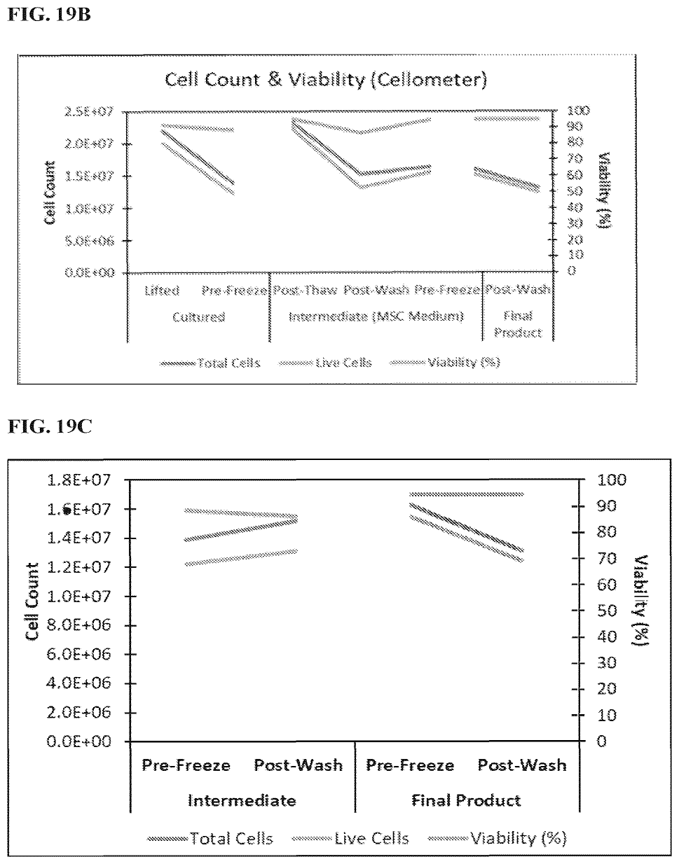

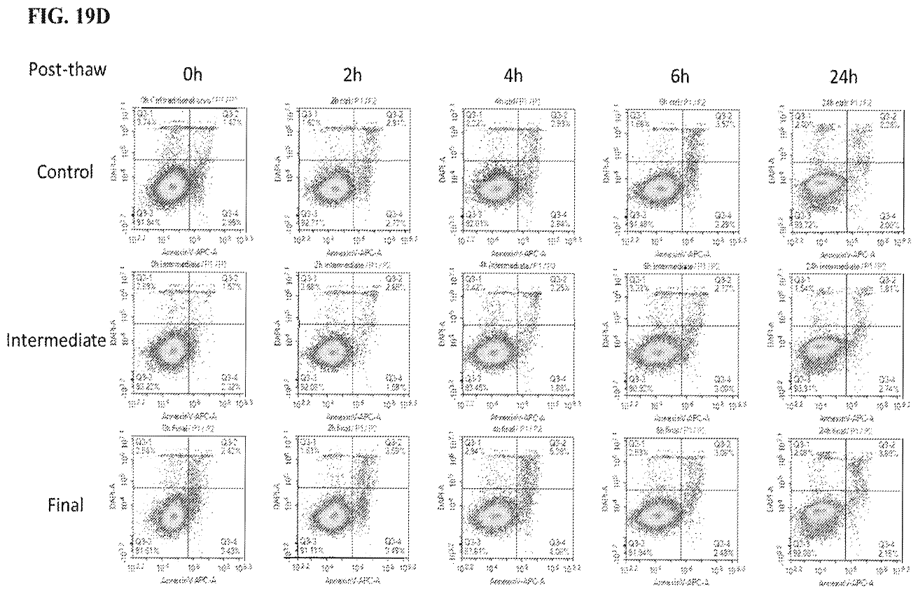

Following Final Product cryopreservation, a vial of intermediate, Intermediate Control, and Final Product MSC were each thawed, diluted, centrifuged, and resuspended in MSC Culture Medium. Apoptosis testing via flow cytometry (Annexin V) was performed on the resuspended MSC (Time Zero). Additionally, the resuspended MSC were plated at 25,000 cells/cm² and incubated for four (4) different post-thaw time points: 2, 4, 6, and 24 hours. After the specified incubation period, the plated MSC were lifted and tested for apoptosis. The apoptosis data is summarized in FIG. 1. The pre-freeze and post-thaw/post-wash cell counts and viabilities, determined via Nexcelom Cellometer automated cell counter with AO/PI staining, are presented in FIG. 19A. The pre-freeze and post-thaw/post-wash cell counts and viabilities, determined via Nexcelom Cellometer automated cell counter with AO/PI staining, are presented in FIG. 19B and FIG. 19C. FIG. 19D are illustrative flow plots showing Apoptosis among cell the various groups and at the specified timepoints (at 0, 2, 4, 6 and 24 hours).

These data demonstrated no difference between control and Intermediate (Rinse media vs Growth media prepared cryosolution), whereas apoptotic and dead cells increased slightly in final product at 4 hour post thaw.

### INCORPORATION BY REFERENCE

All publications, patents, and patent applications mentioned in this specification are herein incorporated by reference to the same extent as if each individual publication, patent, or patent application was specifically and individually indicated to be incorporated by reference. In particular, the following PCT applications that provide teachings relevant to the present disclosure: WO2022226356A1, WO2022221672A1, WO2022159824A1, WO2022140296A1, WO2022140613A1, WO2022133282A1, WO2022081909A1, WO2022081896A1, and WO2022020210A1. To the extent publications and patents or patent applications incorporated by reference contradict the disclosure contained in the specification, the specification is intended to supersede and/or take precedence over any such contradictory material.

### REFERENCES

Galipeau, J., The mesenchymal stromal cells dilemma—does a negative phase III trial of random donor mesenchymal stromal cells in steroid-resistant-graft-versus-host disease represent a death knell or a bump in the road? Cytotherapy, 2013. 15 (1): p. 2-8.
Galipeau, J. and L. Sensebe, Mesenchymal Stromal Cells: Clinical Challenges and Therapeutic Opportunities. Cell Stem Cell, 2018. 22 (6): p. 824-833.
Matthay, M. A., et al., Treatment with allogeneic mesenchymal stromal cells for moderate to severe acute respiratory distress syndrome (START study): a randomised phase 2a safety trial. Lancet RespirMed, 2019. 7 (2): p. 154-162.
Johnstone, B. H., et al., Identification and characterization of a large source of primary mesenchymal stem cells tightly adhered to bone surfaces of human vertebral body marrow cavities. Cytotherapy, 2020. 22 (11): p. 617-628.
Mendicino, M., et al., MSC-based product characterization for clinical trials: an FDA perspective. Cell Stem Cell, 2014. 14 (2): p. 141-5.
Chinnadurai, R., et al., Cryopreserved Mesenchymal Stromal Cells Are Susceptible to T-Cell Mediated Apoptosis Which Is Partly Rescued by IFN gamma Licensing. Stem Cells, 2016. 34 (9): p. 2429-42.
Meneghel, J., P. Kilbride, and G. J. Morris, Cryopreservation as a Key Element in the Successful Delivery of Cell-Based Therapies-A Review. Front Med (Lausanne), 2020. 7: p. 592242.
Bahsoun, S., K. Coopman, and E. C. Akam, Quantitative assessment of the impact of cryopreservation on human bone marrow-derived mesenchymal stem cells: up to 24 h post-thaw and beyond. Stem Cell Res Ther, 2020. 11 (1): p. 540.
Francois, M., et al., Cryopreserved mesenchymal stromal cells display impaired immunosuppressive properties as a result of heat-shock response and impaired interferon-gamma licensing. Cytotherapy, 2012. 14 (2): p. 147-52.
Moll, G., et al., Do cryopreserved mesenchymal stromal cells display impaired immunomodulatory and therapeutic properties? Stem Cells, 2014. 32 (9): p. 2430-42.
Woods, E. J., et al., Off the shelf cellular therapeutics: Factors to consider during cryopreservation and storage of human cells for clinical use. Cytotherapy, 2016. 18 (6): p. 697-711.
Baust, J. M., et al., Assessment of the Impact of Post-Thaw Stress Pathway Modulation on Cell Recovery following Cryopreservation in a Hematopoietic Progenitor Cell Model. Cells, 2022. 11 (2).
Medrano-Trochez, C., et al., Single-cell RNA-seq of out-of-thaw mesenchymal stromal cells shows tissue-of-origin differences and inter-donor cell-cycle variations. Stem Cell Res Ther, 2021. 12 (1): p. 565.
Olmedo-Moreno, L., et al., Heterogeneity of In Vitro Expanded Mesenchymal Stromal Cells and Strategies to Improve Their Therapeutic Actions. Pharmaceutics, 2022. 14 (5).
Harper, J. V. and G. Brooks, The mammalian cell cycle: an overview. Methods Mol Biol, 2005. 296: p. 113-53.
Zlotek-Zlotkiewicz, E., et al., Optical volume and mass measurements show that mammalian cells swell during mitosis. J Cell Biol, 2015. 211 (4): p. 765-74.
Son, S., et al., Resonant microchannel volume and mass measurements show that suspended cells swell during mitosis. J Cell Biol, 2015. 211 (4): p. 757-63.
Cadart, C., et al., Size control in mammalian cells involves modulation of both growth rate and cell cycle duration. Nat Commun, 2018. 9 (1): p. 3275.
Cadart, C., et al., Volume growth in animal cells is cell cycle dependent and shows additive fluctuations. Elife, 2022. 11.
Padovan-Merhar, O., et al., Single mammalian cells compensate for differences in cellular volume and DNA copy number through independent global transcriptional mechanisms. Mol Cell, 2015. 58 (2): p. 339-52.
Bryan, A. K., et al., Measuring single cell mass, volume, and density with dual suspended microchannel resonators. Lab Chip, 2014. 14 (3): p. 569-576.
Tzur, A., et al., Cell growth and size homeostasis in proliferating animal cells. Science, 2009. 325 (5937): p. 167-71.
Chao, H. X., et al., Orchestration of DNA Damage Checkpoint Dynamics across the Human Cell Cycle. Cell Syst, 2017. 5 (5): p. 445-459 e5.
Keren, K., et al., Intracellular fluid flow in rapidly moving cells. Nat Cell Biol, 2009. 11 (10): p. 1219-24.
Moeendarbary, E., et al., The cytoplasm of living cells behaves as a poroelastic material. Nat Mater, 2013. 12 (3): p. 253-61.
Muslimovic, A., et al., An optimized method for measurement of gamma-H2AX in blood mononuclear and cultured cells. Nat Protoc, 2008. 3 (7): p. 1187-93.
Falk, M., et al., Chromatin architecture changes and DNA replication fork collapse are critical features in cryopreserved cells that are differentially controlled by cryoprotectants. Sci Rep, 2018. 8 (1): p. 14694.
Mah, L. J., A. El-Osta, and T. C. Karagiannis, gammaH2AX: a sensitive molecular marker of DNA damage and repair. Leukemia, 2010. 24 (4): p. 679-86.
Rogakou, E. P., et al., DNA double-stranded breaks induce histone H2AX phosphorylation on serine 139. J Biol Chem, 1998. 273 (10): p. 5858-68.
Pardee, A. B., A restriction point for control of normal animal cell proliferation. Proc Natl Acad Sci US A, 1974. 71 (4): p. 1286-90.
Pontarin, G., et al., Deoxyribonucleotide metabolism in cycling and resting human fibroblasts with a missense mutation in p53R2, a subunit of ribonucleotide reductase. J Biol Chem, 2011. 286 (13): p. 11132-40.
Van Rechem, C., et al., Differential regulation of HIC1 target genes by CtBP and NuRD, via an acetylation/SUMOylation switch, in quiescent versus proliferating cells. Mol Cell Biol, 2010. 30 (16): p. 4045-59.
Contreras-Lopez, R., et al., HIFI alpha-dependent metabolic reprogramming governs mesenchymal stem/stromal cell immunoregulatory functions. FASEB J, 2020. 34 (6): p. 8250-8264.
Kadle, R. L., et al., Microenvironmental cues enhance mesenchymal stem cell-mediated immunomodulation and regulatory T-cell expansion. PLOS One, 2018. 13 (3): p. e0193178.
Li, Z. H., et al., Rapamycin-Preactivated Autophagy Enhances Survival and Differentiation of Mesenchymal Stem Cells After Transplantation into Infarcted Myocardium. Stem Cell Rev Rep, 2020. 16 (2): p. 344-356.
McClain-Caldwell, I., et al., Immunogenic potential of human bone marrow mesenchymal stromal cells is enhanced by hyperthermia. Cytotherapy, 2018. 20 (12): p. 1437-1444.
Wise, R. M., et al., Short-Term Autophagy Preconditioning Upregulates the Expression of COX2 and PGE2 and Alters the Immune Phenotype of Human Adipose-Derived Stem Cells In Vitro. Cells, 2022. 11 (9).
Zielniok, K., et al., Vadadustat, a HIF Prolyl Hydroxy lase Inhibitor, Improves Immunomodulatory Properties of Human Mesenchymal Stromal Cells. Cells, 2020. 9 (11).
Wang, Z., Regulation of Cell Cycle Progression by Growth Factor-Induced Cell Signaling. Cells, 2021. 10 (12).
Ho, T. T., et al., Autophagy maintains the metabolism and function of young and old stem cells. Nature, 2017. 543 (7644): p. 205-210.
Liang, R., et al., Restraining Lysosomal Activity Preserves Hematopoietic Stem Cell Quiescence and Potency. Cell Stem Cell, 2020. 26 (3): p. 359-376 e7.
Marescal, O. and I. M. Cheeseman, Cellular Mechanisms and Regulation of Quiescence. Dev Cell, 2020. 55 (3): p. 259-271.
Subramaniam, S., et al., Distinct transcriptional networks in quiescent myoblasts: a role for Wnt signaling in reversible vs. irreversible arrest. PLOS One, 2014. 8 (6): p. e65097.
Zhang, S., et al., Newly Generated CD4 (+) T Cells Acquire Metabolic Quiescence after Thymic Egress. J Immunol, 2018. 200 (3): p. 1064-1077.
Mu, W. C., et al., The mitochondrial metabolic checkpoint in stem cell aging and rejuvenation. Mech Ageing Dev, 2020. 188: p. 111254.
Valcourt, J. R., et al., Staying alive: metabolic adaptations to quiescence. Cell Cycle, 2012. 11 (9): p. 1680-96.
Liu, Y., et al., Commitment to Aerobic Glycolysis Sustains Immunosuppression of Human Mesenchymal Stem Cells. Stem Cells Transl Med, 2019. 8 (1): p. 93-106.
van Velthoven, C. T. J. and T. A. Rando, Stem Cell Quiescence: Dynamism, Restraint, and Cellular Idling. Cell Stem Cell, 2019. 24 (2): p. 213-225.
Alekseenko, L. L., et al., Quiescent Human Mesenchymal Stem Cells Are More Resistant to Heat Stress than Cycling Cells. Stem Cells Int, 2018. 2018: p. 3753547.
Rodgers, J. T., et al., mTORCI controls the adaptive transition of quiescent stem cells from GO to G (Alert). Nature, 2014. 510 (7505): p. 393-6.
Foster, D. A., et al., Regulation of G1 Cell Cycle Progression: Distinguishing the Restriction Point from a Nutrient-Sensing Cell Growth Checkpoint(s). Genes Cancer, 2010. 1 (11): p. 1124-31.
Marguerat, S., et al., Quantitative analysis of fission yeast transcriptomes and proteomes in proliferating and quiescent cells. Cell, 2012. 151 (3): p. 671-83.
Li, B., et al., Autophagy mediates serum starvation-induced quiescence in nucleus pulposus stem cells by the regulation of P27. Stem Cell Res Ther, 2019. 10 (1): p. 118.
Ge, J., et al., The size of mesenchymal stem cells is a significant cause of vascular obstructions and stroke. Stem Cell Rev Rep, 2014. 10 (2): p. 295-303.
Eggenhofer, E., et al., Mesenchymal stem cells are short-lived and do not migrate beyond the lungs after intravenous infusion. Front Immunol, 2012. 3: p. 297.
Schrepfer, S., et al., Stem cell transplantation: the lung barrier. Transplant Proc, 2007. 39 (2): p. 573-6.
Pereira, R F., et al., Cultured adherent cells from marrow can serve as long-lasting precursor cells for bone, cartilage, and lung in irradiated mice. Proc Natl Acad Sci US A, 1995. 92 (11): p. 4857-61.
Gao, J., et al., The dynamic in vivo distribution of bone marrow-derived mesenchymal stem cells after infusion. Cells Tissues Organs, 2001. 169 (1): p. 12-20.
Vilenchik, M. M. and A. G. Knudson, Endogenous DNA double-strand breaks: production, fidelity of repair, and induction of cancer. Proc Natl Acad Sci US A, 2003. 100 (22): p. 12871-6.
Nyberg, K. A., et al., Toward maintaining the genome: DNA damage and replication checkpoints. Annu Rev Genet, 2002. 36: p. 617-56.
Lambert, S., et al., Gross chromosomal rearrangements and elevated recombination at an inducible site-specific replication fork barrier. Cell, 2005. 121 (5): p. 689-702.
Salvadori, M., et al., Dissecting the Pharmacodynamics and Pharmacokinetics of MSCs to Overcome Limitations in Their Clinical Translation. Mol Ther Methods Clin Dev, 2019. 14: p. 1-15.

What is claimed is:

1. A composition comprising a population of isolated mesenchymal stromal cells (MSCs), wherein no more than 25% of the population of isolated MSCs are in the S phase of the cell cycle.
